(19) United States
(12) Patent Application Publication
(10) Pub. No.: US 2023/0416232 A1
(43) Pub. Date: Dec. 28, 2023

(54) PYRIMIDINE COMPOUNDS, COMPOSITIONS, AND MEDICINAL APPLICATIONS THEREOF

(71) Applicant: Blueprint Medicines Corporation, Cambridge, MA (US)

(72) Inventors: Gurulingappa Hallur, Bangalore, Karnataka (IN); Naveena Madhyastha, Bangalore, Karnataka (IN); Michael Rajesh Stephen, Bangalore, Karnataka (IN); Bruce Roth, Menlo Park, CA (US); Anjali Pandey, Menlo Park, CA (US); Tracy Saxton, Menlo Park, CA (US)

(21) Appl. No.: 18/033,931

(22) PCT Filed: Oct. 30, 2021
(86) PCT No.: PCT/US2021/057474

§ 371 Date: Apr. 26, 2023

Related U.S. Application Data
(60) Provisional application No. 63/271,993, filed on Oct. 26, 2021.
(60) Provisional application No. 63/236,194, filed on Aug. 23, 2021.
(60) Provisional application No. 63/108,185, filed on Oct. 30, 2020.

(51) Int. Cl. C07D 403/12; C07D 401/14; C07D 405/14; C07D 403/14

(52) U.S. Cl. CPC C07D 403/12; C07D 401/14; C07D 405/14; C07D 403/14

(57) ABSTRACT

The present disclosure relates to a class of pyrimidine compounds, their stereoisomers, tautomers, pharmaceutically acceptable salts, stereoisomers, solvates, and hydrates thereof. The present disclosure also relates to a process of preparation of these pyrimidine compounds, pharmaceutical compositions containing them, and medicinal applications thereof.

### CROSS-REFERENCE

This application claims the benefit of U.S. Provisional Patent Application No. 63/108,185 filed Oct. 30, 2020; U.S. Provisional Patent Application No. 63/236,194 filed Aug. 23, 2021; and U.S. Provisional Patent Application No. 63/271,993 filed Oct. 26, 2021; each of which is incorporated herein by reference in its entirety.

### BACKGROUND OF THE DISCLOSURE

Lung cancer accounts for the greatest number of cancer deaths, and approximately 85% of lung cancer cases are non-small cell lung cancer (NSCLC). The development of targeted therapies for lung cancer has primarily focused on tumors displaying specific oncogenic drivers, namely mutations in epidermal growth factor receptor (EGFR) and anaplastic lymphoma kinase (ALK). Three generations of tyrosine kinase inhibitors (TKIs) have been developed for cancers with the most frequently observed EGFR mutations, however, other oncogenic drivers in the EGFR family of receptor tyrosine kinases have received less research and development focus and several oncogenic drivers, including insertions in the exon 20 gene of EGFR, have no currently approved therapeutics to treat their cancers.

The mutation, amplification and/or overexpression of human epidermal growth factor receptor 2 (HER2), another member of the human epidermal growth factor receptor family of receptor tyrosine kinases, has been implicated in the oncogenesis of several cancers, including lung, breast, ovarian, and gastric cancers. Although targeted therapies such as trastuzumab and lapatinib have shown clinical efficacy especially in breast tumors, their utility in lung cancer has been limited. It is likely that this variation is due to tissue-specific factors, including the low potency of kinase inhibitors like lapatinib for the mutagenic alterations in HER2 that are observed in the lung cancer patient population, including insertions in the exon 20 gene of HER2.

Given that many patients with mutations in EGFR and HER2 do not derive clinical benefit from currently available therapies against these targets, there remains a significant unmet need for the development of novel therapies for the treatment of cancers associated with EGFR and HER2 mutations.

### SUMMARY OF THE DISCLOSURE

In one aspect, provided herein is a compound of Formula I.

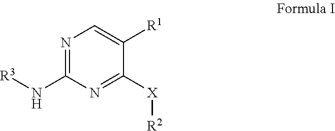

or a pharmaceutically acceptable salt or stereoisomer thereof, wherein:
X is —NH— or —O—;
R¹ is —(C(R⁴)₂)ₙR⁵, wherein R⁵ is unsubstituted or substituted with 2 or 3 R⁵;
   n is 0, 1, 2, or 3;
   each R⁴ is independently hydrogen, alkyl, halo, haloalkyl, hydroxy, alkoxy, or heteroalkyl;
   R⁵ is C₄₋₁₀cycloalkyl, aryl, or heteroaryl;
   each R^{5′} is independently deuterium, aryl, heteroaryl, alkyl, C₃-C₆ cycloalkyl, 3-8 membered heterocycloalkyl, oxo, halo, heteroalkyl, haloalkyl, cyano, hydroxy, amino, —NH₂, —NHR⁶, —N(CH₃)R₆, —N(R⁶)₂, —C(═O)NH₂, —C(═O)NHR⁶, —C(═O)N(R⁶)₂, —NR⁶C(═O)R⁶, —NHC(═O)R⁶, —S(═O)₂alkyl, —S(═O)₂aryl, —S(═O)₂NH₂, —S(═O)₂NHR⁶, —S(═O)₂N(R⁶)₂, —S(═O)₂heteroaryl, alkoxy, or haloalkoxy; or
   two adjacent R^{5′} groups come together to form a 5- to 10-membered heterocycle, wherein the 5- to 10-membered heterocycle is unsubstituted or substituted with alkyl; each R⁶ is independently alkyl, aminoalkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
   each R⁷ is independently

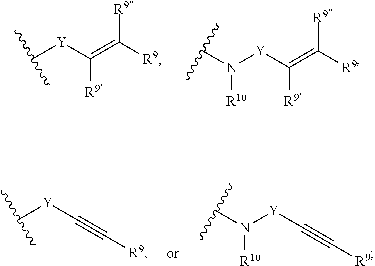

Y is —C(═O)—, —S(═O)—, or —S(═O)₂—;
R⁹, R^{9′}, and R^{9″} are independently hydrogen, deuterium, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently unsubstituted or substituted with 0, 1, or 2 R¹⁴;
      each R¹³ is independently hydrogen, alkyl, cycloalkyl, aryl, or heteroaryl;
      each R¹⁴ is independently deuterium, aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

In some embodiments, X is —NH—.

In some embodiments, n is 0.

In some embodiments, R⁵ is phenyl, naphthyl, anthracenyl, phenanthrenyl, C-linked pyridyl, C-linked pyrimidinyl, C-linked pyrazolyl, C-linked imidazolyl, or C-linked indolyl; wherein R⁵ is substituted with 2 or 3 R^{5′}. In some embodiments, R⁵ is substituted with 2 R^{5′}. In some embodiments, R⁵ is substituted with 3 R^{5′}.

In some embodiments, R⁵ is phenyl or C-linked pyridyl; wherein the phenyl or C-linked pyridyl is substituted with 2 or 3 R⁵. In some embodiments, R⁵ is phenyl or C-linked pyridyl; wherein the phenyl or C-linked pyridyl is substituted with 2 R⁵. In some embodiments, R⁵ is phenyl or C-linked pyridyl; wherein the phenyl or C-linked pyridyl is substituted with 3 R⁵. In some embodiments, two adjacent R^{5′} groups come together to form a 5- to 10-membered heterocycle.

In some embodiments, each R^{5′} is independently alkyl, haloalkyl, 3-8 membered heterocycloalkyl, halo, cyano, hydroxy, —N(R⁶)₂, —N(CH₃)R₆, —C(═O)NHR⁶, —NHC(═O)R⁶, —S(═O)₂NH₂, alkoxy, or haloalkoxy. In some embodiments, each R^{5′} is independently methyl, ethyl, tert-butyl, pyrrolidinyl, piperidinyl, piperazinyl, morpholinyl, fluoro, chloro, cyano, hydroxy, —N(R⁶)₂, —C(═O)NHR⁶, —NHC(═O)R⁶, —S(═O)₂NH₂, methoxy, ethoxy, fluoromethyl, difluoromethyl, trifluoromethyl, fluoromethoxy, difluoromethoxy, or trifluoromethoxy. In some embodiments, each R^{5′} is independently methyl, morpholinyl, fluoro, chloro, cyano, —C(═O)NHMe, —NHC(═O)Me, —S(═O)₂NH₂, methoxy, fluoromethyl, difluoromethyl, trifluoromethyl, difluoromethoxy, or trifluoromethoxy.

In some embodiments, each R⁶ is independently alkyl or aryl. In some embodiments, each R⁶ is independently methyl, ethyl, iso-propyl, tert-butyl, phenyl, or naphthyl. In some embodiments, each R⁶ is independently methyl or phenyl.

In some embodiments, R² is monocyclic. In some embodiments, R² is phenyl, cyclopropyl, cyclobutyl, cyclopentyl, cyclohexyl, pyrrolyl, imidazolyl, pyrazolyl, triazolyl, tetrazolyl, thiazolyl, isothiazolyl, oxazolyl, isoxazolyl, pyridinyl, pyrimidinyl, pyridazinyl, pyrazinyl, or triazinyl; wherein R² is substituted with at least one R⁷ and 0, 1, or 2 R⁸. In some embodiments, R² is phenyl, cyclohexyl, or pyrrolyl; wherein R² is substituted with at least one R⁷ and 0, 1, or 2 R⁸.

In some embodiments, R⁷ is

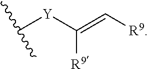

In some embodiments, R⁷ is R¹⁰

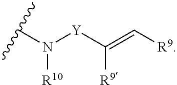

In some embodiments, R⁷ is

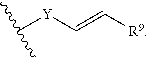

In some embodiments, R⁷ is R¹⁰

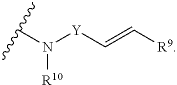

In some embodiments, Y is —C(═O)—. In some embodiments, Y is —S(═O)₂—.

In some embodiments, R⁹, R^{9′} and R^{9″} are independently hydrogen, halo, alkyl, heteroalkyl, haloalkyl, or (alkyl)heterocycloalkyl. In some embodiments, R⁹, R^{9′} and R^{9″} are independently hydrogen, fluoro, chloro, methyl, hydroxyethyl, methoxyethyl, methoxymethyl, dimethylaminomethyl, 1-piperidinylmethyl, 1-morpholinylmethyl, or fluoromethyl. In some embodiments, R⁹ and R^{9′} are independently hydrogen, halo, alkyl, heteroalkyl, haloalkyl, or (alkyl)heterocycloalkyl. In some embodiments, R⁹ and R^{9′} are independently hydrogen, fluoro, chloro, methyl, hydroxyethyl, methoxyethyl, methoxymethyl, dimethylaminomethyl, 1-piperidinylmethyl, 1-morpholinylmethyl, or fluoromethyl.

In some embodiments, R¹⁰ is hydrogen, methyl, ethyl n-propyl, iso-propyl, n-butyl, sec-butyl, tert-butyl, trifluoromethyl, or cyclopropyl. In some embodiments, R¹⁰ is hydrogen or methyl.

In some embodiments, R² is substituted with 1 or 2 R⁸. In some embodiments, each R⁸ is independently methyl, ethyl, n-propyl, iso-propyl, n-butyl, iso-butyl, sec-butyl, tert-butyl, fluoro, chloro, heteroalkyl, cyano, hydroxy, amino, —N(R¹¹)₂, methoxy, ethoxy, or trifluoromethoxy. In some embodiments, each R⁸ is independently methyl, ethyl, iso-propyl, tert-butyl, fluoro, chloro, —N(R¹¹)₂, hydroxyethyl, methoxyethyl, or cyano.

In some embodiments, each R¹¹ is independently alkyl or aryl. In some embodiments, each R¹¹ is independently methyl, ethyl, n-propyl, iso-propyl, n-butyl, iso-butyl, sec-butyl, tert-butyl, phenyl, naphthyl, anthracenyl, or phenanthrenyl. In some embodiments, each R¹¹ is independently methyl, ethyl, iso-propyl, tert-butyl, phenyl, or naphthyl. In some embodiments, each R¹¹ is independently methyl or phenyl.

In some embodiments, R² is not substituted with R⁸.

In some embodiments, R³ is pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl; wherein R³ is substituted with 0, 1, 2, or 3 R¹². In some embodiments, R³ is imidazolyl, pyrazolyl, triazolyl, indolyl, indazolyl, thiazolyl, isothiazolyl, or pyridinyl; wherein R³ is substituted with 0, 1, 2, or 3 R¹².

In some embodiments, R³ is:

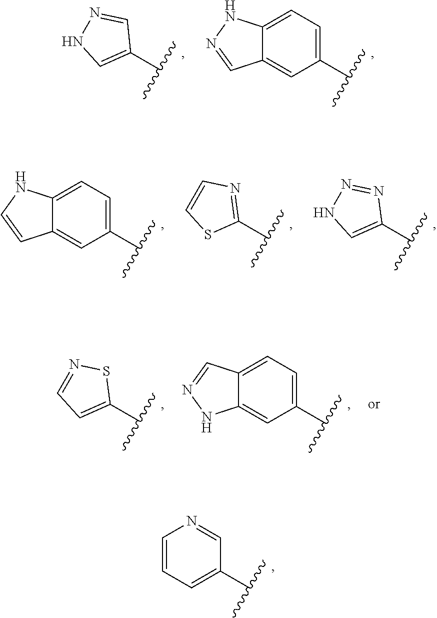

wherein R³ is substituted with 0 to 3 R¹².

In some embodiments, R³ is:

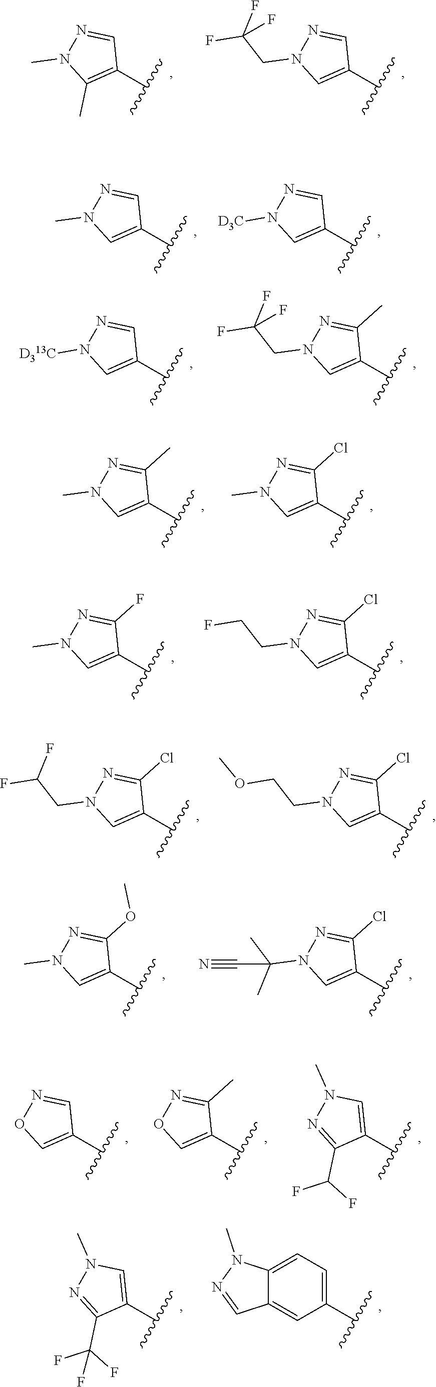

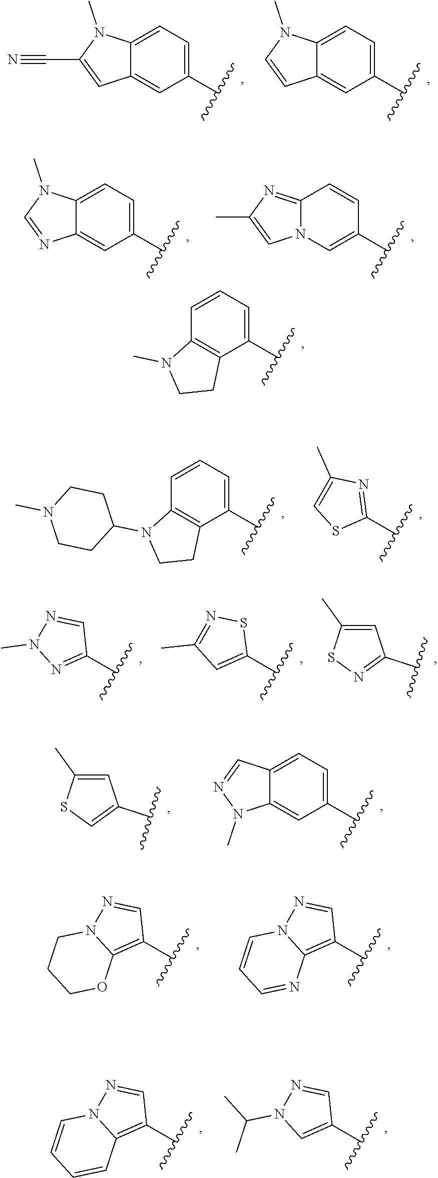

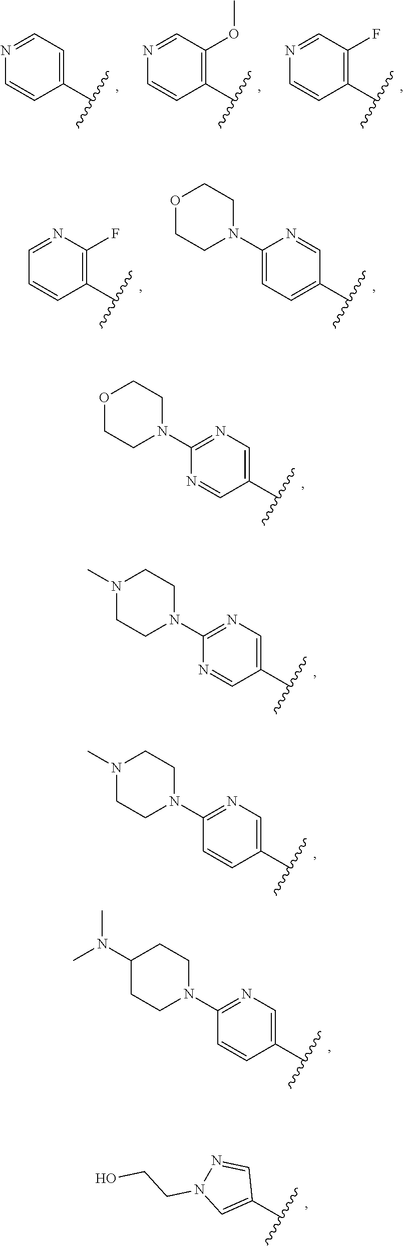

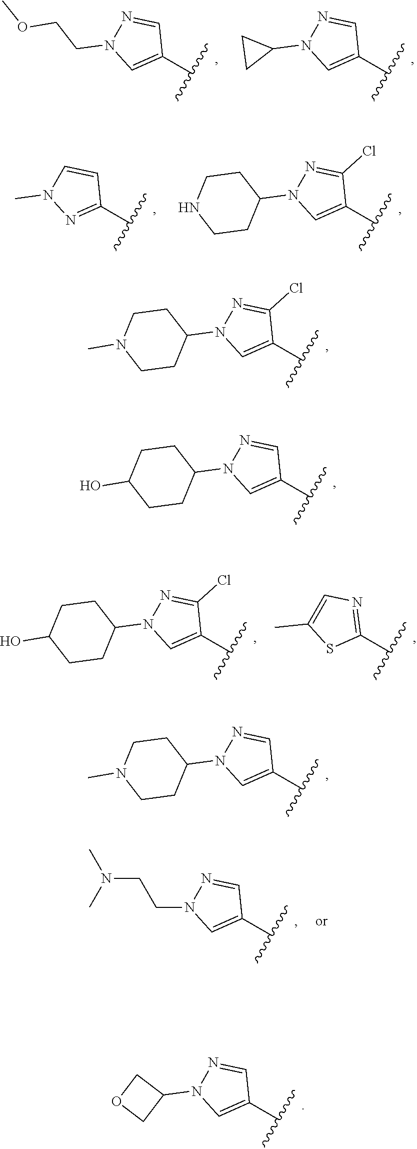

In some embodiments, R³ is:

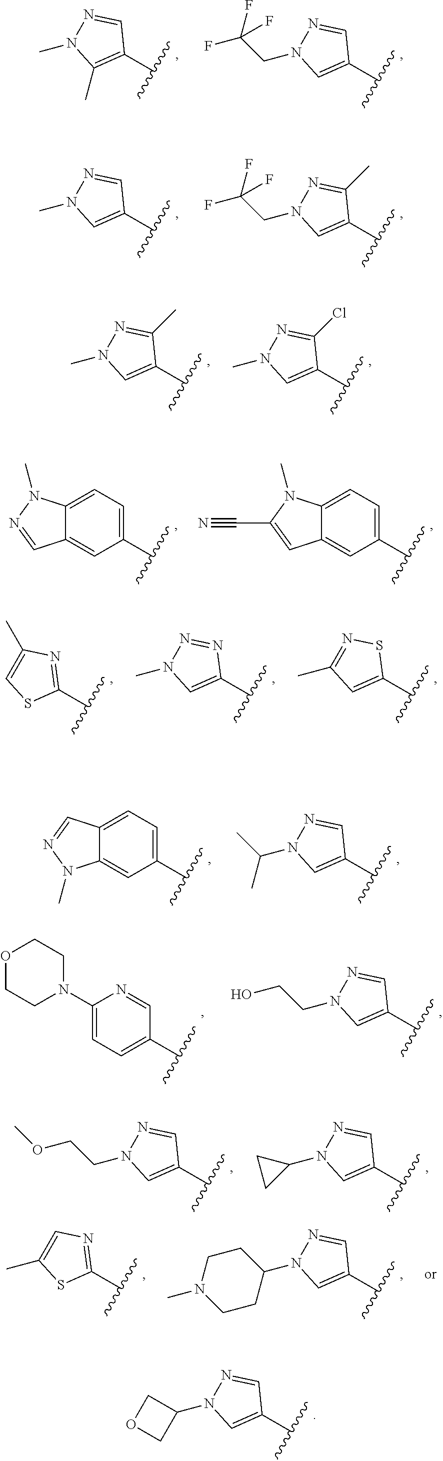

In some embodiments, R³ is unsubstituted. In some embodiments, R³ is substituted with at least 1 R¹². In some embodiments, R³ is substituted with at least 2 R¹².

In some embodiments, each R¹² is independently aryl, heteroaryl, alkyl, heteroalkyl, haloalkyl, halo, cyano, heterocycloalkyl, —N(R¹³)₂, —S(═O)₂NH₂, or cycloalkyl. In some embodiments, each R¹² is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, azetidinyl, oxetanyl, pyrrolidinyl, imidazolidinyl, tetrahydrofuranyl, piperidinyl, piperazinyl, tetrahydropyranyl, morpholinyl, —N(R¹³)₂, cyclopropyl, cyclobutyl, cyclopentyl, or cyclohexyl. In some embodiments, each R¹² is independently methyl, iso-propyl, tert-butyl, hydroxyethyl, methoxyethyl, trifluoromethyl, trifluoroethyl, chloro, cyano, morpholinyl, or cyclopropyl. In some embodiments, each R¹² is independently methyl, hydroxyethyl, methoxyethyl, trifluoroethyl, or chloro. In some embodiments, each R¹² is independently methyl or chloro.

In some embodiments, each R¹³ is independently alkyl or cycloalkyl. In some embodiments, each R¹³ is independently methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹³ is independently methyl, ethyl, iso-propyl, tert-butyl, cyclopropyl, cyclopentyl, or cyclohexyl. In some embodiments, each R¹³ is independently methyl, cyclopropyl, or cyclohexyl.

In some embodiments, the aryl, heteroaryl, heterocycloalkyl, or cycloalkyl of R¹² is unsubstituted. In some embodiments, aryl, heteroaryl, heterocycloalkyl, or cycloalkyl of R¹² is substituted with 1 or 2 R¹⁴.

In some embodiments, each R¹⁴ is independently alkyl, cycloalkyl, heterocycloalkyl, halo, cyano, —N(R¹¹)₂, or alkoxy. In some embodiments, each R¹⁴ is independently methyl, ethyl, n-propyl, iso-propyl, n-butyl, iso-butyl, sec-butyl, tert-butyl, cyclopropyl, cyclobutyl, cyclopentyl, cyclohexyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, —N(R¹¹)₂ methoxy, ethoxy, or trifluoromethoxy. In some embodiments, each R¹⁴ is independently methyl, ethyl, iso-propyl, tert-butyl, pyrrolidinyl, piperidinyl, morpholinyl, fluoro, chloro, —N(R¹¹)₂, or methoxy.

In some embodiments, each R¹⁵ is independently alkyl or cycloalkyl. In some embodiments, each R¹⁵ is methyl, ethyl, n-propyl, iso-propyl, n-butyl, iso-butyl, sec-butyl, tert-butyl, cyclopropyl, cyclobutyl, cyclopentyl, or cyclohexyl.

In some embodiments, each R¹³ is independently methyl, ethyl, iso-propyl, tert-butyl, cyclopropyl, cyclopentyl, or cyclohexyl. In some embodiments, each R¹³ is independently methyl, cyclopropyl, or cyclohexyl.

In some embodiments:
X is —NH— or —O—;
n is 0;
R⁵ is phenyl substituted with 2 or 3 R^{5′};
R² is phenyl substituted with at least one R⁷ and 0, 1, or 2 R⁸; and
R³ is pyrazolyl substituted with 0, 1, 2, or 3 R¹².

In some embodiments, X is —NH—.

In some embodiments, R^{5′} is fluoromethyl, difluoromethyl, or trifluoromethyl.

In some embodiments:
R⁷ is

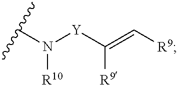

and
R⁸ is halo.

In some embodiments:
R⁸ is fluoro;
Y is —C(═O)—;
R⁹ and R^{9′} are hydrogen; and
R¹⁰ is hydrogen.

In some embodiments, R¹² is alkyl.

In some embodiments, R¹² is methyl.

In some embodiments, the compound is of Formula I-A, Formula I-B, Formula I-C, Formula I-D, Formula I-E, Formula I-F or Formula I-G:

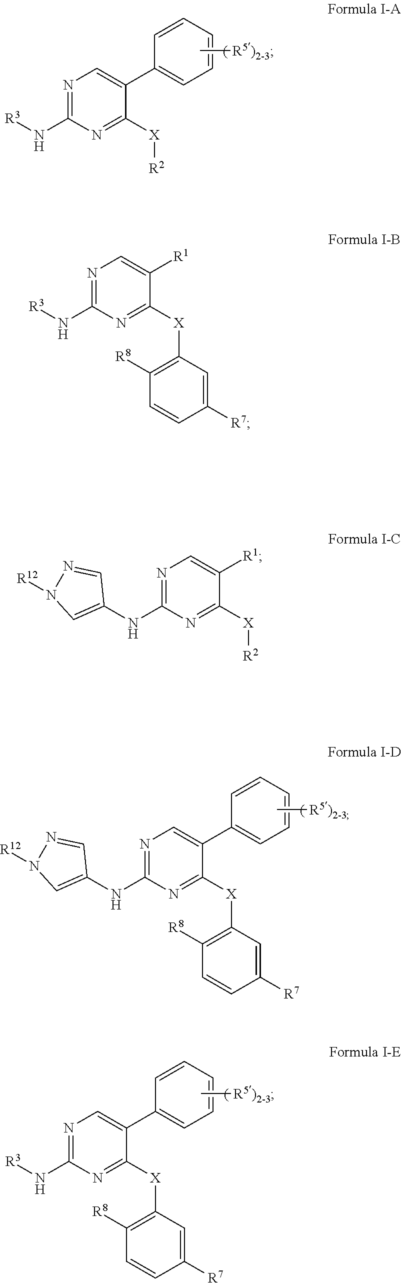

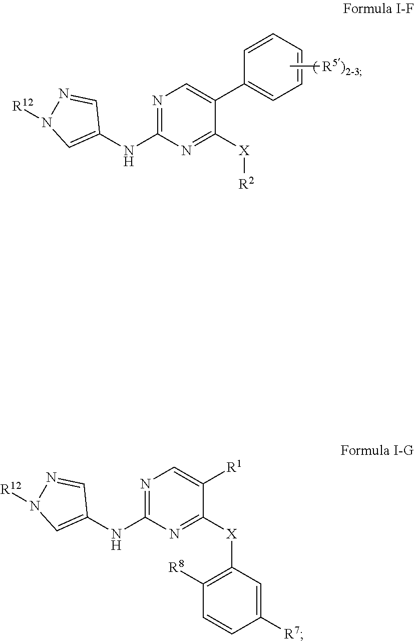

or a pharmaceutically acceptable salt or stereoisomer thereof.

In some embodiments, the compound is of Formula I-B:

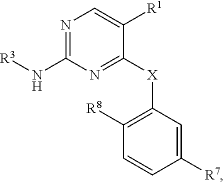

or a pharmaceutically acceptable salt or stereoisomer thereof. In some embodiments of the compound of Formula I-B, wherein R¹ is R⁵. In some embodiments of the compound of Formula I-B, wherein R¹ is R⁵; and R⁵ is substituted with 2 R^{5′}. In some embodiments of the compound of Formula I-B, wherein R¹ is R⁵; and R⁵ is substituted with 3 R^{5′}. In some embodiments of the compound of Formula I-B, R⁵ is phenyl or C-linked pyridyl; wherein the phenyl or C-linked pyridyl is substituted with 2 or 3 R^{5′}. In some embodiments of the compound of Formula I-B, R⁵ is phenyl or C-linked pyridyl; wherein the phenyl or C-linked pyridyl is substituted with 2 R^{5′}. In some embodiments of the compound of Formula I-B, R⁵ is phenyl or C-linked pyridyl; wherein the phenyl or C-linked pyridyl is substituted with 3 R^{5′}. In some embodiments of the compound of Formula I-B, two adjacent R^{5′} groups come together to form a 5- to 10-membered heterocycle.

In some embodiments, the compound is of Formula I-C:

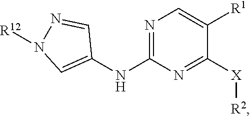

or a pharmaceutically acceptable salt or stereoisomer thereof. In some embodiments of the compound of Formula I-C, wherein R¹ is R⁵. In some embodiments of the compound of Formula I-C, wherein R¹ is R⁵; and R⁵ is substituted with 2 R^{5′}. In some embodiments of the compound of Formula I-C, wherein R¹ is R⁵; and R⁵ is substituted with 3 R^{5′}. In some embodiments of the compound of Formula I-C, R⁵ is phenyl or C-linked pyridyl; wherein the phenyl or C-linked pyridyl is substituted with 2 or 3 R^{5′}. In some embodiments of the compound of Formula I-C, R⁵ is phenyl or C-linked pyridyl; wherein the phenyl or C-linked pyridyl is substituted with 2 R^{5′}. In some embodiments of the compound of Formula I-C, R⁵ is phenyl or C-linked pyridyl; wherein the phenyl or C-linked pyridyl is substituted with 3 R^{5′}. In some embodiments of the compound of Formula I-C, two adjacent R^{5′} groups come together to form a 5- to 10-membered heterocycle.

In some embodiments, the compound is:

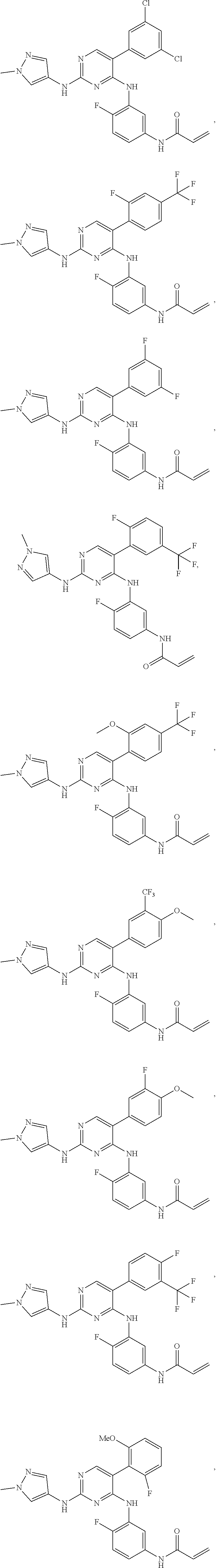

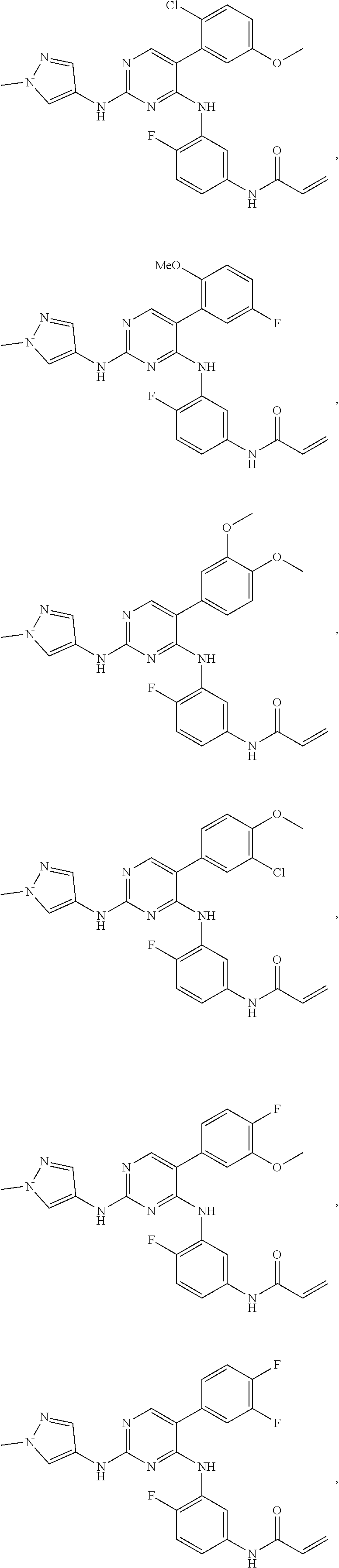

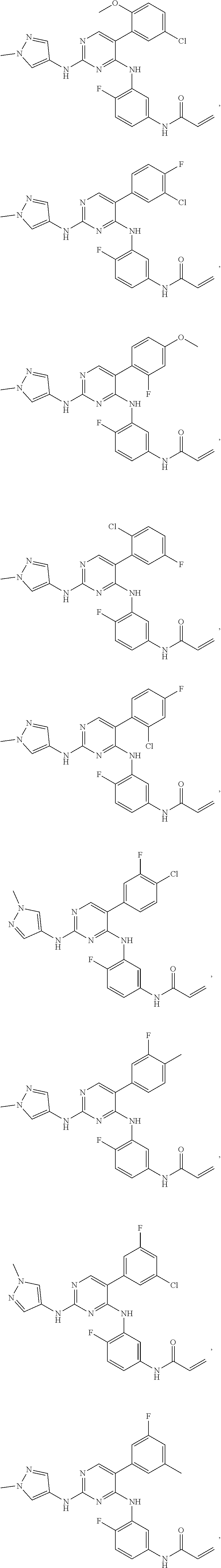

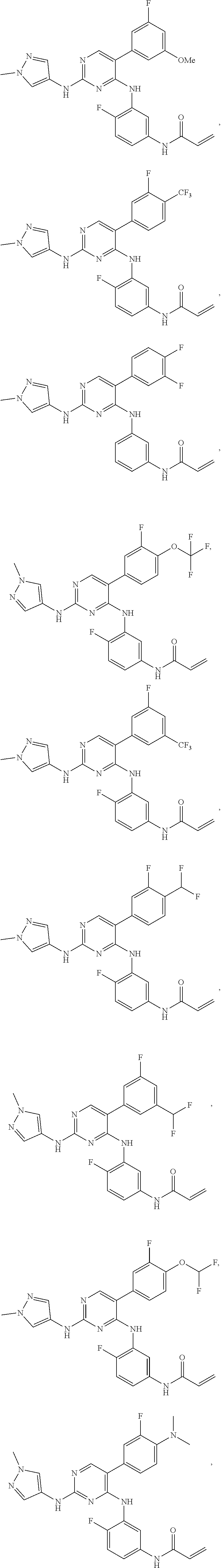

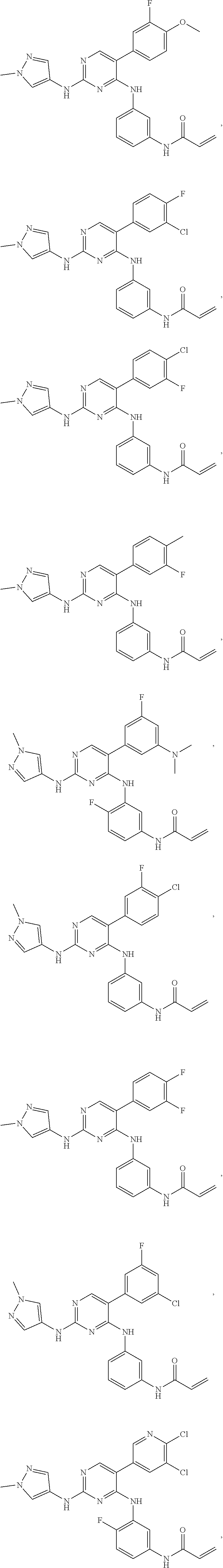

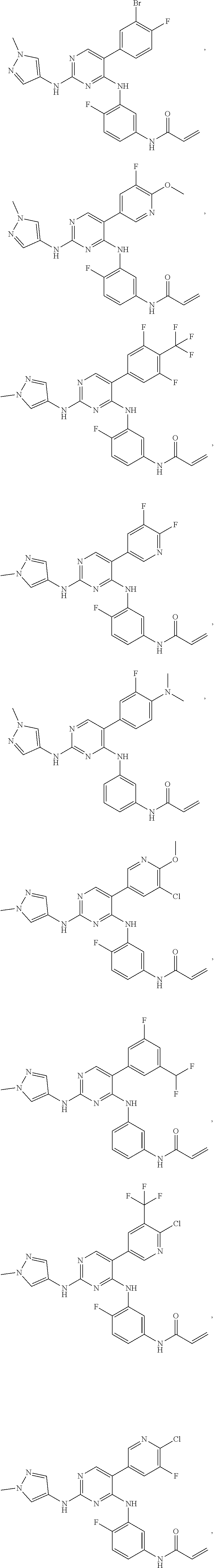

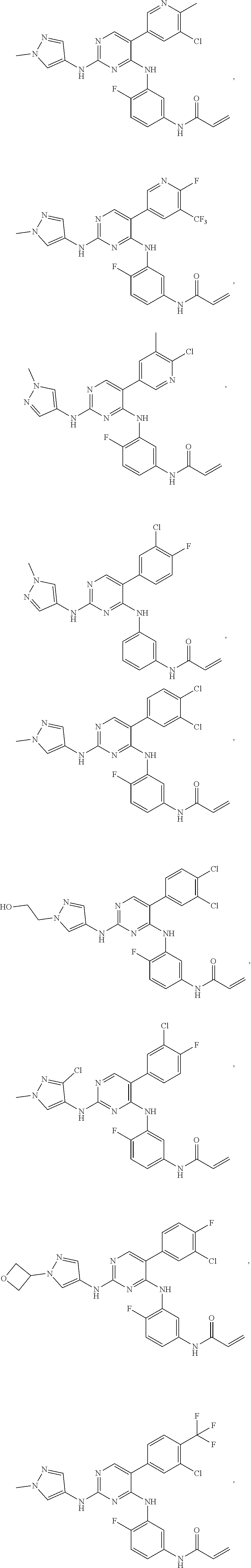

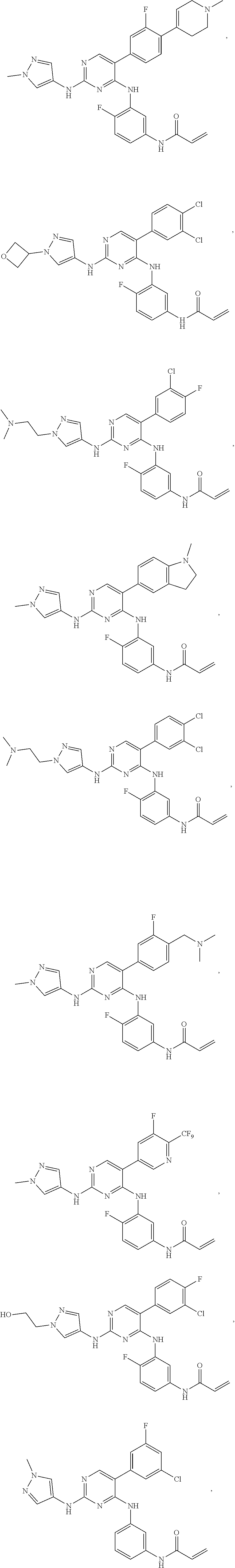

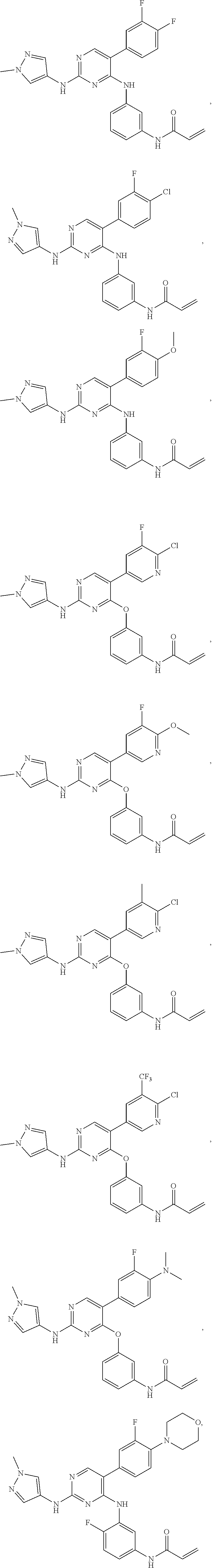

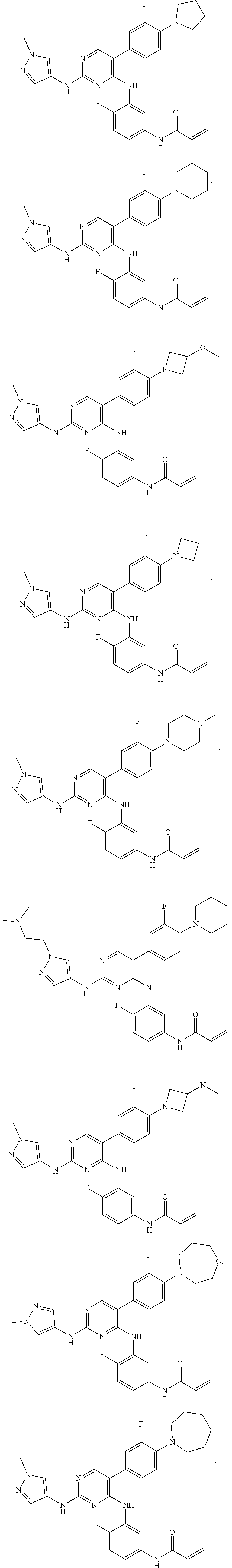

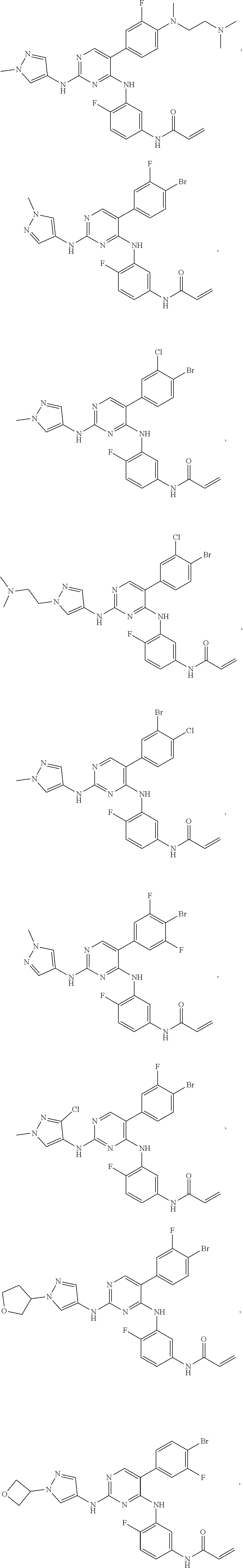

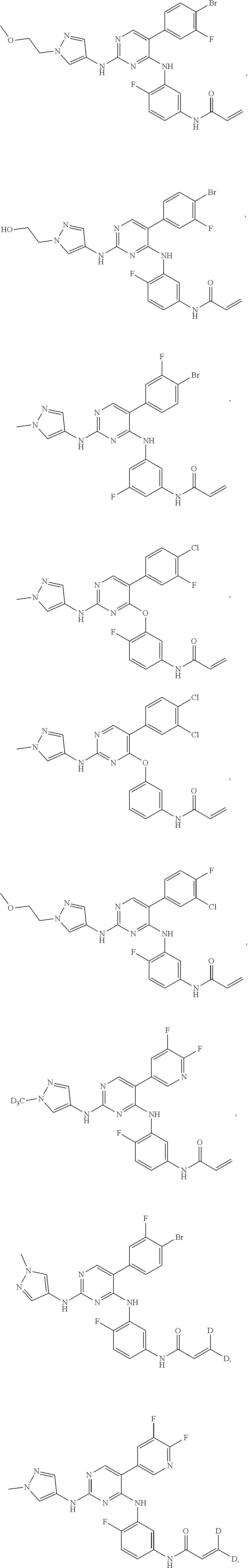

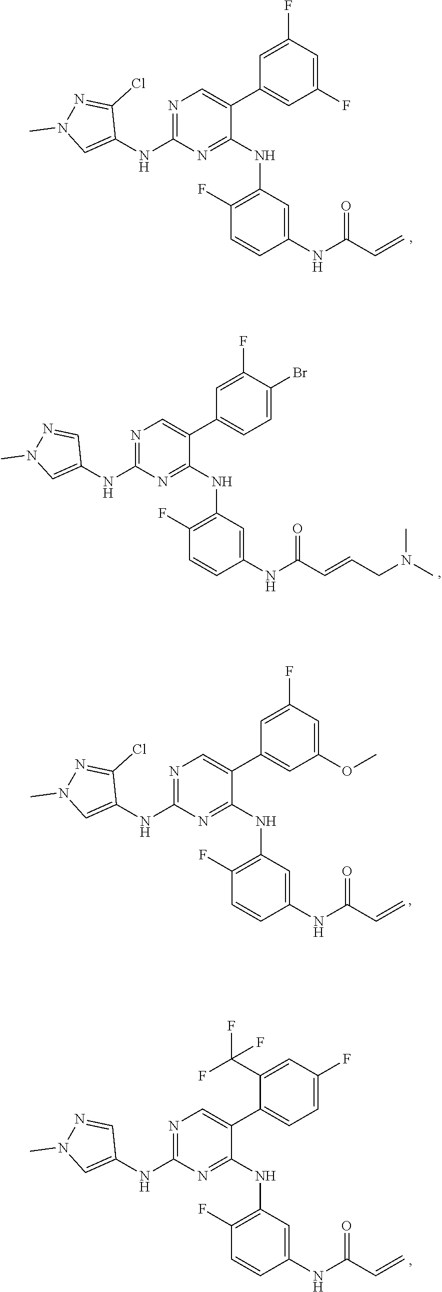

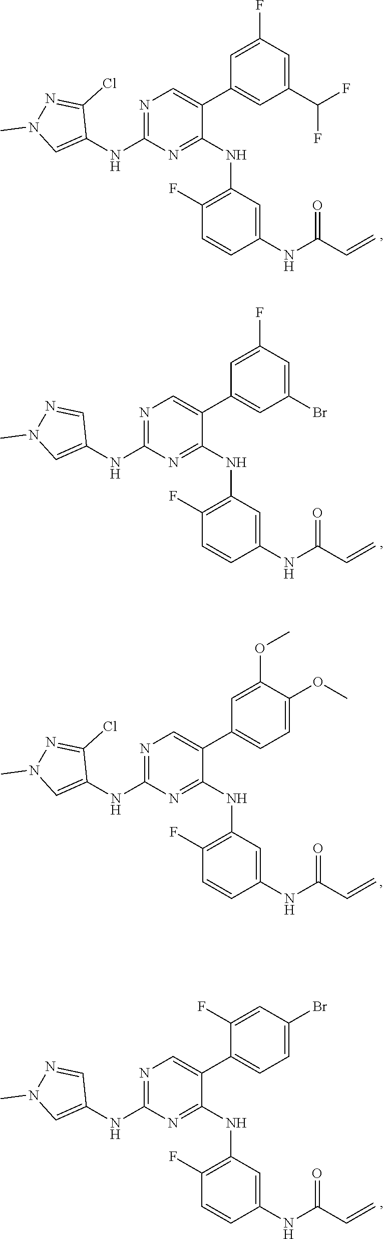

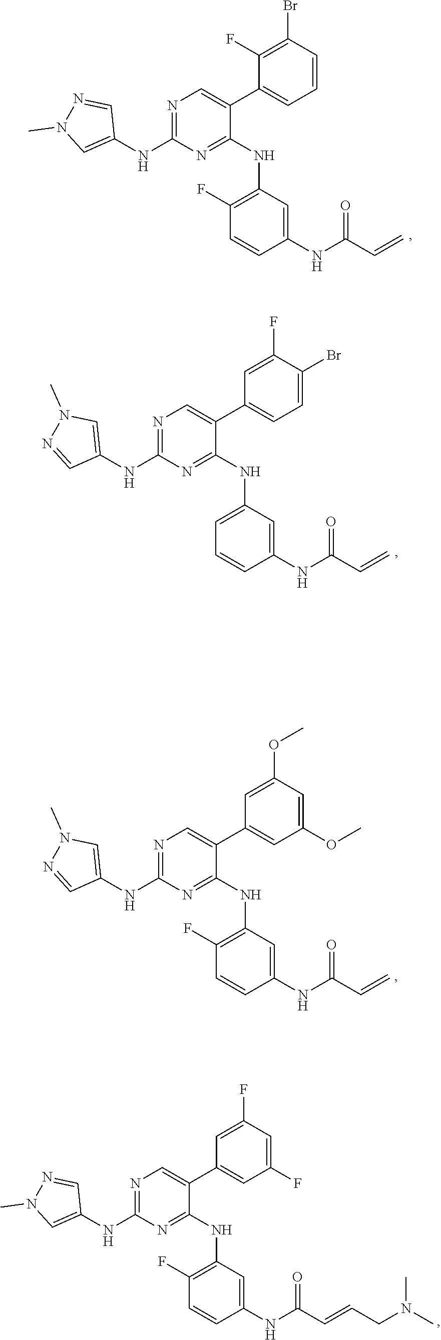

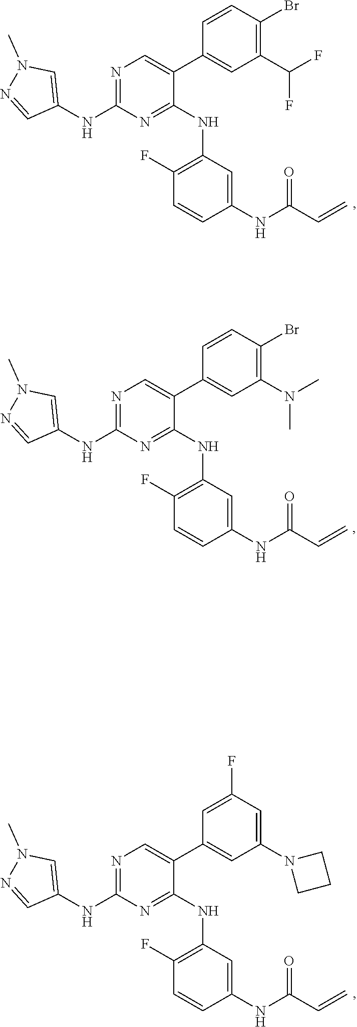 [SMILES: C=CC(=O)Nc1ccc(F)c(Nc2nc(Nc3cnn(C)c3)ncc2-c2ccc(Br)c(C(F)F)c2)c1 ; C=CC(=O)Nc1ccc(F)c(Nc2nc(Nc3cnn(C)c3)ncc2-c2ccc(Br)c(N(C)C)c2)c1 ; C=CC(=O)Nc1ccc(F)c(Nc2nc(Nc3cnn(C)c3)ncc2-c2cc(F)cc(N3CCC3)c2)c1]

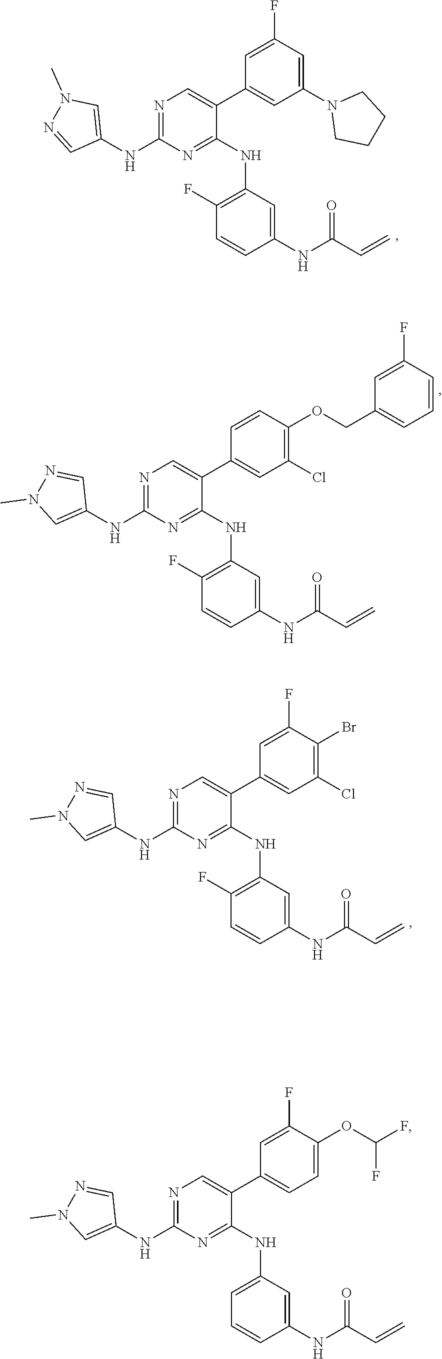

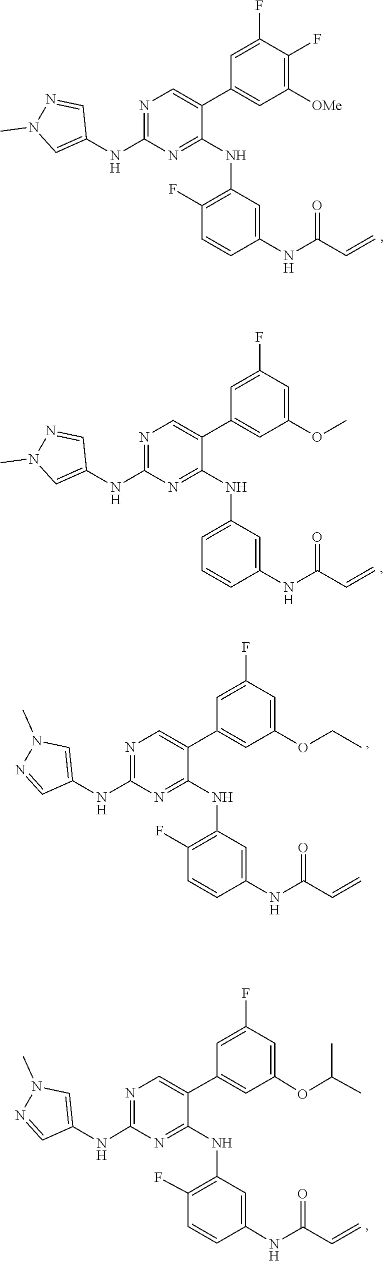

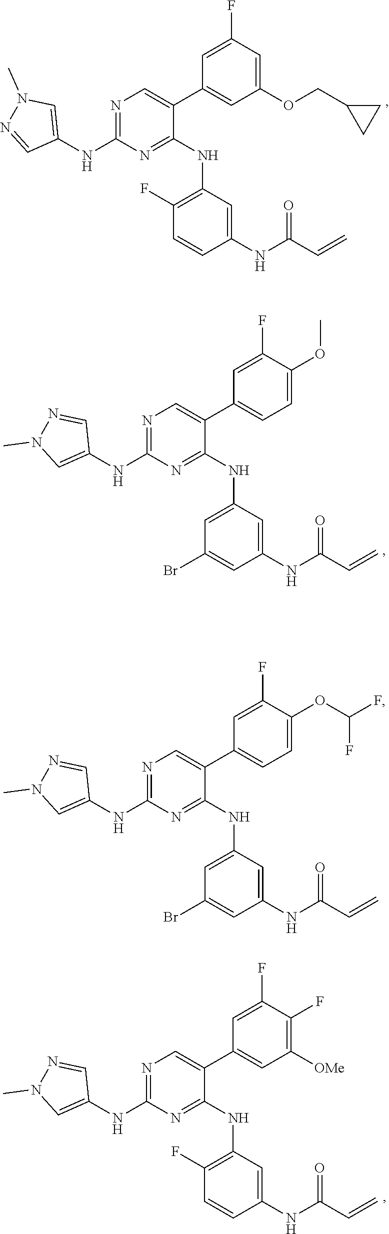

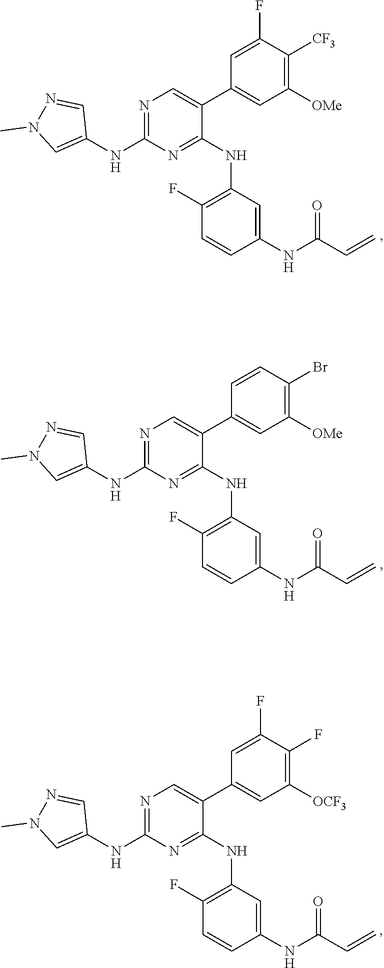

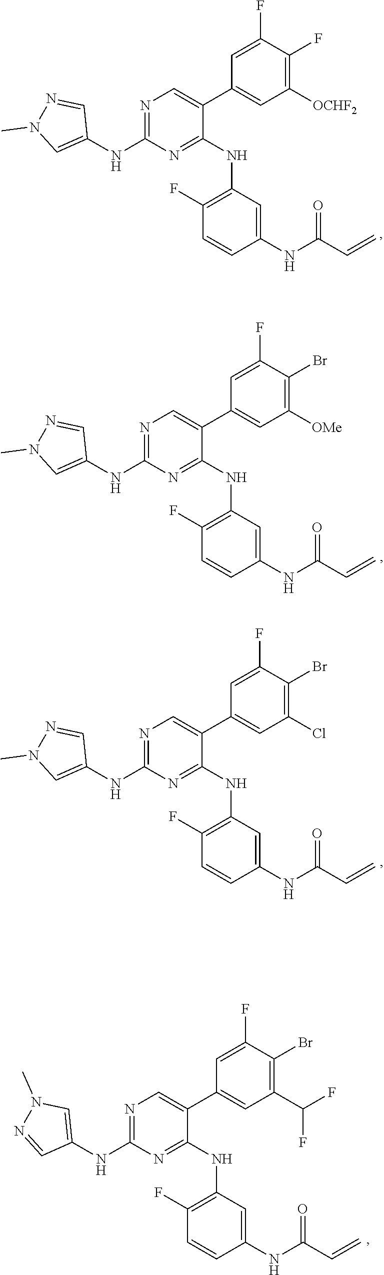

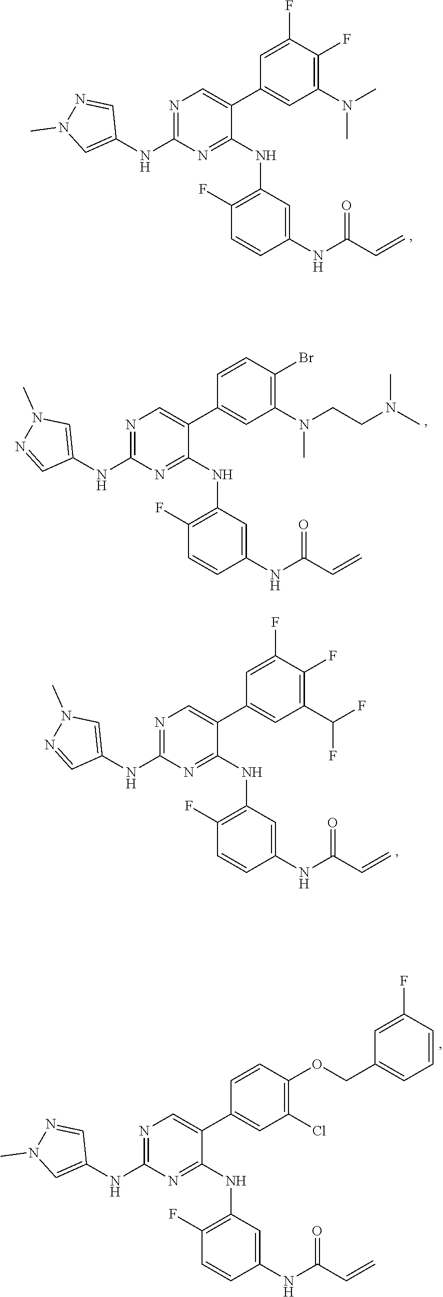

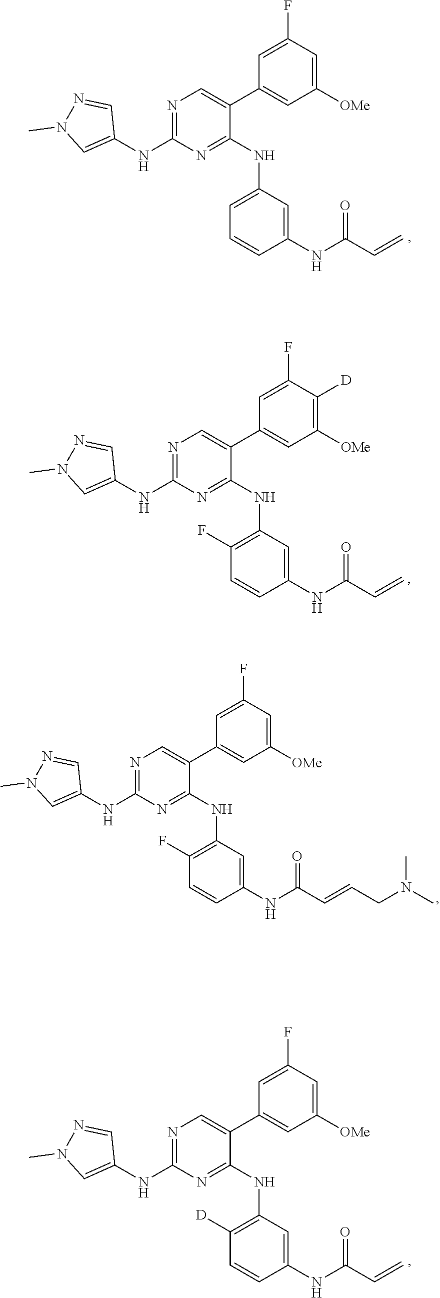

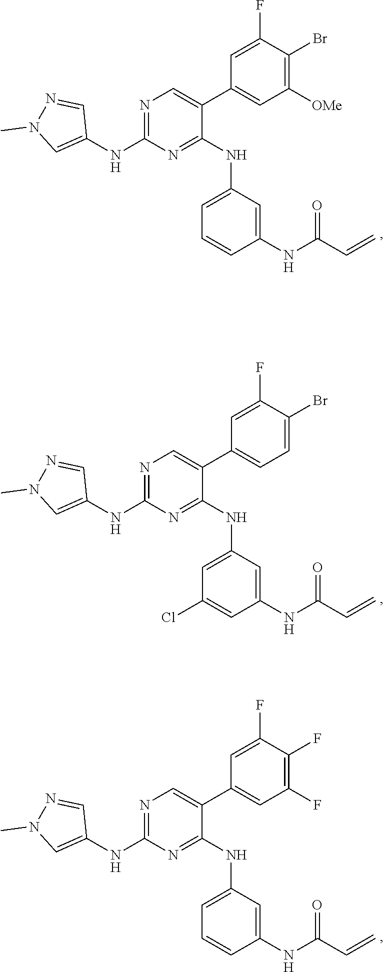

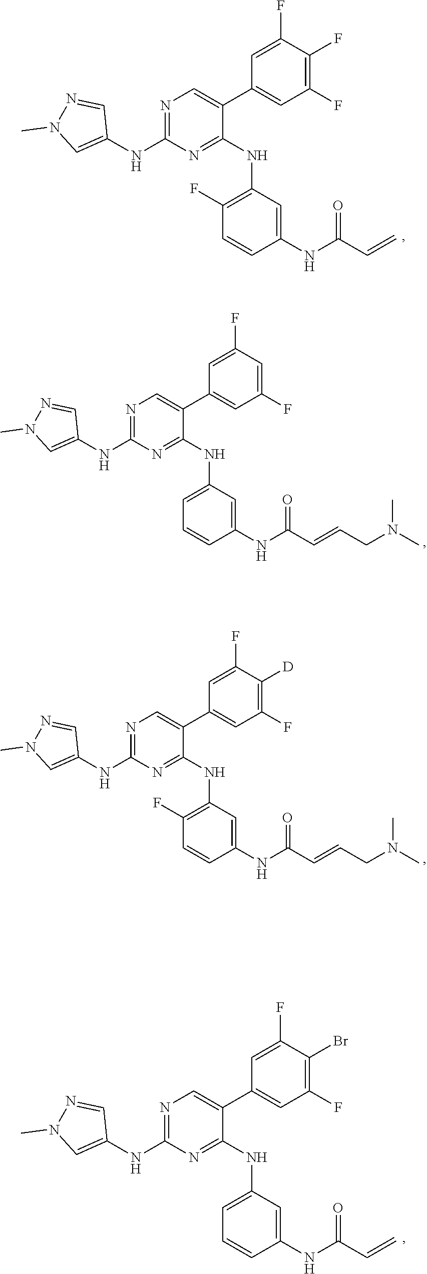

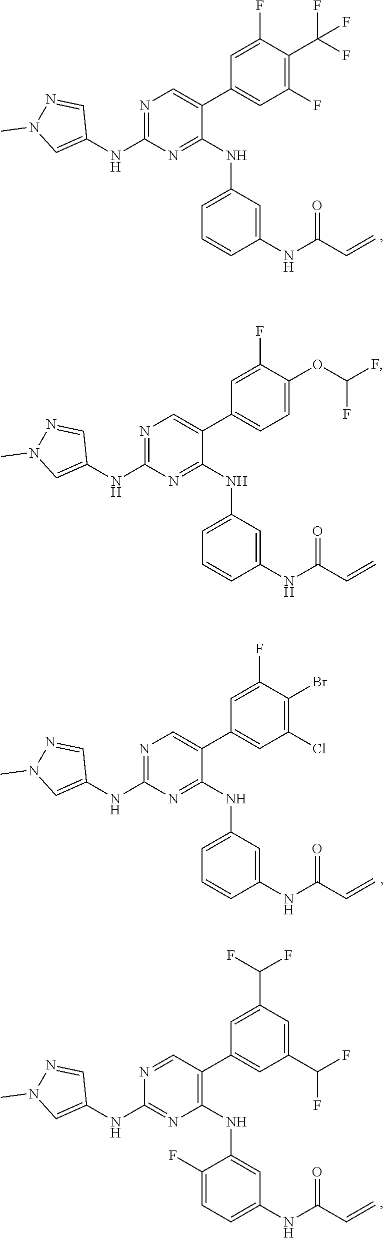

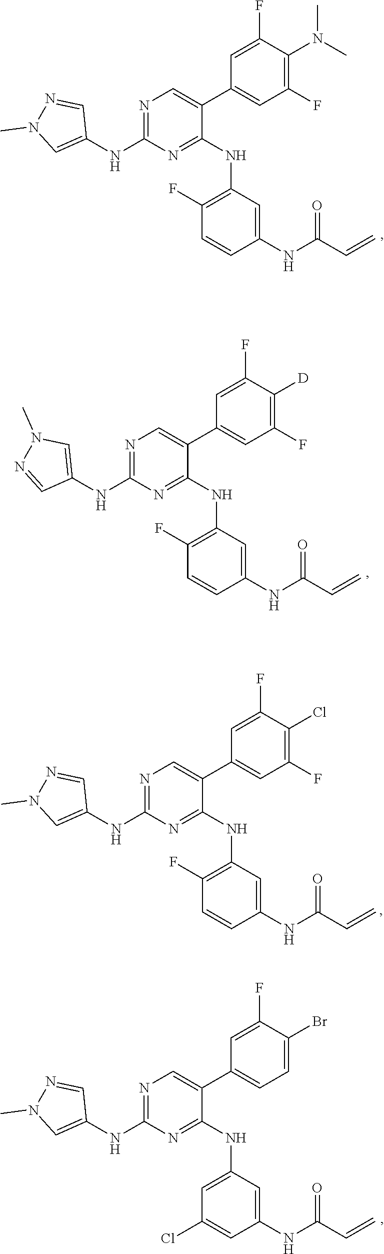

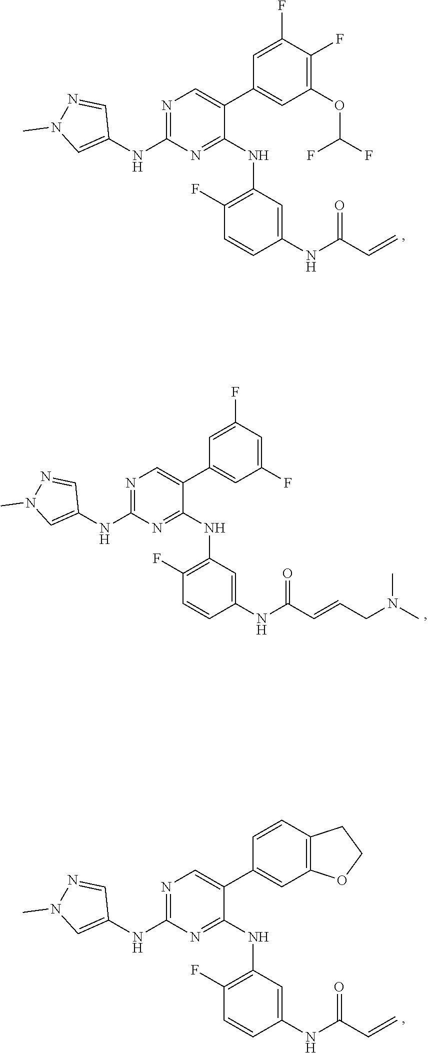

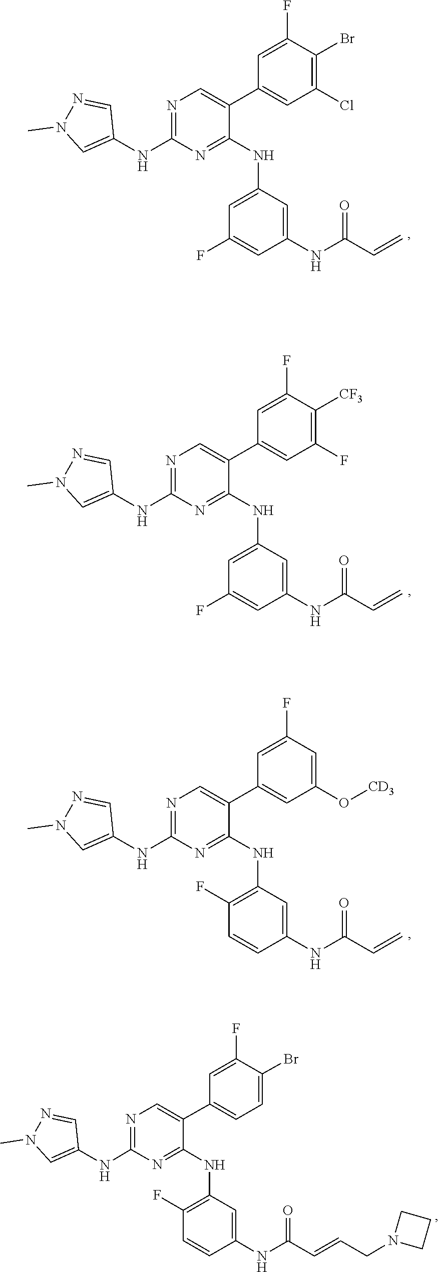

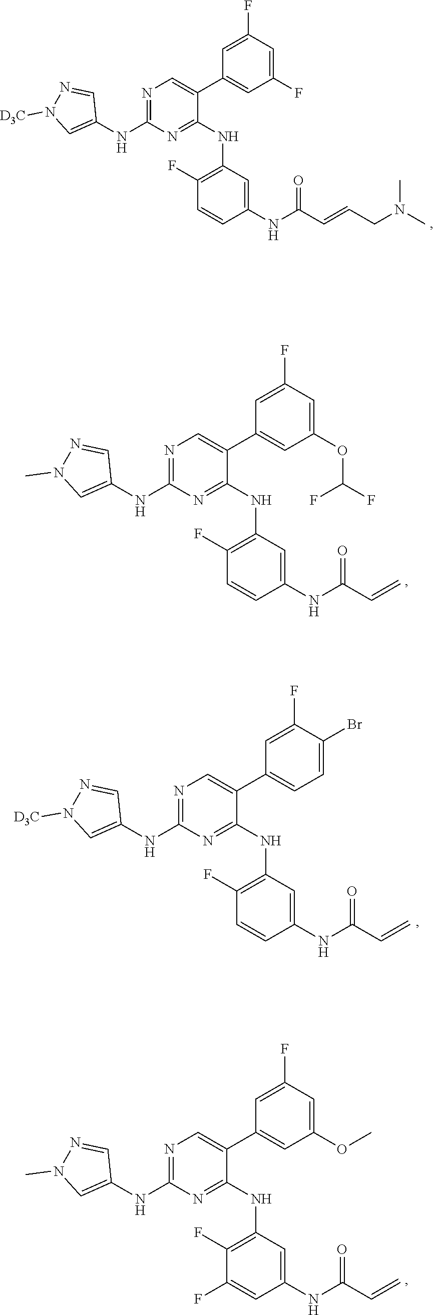

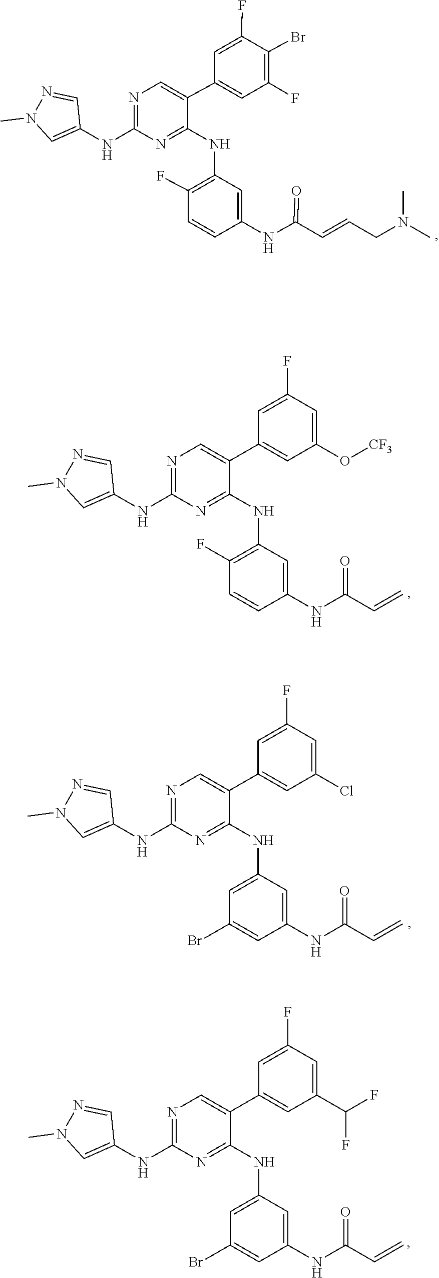

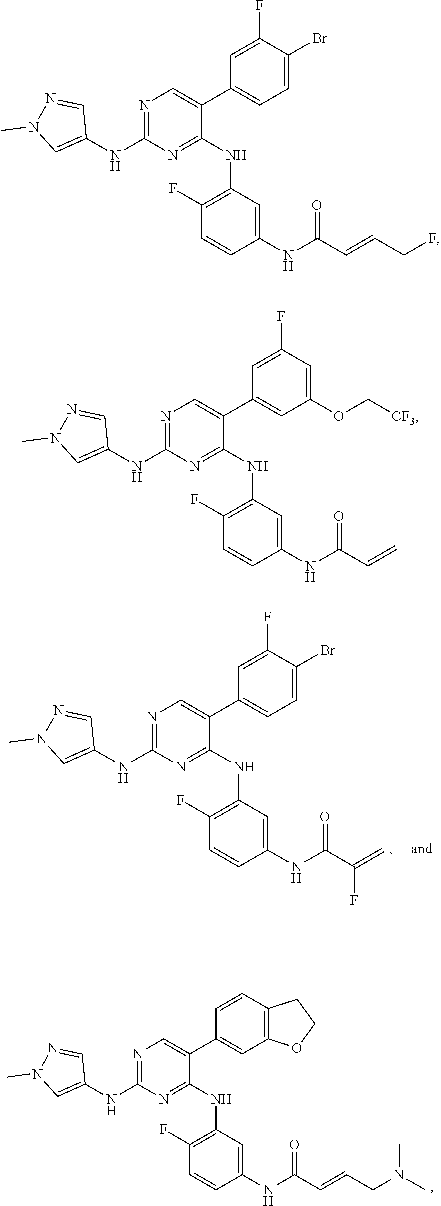

or a pharmaceutically acceptable salt or stereoisomer thereof.

In some embodiments, the compounds described herein have improved potency and increased efficacy. In some embodiments, the compounds described herein are useful as inhibitors of both EGFR and HER2. In some embodiments, the compounds described herein are dual inhibitors of EGFR and HER2. In some embodiments, the compounds described herein are dual inhibitors of mutant forms of EGFR and HER2. In some embodiments, the compounds described herein are dual inhibitors of wild type EGFR and a mutant form of HER2. In some embodiments, the compounds described herein have improved potency and increased efficacy through the inhibition of both EGFR and HER2.

In another aspect, provided herein is a pharmaceutical composition comprising a compound of Formula I, or a pharmaceutically acceptable salt or stereoisomer thereof, and a pharmaceutically acceptable carrier.

In another aspect, provided herein is a method of inhibiting a human epidermal growth factor receptor 2 (HER2) mutant and an epidermal growth factor receptor (EGFR) mutant in a subject in need thereof, comprising administering to the subject a therapeutically effective amount of a compound of Formula I, or a pharmaceutically acceptable salt or stereoisomer thereof. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is selected from A775_G776insYVMA, A775_G776insSVMA, A775_G776insVVMA, G776del insVC, G776del insLC, G776del insAV, G776del insAVGC, S310F, S310Y, p95, V842I, P780_Y781insGSP, and any combination thereof.

In some embodiments, the EGFR mutant comprises a substitution in exon 18, a deletion in exon 19, a substitution in exon 20, an insertion in exon 20, a mutation in the extracellular domain, or a substitution in exon 21. In some embodiments, the EGFR mutant is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutant is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutant is del19/T790M EGFR or L858R/T790M EGFR.

In another aspect, provided herein is a method of treating one or more cancer cells in a subject in need thereof, comprising administering to the subject a therapeutically effective amount of a compound of Formula I, or a pharmaceutically acceptable salt or stereoisomer thereof.

In another aspect, provided herein is a method of treating cancer in a subject in need thereof, comprising administering to the subject a therapeutically effective amount of a compound of Formula I, or a pharmaceutically acceptable salt or stereoisomer thereof.

In some embodiments, the cancer is bladder cancer, prostate cancer, breast cancer, cervical cancer, colorectal cancer, endometrial cancer, gastric cancer, glioblastoma, head and neck cancer, lung cancer, or non-small cell lung cancer. In some embodiments, the cancer is non-small cell lung cancer, prostate cancer, head and neck cancer, breast cancer, colorectal cancer, or glioblastoma.

In some embodiments, the cancer in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is selected from A775 G776insYVMA, A775_G776insSVMA, A775_G776insVVMA, G776del insVC, G776del insLC, G776del insAV, G776del insAVGC, S310F, S310Y, p95, V842I, P780_Y781insGSP, and any combination thereof.

In some embodiments, the cancer in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutation is del19/T790M EGFR or L858R/T790M EGFR.

In another aspect, the present disclosure provides a method of treating an inflammatory disease in a subject in need thereof, comprising administering to the subject a therapeutically effective amount of a compound of Formula I, or a pharmaceutically acceptable salt or stereoisomer thereof.

In some embodiments, the inflammatory disease is psoriasis, eczema, or atherosclerosis.

In some embodiments, the inflammatory disease in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is selected from A775_G776insYVMA, A775_G776insSVMA, A775_G776insVVMA, G776del insVC, G776del insLC, G776del insAV, G776del insAVGC, S310F, S310Y, p95, V842I, P780_Y781insGSP, or any combination thereof.

In some embodiments, the inflammatory disease in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutation is del19/T790M EGFR or L858R/T790M EGFR.

The present disclosure discloses a process of preparation of compounds of Formula I, or its stereoisomers, tautomers, pharmaceutically acceptable salts, stereoisomers, solvates, and hydrates thereof, and to pharmaceutical compositions containing them.

The compounds of the present disclosure may be useful in the treatment, prevention or suppression of diseases and disorders mediated by epidermal growth factor receptor (EGFR) and human epidermal growth factor receptor 2 (HER2).

These and other features, aspects, and advantages of the present disclosure will become better understood with reference to the following description. This statement is provided to introduce a selection of concepts in simplified form. This statement is not intended to identify key features or essential features of the subject matter, nor is it intended to be used to limit the scope of the subject matter.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE DISCLOSURE

### Definitions

In the structural formulae given herein and throughout the present disclosure, the following terms have the indicated meaning, unless specifically stated otherwise.

The term “optionally substituted” as used herein means that the group in question is either unsubstituted or substituted with one or more of the substituents specified. In some embodiments, when the group in question is substituted with more than one substituent, the substituent is the same. In some embodiments, when the group in question is substituted with more than one substituent, the substituent is different. In some embodiments, the reference group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O)

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl

The term “alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “cycloalkyl” refers to unless otherwise mentioned, carbocyclic groups of from 3 to 6 carbon atoms having a single cyclic ring or multiple condensed rings or spirocyclic rings or bridged rings. This definition encompasses rings that are saturated or partially unsaturated.

Such cycloalkyl groups include, by way of example, single ring structures such as cyclopropyl, cyclobutyl, cyclopentyl, cyclopentenyl, cyclohexyl, cyclohexenyl, and the like.

“Halo” or “Halogen”, alone or in combination with any other term means halogens such as chloro (Cl), fluoro (F), bromo (Br) and iodo (I).

The term “aryl” refers to a radical derived from a hydrocarbon ring system comprising hydrogen, 6 to 30 carbon atoms and at least one aromatic ring. This definition encompasses monocyclic, bicyclic, tricyclic or tetracyclic ring system, as well as fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

The term “phenyl” refers to an aromatic carbocyclic group of 6 carbon atoms having a single ring.

The term “phenyl alkyl” refers to a monoradical branched or unbranched saturated hydrocarbon chain having 1, 2, 3, 4, 5, or 6 carbon atoms substituted with an aromatic carbocyclic group of 6 carbon atoms having a single ring.

The term “heteroaryl” refers to an aromatic cyclic group having 5, or 6 carbon atoms and 1, 2, or 3 heteroatoms selected from oxygen, nitrogen and sulfur within at least one ring. An “X-linked heteroaryl” refers to a heteroaryl connected to the rest of the molecule via an X atom. For example,

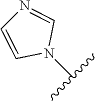 [SMILES: *n1ccnc1]

is an N-linked imidazolyl, while

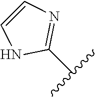 [SMILES: *c1ncc[nH]1]

is a C-linked imidazolyl.

The term “heterocycloalkyl” refers to a saturated, partially unsaturated, or unsaturated group having a single ring or multiple condensed rings or spirocyclic rings, or bridged rings unless otherwise mentioned, having from 2 to 10 carbon atoms and from 1 to 3 hetero atoms, selected from nitrogen, sulfur, phosphorus, and/or oxygen within the ring.

The term “alkenyl” refers to unsaturated aliphatic groups having at least one double bond.

The term “alkynyl” refers to unsaturated aliphatic groups having at least one triple bond.

The term “amino” refers to the —NH₂ radical.

The term “cyano” refers to the —CN radical.

The term “hydroxy” or “hydroxyl” refers to the —OH radical.

The term “heteroalkyl” refers to an alkyl radical as described above where one or more carbon atoms of the alkyl is replaced with an O, N or S atom. Unless stated otherwise specifically in the specification, the heteroalkyl group is optionally substituted as described below. Representative heteroalkyl groups include, but are not limited to —OCH₂CH₂OMe, —OCH₂CH₂OCH₂CH₂NH₂, and —OCH₂CH₂OCH₂CH₂OCH₂CH₂N(Me)₂.

A “hetercycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “haloalkyl” refers to an alkyl radical as described above where one or more carbon atoms of the alkyl is replaced with a halogen atom. In some embodiments, the haloalkyl group is optionally substituted as described below. Representative haloalkyl groups include, but are not limited to, fluoromethyl, difluoromethyl, trifluoromethyl, difluoroethyl, and trifluoroethyl.

The term “aminoalkyl” refers to an alkyl group substituted with an amino (NH2) group.

In some embodiments, the aminoalkyl group is unsubstituted or substituted with alkyl on the nitrogen atom.

The term “alkoxy” refers to the group R—O—, where R is optionally substituted alkyl or optionally substituted cycloalkyl, or optionally substituted alkenyl or optionally substituted alkynyl; or optionally substituted cycloalkenyl, where alkyl, alkenyl, alkynyl, cycloalkyl and cycloalkenyl are as defined herein. Representative examples of alkoxy groups include but are not limited to methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. In some embodiments, the present compounds include isotopes of hydrogen, carbon, nitrogen, oxygen, sulfur, fluorine chlorine, iodine, phosphorus, such as, for example, ²H, ³H, ¹³C, 14C, ¹⁵N, ¹⁸O, ¹⁷O, ³⁵, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, the compounds described herein exist as isotopic variants. In some embodiments, an isotopic variant of a compound described herein has one or more hydrogen atoms replaced by deuterium.

In some embodiments, the compounds described herein contain one or more chiral centers and/or double bonds and therefore, exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. In some embodiments, enantiomeric and stereoisomeric mixtures are resolved into their component enantiomers or stereoisomers using separation techniques or chiral synthesis techniques well known to the person skilled in the art. In some embodiments, the compounds also exist in several tautomeric forms including the enol form, the keto form and mixtures thereof. Accordingly, the chemical structures depicted herein encompass all possible tautomeric forms of the illustrated or identified compounds.

In some embodiments, a compound disclosed herein is a free base, salt, hydrate, isomer, diastereomer, prodrug (e.g., ester), metabolite, ion pair complex, or chelate form. In some embodiments, compounds exist in unsolvated forms as well as solvated forms, including hydrated forms and as N-oxides. In some embodiments, compounds are hydrated, solvated or N-oxides. Also contemplated within the scope of the disclosure are congeners, analogs, hydrolysis products, metabolites and precursor or prodrugs of the compound. In general, unless otherwise indicated, all physical forms are equivalent for the uses contemplated herein and are intended to be within the scope of the present disclosure.

“Pharmaceutically acceptable salt” embraces salts with a pharmaceutically acceptable acid or base. Pharmaceutically acceptable acids include both inorganic acids, for example hydrochloric, sulfuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulfonic, ethanesulfonic, benzenesulfonic or p-toluenesulfonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines. In some embodiments, the compound is a pharmaceutically acceptable salt derived from acids including, but not limited to, the following: acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, or p-toluenesulfonic acid.

“Pharmaceutical composition” refers to one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition comprising a compound of the present disclosure and a pharmaceutically acceptable carrier.

“Carrier” refers to a diluent, adjuvant, excipient, or vehicle with which the therapeutic is administered. In some embodiments, such pharmaceutical carriers are sterile liquids, such as water and oils, including those of petroleum, animal, vegetable or synthetic origin, including but not limited to peanut oil, soybean oil, mineral oil, sesame oil and the like. In some embodiments, water is a carrier when the pharmaceutical composition is administered orally. In some embodiments, saline and aqueous dextrose are exemplary carriers when the pharmaceutical composition is administered intravenously. In some embodiments, saline solutions and aqueous dextrose and glycerol solutions are employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In some embodiments, the composition comprises minor amounts of wetting or emulsifying agents, or pH buffering agents. In some embodiments, these compositions take the form of solutions, suspensions, emulsions, tablets, pills, capsules, powders, sustained-release formulations and the like. In some embodiments, the composition is formulated as a suppository, with traditional binders and carriers such as triglycerides. In some embodiments, an oral formulation comprises carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, for example in purified form, together with a suitable amount of carrier so as to provide the form for proper administration to the patient. The formulation should suit the mode of administration.

“Combined” or “in combination” or “combination” should be understood as a functional coadministration, encompassing scenarios wherein compounds are administered separately, in different formulations, different modes of administration (for example subcutaneous, intravenous or oral) and different times of administration. In some embodiments, the individual compounds of such combinations are administered sequentially in separate pharmaceutical compositions. In some embodiments, the individual compounds of such combinations are administered simultaneously in combined pharmaceutical compositions.

### Compounds

In one aspect, provided herein is a compound of Formula I.

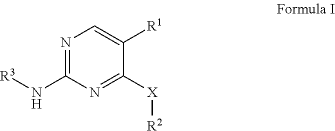

or a pharmaceutically acceptable salt or stereoisomer thereof, wherein:
X is —NH— or —O—;
R¹ is —(C(R⁴)₂)ₙR⁵, wherein R⁵ is substituted with 2 or 3 R^{5′};
   n is 0, 1, 2, or 3;
   each R⁴ is independently hydrogen, alkyl, halo, haloalkyl, hydroxy, alkoxy, or heteroalkyl;
   R⁵ is C₄₋₁₀cycloalkyl, C-linked heterocycloalkyl, aryl, or heteroaryl;
   each R^{5′} is independently aryl, heteroaryl, alkyl, cycloalkyl, heterocycloalkyl, oxo, halo, heteroalkyl, haloalkyl, cyano, hydroxy, amino, —NH₂, —NHR⁶, —N(R⁶)₂, —C(═O)NH₂, —C(═O)NR⁶, —C(═O)N(R⁶)₂, —NR⁶C(═O)R⁶, —NHC(═O)R⁶, —S(═O)₂alkyl, —S(═O)₂aryl, —S(═O)₂NH₂, —S(═O)₂NHR⁶, —S(═O)₂N(R⁶)₂, —S(═O)₂heteroaryl, alkoxy, or haloalkoxy;
      each R⁶ is independently alkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
   each R⁷ is independently

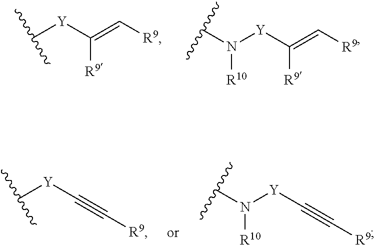

Y is —C(═O)—, —S(═O)—, or —S(═O)₂—;
R⁹ and R^{9′} are independently hydrogen, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl;
      each R¹⁴ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy; and
         each R¹⁵ is independently alkyl, cycloalkyl, aryl, or heteroaryl;
            provided that when X is —O—, R⁵ is not C-linked heterocycloalkyl.

In one aspect, provided herein is a compound of Formula I:

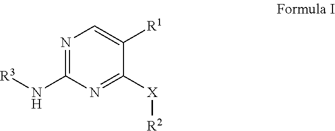

or a pharmaceutically acceptable salt or stereoisomer thereof, wherein:
X is —NH— or —O—;
R¹ is —(C(R⁴)₂)ₙR⁵, wherein R⁵ is 2 or 3 R^{5′};
   n is 0, 1, 2, or 3;
   each R⁴ is independently hydrogen, alkyl, halo, haloalkyl, hydroxy, alkoxy, or heteroalkyl;
   R⁵ is C₄₋₁₀cycloalkyl, aryl, or heteroaryl;
   each R^{5′} is independently deuterium, aryl, heteroaryl, alkyl, C₃-C₆ cycloalkyl, 3-8 membered heterocycloalkyl, oxo, halo, heteroalkyl, haloalkyl, cyano, hydroxy, amino, —NH₂, —NHR⁶, —N(CH₃)R₆, —N(R⁶)₂, —C(═O)NH₂, —C(═O)NHR⁶, —C(═O)N(R⁶)₂, —NR⁶C(═O)R⁶, —NHC(═O)R⁶, —S(═O)₂alkyl, —S(═O)₂aryl, —S(═O)₂NH₂, —S(═O)₂NHR⁶, —S(═O)₂N(R⁶)₂, —S(═O)₂heteroaryl, alkoxy, or haloalkoxy; or
   two adjacent R^{5′} groups come together to form a 5- to 10-membered heterocycle, wherein the 5- to 10-membered heterocycle is unsubstituted or substituted with alkyl; each R⁶ is independently alkyl, aminoalkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
   each R⁷ is independently

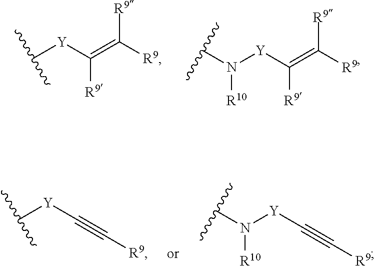

Y is —C(═O)—, —S(═O)—, or —S(═O)₂—;
R⁹, R^{9′}, and R^{9″} are independently hydrogen, deuterium, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently unsubstituted or substituted with 0, 1, or 2 R¹⁴;
      each R¹³ is independently hydrogen, alkyl, cycloalkyl, aryl, or heteroaryl;
      each R¹⁴ is independently deuterium, aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

For any and all of the embodiments, substituents are selected from among a subset of the listed alternatives. For example, in some embodiments, X is —NH—. In some embodiments, X is —O—.

In some embodiments, n is 0, 1, 2, or 3. In some embodiments, n is 0, 1, or 2. In some embodiments, n is 0, 1, or 3. In some embodiments, n is 0, 2, or 3. In some embodiments, n is 1, 2, or 3. In some embodiments, n is 0 or 1. In some embodiments, n is 1 or 2. In some embodiments, n is 2 or 3. In some embodiments, n is 0 or 2. In some embodiments, n is 0 or 3. In some embodiments, n is 1 or 3. In some embodiments, n is 0. In some embodiments, n is 1. In some embodiments, n is 2. In some embodiments, n is 3.

In some embodiments, R⁵ is phenyl, naphthyl, anthracenyl, phenanthrenyl, chrysenyl, pyrenyl, C-linked pyridyl, C-linked pyrimidinyl, C-linked pyrazolyl, C-linked imidazolyl, or C-linked indolyl; wherein R⁵ is substituted with 2 or 3 R^{5′}. In some embodiments, R⁵ is phenyl, naphthyl, anthracenyl, phenanthrenyl, C-linked pyridyl, C-linked pyrimidinyl, C-linked pyrazolyl, or C-linked imidazolyl; wherein R⁵ is substituted with 2 or 3 R^{5′}. In some embodiments, R⁵ is phenyl; wherein the phenyl is substituted with 2 or 3 R^{5′}. In some embodiments, R⁵ is naphthyl; wherein the naphthyl is substituted with 2 or 3 R^{5′}. In some embodiments, R⁵ is anthracenyl; wherein the anthracenyl is substituted with 2 or 3 R^{5′}. In some embodiments, R⁵ is phenanthrenyl; wherein the phenanthrenyl is substituted with 2 or 3 R^{5′}. In some embodiments, R⁵ is chrysenyl; wherein the chrysenyl is substituted with 2 or 3 R^{5′}. In some embodiments, R⁵ is pyrenyl; wherein the pyrenyl is substituted with 2 or 3 R^{5′}. In some embodiments, R⁵ is C-linked pyridyl; wherein the pyridyl is substituted with 2 or 3 R^{5′}. In some embodiments, R⁵ is C-linked pyrimidinyl; wherein the C-linked pyrimidinyl is substituted with 2 or 3 R^{5′}. In some embodiments, R⁵ is C-linked pyrazolyl; wherein the C-linked pyrazolyl is substituted with 2 or 3 R^{5′}. In some embodiments, R⁵ is C-linked imidazolyl; wherein C-linked imidazolyl is substituted with 2 or 3 R^{5′}. In some embodiments, R⁵ is C-linked indolyl; wherein the C-linked indolyl is substituted with 2 or 3 R^{5′}.

In some embodiments, R⁵ is substituted with 2 or 3 R^{5′}. In some embodiments, R⁵ is substituted with 2 R^{5′}. In some embodiments, R⁵ is substituted with 3 R^{5′}.

In some embodiments, R⁵ is phenyl or C-linked pyridyl; wherein the phenyl or C-linked pyridyl is substituted with 2 or 3 R^{5′}. In some embodiments, two adjacent R^{5′} groups come together to form a 5- to 10-membered heterocycle.

In some embodiments, each R⁴ is independently hydrogen, alkyl, halo, haloalkyl, hydroxy, alkoxy, or heteroalkyl. In some embodiments, each R⁴ is independently hydrogen, alkyl, halo, haloalkyl, or alkoxy. In some embodiments, each R⁴ is independently hydrogen, methyl, ethyl, n-propyl, iso-propyl, n-butyl, iso-butyl, sec-butyl, tert-butyl, fluoro, chloro, trifluoromethyl, trifluoroethyl, pentafluoroethyl, methoxy, ethoxy, or trifluoromethoxy. In some embodiments, each R⁴ is independently hydrogen, methyl, fluoro, trifluoromethyl, methoxy, or trifluoromethoxy. In some embodiments, each R⁴ is hydrogen. In some embodiments, each R⁴ is independently alkyl. In some embodiments, each R⁴ is independently halo. In some embodiments, each R⁴ is independently haloalkyl. In some embodiments, each R⁴ is hydroxy. In some embodiments, each R⁴ is independently alkoxy. In some embodiments, each R⁴ is independently heteroalkyl. In some embodiments, each R⁴ is methyl. In some embodiments, each R⁴ is ethyl. In some embodiments, each R⁴ is n-propyl. In some embodiments, each R⁴ is iso-propyl. In some embodiments, each R⁴ is n-butyl. In some embodiments, each R⁴ is iso-butyl. In some embodiments, each R⁴ is sec-butyl. In some embodiments, each R⁴ is tert-butyl.

In some embodiments, each R⁴ is fluoro. In some embodiments, each R⁴ is chloro. In some embodiments, each R⁴ is trifluoromethyl. In some embodiments, each R⁴ is trifluoroethyl. In some embodiments, each R⁴ is pentafluoroethyl. In some embodiments, each R⁴ is methoxy. In some embodiments, each R⁴ is ethoxy. In some embodiments, each R⁴ is trifluoromethoxy.

In some embodiments, each R^{5′} is independently alkyl, haloalkyl, heterocycloalkyl, halo, cyano, hydroxy, —N(R⁶)₂, —C(═O)NR⁶, —NHC(═O)R⁶, —S(═O)₂NH₂, alkoxy, or haloalkoxy. In some embodiments, each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy. In some embodiments, each R^{5′} is independently aryl, heteroaryl, alkyl, heterocycloalkyl, halo, cyano, hydroxy, —N(R⁶)₂, or alkoxy. In some embodiments, each R^{5′} is independently aryl. In some embodiments, each R^{5′} is independently heteroaryl. In some embodiments, each R^{5′} is independently alkyl. In some embodiments, each R^{5′} is independently cycloalkyl. In some embodiments, each R^{5′} is independently heterocycloalkyl. In some embodiments, each R^{5′} is independently halo. In some embodiments, each R^{5′} is independently heteroalkyl. In some embodiments, each R^{5′} is independently haloalkyl. In some embodiments, each R^{5′} is cyano. In some embodiments, each R^{5′} is hydroxy.

In some embodiments, each R^{5′} is amino. In some embodiments, each R^{5′} is independently —N(R⁶)₂. In some embodiments, each R^{5′} is independently —S(═O)₂alkyl. In some embodiments, each R^{5′} is independently —S(═O)₂aryl. In some embodiments, each R^{5′} is independently-S(═O)₂heteroaryl. In some embodiments, each R^{5′} is independently alkoxy. In some embodiments, each R^{5′} is independently phenyl, naphthyl, anthracenyl, phenanthrenyl, chrysenyl, pyrenyl, pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, naphthyridinyl, methyl, ethyl, n-propyl, iso-propyl, n-butyl, iso-butyl, sec-butyl, tert-butyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, hydroxy, —N(R⁶)₂, methoxy, ethoxy, or trifluoromethoxy. In some embodiments, each R^{5′} is independently phenyl, pyrrolyl, imidazolyl, thiazolyl, isothiazolyl, oxazolyl, isoxazolyl, pyridinyl, pyrimidinyl, methyl, ethyl, tert-butyl, pyrrolidinyl, piperidinyl, piperazinyl, morpholinyl, fluoro, chloro, cyano, hydroxy, —N(R⁶)₂, methoxy, ethoxy, or trifluoromethoxy. In some embodiments, each R^{5′} is independently methyl, ethyl, tert-butyl, pyrrolidinyl, piperidinyl, piperazinyl, morpholinyl, fluoro, chloro, cyano, hydroxy, —N(R⁶)₂, —C(═O)NR⁶, —NHC(═O)R⁶, —S(═O)₂NH₂, methoxy, ethoxy, fluoromethyl, difluoromethyl, trifluoromethyl, fluoromethoxy, difluoromethoxy, or trifluoromethoxy. In some embodiments, each R^{5′} is independently methyl, morpholinyl, fluoro, chloro, cyano, —C(═O)NHMe, —NHC(═O)Me, —S(═O)₂NH₂, methoxy, fluoromethyl, difluoromethyl, trifluoromethyl, difluoromethoxy, or trifluoromethoxy. In some embodiments, each R^{5′} is independently phenyl, imidazolyl, pyridinyl, methyl, tert-butyl, pyrrolidinyl, morpholinyl, fluoro, cyano, hydroxy, —N(R⁶)₂, or methoxy. In some embodiments, each R^{5′} is phenyl. In some embodiments, each R^{5′} is naphthyl. In some embodiments, each R^{5′} is anthracenyl. In some embodiments, each R^{5′} is phenanthrenyl. In some embodiments, each R^{5′} is chrysenyl. In some embodiments, each R^{5′} is pyrenyl. In some embodiments, each R^{5′} is pyrrolyl. In some embodiments, each R^{5′} is imidazolyl. In some embodiments, each R^{5′} is pyrazolyl. In some embodiments, each R^{5′} is triazolyl. In some embodiments, each R^{5′} is tetrazolyl. In some embodiments, each R^{5′} is indolyl. In some embodiments, each R^{5′} is indazolyl. In some embodiments, each R^{5′} is benzimidazolyl. In some embodiments, each R^{5′} is azaindolyl. In some embodiments, each R^{5′} is thiazolyl. In some embodiments, each R^{5′} is isothiazolyl. In some embodiments, each R^{5′} is oxazolyl. In some embodiments, each R^{5′} is isoxazolyl. In some embodiments, each R^{5′} is pyridinyl. In some embodiments, each R^{5′} is pyrimidinyl. In some embodiments, each R^{5′} is pyridazinyl. In some embodiments, each R^{5′} is pyrazinyl. In some embodiments, each R^{5′} is triazinyl. In some embodiments, each R^{5′} is quinolinyl. In some embodiments, each R^{5′} is isoquinolinyl. In some embodiments, each R^{5′} is quinoxalinyl. In some embodiments, each R^{5′} is quinazolinyl. In some embodiments, each R^{5′} is cinnolinyl. In some embodiments, each R^{5′} is naphthyridinyl. In some embodiments, each R^{5′} is methyl. In some embodiments, each R^{5′} is ethyl. In some embodiments, each R^{5′} is n-propyl. In some embodiments, each R^{5′} is iso-propyl. In some embodiments, each R^{5′} is n-butyl. In some embodiments, each R^{5′} is iso-butyl. In some embodiments, each R^{5′} is sec-butyl. In some embodiments, each R^{5′} is tert-butyl. In some embodiments, each R^{5′} is azetidinyl. In some embodiments, each R^{5′} is oxetanyl. In some embodiments, each R^{5′} is pyrrolidinyl. In some embodiments, each R^{5′} is imidazolidinyl. In some embodiments, each R^{5′} is tetrahydrofuranyl. In some embodiments, each R^{5′} is piperidinyl. In some embodiments, each R^{5′} is piperazinyl. In some embodiments, each R^{5′} is tetrahydropyranyl. In some embodiments, each R^{5′} is morpholinyl. In some embodiments, each R^{5′} is fluoro. In some embodiments, each R^{5′} is chloro.

In some embodiments, each R^{5′} is methoxy. In some embodiments, each R^{5′} is ethoxy. In some embodiments, each R^{5′} is trifluoromethoxy. In some embodiments, each R^{5′} is —C(═O)NHMe. In some embodiments, each R^{5′} is —NHC(═O)Me. In some embodiments, each R^{5′} is —S(═O)₂NH₂. In some embodiments, each R^{5′} is difluoromethoxy.

In some embodiments, each R⁶ is independently alkyl, cycloalkyl, aryl, or heteroaryl. In some embodiments, each R⁶ is independently alkyl or aryl. In some embodiments, each R⁶ is independently methyl, ethyl, n-propyl, iso-propyl, n-butyl, iso-butyl, sec-butyl, tert-butyl, phenyl, naphthyl, anthracenyl, phenanthrenyl, chrysenyl, or pyrenyl. In some embodiments, each R⁶ is independently methyl, ethyl, iso-propyl, tert-butyl, phenyl, or naphthyl. In some embodiments, each R⁶ is independently methyl or phenyl. In some embodiments, each R⁶ is methyl. In some embodiments, each R⁶ is ethyl. In some embodiments, each R⁶ is n-propyl. In some embodiments, each R⁶ is iso-propyl. In some embodiments, each R⁶ is n-butyl. In some embodiments, each R⁶ is iso-butyl. In some embodiments, each R⁶ is sec-butyl. In some embodiments, each R⁶ is tert-butyl. In some embodiments, each R⁶ is phenyl. In some embodiments, each R⁶ is naphthyl. In some embodiments, each R⁶ is anthracenyl. In some embodiments, each R⁶ is phenanthrenyl. In some embodiments, each R⁶ is chrysenyl. In some embodiments, each R⁶ is pyrenyl.

In some embodiments, R² is aryl, heteroaryl, cycloalkyl, or heterocycloalkyl; wherein R² is substituted with at least one R⁷ and 0, 1, or 2 R⁸. In some embodiments, R² is aryl; wherein R² is substituted with at least one R⁷ and 0, 1, or 2 R⁸. In some embodiments, R² is heteroaryl; wherein R² is substituted with at least one R⁷ and 0, 1, or 2 R⁸. In some embodiments, R² is cycloalkyl; wherein R² is substituted with at least one R⁷ and 0, 1, or 2 R⁸. In some embodiments, R² is heterocycloalkyl; wherein R² is substituted with at least one R⁷ and 0, 1, or 2 R⁸. In some embodiments, R² is monocyclic. In some embodiments, R² is phenyl, cyclopropyl, cyclobutyl, cyclopentyl, cyclohexyl, pyrrolyl, imidazolyl, pyrazolyl, triazolyl, tetrazolyl, thiazolyl, isothiazolyl, oxazolyl, isoxazolyl, pyridinyl, pyrimidinyl, pyridazinyl, pyrazinyl, or triazinyl; wherein R² is substituted with at least one R⁷ and 0, 1, or 2 R⁸. In some embodiments, R² is phenyl, cyclohexyl, or pyrrolyl; wherein R² is substituted with at least one R⁷ and 0, 1, or 2 R⁸. In some embodiments, R² is phenyl; wherein R² is substituted with at least one R⁷ and 0, 1, or 2 R⁸. In some embodiments, R² is cyclopropyl; wherein R² is substituted with at least one R⁷ and 0, 1, or 2 R⁸. In some embodiments, R² is cyclobutyl; wherein R² is substituted with at least one R⁷ and 0, 1, or 2 R⁸. In some embodiments, R² is cyclopentyl; wherein R² is substituted with at least one R⁷ and 0, 1, or 2 R⁸. In some embodiments, R² is cyclohexyl; wherein R² is substituted with at least one R⁷ and 0, 1, or 2 R⁸. In some embodiments, R² is pyrrolyl; wherein R² is substituted with at least one R⁷ and 0, 1, or 2 R⁸. In some embodiments, R² is imidazolyl; wherein R² is substituted with at least one R⁷ and 0, 1, or 2 R⁸. In some embodiments, R² is pyrazolyl; wherein R² is substituted with at least one R⁷ and 0, 1, or 2 R⁸. In some embodiments, R² is triazolyl; wherein R² is substituted with at least one R⁷ and 0, 1, or 2 R⁸. In some embodiments, R² is tetrazolyl; wherein R² is substituted with at least one R⁷ and 0, 1, or 2 R⁸. In some embodiments, R² is thiazolyl; wherein R² is substituted with at least one R⁷ and 0, 1, or 2 R⁸. In some embodiments, R² is isothiazolyl; wherein R² is substituted with at least one R⁷ and 0, 1, or 2 R⁸. In some embodiments, R² is oxazolyl; wherein R² is substituted with at least one R⁷ and 0, 1, or 2 R⁸. In some embodiments, R² is isoxazolyl; wherein R² is substituted with at least one R⁷ and 0, 1, or 2 R⁸. In some embodiments, R² is pyridinyl; wherein R² is substituted with at least one R⁷ and 0, 1, or 2 R⁸. In some embodiments, R² is pyrimidinyl; wherein R² is substituted with at least one R⁷ and 0, 1, or 2 R⁸. In some embodiments, R² is pyridazinyl; wherein R² is substituted with at least one R⁷ and 0, 1, or 2 R⁸. In some embodiments, R² is pyrazinyl; wherein R² is substituted with at least one R⁷ and 0, 1, or 2 R⁸. In some embodiments, R² is triazinyl; wherein R² is substituted with at least one R⁷ and 0, 1, or 2 R⁸.

In some embodiments, R⁷ is

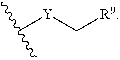

In some embodiments, R⁷ is

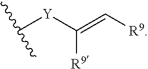

In some embodiments, R⁷ is

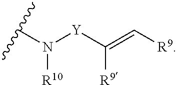

In some embodiments, R⁷ is

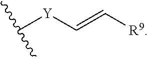

embodiments, R⁷ is

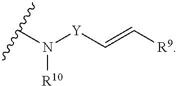

In some embodiments, Y is —C(═O)—. In some embodiments, Y is —S(═O)—. In some embodiments, Y is —S(═O)₂—.

In some embodiments, R⁷ is

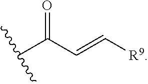

In some embodiments, R⁷ is

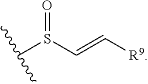

In some embodiments, R⁷ is

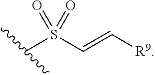

In some embodiments, R⁷ is

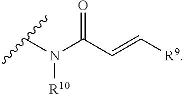

In some embodiments, R⁷ is

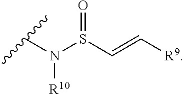

In some embodiments, R⁷ is

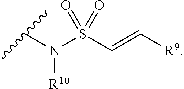

In some embodiments, R⁷ is

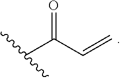 [SMILES: *C(=O)C=C]

In some embodiments, R⁷ is

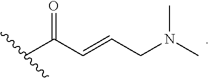 [SMILES: *C(=O)/C=C/CN(C)C]

In some embodiments, R⁷ is

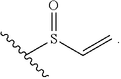 [SMILES: *S(=O)C=C]

In some embodiments, R⁷ is

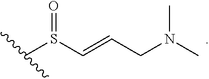 [SMILES: *S(=O)/C=C/CN(C)C]

In some embodiments, R⁷ is

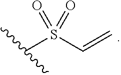 [SMILES: *S(=O)(=O)C=C]

In some embodiments, R⁷ is

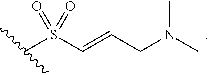 [SMILES: *S(=O)(=O)/C=C/CN(C)C]

In some embodiments, R⁷ is

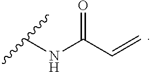 [SMILES: *NC(=O)C=C]

In some embodiments, R⁷ is

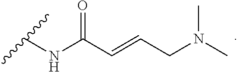 [SMILES: *NC(=O)/C=C/CN(C)C]

In some embodiments, R⁷ is

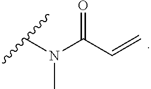 [SMILES: *N(C)C(=O)C=C]

In some embodiments, R⁷ is

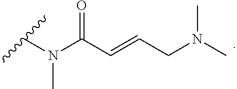 [SMILES: *N(C)C(=O)/C=C/CN(C)C]

In some embodiments, R⁷ is

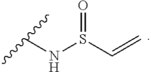 [SMILES: *NS(=O)C=C]

In some embodiments, R⁷ is

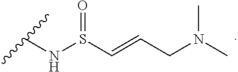 [SMILES: *NS(=O)/C=C/CN(C)C]

In some embodiments, R⁷ is

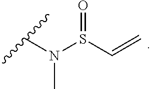 [SMILES: *N(C)S(=O)C=C]

In some embodiments, R⁷ is

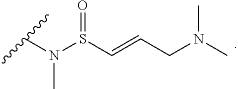 [SMILES: *N(C)S(=O)/C=C/CN(C)C]

In some embodiments, R⁷ is

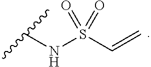 [SMILES: *NS(=O)(=O)C=C]

In some embodiments, R⁷ is

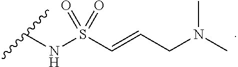 [SMILES: *NS(=O)(=O)/C=C/CN(C)C]

embodiments, R⁷ is

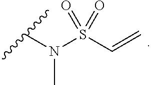 [SMILES: *N(C)S(=O)(=O)C=C]

In some embodiments, R⁷ is

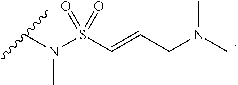 [SMILES: *N(C)S(=O)(=O)/C=C/CN(C)C]

In some embodiments, R⁹ and R^{9′} are independently hydrogen, halo, alkyl, heteroalkyl, haloalkyl, or (alkyl)heterocycloalkyl. In some embodiments, R⁹ is hydrogen, halo, alkyl, cycloalkyl, or heteroalkyl. In some embodiments, R⁹ is hydrogen, halo, or heteroalkyl. In some embodiments, R⁹ and R^{9′} are independently hydrogen, fluoro, chloro, methyl, hydroxyethyl, methoxyethyl, methoxymethyl, dimethylaminomethyl, 1-piperidinylmethyl, 1-morpholinylmethyl, or fluoromethyl. In some embodiments, R⁹ is hydrogen, fluoro, chloro, hydroxyethyl, or methoxyethyl. In some embodiments, R⁹ is hydrogen. In some embodiments, R⁹ is fluoro. In some embodiments, R⁹ is chloro. In some embodiments, R⁹ is hydroxyethyl. In some embodiments, R⁹ is methoxyethyl. In some embodiments, R⁹ is methyl. In some embodiments, R⁹ is methoxymethyl. In some embodiments, R⁹ is dimethylaminomethyl. In some embodiments, R⁹ is 1-piperidinylmethyl. In some embodiments, R⁹ is 1-morpholinomethyl. In some embodiments, R⁹ is fluoromethyl. In some embodiments, R^{9′} is hydrogen. In some embodiments, R^{9′} is fluoro. In some embodiments, R^{9′} is chloro. In some embodiments, R^{9′} is hydroxyethyl. In some embodiments, R^{9′} is methoxyethyl. In some embodiments, R^{9′} is methyl. In some embodiments, R^{9′} is methoxymethyl. In some embodiments, R^{9′} is dimethylaminomethyl. In some embodiments, R^{9′} is 1-piperidinylmethyl. In some embodiments, R^{9′} is 1-morpholinomethyl. In some embodiments, R^{9′} is fluoromethyl.

In some embodiments, R¹⁰ is hydrogen or alkyl. In some embodiments, R¹⁰ is hydrogen, methyl, ethyl, n-propyl, iso-propyl, n-butyl, iso-butyl, sec-butyl, or tert-butyl. In some embodiments, R¹⁰ is hydrogen. In some embodiments, R¹⁰ is methyl. In some embodiments, R¹⁰ is ethyl. In some embodiments, R¹⁰ is n-propyl. In some embodiments, R¹⁰ is iso-propyl. In some embodiments, R¹⁰ is n-butyl. In some embodiments, R¹⁰ is iso-butyl. In some embodiments, R¹⁰ is sec-butyl. In some embodiments, R¹⁰ is tert-butyl.

In some embodiments, R² is not substituted with R⁸. In some embodiments, R² is substituted with 1 or 2 R⁸. In some embodiments, R² is substituted with 1 R⁸. In some embodiments, R² is substituted with 2 R⁸.

In some embodiments, each R⁸ is independently methyl, ethyl, n-propyl, iso-propyl, n-butyl, iso-butyl, sec-butyl, tert-butyl, fluoro, chloro, heteroalkyl, cyano, hydroxy, amino, —N(R¹¹)₂, methoxy, ethoxy, or trifluoromethoxy. In some embodiments, each R⁸ is independently methyl, ethyl, iso-propyl, tert-butyl, fluoro, chloro, —N(R¹¹)₂, hydroxyethyl, methoxyethyl, or cyano. In some embodiments, each R⁸ is methyl. In some embodiments, each R⁸ is ethyl. In some embodiments, each R⁸ is n-propyl. In some embodiments, each R⁸ is iso-propyl. In some embodiments, each R⁸ is n-butyl. In some embodiments, each R⁸ is iso-butyl. In some embodiments, each R⁸ is sec-butyl. In some embodiments, each R⁸ is tert-butyl. In some embodiments, each R⁸ is fluoro. In some embodiments, each R⁸ is chloro. In some embodiments, each R⁸ is independently —N(R¹¹)₂. In some embodiments, each R⁸ is hydroxyethyl. In some embodiments, each R⁸ is methoxyethyl. In some embodiments, each R⁸ is cyano.

In some embodiments, each R¹¹ is independently alkyl, cycloalkyl, aryl, or heteroaryl. In some embodiments, each R¹¹ is independently alkyl or aryl. In some embodiments, each R¹¹ is independently alkyl. In some embodiments, each R¹¹ is independently cycloalkyl. In some embodiments, each R¹¹ is independently aryl. In some embodiments, each R¹¹ is independently heteroaryl. In some embodiments, each R¹¹ is independently methyl, ethyl, n-propyl, iso-propyl, n-butyl, iso-butyl, sec-butyl, tert-butyl, phenyl, naphthyl, anthracenyl, phenanthrenyl, chrysenyl, or pyrenyl. In some embodiments, each R¹¹ is independently methyl, ethyl, iso-propyl, tert-butyl, phenyl, or naphthyl. In some embodiments, each R¹¹ is independently methyl or phenyl.

In some embodiments, each R¹¹ is methyl. In some embodiments, each R¹¹ is ethyl. In some embodiments, each R¹¹ is n-propyl. In some embodiments, each R¹¹ is iso-propyl. In some embodiments, each R¹¹ is n-butyl. In some embodiments, each R¹¹ is iso-butyl. In some embodiments, each R¹¹ is sec-butyl. In some embodiments, each R¹¹ is tert-butyl. In some embodiments, each R¹¹ is phenyl. In some embodiments, each R¹¹ is naphthyl. In some embodiments, each R¹¹ is anthracenyl. In some embodiments, each R¹¹ is phenanthrenyl. In some embodiments, each R¹¹ is chrysenyl. In some embodiments, each R¹¹ is pyrenyl.

In some embodiments, R³ is pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl; wherein R³ is substituted with 0, 1, 2, or 3 R¹². In some embodiments, R³ is imidazolyl, pyrazolyl, triazolyl, indolyl, indazolyl, thiazolyl, isothiazolyl, or pyridinyl; wherein R³ is substituted with 0, 1, 2, or 3 R¹². In some embodiments, R³ is pyrrolyl; wherein R³ is substituted with 0, 1, 2, or 3 R¹². In some embodiments, R³ is imidazolyl; wherein R³ is substituted with 0, 1, 2, or 3 R¹². In some embodiments, R³ is pyrazolyl; wherein R³ is substituted with 0, 1, 2, or 3 R¹². In some embodiments, R³ is triazolyl; wherein R³ is substituted with 0, 1, 2, or 3 R¹². In some embodiments, R³ is tetrazolyl; wherein R³ is substituted with 0, 1, 2, or 3 R¹². In some embodiments, R³ is indolyl; wherein R³ is substituted with 0, 1, 2, or 3 R¹². In some embodiments, R³ is indazolyl; wherein R³ is substituted with 0, 1, 2, or 3 R¹². In some embodiments, R³ is benzimidazolyl; wherein R³ is substituted with 0, 1, 2, or 3 R¹². In some embodiments, R³ is azaindolyl; wherein R³ is substituted with 0, 1, 2, or 3 R¹². In some embodiments, R³ is thiazolyl; wherein R³ is substituted with 0, 1, 2, or 3 R¹². In some embodiments, R³ is isothiazolyl; wherein R³ is substituted with 0, 1, 2, or 3 R¹². In some embodiments, R³ is oxazolyl; wherein R³ is substituted with 0, 1, 2, or 3 R¹². In some embodiments, R³ is isoxazolyl; wherein R³ is substituted with 0, 1, 2, or 3 R¹². In some embodiments, R³ is pyridinyl; wherein R³ is substituted with 0, 1, 2, or 3 R¹². In some embodiments, R³ is pyrimidinyl; wherein R³ is substituted with 0, 1, 2, or 3 R¹². In some embodiments, R³ is pyridazinyl; wherein R³ is substituted with 0, 1, 2, or 3 R¹². In some embodiments, R³ is pyrazinyl; wherein R³ is substituted with 0, 1, 2, or 3 R¹². In some embodiments, R³ is triazinyl; wherein R³ is substituted with 0, 1, 2, or 3 R¹². In some embodiments, R³ is quinolinyl; wherein R³ is substituted with 0, 1, 2, or 3 R¹². In some embodiments, R³ is isoquinolinyl; wherein R³ is substituted with 0, 1, 2, or 3 R¹². In some embodiments, R³ is quinoxalinyl; wherein R³ is substituted with 0, 1, 2, or 3 R¹². In some embodiments, R³ is quinazolinyl; wherein R³ is substituted with 0, 1, 2, or 3 R¹². In some embodiments, R³ is cinnolinyl; wherein R³ is substituted with 0, 1, 2, or 3 R¹². In some embodiments, R³ is naphthyridinyl; wherein R³ is substituted with 0, 1, 2, or 3 R¹².

In some embodiments, R³ is unsubstituted. In some embodiments, R³ is substituted with at least 1 R¹². In some embodiments, R³ is substituted with at least 2 R¹². In some embodiments, R³ is substituted with 1 R¹². In some embodiments, R³ is substituted with 2 R¹². In some embodiments, R³ is substituted with 3 R¹².

In some embodiments, R³ is

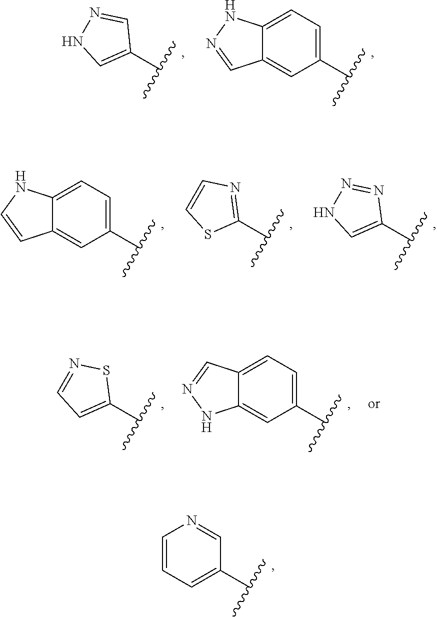

wherein R³ is substituted with 0 to 3 R¹². In some embodiments, R³ is

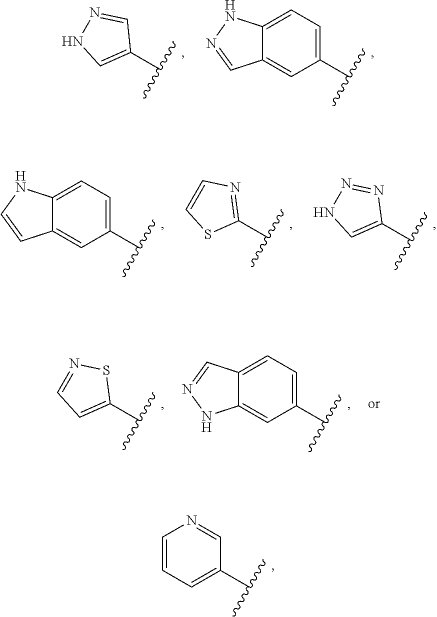

wherein R³ is substituted with 1 or 2 R¹².

In some embodiments, R³ is:

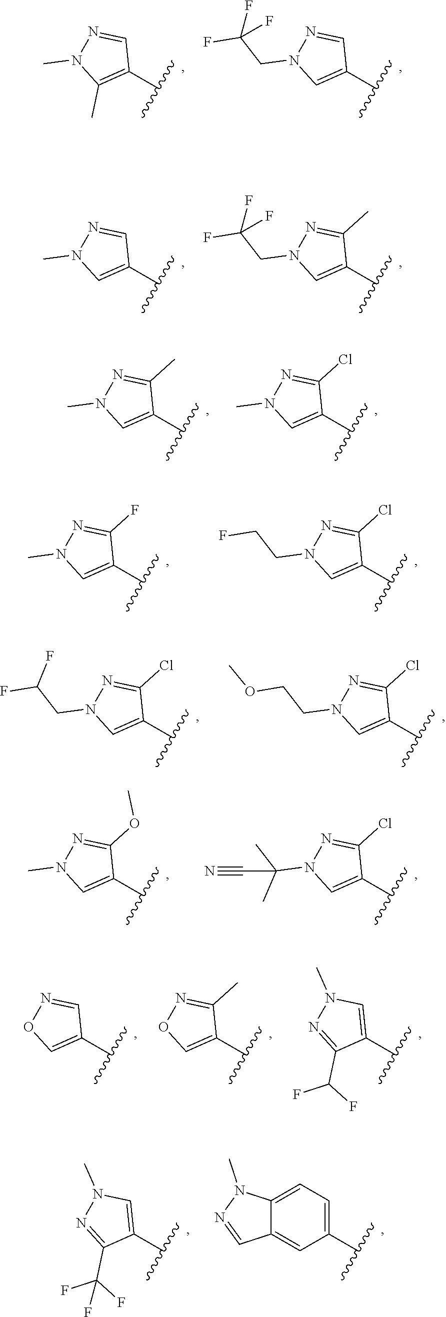

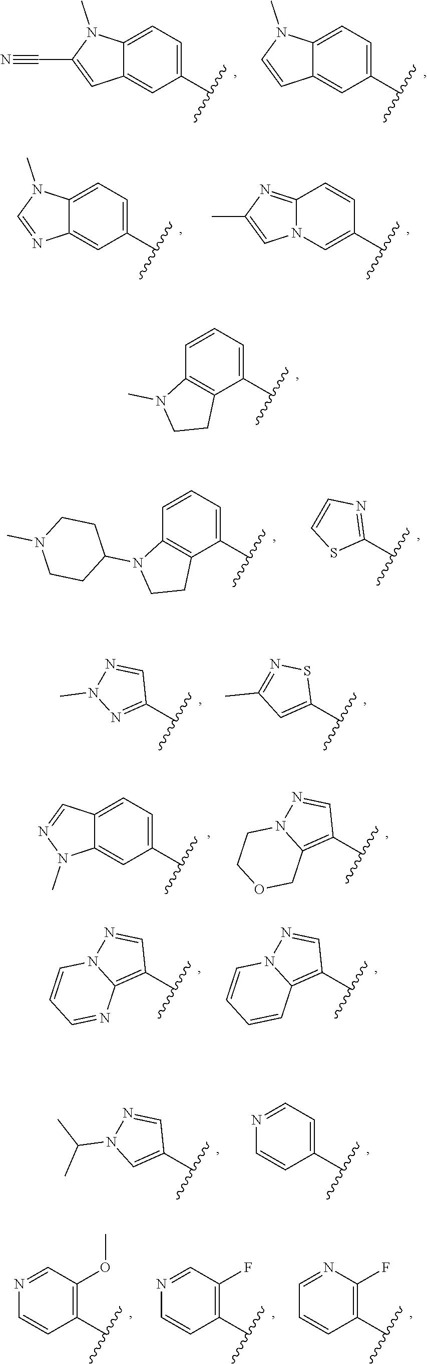

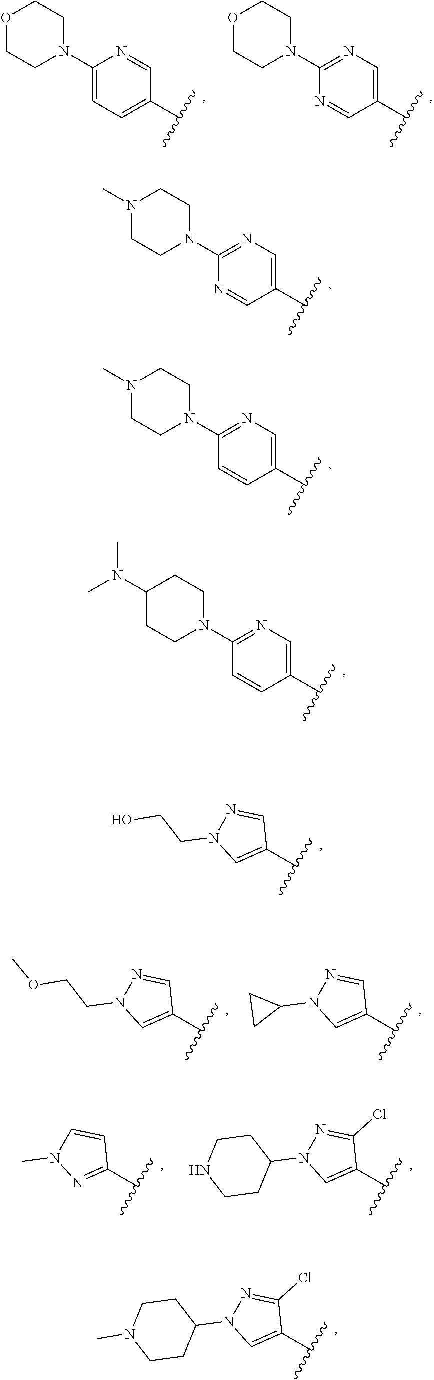

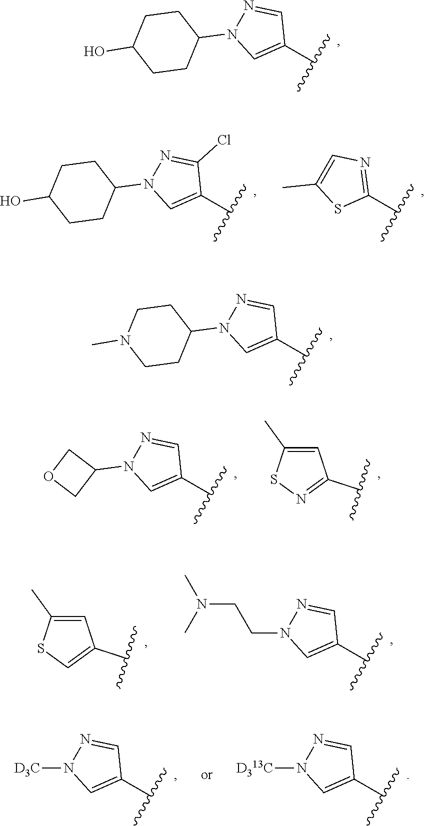

In some embodiments, R³ is:

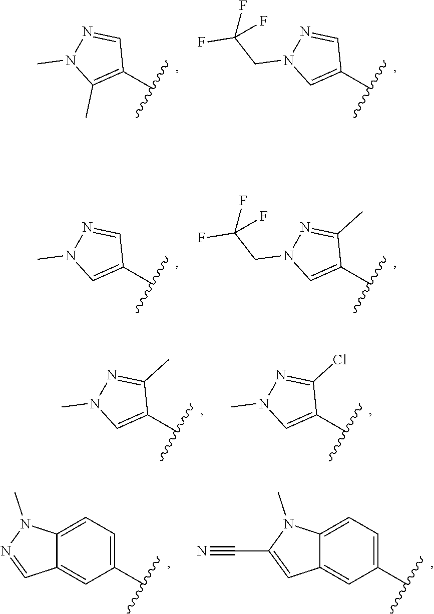

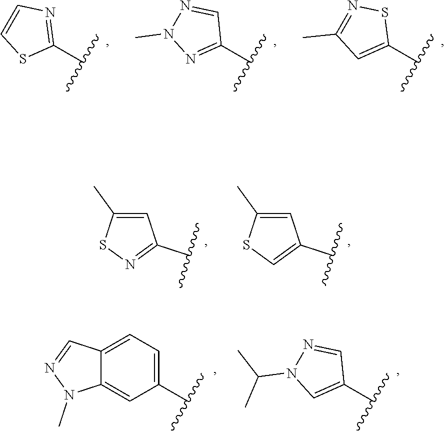

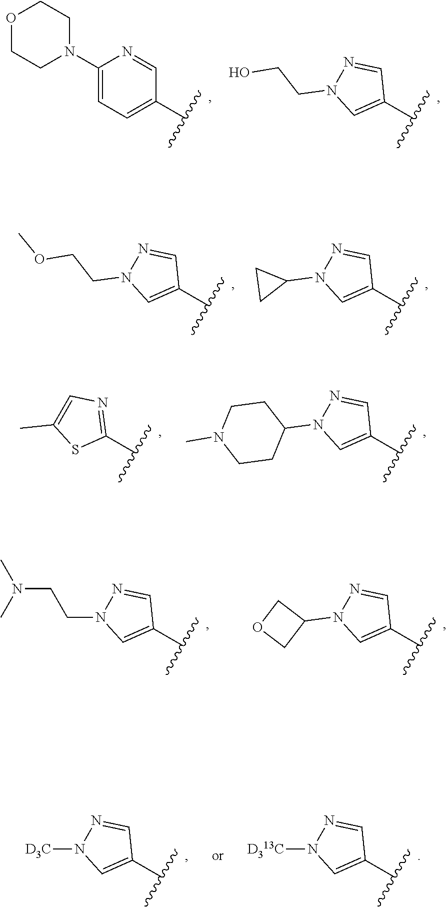

In some embodiments, R³ is:

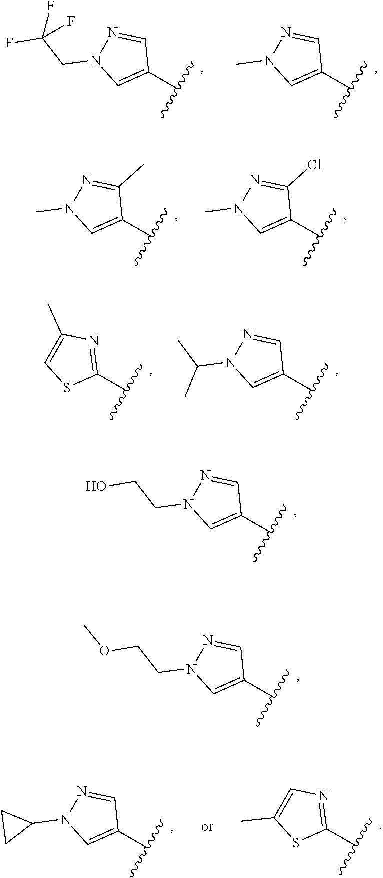

In some embodiments, R³ is

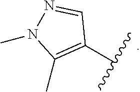 [SMILES: *c1cnn(C)c1C]

In some embodiments, R³ is

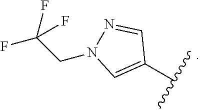 [SMILES: *c1cnn(CC(F)(F)F)c1]

In some embodiments, R³ is

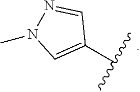 [SMILES: *c1cnn(C)c1]

In some embodiments, R³ is

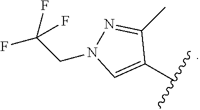 [SMILES: *c1cn(CC(F)(F)F)nc1C]

In some embodiments, R³ is

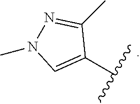 [SMILES: *c1cn(C)nc1C]

In some embodiments, R³ is

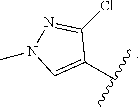 [SMILES: *c1cn(C)nc1Cl]

In some embodiments, R³ is

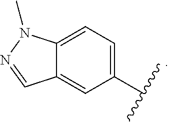 [SMILES: *c1ccc2c(cnn2C)c1]

In some embodiments, R³ is

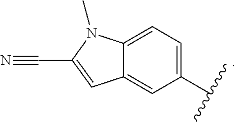 [SMILES: *c1ccc2c(c1)cc(C#N)n2C]

In some embodiments, R³ is

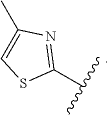 [SMILES: *c1nc(C)cs1]

In some embodiments R³ is

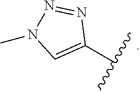 [SMILES: *c1cn(C)nn1]

In some embodiments, R³ is

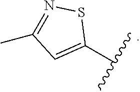 [SMILES: *c1cc(C)ns1]

In some embodiments, R³ is

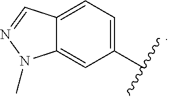 [SMILES: *c1ccc2cnn(C)c2c1]

In some embodiments, R³ is

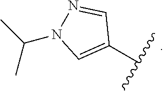 [SMILES: *c1cnn(C(C)C)c1]

In some embodiments, R³ is

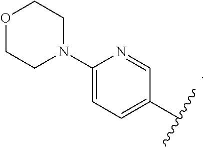 [SMILES: *c1ccc(N2CCOCC2)nc1]

In some embodiments, R³

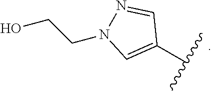 [SMILES: *c1cnn(CCO)c1]

In some embodiments, R³ is

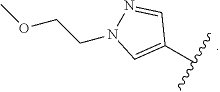 [SMILES: *c1cnn(CCOC)c1]

In some embodiments, R³ is

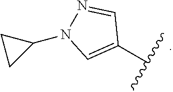 [SMILES: *c1cnn(C2CC2)c1]

In some embodiments, R³ is

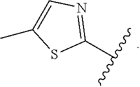 [SMILES: *c1ncc(C)s1]

In some embodiments, R³ is

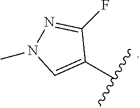 [SMILES: *c1cn(C)nc1F]

In some embodiments, R³ is

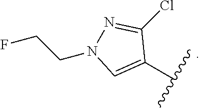 [SMILES: *c1cn(CCF)nc1Cl]

In some embodiments, R³ is

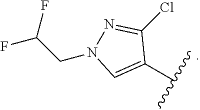 [SMILES: *c1cn(CC(F)F)nc1Cl]

In some embodiments, R³ is

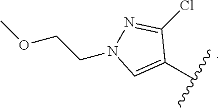 [SMILES: *c1cn(CCOC)nc1Cl]

In some embodiments, R³ is

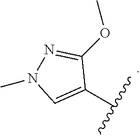 [SMILES: *c1cn(C)nc1OC]

In some embodiments, R³ is

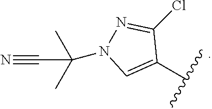 [SMILES: *c1cn(C(C)(C)C#N)nc1Cl]

In some embodiments, R³ is

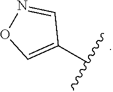 [SMILES: *c1cnoc1]

In some embodiments, R³ is

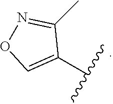 [SMILES: *c1conc1C]

In some embodiments, R³ is

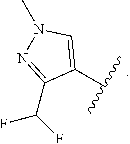 [SMILES: *c1cn(C)nc1C(F)F]

In some embodiments, R³ is

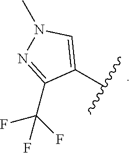 [SMILES: *c1cn(C)nc1C(F)(F)F]

In some embodiments, R³ is

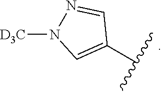 [SMILES: *c1cnn(C([2H])([2H])[2H])c1]

In some embodiments, R³ is

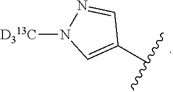

In some embodiments, R³ is

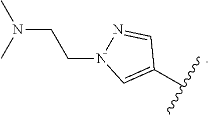 [SMILES: *c1cnn(CCN(C)C)c1]

In some embodiments, R³ is

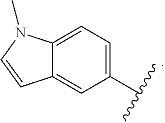 [SMILES: *c1ccc2c(ccn2C)c1]

In some embodiments, R³ is

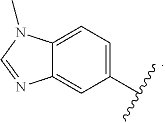 [SMILES: *c1ccc2c(c1)ncn2C]

In some embodiments, R³ is

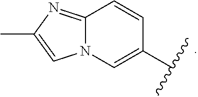 [SMILES: *c1ccc2nc(C)cn2c1]

In some embodiments, R³ is

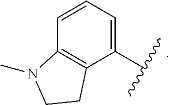 [SMILES: *c1cccc2c1CCN2C]

In some embodiments, R³ is

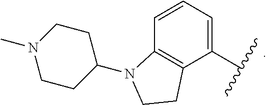 [SMILES: *c1cccc2c1CCN2C1CCN(C)CC1]

In some embodiments, R³ is

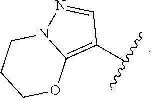 [SMILES: *c1cnn2c1OCCC2]

In some embodiments, R³ is

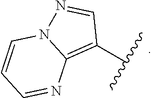 [SMILES: *c1cnn2cccnc12]

In some embodiments, R³ is

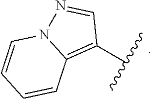 [SMILES: *c1cnn2ccccc12]

In some embodiments, R³ is

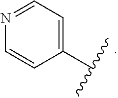 [SMILES: *c1ccncc1]

In some embodiments, R³ is

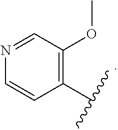 [SMILES: *c1ccncc1OC]

In some embodiments, R³ is

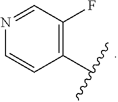 [SMILES: *c1ccncc1F]

In some embodiments, R³ is

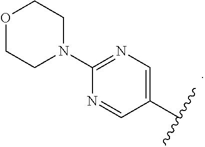 [SMILES: *c1cnc(N2CCOCC2)nc1]

In some embodiments, R³ is

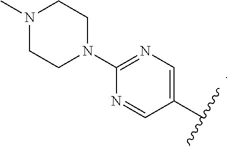 [SMILES: *c1cnc(N2CCN(C)CC2)nc1]

In some embodiments, R³ is

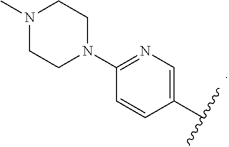 [SMILES: *c1ccc(N2CCN(C)CC2)nc1]

In some embodiments, R³ is

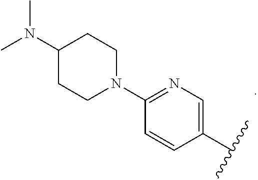 [SMILES: *c1ccc(N2CCC(N(C)C)CC2)nc1]

In some embodiments, R³ is

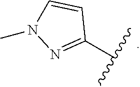 [SMILES: *c1ccn(C)n1]

In some embodiments, R³ is

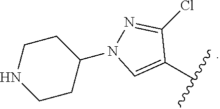 [SMILES: *c1cn(C2CCNCC2)nc1Cl]

In some embodiments, R³ is

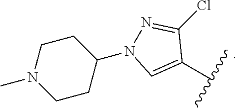 [SMILES: *c1cn(C2CCN(C)CC2)nc1Cl]

In some embodiments, R³ is

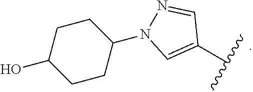 [SMILES: *c1cnn(C2CCC(O)CC2)c1]

In some embodiments, R³ is

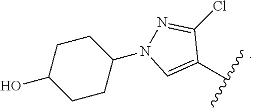 [SMILES: *c1cn(C2CCC(O)CC2)nc1Cl]

In some embodiments, R³ is

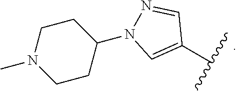 [SMILES: *c1cnn(C2CCN(C)CC2)c1]

In some embodiments, R³ is

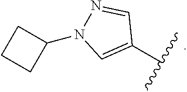 [SMILES: *c1cnn(C2CCC2)c1]

In some embodiments, each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl. In some embodiments, each R¹² is independently alkyl, heteroalkyl, haloalkyl, halo, cyano, heterocycloalkyl, —N(R¹³)₂, or cycloalkyl. In some embodiments, each R¹² is independently aryl. In some embodiments, each R¹² is independently heteroaryl. In some embodiments, each R¹² is independently alkyl. In some embodiments, each R¹² is independently heteroalkyl. In some embodiments, each R¹² is independently haloalkyl. In some embodiments, each R¹² is independently halo. In some embodiments, each R¹² is cyano. In some embodiments, each R¹² is independently alkoxy. In some embodiments, each R¹² is independently heterocycloalkyl. In some embodiments, each R¹² is independently —N(R¹³)₂. In some embodiments, each R¹² is independently —S(═O)₂NH₂. In some embodiments, each R¹² is independently —S(═O)₂alkyl. In some embodiments, each R¹² is independently —S(═O)₂aryl. In some embodiments, each R¹² is independently —S(═O)₂heteroaryl. In some embodiments, each R¹² is independently cycloalkyl. In some embodiments, each R¹² is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, azetidinyl, oxetanyl, pyrrolidinyl, imidazolidinyl, tetrahydrofuranyl, piperidinyl, piperazinyl, tetrahydropyranyl, morpholinyl, —N(R¹³)₂, cyclopropyl, cyclobutyl, cyclopentyl, or cyclohexyl. In some embodiments, each R¹² is independently methyl, iso-propyl, tert-butyl, hydroxyethyl, methoxyethyl, trifluoromethyl, trifluoroethyl, chloro, cyano, morpholinyl, or cyclopropyl. In some embodiments, each R¹² is independently methyl, hydroxyethyl, methoxyethyl, trifluoroethyl, or chloro. In some embodiments, each R¹² is independently methyl or chloro. In some embodiments, each R¹² is methyl. In some embodiments, each R¹² is ethyl. In some embodiments, each R¹² is n-propyl. In some embodiments, each R¹² is iso-propyl. In some embodiments, each R¹² is n-butyl. In some embodiments, each R¹² is iso-butyl. In some embodiments, each R¹² is sec-butyl. In some embodiments, each R¹² is tert-butyl. In some embodiments, each R¹² is hydroxyethyl. In some embodiments, each R¹² is methoxyethyl. In some embodiments, each R¹² is trifluoromethyl. In some embodiments, each R¹² is trifluoroethyl. In some embodiments, each R¹² is pentafluoroethyl. In some embodiments, each R¹² is fluoro. In some embodiments, each R¹² is chloro. In some embodiments, each R¹² is azetidinyl. In some embodiments, each R¹² is oxetanyl. In some embodiments, each R¹² is pyrrolidinyl. In some embodiments, each R¹² is imidazolidinyl. In some embodiments, each R¹² is tetrahydrofuranyl. In some embodiments, each R¹² is piperidinyl. In some embodiments, each R¹² is piperazinyl. In some embodiments, each R¹² is tetrahydropyranyl. In some embodiments, each R¹² is morpholinyl. In some embodiments, each R¹² is cyclopropyl. In some embodiments, each R¹² is cyclobutyl. In some embodiments, each R¹² is cyclopentyl. In some embodiments, each R¹² is cyclohexyl.

In some embodiments, each R¹³ is independently alkyl, cycloalkyl, aryl, or heteroaryl. In some embodiments, each R¹³ is independently alkyl or cycloalkyl. In some embodiments, each R¹³ is independently alkyl. In some embodiments, each R¹³ is independently cycloalkyl. In some embodiments, each R¹³ is independently aryl. In some embodiments, each R¹³ is independently heteroaryl. In some embodiments, each R¹³ is independently methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹³ is independently methyl, ethyl, iso-propyl, tert-butyl, cyclopropyl, cyclopentyl, or cyclohexyl. In some embodiments, each R¹³ is independently methyl, cyclopropyl, or cyclohexyl. In some embodiments, each R¹³ is methyl. In some embodiments, each R¹³ is ethyl. In some embodiments, each R¹³ is n-propyl. In some embodiments, each R¹³ is iso-propyl. In some embodiments, each R¹³ is n-butyl. In some embodiments, each R¹³ is iso-butyl. In some embodiments, each R¹³ is sec-butyl. In some embodiments, each R¹³ is tert-butyl.

In some embodiments, each R¹³ is cyclopropyl. In some embodiments, each R¹³ is cyclobutyl. In some embodiments, each R¹³ is cyclopentyl. In some embodiments, each R¹³ is cyclohexyl.

In some embodiments, the aryl, heteroaryl, heterocycloalkyl, or cycloalkyl of R¹² is unsubstituted. In some embodiments, the aryl, heteroaryl, heterocycloalkyl, or cycloalkyl of R¹² is substituted with 1 or 2 R¹⁴. In some embodiments, the aryl, heteroaryl, heterocycloalkyl, or cycloalkyl of R¹² is substituted with 1 R¹⁴. In some embodiments, the aryl, heteroaryl, heterocycloalkyl, or cycloalkyl of R¹² is substituted with 2 R¹⁴.

In some embodiments, each R¹⁴ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy. In some embodiments, each R¹⁴ is independently alkyl, cycloalkyl, heterocycloalkyl, halo, cyano, —N(R¹¹)₂, or alkoxy. In some embodiments, each R¹⁴ is independently aryl. In some embodiments, each R¹⁴ is independently heteroaryl. In some embodiments, each R¹⁴ is independently alkyl. In some embodiments, each R¹⁴ is independently cycloalkyl. In some embodiments, each R¹⁴ is independently heterocycloalkyl. In some embodiments, each R¹⁴ is independently halo. In some embodiments, each R¹⁴ is independently heteroalkyl. In some embodiments, each R¹⁴ is independently haloalkyl. In some embodiments, each R¹⁴ is cyano. In some embodiments, each R¹⁴ is hydroxy. In some embodiments, each R¹⁴ is amino. In some embodiments, each R¹⁴ is independently —N(R¹⁵)₂. In some embodiments, each R¹⁴ is independently —S(═O)₂alkyl. In some embodiments, each R¹⁴ is independently —S(═O)₂aryl. In some embodiments, each R¹⁴ is independently —S(═O)₂heteroaryl. In some embodiments, each R¹⁴ is independently alkoxy. In some embodiments, each R¹⁴ is independently methyl, ethyl, n-propyl, iso-propyl, n-butyl, iso-butyl, sec-butyl, tert-butyl, cyclopropyl, cyclobutyl, cyclopentyl, cyclohexyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, —N(R¹¹)₂, methoxy, ethoxy, or trifluoromethoxy. In some embodiments, each R¹⁴ is independently methyl, ethyl, iso-propyl, tert-butyl, pyrrolidinyl, piperidinyl, morpholinyl, fluoro, chloro, —N(R¹¹)₂, or methoxy. In some embodiments, each R¹⁴ is methyl. In some embodiments, each R¹⁴ is ethyl. In some embodiments, each R¹⁴ is n-propyl. In some embodiments, each R¹⁴ is iso-propyl. In some embodiments, each R¹⁴ is n-butyl. In some embodiments, each R¹⁴ is iso-butyl. In some embodiments, each R¹⁴ is sec-butyl. In some embodiments, each R¹⁴ is tert-butyl. In some embodiments, each R¹⁴ is cyclopropyl. In some embodiments, each R¹⁴ is cyclobutyl. In some embodiments, each R¹⁴ is cyclopentyl. In some embodiments, each R¹⁴ is cyclohexyl. In some embodiments, each R¹⁴ is azetidinyl. In some embodiments, each R¹⁴ is oxetanyl. In some embodiments, each R¹⁴ is pyrrolidinyl. In some embodiments, each R¹⁴ is imidazolidinyl. In some embodiments, each R¹⁴ is tetrahydrofuranyl. In some embodiments, each R¹⁴ is piperidinyl. In some embodiments, each R¹⁴ is piperazinyl. In some embodiments, each R¹⁴ is tetrahydropyranyl. In some embodiments, each R¹⁴ is morpholinyl. In some embodiments, each R¹⁴ is fluoro. In some embodiments, each R¹⁴ is chloro. In some embodiments, each R¹⁴ is methoxy. In some embodiments, each R¹⁴ is ethoxy. In some embodiments, each R¹⁴ is trifluoromethoxy.

In some embodiments, each R¹¹ is independently alkyl, cycloalkyl, aryl, or heteroaryl. In some embodiments, each R¹¹ is independently alkyl or cycloalkyl. In some embodiments, each R¹¹ is methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹¹ is methyl. In some embodiments, each R¹¹ is ethyl. In some embodiments, each R¹¹ is n-propyl. In some embodiments, each R¹¹ is iso-propyl. In some embodiments, each R¹¹ is n-butyl. In some embodiments, each R¹¹ is iso-butyl. In some embodiments, each R¹¹ is sec-butyl. In some embodiments, each R¹⁵ is tert-butyl. In some embodiments, each R¹⁵ is cyclopropyl. In some embodiments, each R¹⁵ is cyclobutyl. In some embodiments, each R¹⁵ is cyclopentyl. In some embodiments, each R¹⁵ is cyclohexyl.

In some embodiments:
X is —NH— or —O—;
n is 0;
R⁵ is phenyl substituted with 2 or 3 R^{5′};
R² is phenyl substituted with at least one R⁷ and 0, 1, or 2 R⁸; and
R³ is pyrazolyl substituted with 0, 1, 2, or 3 R¹².

In some embodiments, X is —NH—.

In some embodiments, R^{5′} is fluoromethyl, difluoromethyl, or trifluoromethyl.

In some embodiments:
R⁷ is

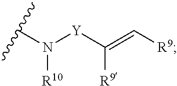

and
R⁸ is halo.

In some embodiments:
R⁸ is fluoro;
Y is —C(═O)—;
R⁹ and R^{9′} are hydrogen; and
R¹⁰ is hydrogen.

In some embodiments, R¹² is alkyl.

In some embodiments, R¹² is methyl.

In some embodiments, the compound is of Formula I-A, Formula I-B, Formula I-C, Formula I-D, Formula I-E, Formula I-F, or Formula I-G:

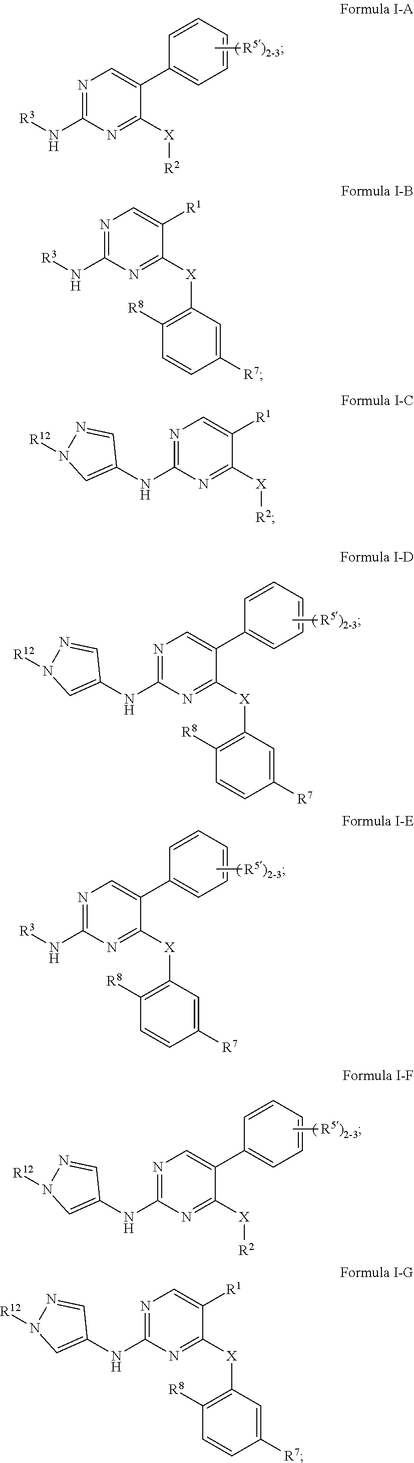

or a pharmaceutically acceptable salt or stereoisomer thereof.

In some embodiments, the compound is of Formula I-B:

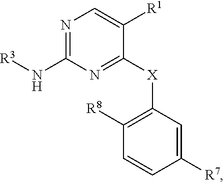

or a pharmaceutically acceptable salt or stereoisomer thereof. In some embodiments of the compound of Formula I-B, wherein R¹ is R⁵. In some embodiments of the compound of Formula I-B, wherein R¹ is R⁵; and R⁵ is substituted with 2 R^{5′}. In some embodiments of the compound of Formula I-B, wherein R¹ is R⁵; and R⁵ is substituted with 3 R^{5′}. In some embodiments of the compound of Formula I-B, R⁵ is phenyl or C-linked pyridyl; wherein the phenyl or C-linked pyridyl is substituted with 2 or 3 R^{5′}. In some embodiments of the compound of Formula I-B, R⁵ is phenyl or C-linked pyridyl; wherein the phenyl or C-linked pyridyl is substituted with 2 R^{5′}. In some embodiments of the compound of Formula I-B, R⁵ is phenyl or C-linked pyridyl; wherein the phenyl or C-linked pyridyl is substituted with 3 R^{5′}. In some embodiments of the compound of Formula I-B, two adjacent R^{5′} groups come together to form a 5- to 10-membered heterocycle.

In some embodiments, the compound is of Formula I-C:

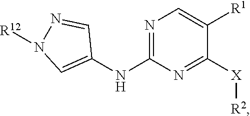

or a pharmaceutically acceptable salt or stereoisomer thereof. In some embodiments of the compound of Formula I-C, wherein R¹ is R⁵; and R⁵ is substituted with 2 R^{5′}. In some embodiments of the compound of Formula I-C, wherein R¹ is R⁵; and R⁵ is substituted with 3 R^{5′}. In some embodiments of the compound of Formula I-C, R⁵ is phenyl or C-linked pyridyl; wherein the phenyl or C-linked pyridyl is substituted with 2 or 3 R^{5′}. In some embodiments of the compound of Formula I-C, R⁵ is phenyl or C-linked pyridyl; wherein the phenyl or C-linked pyridyl is substituted with 2 R^{5′}. In some embodiments of the compound of Formula I-C, R⁵ is phenyl or C-linked pyridyl; wherein the phenyl or C-linked pyridyl is substituted with 3 R^{5′}. In some embodiments of the compound of Formula I-C, two adjacent R^{5′} groups come together to form a 5- to 10-membered heterocycle.

In some embodiments, the compound of Formula I is:

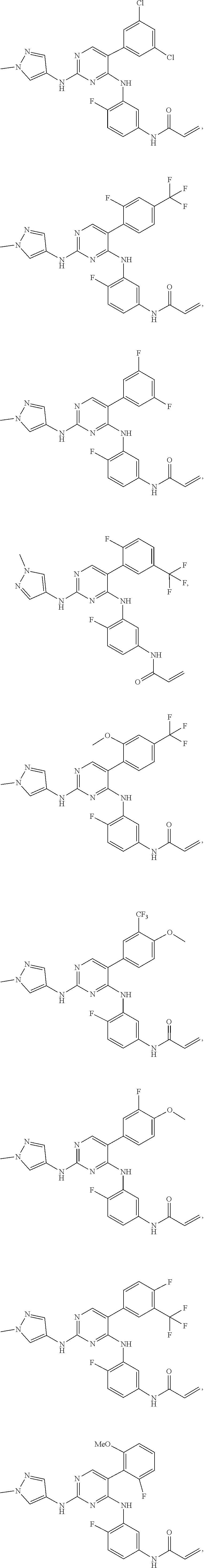

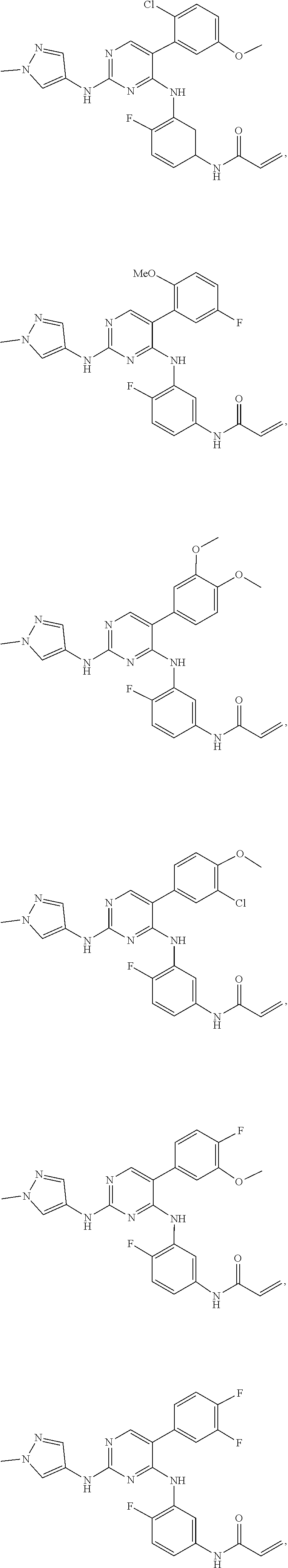

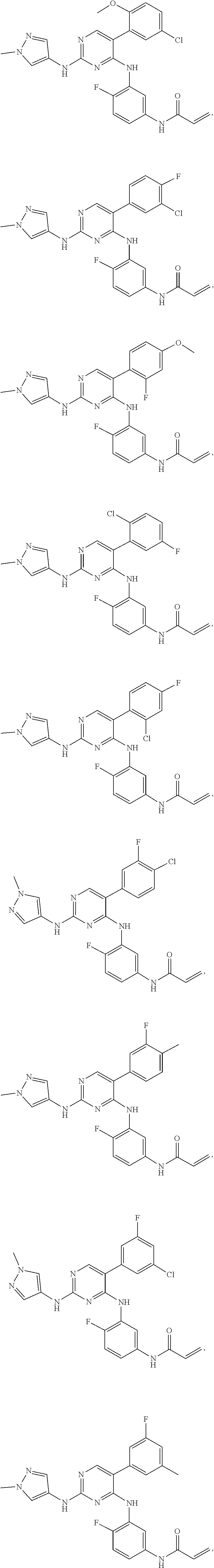

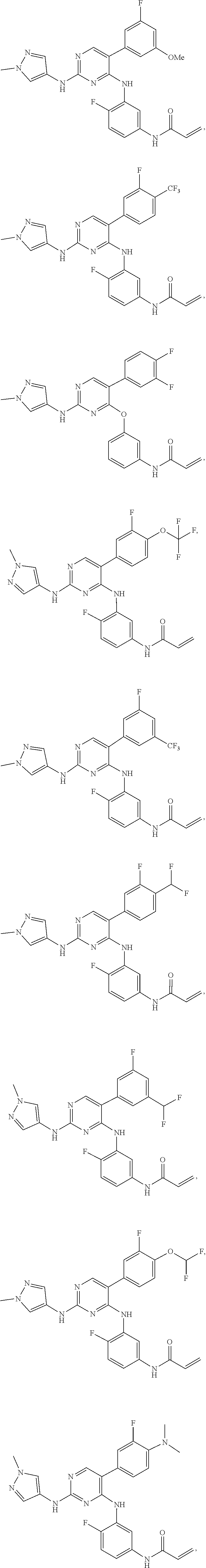

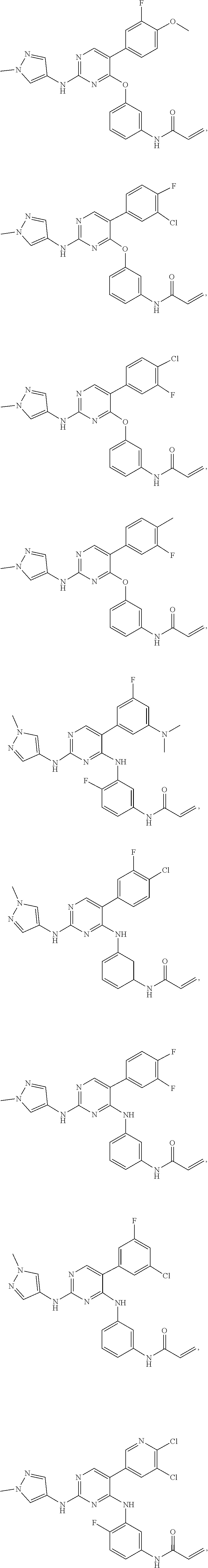

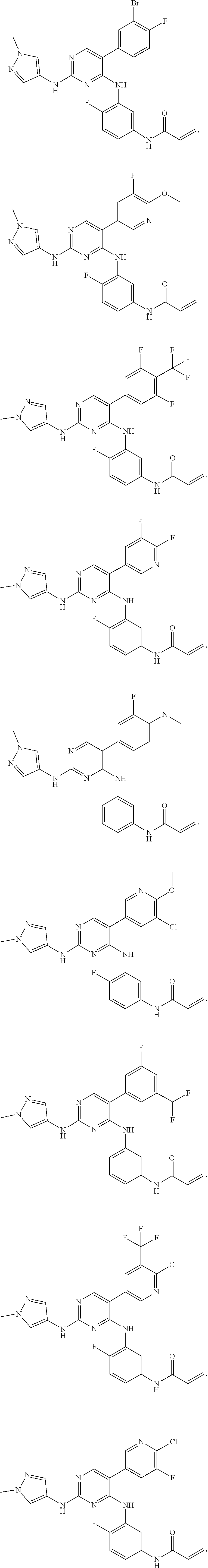

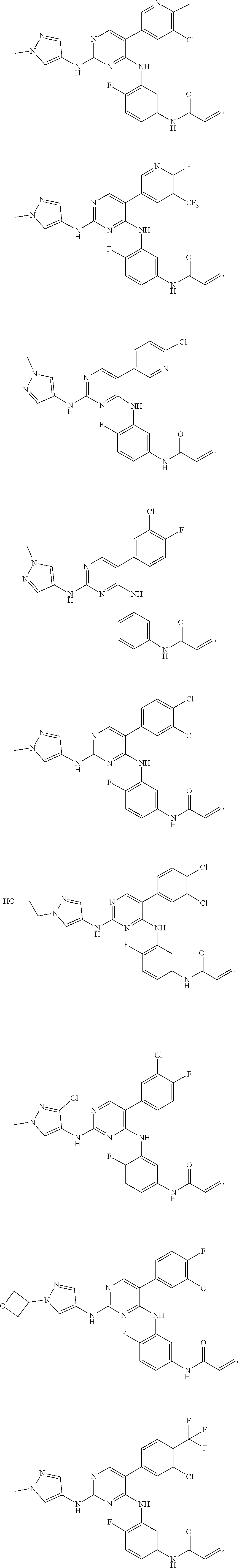

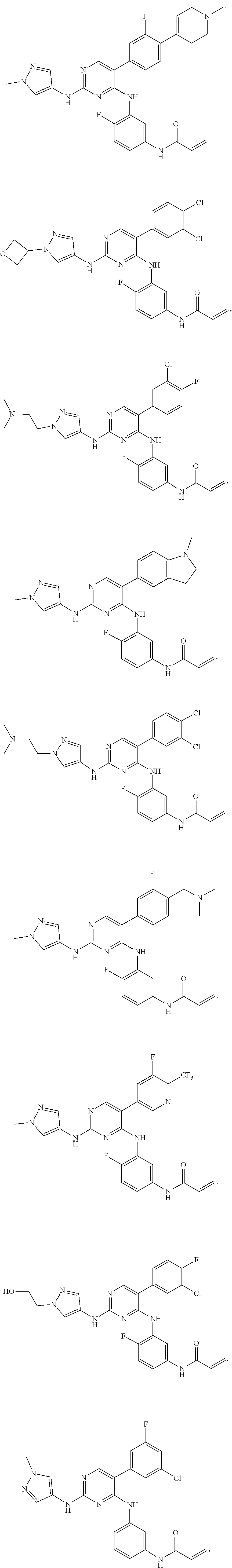

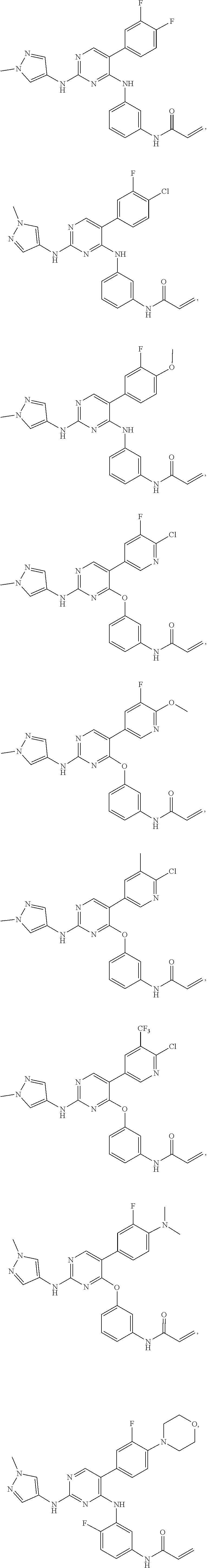

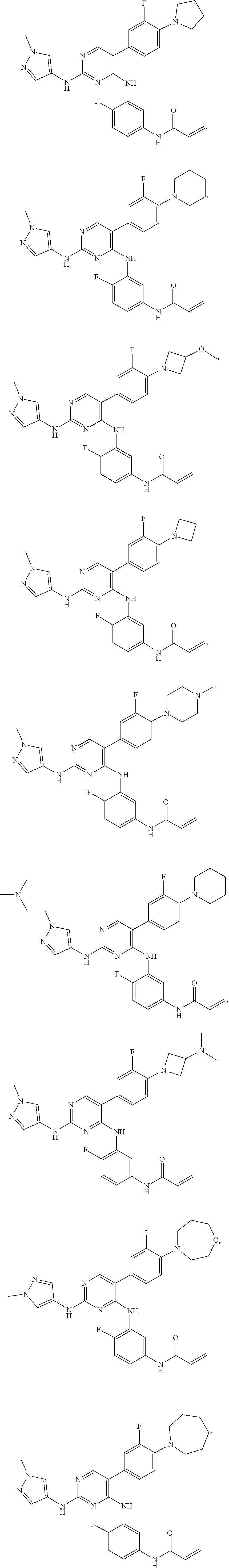

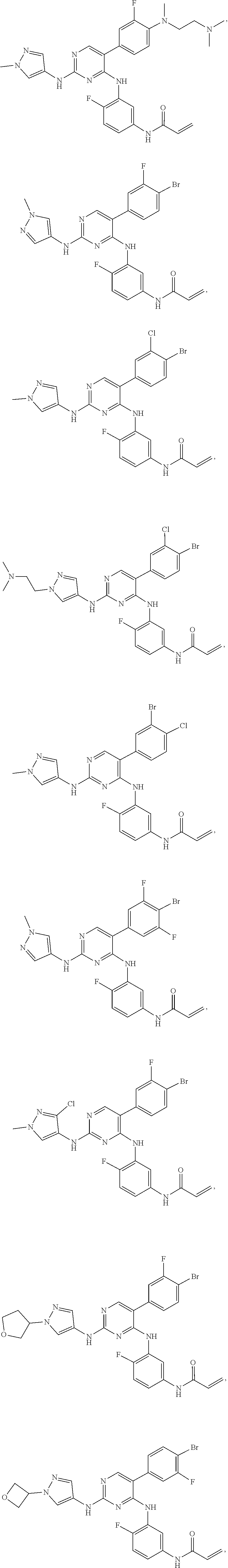

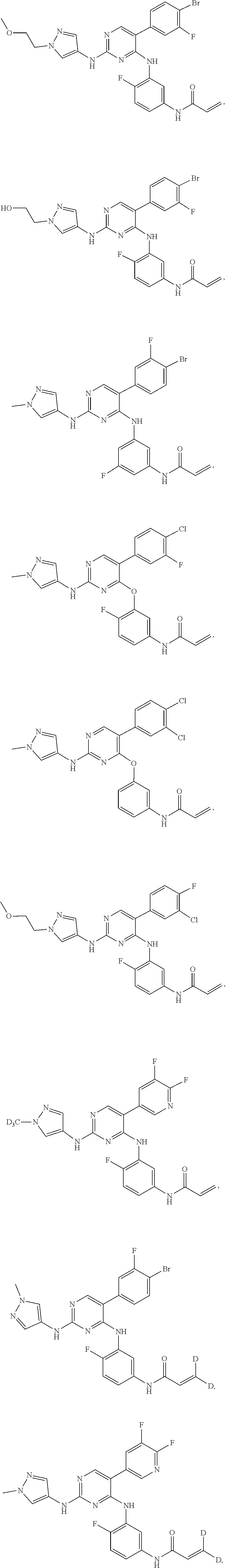

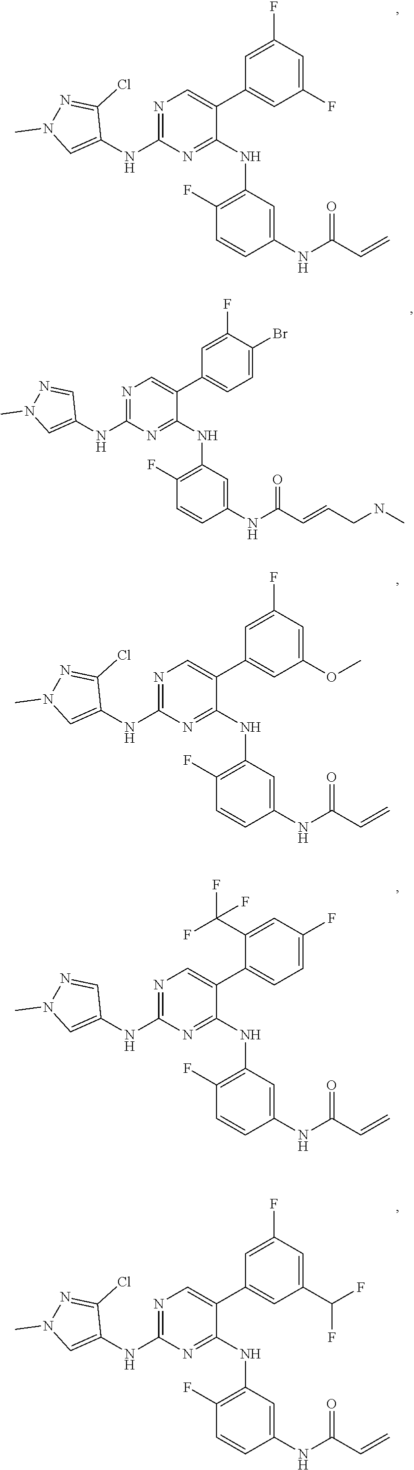

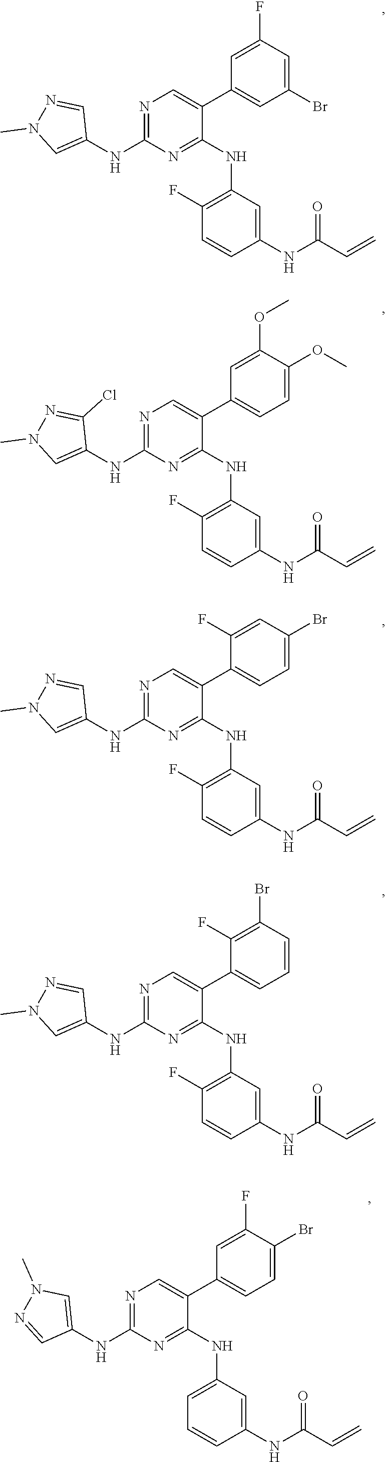

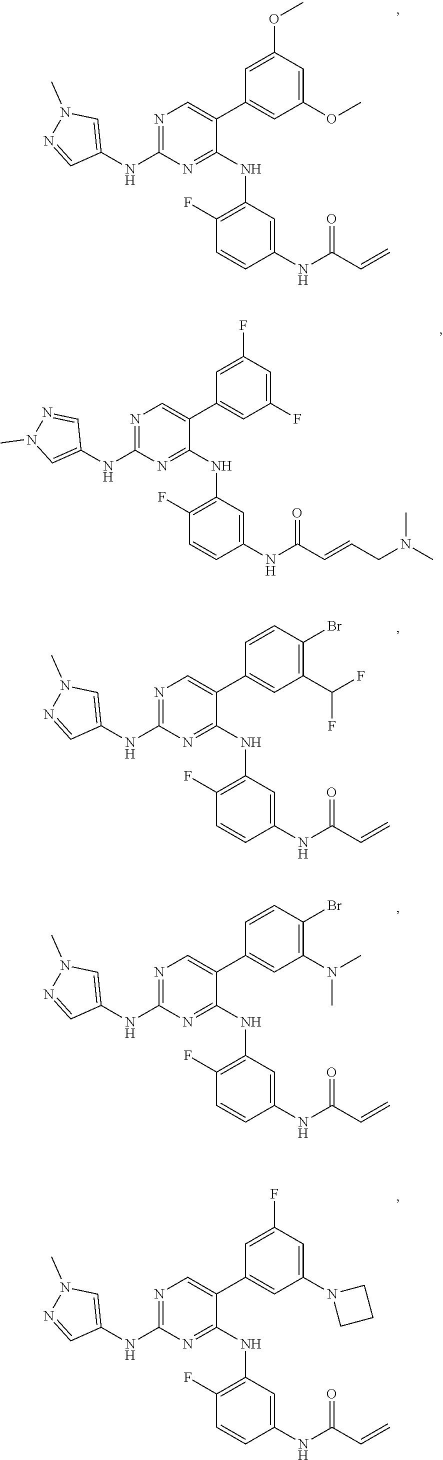

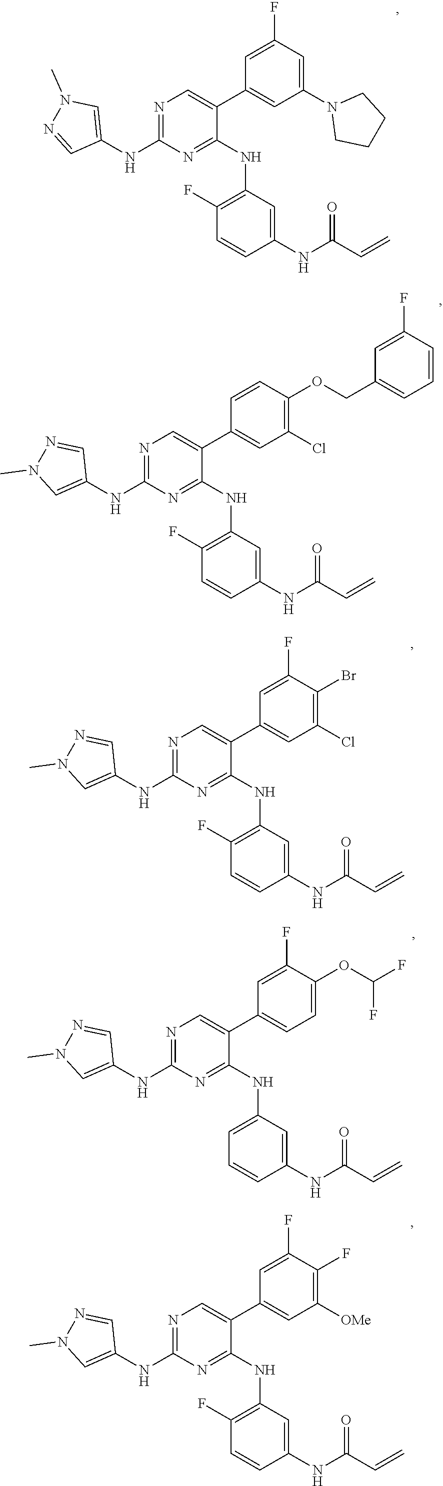

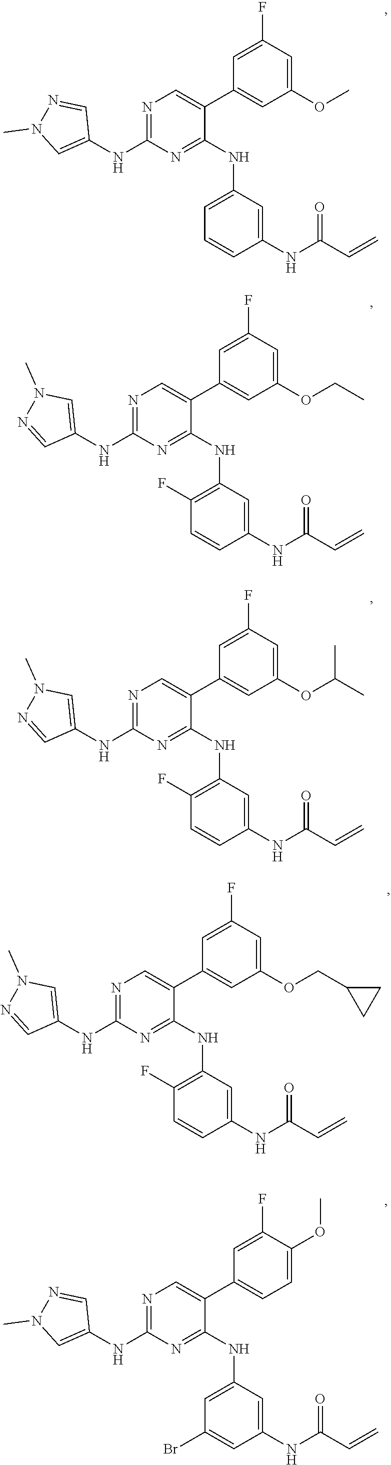

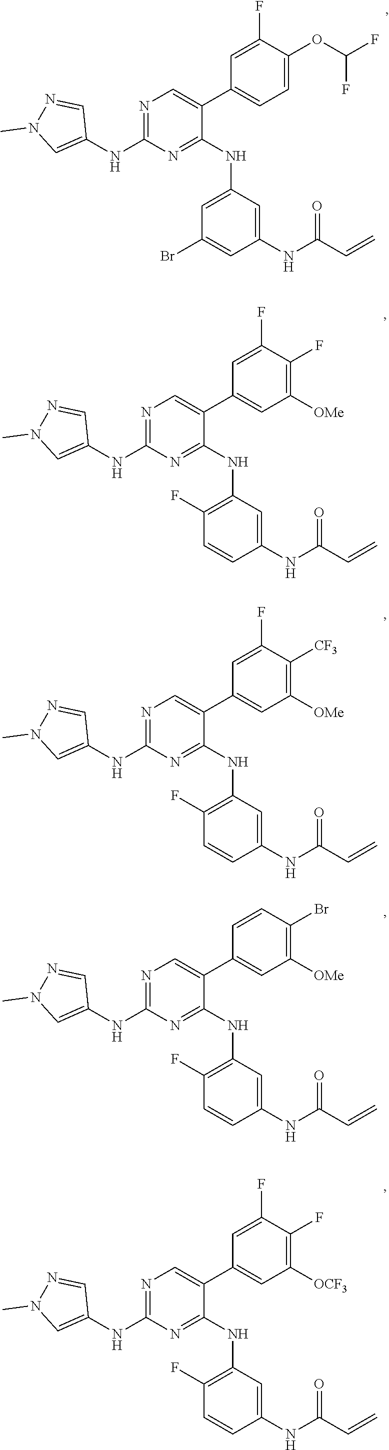

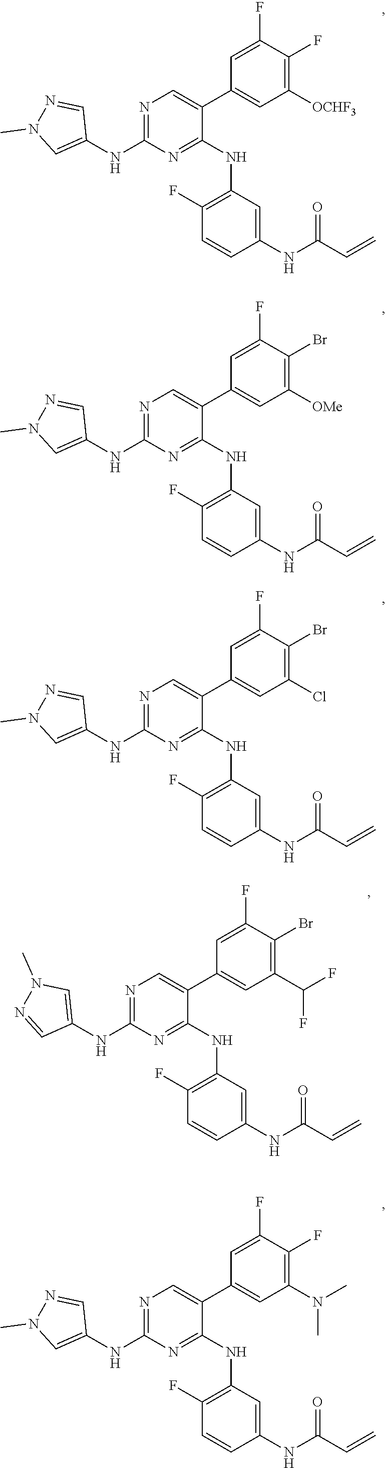

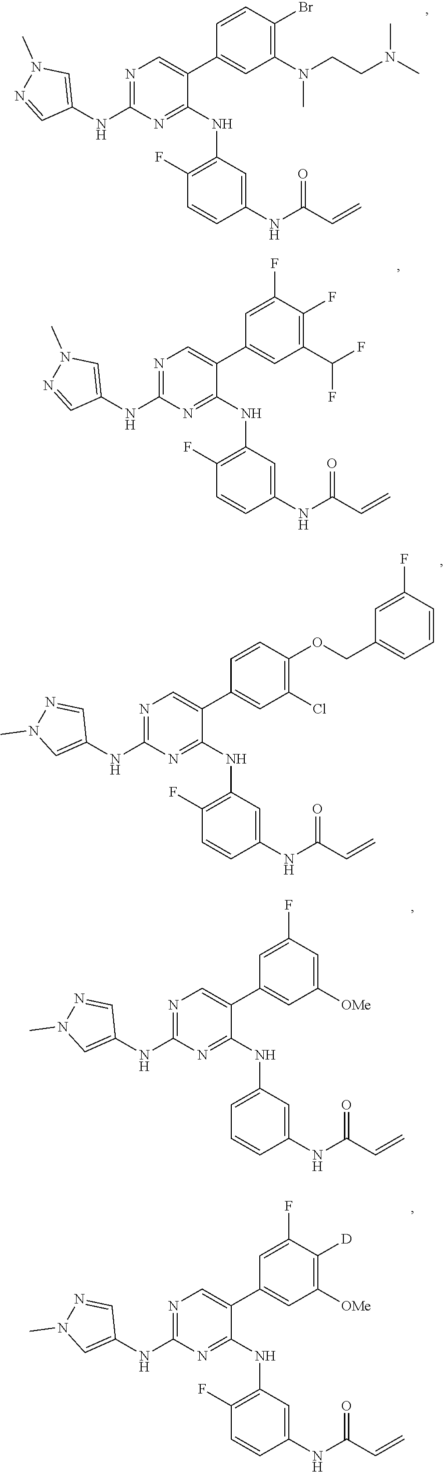

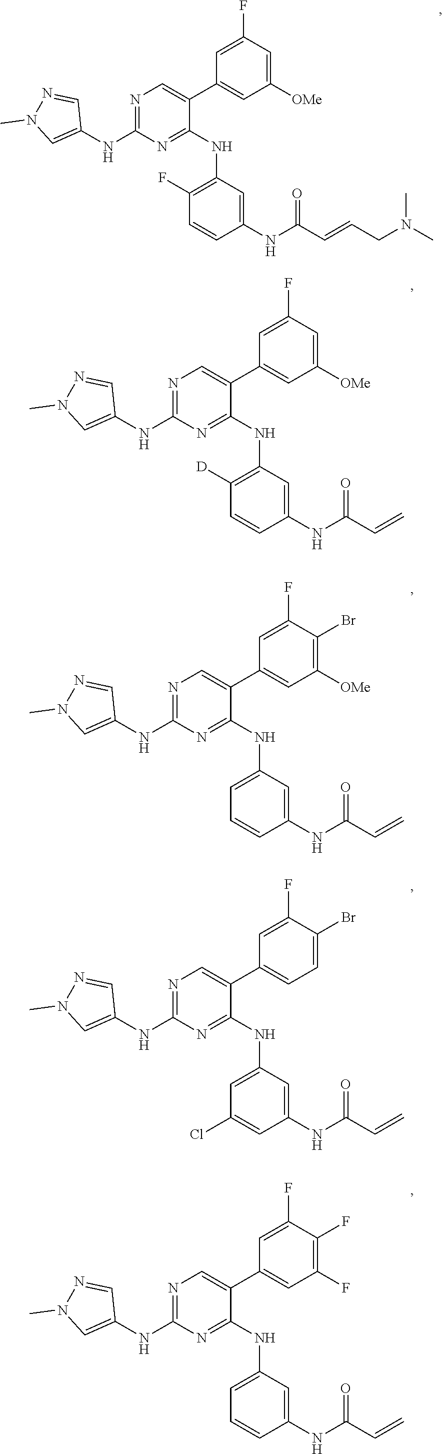

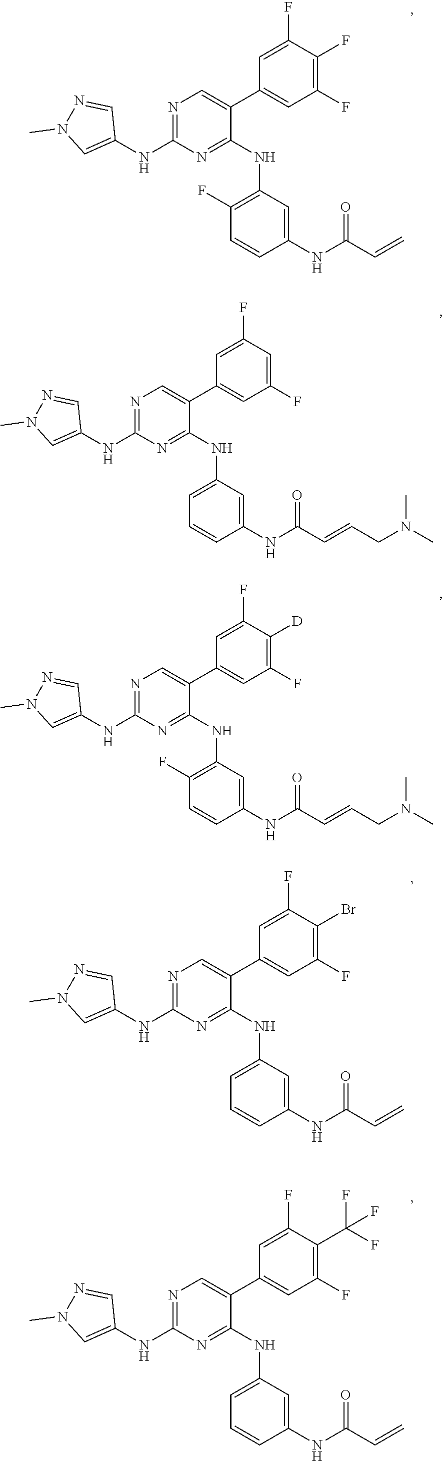

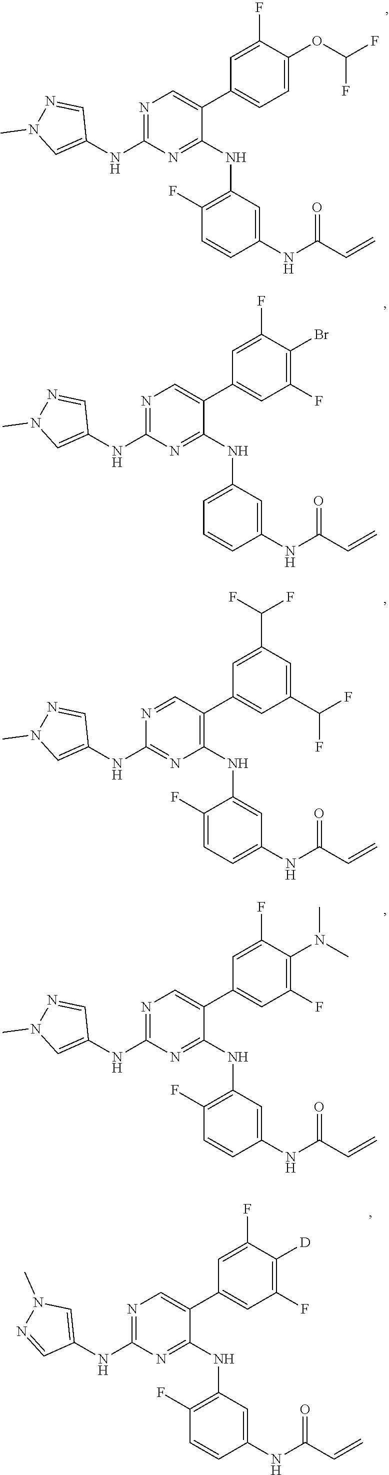

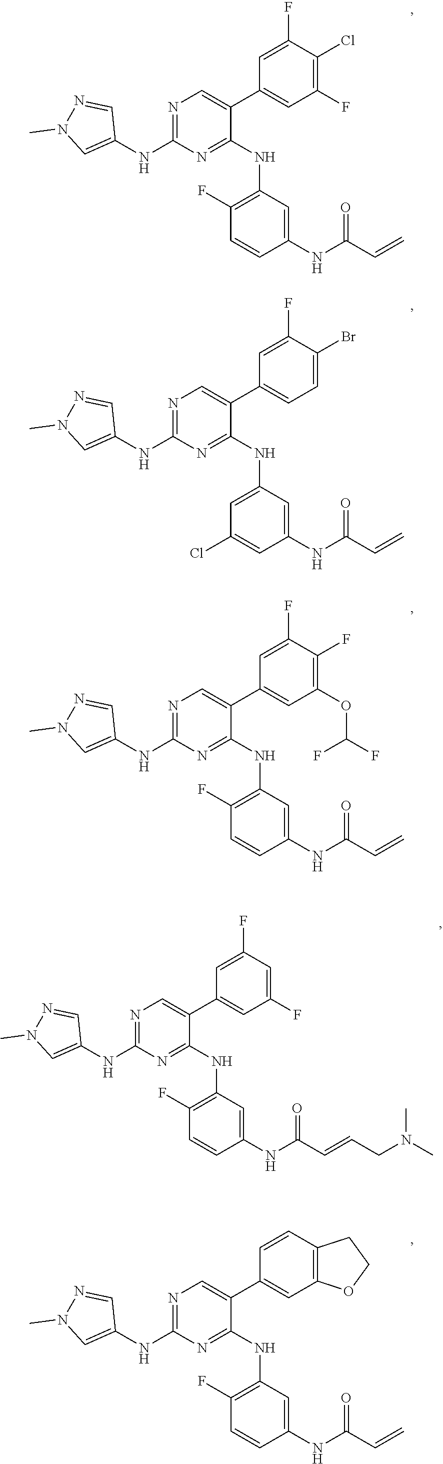

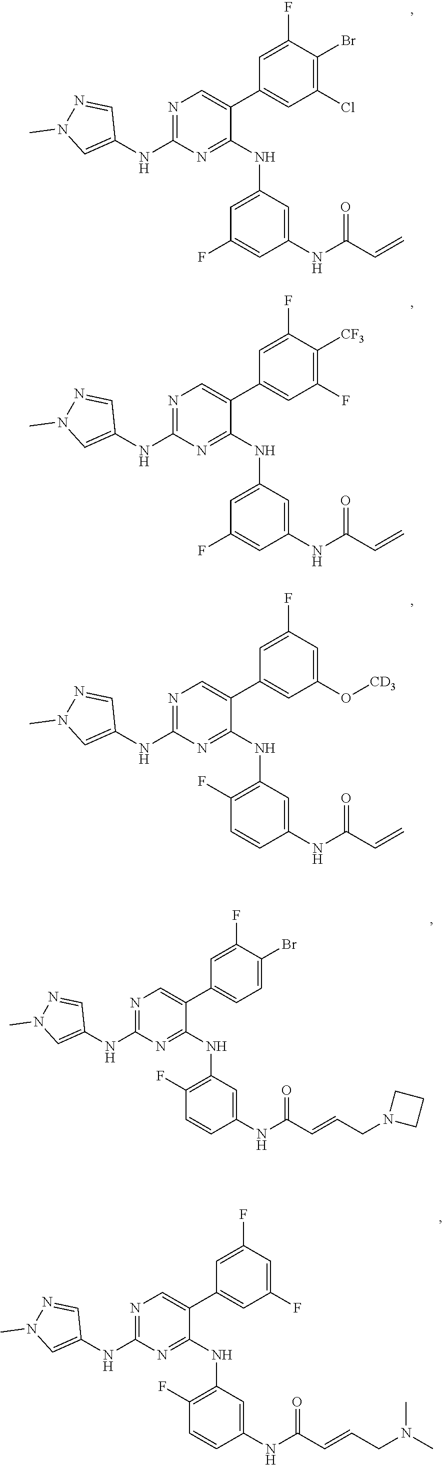

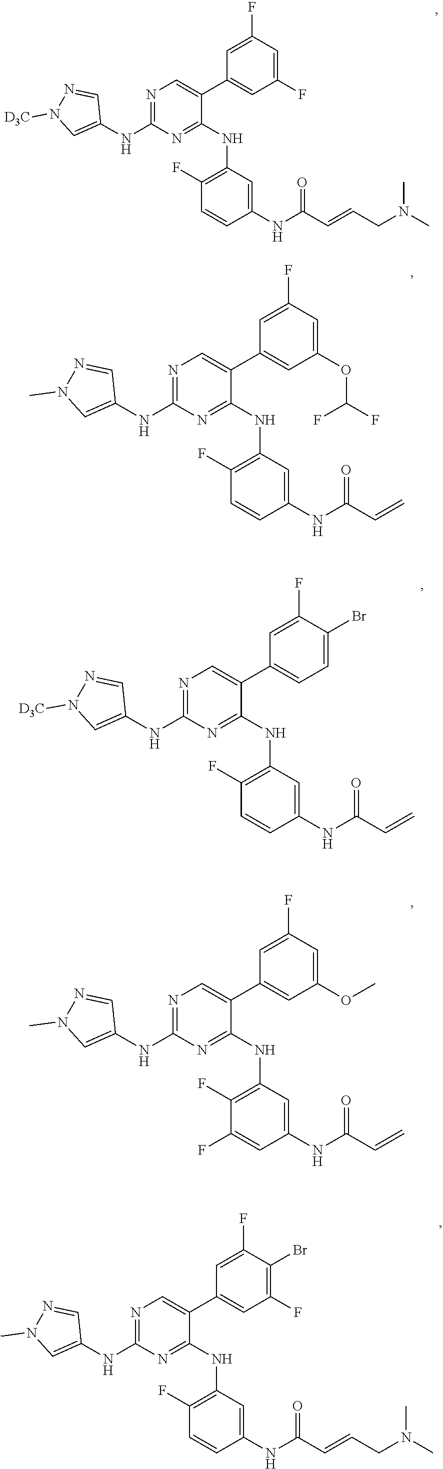

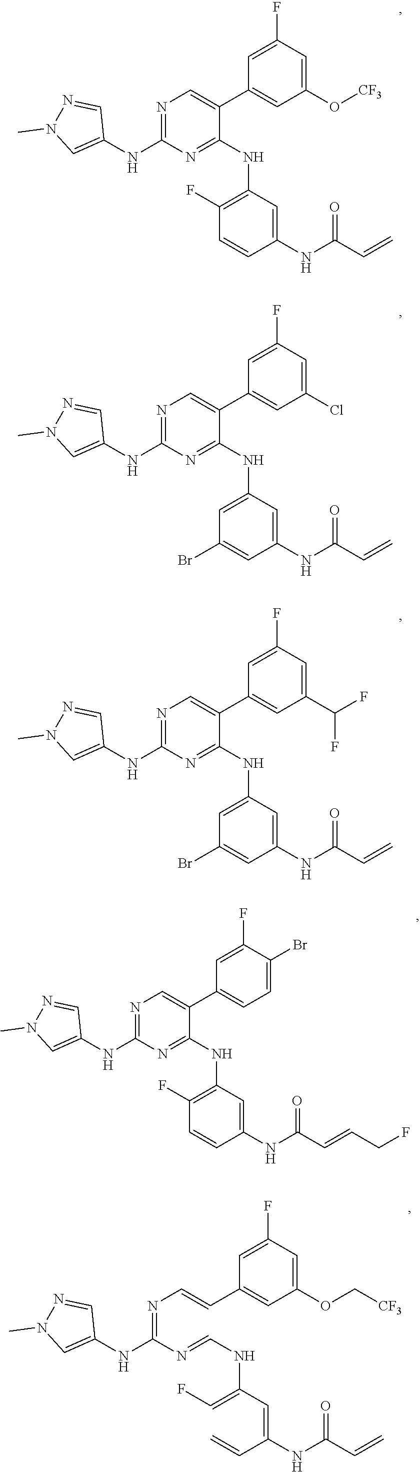

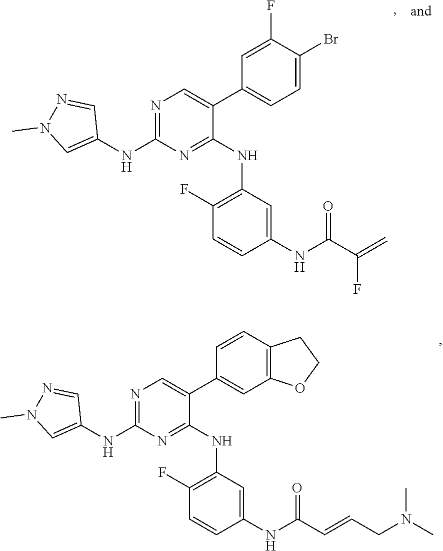

or a pharmaceutically acceptable salt or stereoisomer thereof.

In another aspect, the present disclosure provides a pharmaceutical composition comprising a compound of Formula I, or a pharmaceutically acceptable salt or stereoisomer thereof, and a pharmaceutically acceptable carrier.

Particular embodiments of the present disclosure are compounds of Formula I or its stereoisomers, tautomers, pharmaceutically acceptable salts, stereoisomers, solvates, and hydrates thereof, selected from the group consisting of,
N-(4-fluoro-3-((5-(3-fluoro-4-methoxyphenyl)-2-((1-methyl-1H-pyrazol-4-yl)amino)pyrimidin-4-yl)amino)phenyl)acrylamide (Compound 1),
N-(3-((5-(3,4-dimethoxyphenyl)-2-((1-methyl-1H-pyrazol-4-yl)amino)pyrimidin-4-yl)amino)-4-fluorophenyl)acrylamide (Compound 2)
N-(3-((5-(3-chloro-4-methoxyphenyl)-2-((1-methyl-1H-pyrazol-4-yl)amino)pyrimidin-4-yl)amino)-4-fluorophenyl)acrylamide (Compound 3),
N-(4-fluoro-3-((5-(4-fluoro-3-methoxyphenyl)-2-((1-methyl-1H-pyrazol-4-yl)amino)pyrimidin-4-yl)amino)phenyl)acrylamide (Compound 4),
N-(3-((5-(3,4-difluorophenyl)-2-((1-methyl-1H-pyrazol-4-yl)amino)pyrimidin-4-yl)amino)-4-fluorophenyl)acrylamide (Compound 5),
N-(3-((5-(3-chloro-4-fluorophenyl)-2-((1-methyl-1H-pyrazol-4-yl)amino)pyrimidin-4-yl)amino)-4-fluorophenyl)acrylamide (Compound 6),
N-(4-fluoro-3-((5-(2-fluoro-4-methoxyphenyl)-2-((1-methyl-1H-pyrazol-4-yl)amino)pyrimidin-4-yl)amino)phenyl)acrylamide (Compound 7),
N-(3-((5-(2-chloro-5-methoxyphenyl)-2-((1-methyl-1H-pyrazol-4-yl)amino)pyrimidin-4-yl)amino)-4-fluorophenyl)acrylamide (Compound 8),
N-(4-fluoro-3-((5-(5-fluoro-2-methoxyphenyl)-2-((1-methyl-1H-pyrazol-4-yl)amino)pyrimidin-4-yl)amino)phenyl)acrylamide (Compound 9),
N-(4-fluoro-3-((5-(2-methoxy-4-(trifluoromethyl)phenyl)-2-((1-methyl-1H-pyrazol-4-yl)amino)pyrimidin-4-yl)amino)phenyl)acrylamide (Compound 10),
N-(3-((5-(2-chloro-5-fluorophenyl)-2-((1-methyl-1H-pyrazol-4-yl)amino)pyrimidin-4-yl)amino)-4-fluorophenyl)acrylamide (Compound 11),
N-(3-((5-(2-chloro-4-fluorophenyl)-2-((1-methyl-1H-pyrazol-4-yl)amino)pyrimidin-4-yl)amino)-4-fluorophenyl)acrylamide (Compound 12),
N-(4-fluoro-3-((5-(3-fluoro-4-methylphenyl)-2-((1-methyl-1H-pyrazol-4-yl)amino)pyrimidin-4-yl)amino)phenyl)acrylamide (Compound 13),
N-(4-fluoro-3-((5-(3-fluoro-4-(trifluoromethoxy)phenyl)-2-((1-methyl-1H-pyrazol-4-yl)amino)pyrimidin-4-yl)amino)phenyl)acrylamide (Compound 14),
N-(3-((5-(3-(difluoromethyl)-5-fluorophenyl)-2-((1-methyl-1H-pyrazol-4-yl)amino)pyrimidin-4-yl)amino)-4-fluorophenyl)acrylamide (Compound 15),
N-(3-((5-(4-(difluoromethoxy)-3-fluorophenyl)-2-((1-methyl-1H-pyrazol-4-yl)amino)pyrimidin-4-yl)amino)-4-fluorophenyl)acrylamide (Compound 16),
N-(3-((5-(5,6-dichloropyridin-3-yl)-2-((1-methyl-1H-pyrazol-4-yl)amino)pyrimidin-4-yl)amino)-4-fluorophenyl)acrylamide (Compound 17),
N-(3-((5-(3-bromo-4-fluorophenyl)-2-((1-methyl-1H-pyrazol-4-yl)amino)pyrimidin-4-yl)amino)-4-fluorophenyl)acrylamide trifluoroacetate (Compound 18),
N-(4-fluoro-3-((5-(5-fluoro-6-methoxypyridin-3-yl)-2-((1-methyl-1H-pyrazol-4-yl)amino)pyrimidin-4-yl)amino)phenyl)acrylamide trifluoroacetate (Compound 19),
N-(3-((5-(3,5-difluoro-4-(trifluoromethyl)phenyl)-2-((1-methyl-1H-pyrazol-4-yl)amino)pyrimidin-4-yl)amino)-4-fluorophenyl)acrylamide trifluoroacetate (Compound 20),
N-(4-fluoro-3-((5-(2-fluoro-4-(trifluoromethyl)phenyl)-2-((1-methyl-1H-pyrazol-4-yl)amino)pyrimidin-4-yl)amino)phenyl)acrylamide (Compound 21),
N-(4-fluoro-3-((5-(4-methoxy-3-(trifluoromethyl)phenyl)-2-((1-methyl-1H-pyrazol-4-yl)amino)pyrimidin-4-yl)amino)phenyl)acrylamide (Compound 22),
N-(3-((5-(5-chloro-2-methoxyphenyl)-2-((1-methyl-1H-pyrazol-4-yl)amino)pyrimidin-4-yl)amino)-4-fluorophenyl)acrylamide (Compound 23),
N-(4-fluoro-3-((5-(2-fluoro-5-(trifluoromethyl)phenyl)-2-((1-methyl-1H-pyrazol-4-yl)amino)pyrimidin-4-yl)amino)phenyl)acrylamide (Compound 24),
N-(4-fluoro-3-((5-(4-fluoro-3-(trifluoromethyl)phenyl)-2-((1-methyl-1H-pyrazol-4-yl)amino)pyrimidin-4-yl)amino)phenyl)acrylamide (Compound 25),
N-(3-((5-(3,5-difluorophenyl)-2-((1-methyl-1H-pyrazol-4-yl)amino)pyrimidin-4-yl)amino)-4-fluorophenyl)acrylamide (Compound 26),
N-(4-fluoro-3-((5-(3-fluoro-5-(trifluoromethyl)phenyl)-2-((1-methyl-1H-pyrazol-4-yl)amino)pyrimidin-4-yl)amino)phenyl)acrylamide (Compound 27),
N-(3-((5-(4-(dimethylamino)-3-fluorophenyl)-2-((1-methyl-1H-pyrazol-4-yl)amino)pyrimidin-4-yl)amino)-4-fluorophenyl)acrylamide (Compound 28),
N-(3-((5-(5,6-difluoropyridin-3-yl)-2-((1-methyl-1H-pyrazol-4-yl)amino)pyrimidin-4-yl)amino)-4-fluorophenyl)acrylamide (Compound 29),
N-(3-((5-(4-(dimethylamino)-3-fluorophenyl)-2-((1-methyl-1H-pyrazol-4-yl)amino)pyrimidin-4-yl)amino)phenyl)acrylamide (Compound 30),
N-(3-((5-(5-chloro-6-methoxypyridin-3-yl)-2-((1-methyl-1H-pyrazol-4-yl)amino)pyrimidin-4-yl)amino)-4-fluorophenyl)acrylamide trifluoroacetate (Compound 31),
N-(3-((5-(3,5-dichlorophenyl)-2-((1-methyl-1H-pyrazol-4-yl)amino)pyrimidin-4-yl)amino)-4-fluorophenyl)acrylamide (Compound 32),
N-(3-((5-(4-chloro-3-fluorophenyl)-2-((1-methyl-1H-pyrazol-4-yl)amino)pyrimidin-4-yl)amino)-4-fluorophenyl)acrylamide (Compound 33),
N-(3-((5-(3-chloro-5-fluorophenyl)-2-((1-methyl-1H-pyrazol-4-yl)amino)pyrimidin-4-yl)amino)-4-fluorophenyl)acrylamide (Compound 34),
N-(4-fluoro-3-((5-(3-fluoro-4-(trifluoromethyl)phenyl)-2-((1-methyl-1H-pyrazol-4-yl)amino)pyrimidin-4-yl)amino)phenyl)acrylamide (Compound 35),
N-(3-((5-(3,4-difluorophenyl)-2-((1-methyl-1H-pyrazol-4-yl)amino)pyrimidin-4-yl)oxy)phenyl)acrylamide (Compound 36),
N-(3-((5-(4-(difluoromethyl)-3-fluorophenyl)-2-((1-methyl-1H-pyrazol-4-yl)amino)pyrimidin-4-yl)amino)-4-fluorophenyl)acrylamide (Compound 37),
N-(3-((5-(3-fluoro-4-methoxyphenyl)-2-((1-methyl-1H-pyrazol-4-yl)amino)pyrimidin-4-yl)oxy)phenyl)acrylamide (Compound 38),
N-(3-((5-(3-chloro-4-fluorophenyl)-2-((1-methyl-1H-pyrazol-4-yl)amino)pyrimidin-4-yl)oxy)phenyl)acrylamide (Compound 39),
N-(3-((5-(4-chloro-3-fluorophenyl)-2-((1-methyl-1H-pyrazol-4-yl)amino)pyrimidin-4-yl)oxy)phenyl)acrylamide (Compound 40),
N-(3-((5-(3-fluoro-4-methylphenyl)-2-((1-methyl-1H-pyrazol-4-yl)amino)pyrimidin-4-yl)oxy)phenyl)acrylamide (Compound 41),
N-(3-((5-(3-(dimethylamino)-5-fluorophenyl)-2-((1-methyl-1H-pyrazol-4-yl)amino)pyrimidin-4-yl)amino)-4-fluorophenyl)acrylamide (Compound 42),
N-(3-((5-(3-(difluoromethyl)-5-fluorophenyl)-2-((1-methyl-1H-pyrazol-4-yl)amino)pyrimidin-4-yl)amino)phenyl)acrylamide (Compound 43),
N-(3-((5-(6-chloro-5-(trifluoromethyl)pyridin-3-yl)-2-((1-methyl-1H-pyrazol-4-yl)amino)pyrimidin-4-yl)amino)-4-fluorophenyl)acrylamidet (Compound 44),
N-(3-((5-(6-chloro-5-fluoropyridin-3-yl)-2-((1-methyl-1H-pyrazol-4-yl)amino)pyrimidin-4-yl)amino)-4-fluorophenyl)acrylamide (Compound 45),
N-(3-((5-(5-chloro-6-methylpyridin-3-yl)-2-((1-methyl-1H-pyrazol-4-yl)amino)pyrimidin-4-yl)amino)-4-fluorophenyl)acrylamide (Compound 46),
N-(4-fluoro-3-((5-(6-fluoro-5-(trifluoromethyl)pyridin-3-yl)-2-((1-methyl-1H-pyrazol-4-yl)amino)pyrimidin-4-yl)amino)phenyl)acrylamide (Compound 47),
N-(3-((5-(6-chloro-5-methylpyridin-3-yl)-2-((1-methyl-1H-pyrazol-4-yl)amino)pyrimidin-4-yl)amino)-4-fluorophenyl)acrylamide (Compound 48),
N-(3-((5-(3-chloro-4-fluorophenyl)-2-((1-methyl-1H-pyrazol-4-yl)amino)pyrimidin-4-yl)amino)phenyl)acrylamide (Compound 49),
N-(3-((5-(3,4-dichlorophenyl)-2-((1-methyl-1H-pyrazol-4-yl)amino)pyrimidin-4-yl)amino)-4-fluorophenyl)acrylamide (Compound 50),
N-(3-((5-(3,4-dichlorophenyl)-2-((1-(2-hydroxyethyl)-1H-pyrazol-4-yl)amino)pyrimidin-4-yl)amino)-4-fluorophenyl)acrylamide (Compound 51),
N-(3-((2-((3-chloro-1-methyl-1H-pyrazol-4-yl)amino)-5-(3-chloro-4-fluorophenyl)pyrimidin-4-yl)amino)-4-fluorophenyl)acrylamide (Compound 52),
N-(3-((5-(3-chloro-4-fluorophenyl)-2-((1-(oxetan-3-yl)-1H-pyrazol-4-yl)amino)pyrimidin-4-yl)amino)-4-fluorophenyl)acrylamide (Compound 53),
N-(3-((5-(3-chloro-4-(trifluoromethyl)phenyl)-2-((1-methyl-1H-pyrazol-4-yl)amino)pyrimidin-4-yl)amino)-4-fluorophenyl)acrylamide (Compound 54),
N-(4-fluoro-3-((5-(3-fluoro-4-(1-methyl-1,2,3,6-tetrahydropyridin-4-yl)phenyl)-2-((1-methyl-1H-pyrazol-4-yl)amino)pyrimidin-4-yl)amino)phenyl)acrylamide (Compound 55),
N-(3-((5-(3,4-dichlorophenyl)-2-((1-(oxetan-3-yl)-1H-pyrazol-4-yl)amino)pyrimidin-4-yl)amino)-4-fluorophenyl)acrylamide (Compound 56),
N-(3-((5-(3-chloro-4-fluorophenyl)-2-((1-(2-(dimethylamino)ethyl)-1H-pyrazol-4-yl)amino)pyrimidin-4-yl)amino)-4-fluorophenyl)acrylamide (Compound 57),
N-(3-((5-(3,4-dichlorophenyl)-2-((1-(2-(dimethylamino)ethyl)-1H-pyrazol-4-yl)amino)pyrimidin-4-yl)amino)-4-fluorophenyl)acrylamide (Compound 58),
N-(4-fluoro-3-((2-((1-methyl-1H-pyrazol-4-yl)amino)-5-(1-methylindolin-5-yl)pyrimidin-4-yl)amino)phenyl)acrylamide (Compound 59),
N-(3-((5-(4-((dimethylamino)methyl)-3-fluorophenyl)-2-((1-methyl-1H-pyrazol-4-yl)amino)pyrimidin-4-yl)amino)-4-fluorophenyl)acrylamide (Compound 60),
N-(4-fluoro-3-((5-(5-fluoro-6-(trifluoromethyl)pyridin-3-yl)-2-((l-methyl-1H-pyrazol-4-yl)amino)pyrimidin-4-yl)amino)phenyl)acrylamide (Compound 61),
N-(3-((5-(3-chloro-4-fluorophenyl)-2-((1-(2-hydroxyethyl)-1H-pyrazol-4-yl)amino)pyrimidin-4-yl)amino)-4-fluorophenyl)acrylamide (Compound 62),
N-(4-fluoro-3-((5-(2-fluoro-6-methoxyphenyl)-2-((1-methyl-1H-pyrazol-4-yl)amino)pyrimidin-4-yl)amino)phenyl)acrylamide (Compound 63),
N-(4-fluoro-3-((5-(3-fluoro-5-methylphenyl)-2-((1-methyl-1H-pyrazol-4-yl)amino)pyrimidin-4-yl)amino)phenyl)acrylamide (Compound 64),
N-(4-fluoro-3-((5-(3-fluoro-5-methoxyphenyl)-2-((1-methyl-1H-pyrazol-4-yl)amino)pyrimidin-4-yl)amino)phenyl)acrylamide (Compound 65),
N-(3-((5-(3-chloro-5-fluorophenyl)-2-((1-methyl-1H-pyrazol-4-yl)amino)pyrimidin-4-yl)amino)phenyl)acrylamide (Compound 66),
N-(3-((5-(3,4-difluorophenyl)-2-((1-methyl-1H-pyrazol-4-yl)amino)pyrimidin-4-yl)amino)phenyl)acrylamide (Compound 67),
N-(3-((5-(4-chloro-3-fluorophenyl)-2-((1-methyl-1H-pyrazol-4-yl)amino)pyrimidin-4-yl)amino)phenyl)acrylamide (Compound 68),
N-(3-((5-(3-fluoro-4-methoxyphenyl)-2-((1-methyl-1H-pyrazol-4-yl)amino)pyrimidin-4-yl)amino)phenyl)acrylamide (Compound 69),
N-(3-((5-(6-chloro-5-fluoropyridin-3-yl)-2-((1-methyl-1H-pyrazol-4-yl)amino)pyrimidin-4-yl)oxy)phenyl)acrylamide (Compound 70),
N-(3-((5-(5-fluoro-6-methoxypyridin-3-yl)-2-((1-methyl-1H-pyrazol-4-yl)amino)pyrimidin-4-yl)oxy)phenyl)acrylamide (Compound 71),
N-(3-((5-(6-chloro-5-methylpyridin-3-yl)-2-((1-methyl-1H-pyrazol-4-yl)amino)pyrimidin-4-yl)oxy)phenyl)acrylamide (Compound 72),
N-(3-((5-(6-chloro-5-(trifluoromethyl)pyridin-3-yl)-2-((1-methyl-1H-pyrazol-4-yl)amino)pyrimidin-4-yl)oxy)phenyl)acrylamide (Compound 73),
N-(3-((5-(4-(dimethylamino)-3-fluorophenyl)-2-((1-methyl-1H-pyrazol-4-yl)amino)pyrimidin-4-yl)oxy)phenyl)acrylamide (Compound 74),
N-(3-((5-(5,6-difluoropyridin-3-yl)-2-((1-methyl-1H-pyrazol-4-yl)amino)pyrimidin-4-yl)oxy)phenyl)acrylamide (Compound 75),
N-(4-fluoro-3-((5-(3-fluoro-4-morpholinophenyl)-2-((1-methyl-1H-pyrazol-4-yl)amino)pyrimidin-4-yl)amino)phenyl)acrylamide (Compound 76),
N-(4-fluoro-3-((5-(3-fluoro-4-(pyrrolidin-1-yl)phenyl)-2-((1-methyl-1H-pyrazol-4-yl)amino)pyrimidin-4-yl)amino)phenyl)acrylamide (Compound 77),
N-(4-fluoro-3-((5-(3-fluoro-4-(piperidin-1-yl)phenyl)-2-((1-methyl-1H-pyrazol-4-yl)amino)pyrimidin-4-yl)amino)phenyl)acrylamide (Compound 78),
N-(4-fluoro-3-((5-(3-fluoro-4-(3-methoxyazetidin-1-yl)phenyl)-2-((1-methyl-1H-pyrazol-4-yl)amino)pyrimidin-4-yl)amino)phenyl)acrylamide (Compound 79),
N-(3-((5-(4-(azetidin-1-yl)-3-fluorophenyl)-2-((1-methyl-1H-pyrazol-4-yl)amino)pyrimidin-4-yl)amino)-4-fluorophenyl)acrylamide (Compound 80),
N-(4-fluoro-3-((5-(3-fluoro-4-(4-methylpiperazin-1-yl)phenyl)-2-((1-methyl-1H-pyrazol-4-yl)amino)pyrimidin-4-yl)amino)phenyl)acrylamide (Compound 81),
N-(3-((2-((1-(2-(dimethylamino)ethyl)-1H-pyrazol-4-yl)amino)-5-(3-fluoro-4-(piperidin-1-yl)phenyl)pyrimidin-4-yl)amino)-4-fluorophenyl)acrylamide (Compound 82),
N-(3-((5-(4-(3-(dimethylamino)azetidin-1-yl)-3-fluorophenyl)-2-((1-methyl-1H-pyrazol-4-yl)amino)pyrimidin-4-yl)amino)-4-fluorophenyl)acrylamide (Compound 83),
N-(4-fluoro-3-((5-(3-fluoro-4-(1,4-oxazepan-4-yl)phenyl)-2-((1-methyl-1H-pyrazol-4-yl)amino)pyrimidin-4-yl)amino)phenyl)acrylamide (Compound 84),
N-(3-((5-(4-(azepan-1-yl)-3-fluorophenyl)-2-((1-methyl-1H-pyrazol-4-yl)amino)pyrimidin-4-yl)amino)-4-fluorophenyl)acrylamide (Compound 85),
N-(3-((5-(4-((2-(dimethylamino)ethyl)(methyl)amino)-3-fluorophenyl)-2-((1-methyl-1H-pyrazol-4-yl)amino)pyrimidin-4-yl)amino)-4-fluorophenyl)acrylamide (Compound 86),
N-(3-((5-(4-bromo-3-fluorophenyl)-2-((1-methyl-1H-pyrazol-4-yl)amino)pyrimidin-4-yl)amino)-4-fluorophenyl)acrylamide (Compound 87),
N-(3-((5-(4-bromo-3-chlorophenyl)-2-((1-methyl-1H-pyrazol-4-yl)amino)pyrimidin-4-yl)amino)-4-fluorophenyl)acrylamide (Compound 88),
N-(3-((5-(4-bromo-3-chlorophenyl)-2-((1-(2-(dimethylamino)ethyl)-1H-pyrazol-4-yl)amino)pyrimidin-4-yl)amino)-4-fluorophenyl)acrylamide (Compound 89),
N-(3-((5-(3-bromo-4-chlorophenyl)-2-((1-methyl-1H-pyrazol-4-yl)amino)pyrimidin-4-yl)amino)-4-fluorophenyl)acrylamide (Compound 90),
N-(3-((5-(4-bromo-3,5-difluorophenyl)-2-((1-methyl-1H-pyrazol-4-yl)amino)pyrimidin-4-yl)amino)-4-fluorophenyl)acrylamide (Compound 91),
N-(3-((5-(4-bromo-3-fluorophenyl)-2-((3-chloro-1-methyl-1H-pyrazol-4-yl)amino)pyrimidin-4-yl)amino)-4-fluorophenyl)acrylamide (Compound 92),
N-(3-((5-(4-bromo-3-fluorophenyl)-2-((1-(tetrahydrofuran-3-yl)-1H-pyrazol-4-yl)amino)pyrimidin-4-yl)amino)-4-fluorophenyl)acrylamide (Compound 93),
N-(3-((5-(4-bromo-3-fluorophenyl)-2-((1-(oxetan-3-yl)-1H-pyrazol-4-yl)amino)pyrimidin-4-yl)amino)-4-fluorophenyl)acrylamide (Compound 94),
N-(3-((5-(4-bromo-3-fluorophenyl)-2-((1-(2-methoxyethyl)-1H-pyrazol-4-yl)amino)pyrimidin-4-yl)amino)-4-fluorophenyl)acrylamide (Compound 95),
N-(3-((5-(4-bromo-3-fluorophenyl)-2-((1-(2-hydroxyethyl)-1H-pyrazol-4-yl)amino)pyrimidin-4-yl)amino)-4-fluorophenyl)acrylamide (Compound 96),
N-(3-((5-(4-bromo-3-fluorophenyl)-2-((1-methyl-1H-pyrazol-4-yl)amino)pyrimidin-4-yl)amino)-5-fluorophenyl)acrylamide (Compound 97),
N-(3-((5-(4-chloro-3-fluorophenyl)-2-((1-methyl-1H-pyrazol-4-yl)amino)pyrimidin-4-yl)oxy)-4-fluorophenyl)acrylamide (Compound 98),
N-(3-((5-(3,4-dichlorophenyl)-2-((1-methyl-1H-pyrazol-4-yl)amino)pyrimidin-4-yl)oxy)phenyl)acrylamide (Compound 99),
N-(3-((5-(3-chloro-4-fluorophenyl)-2-((1-(2-methoxyethyl)-1H-pyrazol-4-yl)amino)pyrimidin-4-yl)amino)-4-fluorophenyl)acrylamide (Compound 100),
N-(3-((5-(5,6-difluoropyridin-3-yl)-2-((1-(methyl-d₃)-1H-pyrazol-4-yl)amino)pyrimidin-4-yl)amino)-4-fluorophenyl)acrylamide (Compound 101),
N-(3-((5-(4-bromo-3-fluorophenyl)-2-((1-methyl-1H-pyrazol-4-yl)amino)pyrimidin-4-yl)amino)-4-fluorophenyl)acrylamide-3,3-d₂ (Compound 102),
N-(3-((5-(5,6-difluoropyridin-3-yl)-2-((1-methyl-1H-pyrazol-4-yl)amino)pyrimidin-4-yl)amino)-4-fluorophenyl)acrylamide-3,3-d₂ (Compound 103),
N-(3-((2-((3-chloro-1-methyl-1H-pyrazol-4-yl)amino)-5-(3,5-difluorophenyl)pyrimidin-4-yl)amino)-4-fluorophenyl)acrylamide (Compound 104),
(E)-N-(3-((5-(4-bromo-3-fluorophenyl)-2-((1-methyl-1H-pyrazol-4-yl)amino)pyrimidin-4-yl)amino)-4-fluorophenyl)-4-(dimethylamino)but-2-enamide (Compound 105),
N-(3-((2-((3-chloro-1-methyl-1H-pyrazol-4-yl)amino)-5-(3-fluoro-5-methoxyphenyl)pyrimidin-4-yl)amino)-4-fluorophenyl)acrylamide (Compound 106),
N-(4-fluoro-3-((5-(4-fluoro-2-(trifluoromethyl)phenyl)-2-((1-methyl-1H-pyrazol-4-yl)amino)pyrimidin-4-yl)amino)phenyl)acrylamide (Compound 107),
N-(3-((2-((3-chloro-1-methyl-1H-pyrazol-4-yl)amino)-5-(3-(difluoromethyl)-5-fluorophenyl)pyrimidin-4-yl)amino)-4-fluorophenyl)acrylamide (Compound 108),
N-(3-((5-(3-bromo-5-fluorophenyl)-2-((1-methyl-1H-pyrazol-4-yl)amino)pyrimidin-4-yl)amino)-4-fluorophenyl)acrylamide (Compound 109),
N-(3-((2-((3-chloro-1-methyl-1H-pyrazol-4-yl)amino)-5-(3,4-dimethoxyphenyl)pyrimidin-4-yl)amino)-4-fluorophenyl)acrylamide (Compound 110),
N-(3-((5-(2,3-dihydrobenzo[b][1,4]dioxin-6-yl)-2-((1-methyl-1H-pyrazol-4-yl)amino)pyrimidin-4-yl)amino)-4-fluorophenyl)acrylamide (Compound 111),
N-(3-((5-(4-bromo-2-fluorophenyl)-2-((1-methyl-1H-pyrazol-4-yl)amino)pyrimidin-4-yl)amino)-4-fluorophenyl)acrylamide (Compound 112),
N-(3-((5-(3-bromo-2-fluorophenyl)-2-((1-methyl-1H-pyrazol-4-yl)amino)pyrimidin-4-yl)amino)-4-fluorophenyl)acrylamide (Compound 113),
N-(3-((5-(2,2-dimethylbenzo[d][1,3]dioxol-5-yl)-2-((1-methyl-1H-pyrazol-4-yl)amino)pyrimidin-4-yl)amino)-4-fluorophenyl)acrylamide (Compound 114),
N-(3-((5-(4-bromo-3-fluorophenyl)-2-((1-methyl-1H-pyrazol-4-yl)amino)pyrimidin-4-yl)amino)phenyl)acrylamide (Compound 115),
N-(3-((5-(3,5-dimethoxyphenyl)-2-((1-methyl-1H-pyrazol-4-yl)amino)pyrimidin-4-yl)amino)-4-fluorophenyl)acrylamide (Compound 116),
(E)-N-(3-((5-(3,5-difluorophenyl)-2-((1-methyl-1H-pyrazol-4-yl)amino)pyrimidin-4-yl)amino)-4-fluorophenyl)-4-(dimethylamino)but-2-enamide (Compound 117),
N-(3-((5-(4-bromo-3-(difluoromethyl)phenyl)-2-((1-methyl-1H-pyrazol-4-yl)amino)pyrimidin-4-yl)amino)-4-fluorophenyl)acrylamide (Compound 118),
N-(3-((5-(4-bromo-3-(dimethylamino)phenyl)-2-((1-methyl-1H-pyrazol-4-yl)amino)pyrimidin-4-yl)amino)-4-fluorophenyl)acrylamide (Compound 119),
N-(3-((5-(3-(azetidin-1-yl)-5-fluorophenyl)-2-((1-methyl-1H-pyrazol-4-yl)amino)pyrimidin-4-yl)amino)-4-fluorophenyl)acrylamide (Compound 120),
N-(4-fluoro-3-((5-(3-fluoro-5-(pyrrolidin-1-yl)phenyl)-2-((1-methyl-1H-pyrazol-4-yl)amino)pyrimidin-4-yl)amino)phenyl)acrylamide (Compound 121),
N-(3-((5-(3-chloro-4-((3-fluorobenzyl)oxy)phenyl)-2-((1-methyl-1H-pyrazol-4-yl)amino)pyrimidin-4-yl)amino)-4-fluorophenyl)acrylamide (Compound 122),
N-(3-((5-(4-bromo-3-chloro-5-fluorophenyl)-2-((1-methyl-1H-pyrazol-4-yl)amino)pyrimidin-4-yl)amino)-4-fluorophenyl)acrylamide (Compound 123),
N-(3-((5-(4-(difluoromethoxy)-3-fluorophenyl)-2-((1-methyl-1H-pyrazol-4-yl)amino)pyrimidin-4-yl)amino)phenyl)acrylamide (Compound 124),
N-(3-((5-(3,4-difluoro-5-methoxyphenyl)-2-((1-methyl-1H-pyrazol-4-yl)amino)pyrimidin-4-yl)amino)-4-fluorophenyl)acrylamide (Compound 126),
N-(3-((5-(3-fluoro-5-methoxyphenyl)-2-((1-methyl-1H-pyrazol-4-yl)amino)pyrimidin-4-yl)amino)phenyl)acrylamide (Compound 127),
N-(3-((5-(3-ethoxy-5-fluorophenyl)-2-((1-methyl-1H-pyrazol-4-yl)amino)pyrimidin-4-yl)amino)-4-fluorophenyl)acrylamide (Compound 128),
N-(4-fluoro-3-((5-(3-fluoro-5-isopropoxyphenyl)-2-((1-methyl-1H-pyrazol-4-yl)amino)pyrimidin-4-yl)amino)phenyl)acrylamide (Compound 129),
N-(3-((5-(3-(cyclopropylmethoxy)-5-fluorophenyl)-2-((1-methyl-1H-pyrazol-4-yl)amino)pyrimidin-4-yl)amino)-4-fluorophenyl)acrylamide (Compound 130),
N-(3-bromo-5-((5-(3-fluoro-4-methoxyphenyl)-2-((1-methyl-1H-pyrazol-4-yl)amino)pyrimidin-4-yl)amino)phenyl)acrylamide (Compound 131)
N-(3-bromo-5-((5-(4-(difluoromethoxy)-3-fluorophenyl)-2-((1-methyl-1H-pyrazol-4-yl)amino)pyrimidin-4-yl)amino)phenyl)acrylamide (Compound 132)
N-(4-fluoro-3-((5-(3-fluoro-5-methoxy-4-(trifluoromethyl)phenyl)-2-((1-methyl-1H-pyrazol-4-yl)amino)pyrimidin-4-yl)amino)phenyl)acrylamide (Compound 133),
N-(3-((5-(4-bromo-3-methoxyphenyl)-2-((1-methyl-1H-pyrazol-4-yl)amino)pyrimidin-4-yl)amino)-4-fluorophenyl)acrylamide (Compound 134),
N-(3-((5-(3,4-difluoro-5-(trifluoromethoxy)phenyl)-2-((1-methyl-1H-pyrazol-4-yl)amino)pyrimidin-4-yl)amino)-4-fluorophenyl)acrylamide (Compound 135),
N-(3-((5-(3-(difluoromethoxy)-4,5-difluorophenyl)-2-((1-methyl-1H-pyrazol-4-yl)amino)pyrimidin-4-yl)amino)-4-fluorophenyl)acrylamide (Compound 136),
N-(3-((5-(4-bromo-3-fluoro-5-methoxyphenyl)-2-((1-methyl-1H-pyrazol-4-yl)amino)pyrimidin-4-yl)amino)-4-fluorophenyl)acrylamide (Compound 137),
N-(3-((5-(4-bromo-3-(difluoromethyl)-5-fluorophenyl)-2-((1-methyl-1H-pyrazol-4-yl)amino)pyrimidin-4-yl)amino)-4-fluorophenyl)acrylamide (Compound 138),
N-(3-((5-(3-(dimethylamino)-4,5-difluorophenyl)-2-((1-methyl-1H-pyrazol-4-yl)amino)pyrimidin-4-yl)amino)-4-fluorophenyl)acrylamide (Compound 139),
N-(3-((5-(4-bromo-3-((2-(dimethylamino)ethyl)(methyl)amino)phenyl)-2-((1-methyl-1H-pyrazol-4-yl)amino)pyrimidin-4-yl)amino)-4-fluorophenyl)acrylamide (Compound 140),
N-(3-((5-(3-(difluoromethyl)-4,5-difluorophenyl)-2-((1-methyl-1H-pyrazol-4-yl)amino)pyrimidin-4-yl)amino)-4-fluorophenyl)acrylamide (Compound 141),
(E)-N-(3-((5-(4-bromo-3-fluorophenyl)-2-((1-methyl-1H-pyrazol-4-yl)amino)pyrimidin-4-yl)amino)-4-fluorophenyl)-4-(piperidin-1-yl)but-2-enamide (Compound 142),
N-(4-fluoro-3-((5-(3-fluoro-5-methoxyphenyl-4-d)-2-((1-methyl-1H-pyrazol-4-yl)amino)pyrimidin-4-yl)amino)phenyl)acrylamide (Compound 143),
(E)-4-(dimethylamino)-N-(4-fluoro-3-((5-(3-fluoro-5-methoxyphenyl)-2-((1-methyl-1H-pyrazol-4-yl)amino)pyrimidin-4-yl)amino)phenyl)but-2-enamide (Compound 144),
N-(3-((5-(3-fluoro-5-methoxyphenyl)-2-((1-methyl-1H-pyrazol-4-yl)amino)pyrimidin-4-yl)amino)phenyl-4-d)acrylamide (Compound 145),
N-(3-((5-(4-bromo-3-fluoro-5-methoxyphenyl)-2-((1-methyl-1H-pyrazol-4-yl)amino)pyrimidin-4-yl)amino)phenyl)acrylamide (Compound 146),
N-(3-((5-(4-bromo-3-fluorophenyl)-2-((1-methyl-1H-pyrazol-4-yl)amino)pyrimidin-4-yl)amino)-5-chlorophenyl)acrylamide (Compound 147),
N-(3-((2-((1-methyl-1H-pyrazol-4-yl)amino)-5-(3,4,5-trifluorophenyl)pyrimidin-4-yl)amino)phenyl)acrylamide (Compound 148),
N-(4-fluoro-3-((2-((1-methyl-1H-pyrazol-4-yl)amino)-5-(3,4,5-trifluorophenyl)pyrimidin-4-yl)amino)phenyl)acrylamide (Compound 149),
(E)-N-(3-((5-(3,5-difluorophenyl)-2-((1-methyl-1H-pyrazol-4-yl)amino)pyrimidin-4-yl)amino)phenyl)-4-(dimethylamino)but-2-enamide (Compound 150),
(E)-N-(3-((5-(3,5-difluorophenyl-4-d)-2-((1-methyl-1H-pyrazol-4-yl)amino)pyrimidin-4-yl)amino)-4-fluorophenyl)-4-(dimethylamino)but-2-enamide (Compound 151),
N-(3-((5-(4-bromo-3,5-difluorophenyl)-2-((1-methyl-1H-pyrazol-4-yl)amino)pyrimidin-4-yl)amino)phenyl)acrylamide (Compound 152),
N-(3-((5-(3,5-difluoro-4-(trifluoromethyl)phenyl)-2-((1-methyl-1H-pyrazol-4-yl)amino)pyrimidin-4-yl)amino)phenyl)acrylamide (Compound 153),
N-(3-((5-(4-bromo-3-chloro-5-fluorophenyl)-2-((1-methyl-1H-pyrazol-4-yl)amino)pyrimidin-4-yl)amino)phenyl)acrylamide (Compound 154),
N-(3-((5-(3,5-bis(difluoromethyl)phenyl)-2-((1-methyl-1H-pyrazol-4-yl)amino)pyrimidin-4-yl)amino)-4-fluorophenyl)acrylamide (Compound 155),
N-(3-((5-(4-(dimethylamino)-3,5-difluorophenyl)-2-((1-methyl-1H-pyrazol-4-yl)amino)pyrimidin-4-yl)amino)-4-fluorophenyl)acrylamide (Compound 156),
N-(3-((5-(3,5-difluorophenyl-4-d)-2-((1-methyl-1H-pyrazol-4-yl)amino)pyrimidin-4-yl)amino)-4-fluorophenyl)acrylamide (Compound 157)
N-(3-((5-(4-chloro-3,5-difluorophenyl)-2-((1-methyl-1H-pyrazol-4-yl)amino)pyrimidin-4-yl)amino)-4-fluorophenyl)acrylamide (Compound 158),
N-(3-((5-(4-bromo-3-fluorophenyl)-2-((1-methyl-1H-pyrazol-4-yl)amino)pyrimidin-4-yl)amino)-5-chlorophenyl)acrylamide (Compound 159),
N-(3-((5-(3-(difluoromethoxy)-4,5-difluorophenyl)-2-((1-methyl-1H-pyrazol-4-yl)amino)pyrimidin-4-yl)amino)-4-fluorophenyl)acrylamide (Compound 160),
(E)-N-(3-((5-(3,5-difluorophenyl)-2-((1-methyl-1H-pyrazol-4-yl)amino)pyrimidin-4-yl)amino)-4-fluorophenyl)-4-(dimethylamino)but-2-enamide (Compound 161),
N-(3-((5-(2,3-dihydrobenzofuran-6-yl)-2-((1-methyl-1H-pyrazol-4-yl)amino)pyrimidin-4-yl)amino)-4-fluorophenyl)acrylamide (Compound 162),
N-(3-((5-(4-bromo-3-chloro-5-fluorophenyl)-2-((1-methyl-1H-pyrazol-4-yl)amino)pyrimidin-4-yl)amino)-5-fluorophenyl)acrylamide (Compound 163),
N-(3-((5-(3,5-difluoro-4-(trifluoromethyl)phenyl)-2-((1-methyl-1H-pyrazol-4-yl)amino)pyrimidin-4-yl)amino)-5-fluorophenyl)acrylamide (Compound 164),
N-(4-fluoro-3-((5-(3-fluoro-5-(methoxy-d3)phenyl)-2-((1-methyl-1H-pyrazol-4-yl)amino)pyrimidin-4-yl)amino)phenyl)acrylamide (Compound 165),
(E)-4-(azetidin-1-yl)-N-(3-((5-(4-bromo-3-fluorophenyl)-2-((1-methyl-1H-pyrazol-4-yl)amino)pyrimidin-4-yl)amino)-4-fluorophenyl)but-2-enamide (Compound 166),
(E)-N-(3-((5-(3,5-difluorophenyl)-2-((1-(methyl-d3)-1H-pyrazol-4-yl)amino)pyrimidin-4-yl)amino)-4-fluorophenyl)-4-(dimethylamino)but-2-enamide (Compound 167),
N-(3-((5-(3-(difluoromethoxy)-5-fluorophenyl)-2-((1-methyl-1H-pyrazol-4-yl)amino)pyrimidin-4-yl)amino)-4-fluorophenyl)acrylamide (Compound 168),
N-(3-((5-(4-bromo-3-fluorophenyl)-2-((1-(methyl-d3)-1H-pyrazol-4-yl)amino)pyrimidin-4-yl)amino)-4-fluorophenyl)acrylamide (Compound 169),
N-(3,4-difluoro-5-((5-(3-fluoro-5-methoxyphenyl)-2-((1-methyl-1H-pyrazol-4-yl)amino)pyrimidin-4-yl)amino)phenyl)acrylamide (Compound 170),
(E)-N-(3-((5-(4-bromo-3,5-difluorophenyl)-2-((1-methyl-1H-pyrazol-4-yl)amino)pyrimidin-4-yl)amino)-4-fluorophenyl)-4-(dimethylamino)but-2-enamide (Compound 171),
N-(4-fluoro-3-((5-(3-fluoro-5-(trifluoromethoxy)phenyl)-2-((1-methyl-1H-pyrazol-4-yl)amino)pyrimidin-4-yl)amino)phenyl)acrylamide (Compound 172),
N-(3-bromo-5-((5-(3-chloro-5-fluorophenyl)-2-((1-methyl-1H-pyrazol-4-yl)amino)pyrimidin-4-yl)amino)phenyl)acrylamide (Compound 173),
N-(3-bromo-5-((5-(3-(difluoromethyl)-5-fluorophenyl)-2-((1-methyl-1H-pyrazol-4-yl)amino)pyrimidin-4-yl)amino)phenyl)acrylamide (Compound 174),
(E)-N-(3-((5-(4-bromo-3-fluorophenyl)-2-((1-methyl-1H-pyrazol-4-yl)amino)pyrimidin-4-yl)amino)-4-fluorophenyl)-4-fluorobut-2-enamide (Compound 175),
N-(4-fluoro-3-((5-(3-fluoro-5-(2,2,2-trifluoroethoxy)phenyl)-2-((1-methyl-1H-pyrazol-4-yl)amino)pyrimidin-4-yl)amino)phenyl)acrylamide (Compound 176),
N-(3-((5-(4-bromo-3-fluorophenyl)-2-((1-methyl-1H-pyrazol-4-yl)amino)pyrimidin-4-yl)amino)-4-fluorophenyl)-2-fluoroacrylamide (Compound 177), and
(E)-N-(3-((5-(2,3-dihydrobenzofuran-6-yl)-2-((1-methyl-1H-pyrazol-4-yl)amino)pyrimidin-4-yl)amino)-4-fluorophenyl)-4-(dimethylamino)but-2-enamide (Compound 178).

An embodiment of the present disclosure relates to a compound of Formula I or its stereoisomers, tautomers, pharmaceutically acceptable salts, stereoisomers, solvates, and hydrates thereof, for treating disease associated with epidermal growth factor receptor (EGFR) family kinases and HER family kinases.

Another embodiment of the present disclosure relates to a compound of Formula I or its stereoisomers, tautomers, pharmaceutically acceptable salts, stereoisomers, solvates, and hydrates thereof, for treating cancer.

Another embodiment of the present disclosure relates to a compound Formula I, or its stereoisomers, tautomers, pharmaceutically acceptable salts, stereoisomers, solvates, and hydrates thereof, for treating disease or condition associated with non-small cell or small cell lung cancer or prostate cancer or head and neck cancer or breast cancer or colorectal cancer.

The present disclosure relates to a pharmaceutical composition comprising a compound of Formula I or a pharmaceutically acceptable salt or stereoisomer thereof together with a pharmaceutically acceptable carrier, optionally in combination with one or more other pharmaceutical compositions.

The present disclosure further relates to the process of preparation of compounds of Formula I or its stereoisomers, tautomers, pharmaceutically acceptable salts, stereoisomers, solvates, and hydrates thereof.

### Uses

Some embodiments provided herein describe a class of compounds that are useful as epidermal growth factor receptor (EGFR) family kinase inhibitors and/or HER family kinase inhibitors. Some embodiments provided herein describe a class of compounds that are useful as as dual HER2 and EGFR kinase inhibitors.

Some embodiments provided herein describe a method of inhibiting a human epidermal growth factor receptor 2 (HER2) mutant and an epidermal growth factor receptor (EGFR) mutant in a subject in need thereof, comprising administering to the subject a therapeutically effective amount of a compound of Formula I, or a pharmaceutically acceptable salt or stereoisomer thereof. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is selected from A775_G776insYVMA, A775_G776insSVMA, A775_G776insVVMA, G776del insVC, G776del insLC, G776del insAV, G776del insAVGC, S310F, S310Y, p95, V842I, P780_Y781insGSP, and any combination thereof. In some embodiments, the EGFR mutant comprises a substitution in exon 18, a deletion in exon 19, a substitution in exon 20, an insertion in exon 20, a mutation in the extracellular domain, or a substitution in exon 21. In some embodiments, the EGFR mutant is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutant is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutant is del19/T790M EGFR or L858R/T790M EGFR.

Some embodiments provided herein describe a class of compounds that are useful as epidermal growth factor receptor (EGFR) family kinase inhibitors. Some embodiments provided herein describe a class of compounds that are useful as HER2 inhibitors. Some embodiments provided herein describe a class of compounds that are useful as EGFR inhibitors. Some embodiments provided herein describe a class of compounds that are useful as EGFR del19/T790M inhibitors. Some embodiments provided herein describe a class of compounds that are useful as EGFR L858R/T790M inhibitors. In some embodiments, the compounds described herein have improved potency and/or beneficial activity profiles and/or beneficial selectivity profiles and/or increased efficacy and/or improved safety profiles (such as reduced side effects) and/or improved pharmacokinetic properties. In some embodiments, the compounds described herein have improved potency and increased efficacy. In some embodiments, the compounds described herein are selective inhibitors of EGFR del19/T790M over WT EGFR. In some embodiments, the compounds described herein are selective inhibitors of EGFR L858R/T790M over WT EGFR.

In some embodiments, the compounds described herein are useful as inhibitors of both EGFR and HER2. In some embodiments, the compounds described herein have improved potency and increased efficacy through the inhibition of both EGFR and HER2.

In some embodiments, the compounds described herein are useful to treat, prevent or ameliorate a disease or condition which displays drug resistance associated with EGFR del19/T790M activation. In some embodiments, the compounds described herein are useful to treat, prevent or ameliorate a disease or condition which displays drug resistance associated with EGFR L858R/T790M activation.

In some embodiments, EGFR family kinase mutants are detected with a commercially available test kit. In some embodiments, EGFR family kinase mutants are detected with a reverse transcription polymerase chain reaction (RT-PCR)-based method. In some embodiments, EGFR family kinase mutants are detected with a sequencing-based method. In some embodiments, EGFR family kinase mutants are detected with a mass spectrometry genotyping-based method. In some embodiments, EGFR family kinase mutants are detected with an immunohistochemistry-based method. In some embodiments, EGFR family kinase mutants are detected with a molecular diagnostics panel. In some embodiments, EGFR family kinase mutants are detected from a tumor sample. In some embodiments, EGFR family kinase mutants are detected from circulating DNA. In some embodiments, EGFR family kinase mutants are detected from tumor cells.

In one aspect, provided herein is a method of inhibiting an epidermal growth factor receptor (EGFR) family kinase mutant in a subject in need thereof, comprising administering to the subject a therapeutically effective amount of a compound of Formula I, or a pharmaceutically acceptable salt or stereoisomer thereof.

In another aspect, provided herein is a method of inhibiting a human epidermal growth factor receptor 2 (HER2) mutant in a subject in need thereof, comprising administering to the subject a therapeutically effective amount of a compound of Formula I, or a pharmaceutically acceptable salt or stereoisomer thereof. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is selected from A775 G776insYVMA, A775_G776insSVMA, A775_G776insVVMA, G776del insVC, G776del insLC, G776del insAV, G776del insAVGC, S310F, S310Y, p95, V842I, P780_Y781insGSP, and any combination thereof. In some embodiments, the HER2 mutant is A775_G776insYVMA. In some embodiments, the HER2 mutant is A775 G776insSVMA. In some embodiments, the HER2 mutant is A775_G776insVVMA. In some embodiments, the HER2 mutant is G776del insVC. In some embodiments, the HER2 mutant is G776del insLC. In some embodiments, the HER2 mutant is G776del insAV. In some embodiments, the HER2 mutant is G776del insAVGC. In some embodiments, the HER2 mutant is S310F. In some embodiments, the HER2 mutant is S310Y. In some embodiments, the HER2 mutant is p95. In some embodiments, the HER2 mutant is V842I. In some embodiments, the HER2 mutant is P780_Y781insGSP.

In another aspect, provided herein is a method of inhibiting an epidermal growth factor receptor (EGFR) mutant in a subject in need thereof, comprising administering to the subject a therapeutically effective amount of a compound of Formula I, or a pharmaceutically acceptable salt or stereoisomer thereof.

In another aspect, provided herein is a method of inhibiting a drug-resistant epidermal growth factor receptor (EGFR) mutant in a subject in need thereof, comprising administering to the subject a therapeutically effective amount of a compound of Formula I, or a pharmaceutically acceptable salt or stereoisomer thereof. In some embodiments, the drug-resistant EGFR mutant is del19/T790M EGFR or L858R/T790M EGFR.

In another aspect, provided herein is a method of inhibiting human epidermal growth factor receptor 2 (HER2) in a subject in need thereof, comprising administering to the subject a therapeutically effective amount of a compound of Formula I, or a pharmaceutically acceptable salt or stereoisomer thereof, wherein the compound exhibits greater inhibition of a HER2 mutant relative to wild-type EGFR. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is selected from A775_G776insYVMA, A775_G776insSVMA, A775_G776insVVMA, G776del insVC, G776del insLC, G776del insAV, G776del insAVGC, S310F, S310Y, p95, V842I, P780_Y781insGSP, and any combination thereof. In some embodiments, the HER2 mutant is A775_G776insYVMA. In some embodiments, the HER2 mutant is A775_G776insSVMA. In some embodiments, the HER2 mutant is A775_G776insVVMA. In some embodiments, the HER2 mutant is G776del insVC. In some embodiments, the HER2 mutant is G776del insLC. In some embodiments, the HER2 mutant is G776del insAV. In some embodiments, the HER2 mutant is G776del insAVGC. In some embodiments, the HER2 mutant is S310F. In some embodiments, the HER2 mutant is S310Y. In some embodiments, the HER2 mutant is p95. In some embodiments, the HER2 mutant is V842I. In some embodiments, the HER2 mutant is P780_Y781insGSP.

In another aspect, provided herein is a method of inhibiting epidermal growth factor receptor (EGFR) in a subject in need thereof, comprising administering to the subject a therapeutically effective amount of a compound of Formula I, or a pharmaceutically acceptable salt or stereoisomer thereof, wherein the compound exhibits greater inhibition of an EGFR mutant relative to wild-type EGFR.

In some embodiments, the EGFR mutant comprises a substitution in exon 18, a deletion in exon 19, a substitution in exon 20, an insertion in exon 20, a mutation in the extracellular domain, or a substitution in exon 21. In some embodiments, the EGFR mutant is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutant is del19/T790M EGFR or L858R/T790M EGFR. In some embodiments, the EGFR mutant is del19/T790M EGFR. In some embodiments, the EGFR mutant is L858R/T790M EGFR.

In another aspect, provided herein is a method of treating a disease or disorder associated with epidermal growth factor receptor (EGFR) in a subject in need thereof, comprising administering to the subject a therapeutically effective amount of a compound of Formula I, or a pharmaceutically acceptable salt or stereoisomer thereof.

In some embodiments, the disease or disorder in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is selected from A775_G776insYVMA, A775_G776insSVMA, A775_G776insVVMA, G776del insVC, G776del insLC, G776del insAV, G776del insAVGC, S310F, S310Y, p95, V842I, P780_Y781insGSP, and a combination thereof. In some embodiments, the HER2 mutation is A775_G776insYVMA. In some embodiments, the HER2 mutation is A775_G776insSVMA. In some embodiments, the HER2 mutation is A775_G776insVVMA. In some embodiments, the HER2 mutation is G776del insVC. In some embodiments, the HER2 mutation is G776del insLC. In some embodiments, the HER2 mutation is G776del insAV. In some embodiments, the HER2 mutation is G776del insAVGC. In some embodiments, the HER2 mutation is S310F. In some embodiments, the HER2 mutation is S310Y. In some embodiments, the HER2 mutation is p95. In some embodiments, the HER2 mutation is V842I. In some embodiments, the HER2 mutation is P780_Y781insGSP.

In some embodiments, the disease or disorder in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR.

In another aspect, provided herein is a method of treating cancer in a subject in need thereof, comprising administering to the subject a therapeutically effective amount of a compound of Formula I, or a pharmaceutically acceptable salt or stereoisomer thereof. In some embodiments, the cancer displays drug resistance associated with EGFR del19/T790M activation. In some embodiments, the cancer displays drug resistance associated with EGFR L858R/T790M activation. Other embodiments provided herein describe the use of the compounds described herein for treating cancer.

In some embodiments, the cancer is bladder cancer, prostate cancer, breast cancer, cervical cancer, colorectal cancer, endometrial cancer, gastric cancer, glioblastoma, head and neck cancer, lung cancer, or non-small cell lung cancer. In some embodiments, the cancer is non-small cell lung cancer, prostate cancer, head and neck cancer, breast cancer, colorectal cancer, or glioblastoma. In some embodiments, the cancer is non-small cell lung cancer. In some embodiments, the cancer is prostate cancer. In some embodiments, the cancer is head and neck cancer. In some embodiments, the cancer is breast cancer. In some embodiments, the cancer is colorectal cancer. In some embodiments, the cancer is glioblastoma.

In some embodiments, the cancer in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is selected from A775_G776insYVMA, A775_G776insSVMA, A775_G776insVVMA, G776del insVC, G776del insLC, G776del insAV, G776del insAVGC, S310F, S310Y, p95, V842I, P780_Y781insGSP, and a combination thereof. In some embodiments, the HER2 mutation is A775_G776insYVMA. In some embodiments, the HER2 mutation is A775_G776insSVMA. In some embodiments, the HER2 mutation is A775_G776insVVMA. In some embodiments, the HER2 mutation is G776del insVC. In some embodiments, the HER2 mutation is G776del insLC. In some embodiments, the HER2 mutation is G776del insAV. In some embodiments, the HER2 mutation is G776del insAVGC. In some embodiments, the HER2 mutation is S310F. In some embodiments, the HER2 mutation is S310Y. In some embodiments, the HER2 mutation is p95. In some embodiments, the HER2 mutation is V842I. In some embodiments, the HER2 mutation is P780_Y781insGSP.

In some embodiments, the cancer in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR. In some embodiments, the cancer comprises EGFR mutation and HER2 mutation described herein.

In another aspect, provided herein is a method of treating inflammatory disease in a subject in need thereof, comprising administering to the subject a therapeutically effective amount of a compound of Formula I, or a pharmaceutically acceptable salt or stereoisomer thereof. Also described herein is the use of the compounds described herein for treating inflammatory diseases associated with EGFR del19/T790M activation. Also described herein is the use of the compounds described herein for treating inflammatory diseases associated with EGFR L858R/T790M activation.

In some embodiments, the inflammatory disease is psoriasis, eczema, or atherosclerosis. In some embodiments, the inflammatory disease is psoriasis. In some embodiments, the inflammatory disease is eczema. In some embodiments, the inflammatory disease is atherosclerosis.

In some embodiments, the inflammatory disease in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is selected from A775_G776insYVMA, A775 G776insSVMA, A775_G776insVVMA, G776del insVC, G776del insLC, G776del insAV, G776del insAVGC, S310F, S310Y, p95, V842I, P780_Y781insGSP, and any combination thereof. In some embodiments, the HER2 mutation is A775_G776insYVMA. In some embodiments, the HER2 mutation is A775 G776insSVMA. In some embodiments, the HER2 mutation is A775 G776insVVMA. In some embodiments, the HER2 mutation is G776del insVC. In some embodiments, the HER2 mutation is G776del insLC. In some embodiments, the HER2 mutation is G776del insAV. In some embodiments, the HER2 mutation is G776del insAVGC. In some embodiments, the HER2 mutation is S310F. In some embodiments, the HER2 mutation is S310Y. In some embodiments, the HER2 mutation is p95. In some embodiments, the HER2 mutation is V842I. In some embodiments, the HER2 mutation is P780_Y781insGSP.

In some embodiments, the inflammatory disease in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR. In some embodiments, the inflammatory disease in the subject comprises a HER2 mutation and an EGFR mutation described herein.

### Administration and Pharmaceutical Composition

In certain embodiments, the EGFR and/or HER2 inhibitory compound as described herein is administered as a pure chemical. In other embodiments, the EGFR and/or HER2 inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Provided herein is a pharmaceutical composition comprising at least one EGFR and/or HER2 inhibitory compound as described herein, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

One embodiment provides a pharmaceutical composition comprising a compound disclosed herein, or a pharmaceutically acceptable salt, stereoisomer, solvate, or prodrug thereof, and a pharmaceutically acceptable excipient.

In certain embodiments, the EGFR and/or HER2 inhibitory compound disclosed herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21st Ed. Mack Pub. Co., Easton, PA (2005)).

The dose of the composition comprising at least one EGFR and/or HER inhibitory compound as described herein differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### EXAMPLES

### Example 1: Synthetic Procedures

Yields reported herein refer to purified products (unless specified) and are not optimised. Analytical TLC was performed on Merck silica gel 60 F₂₅₄ aluminum-backed plates. Compounds were visualised by UV light and/or stained either with iodine, potassium permanganate or ninhydrin solution. Flash column chromatography was performed on silica gel (100-200 M) or flash chromatography. ¹H-NMR spectra were recorded on a Bruker Avance-400 MHz spectrometer with a BBO (Broad Band Observe) and BBFO (Broad Band Fluorine Observe) probe. Chemical shifts (δ) are expressed in parts per million (ppm) downfield by reference to tetramethylsilane (TMS) as the internal standard. Splitting patterns are designated as s (singlet), d (doublet), t (triplet), q (quartet), m (multiplet) and bs (broad singlet). Coupling constants (J) are given in hertz (Hz). LC-MS analyses were performed on either an Acquity BEH C-18 column (2.10×100 mm, 1.70 μm) or on a Acquity HSS-T3 column (2.10×100 mm, 1.80 μm) using the Electrospray Ionisation (ESI) technique.

The following solvents, reagents or scientific terminology may be referred to by their abbreviations:

### TLC Thin Layer Chromatography

### DCM Dichloromethane

### THF Tetrahydrofuran

### MeOH Methanol

### EtOH Ethanol

IPA Isopropyl alcohol
EtOAc Ethyl acetate
Et₂O Diethyl ether

### DMA N,N-Dimethylacetamide

### DMF N,N-Dimethylformamide

### TEA/Et₃N Triethylamine

### DMSO Dimethylsulfoxide

DIPEA Diisopropylethylamine (Hunig's base)

### Mel Methyliodide

### NBS N-Bromosuccinimide

TBAB Tetrabutylammonium bromide
TBAI Tetrabutylammonium iodide
DIBAL-H Diisobutylaluminum hydride
TFA Trifluoroacetic acid
AcOH Acetic acid
Boc tert-butoxycarbonyl

### Cat Catalytic

mL milliliters
mmol millimoles
h hour or hours
min minute or minutes
g grams
mg milligrams

### μl Microlitres

### eq Equivalents

rt or RT Room temperature, ambient, about 27° C.
MS Mass spectrometry
Boc tert-Butyloxycarbonyl
m-CPBA meta-Chloroperbenzoic acid
T3P Propane phosphonic acid anhydride
BH₃-DMS Borane dimethylsulfide complex
LiBH₄ Lithium aluminum hydride
NaBH₄ Sodium borohydride

### H₂ Hydrogen

Pd/C Palladium on charcoal

### 1,2-DCE 1,2-Dichloroethane

### General Procedure A:

To an ice cold solution of aryl amines (1.0 eq) in tetrahydrofuran was added sodium hydride (60% dispersion in mineral oil, 3.0 eq) portion-wise. The resulting reaction mixture was stirred at room temperature for 30 minutes and followed by the addition of 2,4,5-trichloropyrimidine or 2,4-dichloro-5-bromopyrimidine (1.0 eq). The resulting reaction mixture was heated at 60° C. for 16 hours. After completion (TLC monitoring), quenched with ice, extracted with ethyl acetate (3 times). The combined organic layers were washed with water, brine, dried over anhydrous sodium sulfate, filtered and concentrated under reduced pressure. The crude was triturated with diethyl ether, filtered and dried under vacuum to get desired products.

### General Procedure B:

To a solution of aryl halo (1.0 eq) in 1,4-dioxane or toluene were added cesium carbonate (3.0 eq) and aryl amines (1.2 eq). The resulting reaction mixture degassed under nitrogen for 15 minutes, followed by addition of 2-dicyclohexylphosphino-2′,4′,6′-triisopropylbiphenyl (XPhos, 0.1 eq) and tris(dibenzylideneacetone)dipalladium(0) (0.1 eq) under nitrogen atmosphere. The resulting reaction mixture was again degassed for 15 minutes and then heated at 100° C. for 16 hours. After completion of reaction (TLC monitoring), reaction mixture was cooled, diluted with water, extracted with dichloromethane (3 times). The combined organic layers were washed with brine dried over anhydrous sodium sulfate, filtered and concentrated under reduced pressure. The crude was purified by flash chromatography using 4-8% methanol in dichloromethane as eluent, desired fractions were concentrated under reduced pressure afforded the desired products.

### General Procedure C:

To an ice-cold solution of primary or secondary aryl amines (1.0 eq)) in dichloromethane were added triethylamine (3.0 eq) and acetyl chloride (1.2 eq) drop wise. The resulting reaction mixture was stirred at room temperature for 1 hour. After completion of reaction (TLC monitoring), the reaction mixture was diluted with water and extracted with dichloromethane (3 times). The combined organic layers were washed with brine, dried over anhydrous sodium sulfate, filtered and concentrated under reduced pressure. The crude was purified by combiflash, eluted with 4-5% methanol in dichloromethane, desired fractions were concentrated under reduced pressure to afforded desired products.

### General Procedure D:

To a solution of aldehydes (1.0 eq) in methanol were added respective amines (3.0 eq) and sodium acetate (5.0 eq). The resultant reaction mixture was stirred at room temperature for 16 hours. After completion of reaction (monitored by TLC), the reaction mixture was poured in ice-cold water and resulted solid was filtered. The solid was dried under vacuum to get the desired products.

### General Procedure E:

To a solution of products (1.0 eq) obtained from General Procedure D in methanol (2.5 vol) was added acetic acid (1.0 vol) and followed by addition of sodium borohydride (1.0 eq). The resulting reaction mixture was stirred at room temperature for 16 hours. After completion of reaction (TLC monitoring), the reaction mixture was quenched with ice-cold water and resultant solid was filtered, washed with water. The solid was dried under vacuum to get the desired products.

### General Procedure F:

To an ice-cold solution of products (1.0 eq) obtained from General Procedure E in tetrahydrofuran added di-isopropyl ethylamine (4.0 eq) followed by addition of triphosgene (0.4 eq). The resultant reaction mixture was stirred at room temperature for 16 hours. After completion of reaction (TLC monitoring) saturated sodium bicarbonate solution was added and extracted with dichloromethane (3 times). The organic layer was washed with brine dried over anhydrous sodium sulfate and evaporated under reduced pressure. The crude was triturated with diethyl ether to get the desired products.

### General Procedure G:

To an ice-cold solution of products (1.0 eq) obtained from General Procedure F in dichloromethane was added m-chloroperbenzoic acid (2.0 eq). The resulting reaction mixture was stirred at room temperature for 4 hours. After completion of reaction (TLC monitoring) saturated solution of sodium bicarbonate was added to the reaction mixture and extracted with dichloromethane (3 times). The combined organic layers were washed with brine, dried over anhydrous sodium sulfate and evaporated under reduced pressure. The crude product was triturated with diethyl ether to get the desired products.

### General Procedure H:

To an ice-cold solution of products (1.0 eq) obtained from General Procedure G in isopropanol was added respective amines (1.2 eq) and trifluoroacetic acid (2.0 eq). The reaction mixture was heated at 110° C. for 16 hours. After completion of the reaction (TLC monitoring), the reaction mixture was concentrated under reduced pressure, added saturated solution of sodium bicarbonate and extracted with dichloromethane (3 times). The combined organic layers were washed with brine solution, dried over anhydrous sodium sulfate and evaporated under reduced pressure. The crude residue was triturated with diethyl ether to get the desired products which was used directly for the next step.

### General Procedure I:

An ice-cold solution of products (1.0 eq) obtained from General Procedure H in 20% trifluoroacetic acid in dichloromethane was stirred at room temperature for 3-16 hours. After completion of the reaction (TLC monitoring) the solvent was evaporated. The reaction mass diluted with saturated solution of sodium bicarbonate and extracted with 5% methanol in dichloromethane (3 times). The combined organic layers were washed with brine solution, dried over sodium sulfate and evaporated under reduced pressure. The crude was triturated with ether or purified over combiflash, elution with 5-10% methanol in dichloromethane to get the desired products.

### General Procedure J:

To an ice-cold solution of products (1.0 eq) obtained from General Procedure I in dichloromethane was added triethylamine (3-5 eq) and respective acids (1.1 eq), followed by propylphosphonic anhydride (T₃P, 50% in ethyl acetate, 2.5 eq). The resulting reaction mixture was stirred at room temperature for 16 hours. After completion of reaction (TLC monitoring), reaction mass diluted with saturated solution of sodium bicarbonate and extracted with 5% methanol in dichloromethane. The combined organic layers were washed with brine, dried over anhydrous sodium sulfate, filtered and concentrated under reduced pressure. The crudes were purified over combiflash or Prep-TLC or Prep-HPLC purification to get the final compounds.

### General Procedure K:

To a solution of products (1.0 eq) obtained from General Procedure I in dichloromethane:tetrahydrofuran (1:1) was cooled to −40° C. followed by triethylamine (3-5 eq) and acryloyl chloride (1.0 eq) were added. The mixture was stirred at the same temperature for 2 hours. After completion of reaction (monitored by TLC), added water and extracted with dichloromethane (3 times). The combined organic layers washed with brine, dried over anhydrous sodium sulfate, filtered and concentrated under reduced pressure. The crudes were purified by Prep-HPLC purification to get the final compounds.

### General Procedure K₁:

To a solution of products (1.0 eq) obtained from General Procedure I in tetrahydrofuran and water (3:1) at −0° C. were added triethylamine (5 eq) and acryloyl chloride (1.0 eq). The reaction mixture was stirred at the same temperature for 2 hours. After completion of reaction (monitored by TLC), added water and extracted with ethyl acetate (3 times). The combined organic layers were washed with brine, dried over anhydrous sodium sulfate, filtered and concentrated under reduced pressure. The crude product was purified by Prep-HPLC purification to get the final compounds.

### General Procedure K₂:

To a solution of products (1.0 eq) obtained from General Procedure I in tetrahydrofuran and water (3:1) at −0° C. were added triethylamine (5 eq) and 3-Chloropropionyl chloride (1.2 to 1.5 eq). The reaction mixture was stirred at the same temperature for 20 minutes to one hour. After completion of reaction (monitored by LCMS), added water and extracted with ethyl acetate (3 times). The combined organic layers were washed with brine, dried over anhydrous sodium sulfate, filtered and concentrated under reduced pressure. The crude product was purified by Prep-HPLC purification to get the final compounds.

### General Procedure L:

To an ice cold solution of nitro derivatives (1.0 eq) in methanol:tetrahydrofuran:water (2:2:1) were added zinc-dust or iron powder (5 eq) and ammonium chloride (5 eq). The resultant reaction mixture was stirred at room temperature for 2 hours. After completion of reaction (TLC monitoring), reaction mixture passed through celite bed washed with 5% methanol in dichloromethane. The filtrate was washed with water, brine, dried over anhydrous sodium sulfate, filtered and concentrated to dryness to get the amino derivatives.

### General Procedure L₁:

To a solution of nitro derivatives (1.0 eq) in methanol or ethanol (10 vol) was added 10% palladium on carbon (20% w/w). The reaction mixture was stirred under hydrogen atmosphere for 16 hours. After completion of reaction (TLC monitoring), reaction mixture was filtered through the celite bed and washed with methanol. The combined filtrate was concentrated under reduced pressure to get amino derivatives.

### General Procedure M₁: (Suzuki Coupling):

To a solution of halo derivatives (1.0 eq) in acetonitrile was added respective boronate acid/ester derivatives (1.0 eq), followed by aqueous solution of potassium carbonate (2.0 eq) under argon purging. The resulting reaction mixture was degassed for 15 minutes, followed by [1,1′-Bis (diphenylphosphino)ferrocene]palladium (II) dichloride dichloromethane complex (0.1 eq) was added and the reaction mixture was heated at 80° C. for 16 hours. After completion of reaction (TLC monitoring), the reaction mixture was diluted with ice water and extracted with ethyl acetate (3 times). The combined organic layers were washed with brine, dried over anhydrous sodium sulfate, filtered and concentrated to dryness. The crude was purified over combiflash, eluted with 40-60% ethyl acetate in hexane, desired fractions were concentrated under reduced pressure to get the desired products.

### General Procedure M2:

To a solution of halo derivatives (1.0 eq) and respective boronic acids (1.1 eq) in toluene:ethanol (1:1) or dimethylformamide or dimethoxyethane and water (4:1) was added potassium carbonate (2.0 eq) or sodium bicarbonate (2.0 eq). The resulting reaction mixture was degassed with argon for 15 minutes, followed by addition of [1,1′-bis(diphenylphosphino)ferrocene]dichloropalladium(II) dichloromethane complex (0.05 eq). The resulting reaction mixture was heated at 90° C. for 5-16 hours. After completion of reaction (TLC monitoring), the reaction mixture was cooled to room temperature, water was added and extracted with ethyl acetate (3 times). The combined organic layers were washed with brine, dried over anhydrous sodium sulfate, filtered and concentrated under reduced pressure. The crude was purified over combiflash, elution with 30-50% ethyl acetate in hexane, desired fractions were concentrated under reduced pressure to the desired products.

### General Procedure M3:

To a solution of halo derivatives (1.0 eq) and respective boronate acid/ester derivatives (1.1 eq) in N,N-dimethylformamide:water (4:1) was added sodium carbonate or sodium bicarbonate (2.0 eq). The resulting reaction mixture was degassed under argon atmosphere for 15 minutes, followed by addition of tetrakis(triphenylphosphine)palladium(0) (0.1 eq). The resulting reaction mixture was heated at 90° C. for 16 hours. After completion of reaction (TLC monitoring), the reaction mixture was cooled to room temperature, water was added and extracted with ethyl acetate (3 times). The combined organic layers were washed with brine, dried over anhydrous sodium sulfate, filtered and concentrated under reduced pressure. The crude product was purified by using combiflash, desired fractions were concentrated under reduced pressure to afford the desired products.

### General Procedure N:

To an ice-cold solution of N-(3-(2-chloro-6-fluoroquinazolin-8-yl)phenyl)acrylamide (1.0 eq) in dimethylformamide was added sodium hydride (60% dispersion in mineral oil, 10 eq) portion-wise and stirred at room temperature for 30 minutes, followed by addition of respective amines (1.2 eq). The resultant reaction mixture was stirred at room temperature for 16 hours. After completion of reaction (as per TLC monitoring), reaction mixture was diluted with ice-cold water and extracted with 5% methanol/dichloromethane (3 times). The combined organic layer dried over anhydrous sodium sulfate, filtered and concentrated under reduced pressure. The crude was purified over combiflash or Prep HPLC purification to get desired products.

### General Procedure O:

To a solution of primary or secondary alcohols (1.0 eq) in dichloromethane was added activated manganese dioxide (10 eq) at room temperature under nitrogen atmosphere. The resultant reaction mixture was stirred at same temperature for 16 hours. After completion of reaction (TLC monitoring), the reaction mixture was filtered through celite bed and washed with dichloromethane (3 times). The combined filtrate was dried over anhydrous sodium sulfate and concentrated under reduced pressure to get desired products.

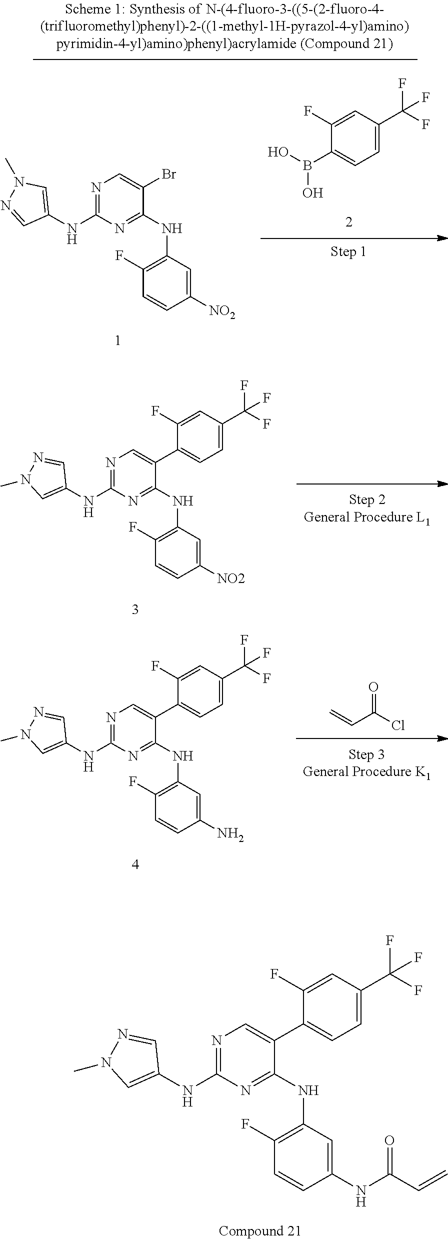

### Step 1: Synthesis of 5-(2-fluoro-4-(trifluoromethyl) phenyl)-N4-(2-fluoro-5-nitrophenyl)-N2-(1-methyl-1H-pyrazol-4-yl)pyrimidine-2,4-diamine (3)

To a solution of 5-bromo-N4-(2-fluoro-5-nitrophenyl)-N2-(1-methyl-1H-pyrazol-4-yl)pyrimidine-2,4-diamine (1) (0.3 g, 0.73 mmol), [2-fluoro-4-(trifluoromethyl)phenyl]boronic acid (2) (0.18 g, 0.88 mmol) in 1,4-dioxane (3.00 mL) and water (1.00 mL) was added sodium hydrogen carbonate (0.18 g, 2.20 mmol). Then the reaction mixture was purged with nitrogen for 10 minutes, added bis(triphenylphosphine)palladium(II) dichloride (0.05 g, 0.73 mmol) and the reaction mixture was heated at 100° C. for 16 hours. Progress of the reaction was monitored by LCMS. After completion of the reaction the reaction mixture was cooled to room temperature and diluted with water (20 mL) and extracted with ethyl acetate (2×50 mL). The combined organic layer was washed with brine (25 mL), dried over anhydrous sodium sulfate and concentrated under vacuo. The crude product was purified by flash column chromatography with 80% ethyl acetate in hexane as eluent to give the title compound (3) (0.27 g, 74.76% yield) as yellow solid. LCMS: [M+H]⁺ 492.4

### Step 2: Synthesis of N4-(5-amino-2-fluorophenyl)-5-(2-fluoro-4-(trifluoromethyl)phenyl)-N2-(1-methyl-1H-pyrazol-4-yl)pyrimidine-2,4-diamine (4)

The title compound was prepared in a manner substantially similar to procedure mentioned in General Procedure Li to afford desired product (4) brown liquid (0.2 g, crude). LCMS: [M+H]⁺ 462.4.

### Step 3: Synthesis of N-(4-fluoro-3-((5-(2-fluoro-4-(trifluoromethyl)phenyl)-2-((1-methyl-1H-pyrazol-4-yl)amino)pyrimidin-4-yl)amino)phenyl)acrylamide (Compound 21)

The title compound was prepared in a manner substantially similar to procedure mentioned in General Procedure K₁ to afford off white solid (0.02 g, 17.9% yield). ¹H NMR (400 MHz, DMSO-d6): δ10.19 (s, 1H), 9.24 (s, 1H), 8.44 (s, 2H), 7.92 (s, 1H), 7.72-7.56 (m, 4H), 7.26-7.16 (m, 3H), 6.42-6.35 (m, 1H), 6.22 (d, J=15.2 Hz, 1H), 5.72 (d, J=11.6 Hz, 1H), 3.53 (s, 3H). LCMS: [M+H]⁺ 516.4.

TABLE 1
The following compounds were prepared using the procedures described above:

| Cmpd. | | General | LCMS | |
|---|---|---|---|---|
| No. | Structure | Procedure | [M + H] | ¹H-NMR (400 MHZ, DMSO-d₆) |
| 1 | 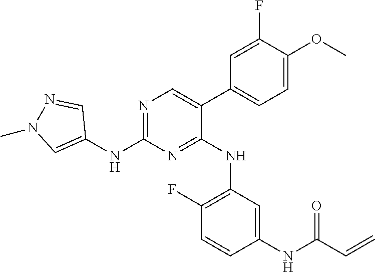 [SMILES: C=CC(=O)Nc1ccc(F)c(Nc2nc(Nc3cnn(C)c3)ncc2-c2ccc(OC)c(F)c2)c1] | K | 478.17 | δ 10.25 (s, 1H), 9.11 (bs, 1H), 8.27 (bs, 1H), 7.89 (s, 1H), 7.61-7.79 (m, 2H), 7.16-7.30 (m, 6H), 6.39- 6.46 (m, 1H), 6.22-6.26 (m, 1H), 5.73 (dd, J = 12.0 Hz & 1.6 Hz, 1H), 3.88 (s, 3H), 3.55 (s, 3H). |
| 2 | 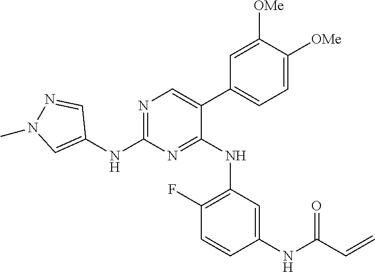 [SMILES: C=CC(=O)Nc1ccc(F)c(Nc2nc(Nc3cnn(C)c3)ncc2-c2ccc(OC)c(OC)c2)c1] | K | 490.15 | δ 10.21 (s, 1H), 9.07 (bs, 1H), 8.13 (bs, 1H), 7.92 (s, 1H), 7.82 (s, 1H), 7.59 (s, 1H), 6.98-7.18 (m, 6H), 6.38-6.45 (m, 1H), 6.22 (dd, J = 15.2 Hz & 2.0 Hz, 1H), 5.74 (d, J = 10.0 Hz, 1H), 3.81 (s, 3H), 3.79 (s, 3H), 3.56 (s, 3H). |
| 3 | 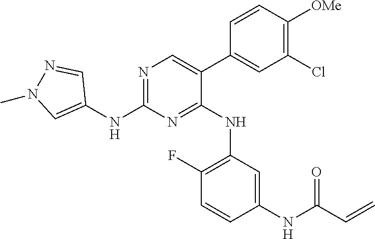 [SMILES: C=CC(=O)Nc1ccc(F)c(Nc2nc(Nc3cnn(C)c3)ncc2-c2ccc(OC)c(Cl)c2)c1] | K | 494.09 | δ 10.21 (s, 1H), 9.12 (bs, 1H), 8.32 (bs, 1H), 7.88 (s, 1H), 7.75-7.76 (m, 1H), 7.49-7.58 (m, 2H), 7.38- 7.41 (m, 1H), 7.16-7.28 (m, 4H), 6.37-6.44 (m, 1H), 6.22 (d, J = 15.2 Hz, 1H), 5.73 (d, J = 10.0 Hz, 1H), 3.89 (s, 3H), 3.55 (s, 3H). |
| 4 | 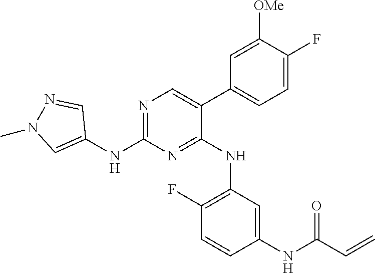 [SMILES: C=CC(=O)Nc1ccc(F)c(Nc2nc(Nc3cnn(C)c3)ncc2-c2ccc(F)c(OC)c2)c1] | K | 478.21 | δ 10.31 (s, 1H), 9.95 (bs, 1H), 9.22 (bs, 1H), 7.95 (s, 1H), 7.86-7.87 (m, 1H), 7.58 (s, 1H), 7.06-7.37 (m, 6H), 6.38-6.45 (m, 1H), 6.22 (dd, J = 16.8 Hz & 2.0 Hz, 1H), 5.75 (dd, J = 10.0 Hz & 2.0 Hz, 1H), 3.90 (s, 3H), 3.57 (s, 3H). |
| 5 | 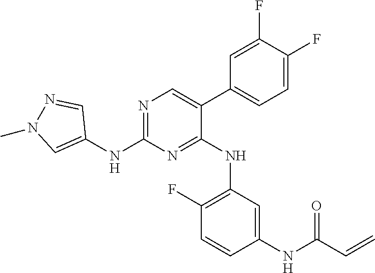 [SMILES: C=CC(=O)Nc1ccc(F)c(Nc2nc(Nc3cnn(C)c3)ncc2-c2ccc(F)c(F)c2)c1] | K | 466.16 | δ 10.23 (s, 1H), 9.20 (bs, 1H), 8.40 (bs, 1H), 7.92 (s, 1H), 7.75-7.77 (m, 1H), 7.47-7.56 (m, 3H), 7.08- 7.30 (m, 4H), 6.37-6.44 (m, 1H), 6.22 (d, J = 16.8 Hz, 1H), 5.74 (d, J = 10.4 Hz, 1H), 3.54 (s, 3H). |
| 6 | 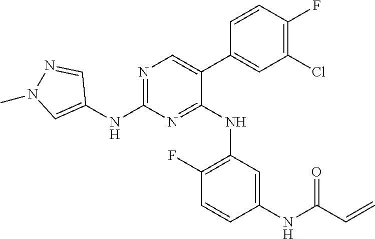 [SMILES: C=CC(=O)Nc1ccc(F)c(Nc2nc(Nc3cnn(C)c3)ncc2-c2ccc(F)c(Cl)c2)c1] | K | 482.16 | δ 10.23 (s, 1H), 9.20 (bs, 1H), 8.44 (bs, 1H), 7.92 (s, 1H), 7.75-7.76 (m, 1H), 7.64-7.66 (m, 1H), 7.57 (s, 1H), 7.44-7.49 (m, 2H), 7.10- 7.22 (m, 3H), 6.38-6.44 (m, 1H), 6.22 (d, J = 15.2 Hz, 1H), 5.73 (d, J = 10.4 Hz, 1H), 3.47 (s, 3H). |
| 7 | 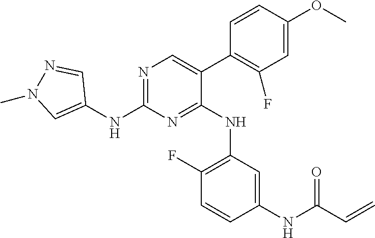 [SMILES: C=CC(=O)Nc1ccc(F)c(Nc2nc(Nc3cnn(C)c3)ncc2-c2ccc(OC)cc2F)c1] | K | 478.17 | δ 10.20 (s, 1H), 9.12 (bs, 1H), 8.17 (s, 1H), 7.82 (s, 1H), 7.75-7.77 (m, 1H), 7.58 (s, 1H), 7.32-7.36 (m, 1H), 7.17-7.26 (m, 3H), 6.87- 6.94 (m, 2H), 6.37-6.44 (m, 1H), 6.21 (d, J = 15.2 Hz, 1H), 5.73 (d, J = 10.0 Hz, 1H), 3.81 (s, 3H), 3.55 (s, 3H). |
| 8 | 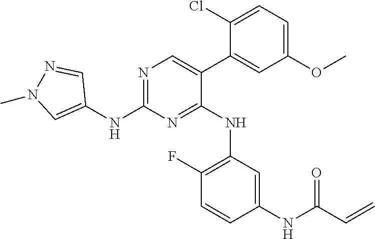 [SMILES: C=CC(=O)Nc1ccc(F)c(Nc2nc(Nc3cnn(C)c3)ncc2-c2cc(OC)ccc2Cl)c1] | K | 494.3 | δ 10.17 (s, 1H), 9.10 (s, 1H), 8.05 (s, 1H), 7.80 (s, 1H), 7.73-7.71 (m, 1H), 7.58 (s, 1H), 7.43 (d, J = 8.0 Hz, 1H), 7.30-7.10 (m, 3H), 6.99-6.96 (m, 2H), 6.42-6.35 (m, 1H), 6.24-6.20 (m, 1H), 5.74-5.71 (m, 1H), 3.78 (s, 3H), 3.54 (s, 3H). |
| 9 | 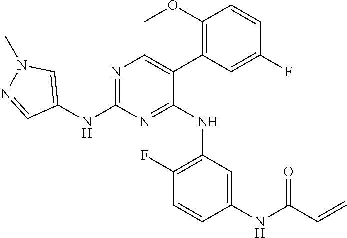 [SMILES: C=CC(=O)Nc1ccc(F)c(Nc2nc(Nc3cnn(C)c3)ncc2-c2cc(F)ccc2OC)c1] | K | 478.3 | δ 10.19 (s, 1H), 9.12 (s, 2H), 7.89- 7.75 (m, 3H), 7.56 (s, 1H), 7.24- 7.05 (m, 4H), 6.50-6.35 (m, 1H), 6.24-6.20 (m, 1H), 5.73 (d, J = 12.0 Hz, 2H), 3.78 (s, 3H), 3.53 (s, 3H). |
| 10 | 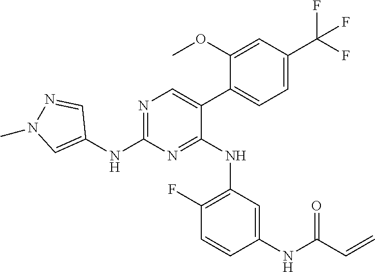 [SMILES: C=CC(=O)Nc1ccc(F)c(Nc2nc(Nc3cnn(C)c3)ncc2-c2ccc(C(F)(F)F)cc2OC)c1] | K | 528.3 | δ 10.17 (s, 1H), 9.08 (s, 1H) ,7.97 (s, 1H), 7.82 (s, 1H) 7.73-7.72 (m, 1H), 7.56 (s, 1H), 7.49-7.47 (m, 1H), 7.37-7.31 (m, 2H), 7.23-7.21 (m, 3H), 6.42-6.40 (m, 1H), 6.25- 6.20 (m, 1H), 5.74-5.72 (m, 1H), 3.88 (s, 3H), 3.54 (s, 3H). |
| 11 | 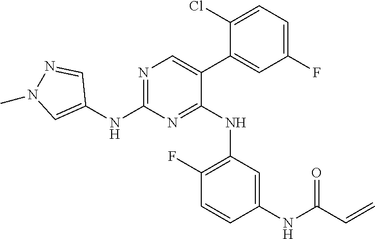 [SMILES: C=CC(=O)Nc1ccc(F)c(Nc2nc(Nc3cnn(C)c3)ncc2-c2cc(F)ccc2Cl)c1] | K | 482.08 | δ 10.22 (bs, 1H), 9.21 (bs, 1H), 8.24 (bs, 1H), 7.83 (s, 1H), 7.72- 7.73 (m, 1H), 7.59-7.62 (m, 2H), 7.32-7.36 (m, 1H), 7.27-7.30 (m, 2H), 7.08-7.17 (m, 2H), 6.37-6.47 (m, 1H), 6.21-6.26 (m, 1H), 5.72 (d, J = 10.0 Hz, 1H), 3.54 (s, 3H). |
| 12 | 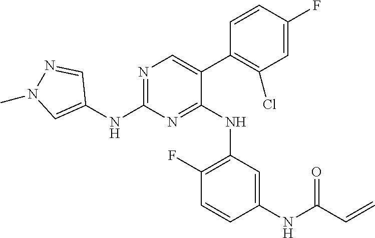 [SMILES: C=CC(=O)Nc1ccc(F)c(Nc2nc(Nc3cnn(C)c3)ncc2-c2ccc(F)cc2Cl)c1] | K | 482.09 | δ 10.25 (bs, 1H), 9.16 (bs, 1H), 8.16 (bs, 1H), 7.79 (s, 1H), 7.73- 7.74 (m, 1H), 7.54-7.60 (m, 3H), 7.29-7.33 (m, 2H), 7.17-7.26 (m, 2H), 6.38-6.44 (m, 1H), 6.21-6.25 (m, 1H), 5.73 (d, J = 10.0 Hz, 1H), 3.54 (s, 3H). |
| 13 | 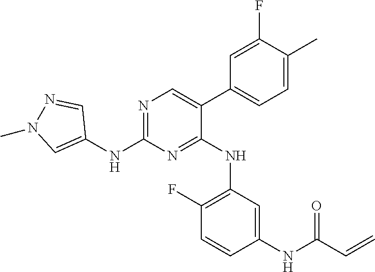 [SMILES: C=CC(=O)Nc1ccc(F)c(Nc2nc(Nc3cnn(C)c3)ncc2-c2ccc(C)c(F)c2)c1] | K | 462.13 | δ 10.21 (bs, 1H), 9.15 (bs, 1H), 8.34 (bs, 1H), 7.92 (s, 1H), 7.75- 7.77 (m, 1H), 7.58 (s, 1H), 7.35- 7.39 (m, 1H), 7.09-7.25 (m, 5H), 6.38-6.44 (m, 1H), 6.22-6.26 (m, 1H), 5.73 (d, J = 10.0 Hz, 1H), 3.35 (s, 3H), 2.28 (s, 3H). |
| 14 | 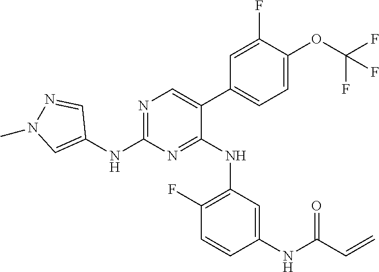 [SMILES: C=CC(=O)Nc1ccc(F)c(Nc2nc(Nc3cnn(C)c3)ncc2-c2ccc(OC(F)(F)F)c(F)c2)c1] | K₁ | 532.2 | δ 10.23 (s, 1H), 9.23 (s, 1H), 8.69 (s, 1H), 8.21 (s, 1H), 7.78-7.64 (m, 4H), 7.42 (d, J = 8.4 Hz, 1H), 7.30-7.11 (m, 3H), 6.46-6.39 (m, 1H), 6.25 (d, J = 16.8 Hz, 1H), 5.76 (d, J = 10.0 Hz, 1H), 3.56 (s, 3H) |
| 15 | 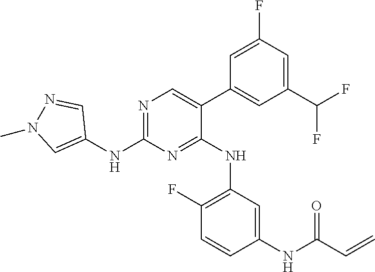 [SMILES: C=CC(=O)Nc1ccc(F)c(Nc2nc(Nc3cnn(C)c3)ncc2-c2cc(F)cc(C(F)F)c2)c1] | K₁ | 498.1 | δ 10.32 (s, 1H), 10.23 (s, 1H), 9.48 (s, 1H), 8.05 (s, 1H), 7.88 (s, 1H), 7.60-7.51 (m, 4H), 7.38 (s, 1H), 7.25-6.98 (m, 3H), 6.46-6.39 (m, 1H), 6.26 (d, J = 16.8 Hz, 1H), 5.77 (d, J = 10.0 Hz, 1H), 3.60 (s, 3H). |
| 16 | 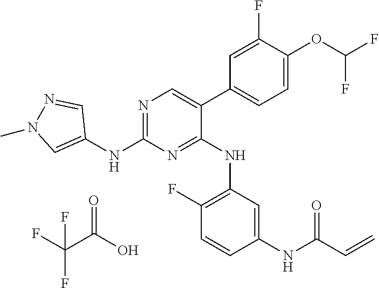 [SMILES: C=CC(=O)Nc1ccc(F)c(Nc2nc(Nc3cnn(C)c3)ncc2-c2ccc(OC(F)F)c(F)c2)c1 ; O=C(O)C(F)(F)F] | K₁ | 514.4 | δ 10.30 (s, 1H), 10.03 (s, 1H), 9.36 (s, 1H), 7.96 (s, 1H), 7.85 (s, 1H), 7.59-7.12 (m, 9H), 6.43-6.36 (m, 1H), 6.24 (d, J = 17.2 Hz, 1H), 5.76 (d, J = 10.0 Hz, 1H), 3.56 (s, 3H) |
| 17 | 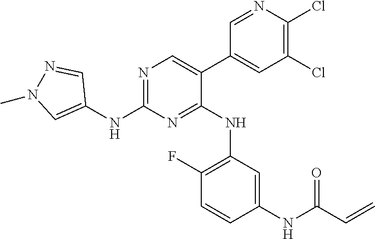 [SMILES: C=CC(=O)Nc1ccc(F)c(Nc2nc(Nc3cnn(C)c3)ncc2-c2cnc(Cl)c(Cl)c2)c1] | K₁ | 499.1 | δ 10.32 (s, 1H), 9.35 (s, 1H), 8.70 (s, 1H), 8.48-8.47 (d, J = 5.2 Hz, 1H), 8.22-8.21 (s, 1H), 7.95 (m, 1H), 7.81-7.78 (m, 1H), 7.65- 7.53 (s, 2H), 7.32 (s, 1H), 7.17- 7.12 (d, J = 17.6 Hz, 1H), 6.47- 6.41 (m, 1H), 6.27-6.22 (m, 1H), 5.77-5.74 (d, J = 12.0 Hz, 1H), 3.54 (s, 3H) |
| 18 | 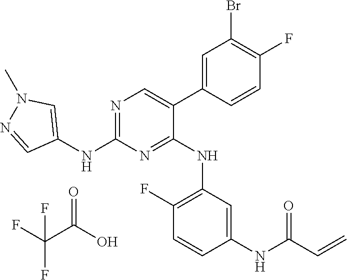 | K₁ | 526.2 | δ 10.31 (s, 1H), 10.05 (s, 1H), 9.34 (bs, 1H), 7.95 (s, 1H), 7.84-7.83 (m, 2H), 7.55-7.48 (m, 3H), 7.40- 7.30 (m, 1H), 7.21 (bs, 1H), 7.13 (bs, 1H), 7.08-6.95 (m, 1H), 6.43- 6.36 (m, 1H), 6.24 (dd, J = 16.4 Hz, 1.6 Hz, 1H), 5.76 (dd, J = 9.6 Hz, 1.6 Hz, 1H), 3.59 (s, 3H) |
| 19 | 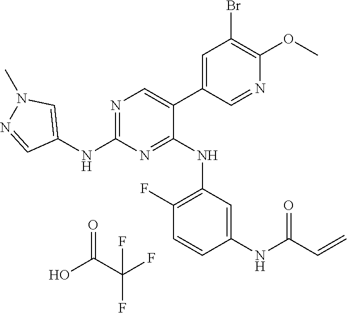 | K₁ | 479.3 | δ 10.27 (s, 1H), 9.924 (s, 1H), 9.26 (s, 1H), 8.08 (s, 1H), 7.94 (bs, 1H), 7.82 (d, J = 11.6 Hz, 2H), 7.56-7.15 (m, 5H), 6.43-6.36 (m, 1H), 6.24 (d, J = 15.6 Hz, 1H), 5.75 (d, J = 11.2 Hz, 1H), 3.99 (s, 3H), 3.44 (s, 3H) |
| 20 | 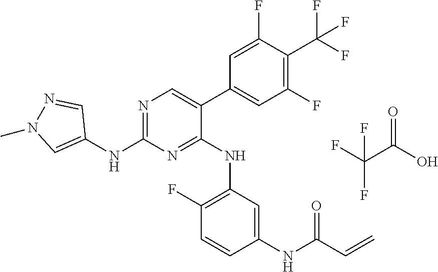 | K₂ | 534.2 | δ 10.31 (s, 1H), 9.96 (bs, 1H), 9.25 (bs, 1H), 8.09 (s, 1H), 7.86 (s, 1H), 7.58-7.55 (m, 3H), 7.45- 7.25 (m, 1H), 7.19-6.90 (m, 3H), 6.44-6.37 (m, 1H), 6.24 (dd, J = 19.2 Hz, 2.0 Hz, 1H), 5.75 (dd, J = 12.0 Hz, 1.6 Hz, 1H), 3.55 (s, 3H). |
| 22 | 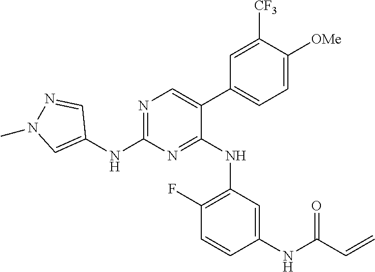 [SMILES: C=CC(=O)Nc1ccc(F)c(Nc2nc(Nc3cnn(C)c3)ncc2-c2ccc(OC)c(C(F)(F)F)c2)c1] | K₁ | 528.5 | δ 10.21 (s, 1H), 9.15 (bs, 1H), 8.38 (bs, 1H), 7.91 (s, 1H), 7.69-7.76 (m, 2H), 7.64 (s, 1H), 7.57 (s, 1H), 7.34 (d, J = 8.4 Hz, 1H), 7.27 (s, 1H), 7.17 (s, 2H), 6.37-6.44 (m, 1H), 6.21 (dd, J = 2.0 & 16.8 Hz, 1H), 5.73 (dd, J = 2.0 & 10.0 Hz, 1H), 3.92 (s, 3H), 3.31 (s, 3H). |

### Step 1: Synthesis of 5-(5-chloro-2-methoxyphenyl)-N4-(2-fluoro-5-nitrophenyl)-N2-(1-methyl-1H-pyrazol-4-yl)pyrimidine-2,4-diamine (6)

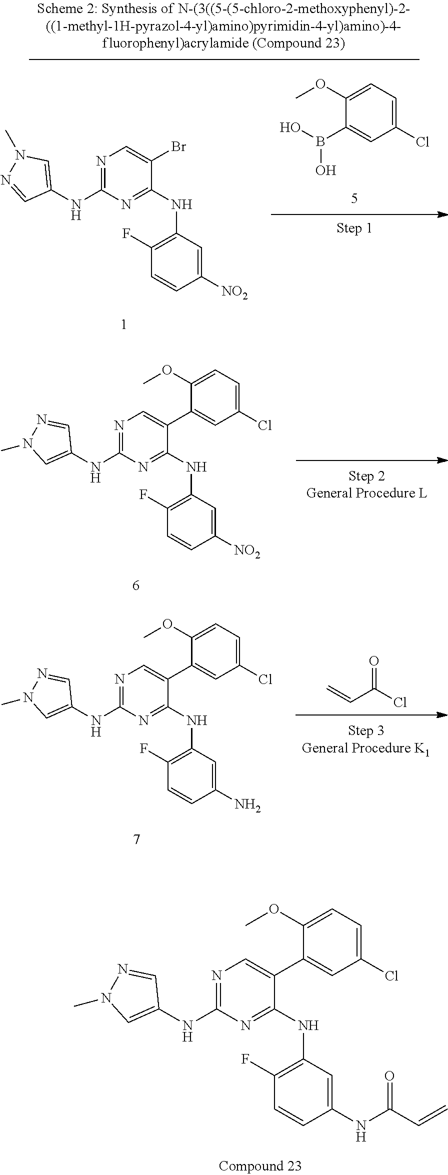

To a solution of 5-bromo-N4-(2-fluoro-5-nitrophenyl)-N2-(1-methyl-1H-pyrazol-4-yl)pyrimidine-2,4-diamine (1) (0.3 g, 0.735 mmol), (5-chloro-2-methoxyphenyl)boronic acid (6) (164 mg, 0.88 mmol) in 1,2-dimethoxyethane (4.00 mL) and water (1.50 mL) was added sodium hydrogen carbonate (0.185 g, 2.20 mmol). Then the reaction mixture was purged with nitrogen for 10 minutes, added bis(triphenylphosphine)palladium(II) dichloride (51.6 mg, 0.073.5 mmol) and the reaction mixture was heated at 80° C. for 16 hours. Progress of the reaction was monitored by LCMS. After completion of the reaction, the reaction mixture was diluted with water (5 mL) and extracted with ethyl acetate (2×50 mL). The combined organic layer was washed with brine (25 mL), dried over anhydrous sodium sulfate and concentrated under vacuo. The crude product was purified by combiflash purifier with 85% ethyl acetate in hexane as eluent to afford the title compound (6) (0.2 g, 0.426 mmol) as yellow solid. LCMS: [M+H]⁺ 470.0.

### Step 2: Preparation of N4-(5-amino-2-fluorophenyl)-5-(5-chloro-2-methoxy-phenyl)-N2-(1-methyl-1H-pyrazol-4-yl)pyrimidine-2,4-diamine (7)

The title compound was prepared in a manner substantially similar to procedure mentioned in General Procedure L to afford brown liquid (0.13 g, crude). LCMS: [M+H]⁺ 440.0

### Step 3: Synthesis of N-(3-((5-(5-chloro-2-methoxyphenyl)-2-((1-methyl-1H-pyrazol-4-yl)amino)pyrimidin-4-yl)amino)-4-fluorophenyl)acrylamide (Compound 23)

The title compound was prepared in a manner substantially similar to procedure mentioned in General Procedure K₁ to afford off white solid (0.01 g, 9 yield). ¹H NMR (400 MHz, DMSO-d6): δ10.19 (s, 1H), 9.10 (s, 1H), 7.92 (s, 1H), 7.80 (s, 1H), 7.73 (d, J 6.0 Hz, 1H), 7.57 (s, 2H), 7.39 (d, J=8.4 Hz, 1H), 7.07-7.29 (m, 4H), 6.35-6.39 (m, 1H), 6.22 (d, J=16.4 Hz, 1H), 5.73 (d, J=9.6 Hz, 1H), 3.79 (s, 3H), 3.53 (s, 3H). LCMS: [M+H]⁺ 494.3.

TABLE 2
The following compounds were prepared using the procedures described above:

| | | General | | |
|---|---|---|---|---|
| Cmpd. | | Pro- | LCMS | |
| No. | Structure | cedure | [M + H] | ¹H-NMR (400 MHz, DMSO-d₆) |
| 24 | 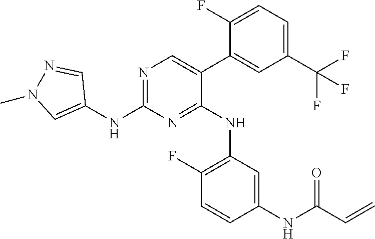 [SMILES: C=CC(=O)Nc1ccc(F)c(Nc2nc(Nc3cnn(C)c3)ncc2-c2cc(C(F)(F)F)ccc2F)c1] | K | 516.3 | δ 10.17 (s, 1H), 9.24 (s, 1H), 8.44 (s, 1H), 7.92 (s, 1H), 7.70 (d, J = 9.6 Hz, 2H), 7.53 (d, J = 15.2 Hz, 1H), 7.49-7.46 (m, 2H), 7.25-7.17 (m, 3H), 6.41-6.34 (m, 1H), 6.24- 6.24 (s, 1H), 5.73 (d, J = 11.6 Hz, 1H), 5.34 (s, 3H). |
| 25 | 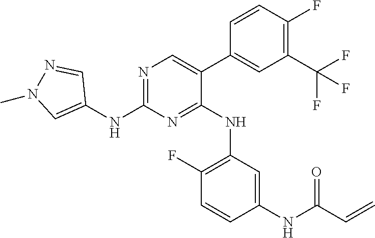 [SMILES: C=CC(=O)Nc1ccc(F)c(Nc2nc(Nc3cnn(C)c3)ncc2-c2ccc(F)c(C(F)(F)F)c2)c1] | K | 516.0 | δ 10.19 (bs, 1H), 9.19 (bs, 1H), 8.46 (bs, 1H), 7.94 (s, 1H), 7.78- 7.72 (m, 3H), 7.58-7.53 (m, 2H), 7.26-7.16 (m, 3H), 6.42-6.35 (m, 1H), 6.24-6.20 (m, 1H), 5.73 (d, J = 11.2 Hz, 1H), 3.55 (s, 3H). |
| 26 | 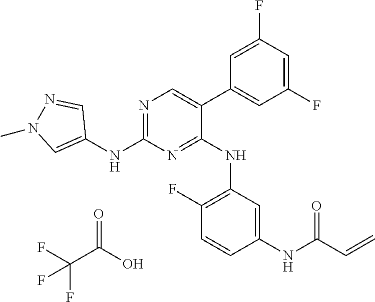 [SMILES: C=CC(=O)Nc1ccc(F)c(Nc2nc(Nc3cnn(C)c3)ncc2-c2cc(F)cc(F)c2)c1 ; O=C(O)C(F)(F)F] | K | 466.2 | δ 10.25 (s, 1H), 9.73 (s, 1H), 9.07 (s, 1H), 8.35 (s, 1H), 7.98 (s, 1H), 7.82 (d, J = 4.8 Hz, 1H), 7.57 (s, 1H), 7.37-7.19 (m, 5H), 7.10-6.93 (m, 1H), 6.43-6.36 (m, 1H), 6.25- 6.21 (m, 1H), 5.76-5.73 (m, 1H), 3.5 (s, 3H). |
| 27 | 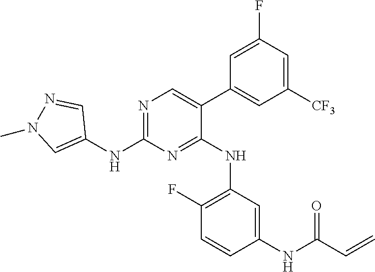 [SMILES: C=CC(=O)Nc1ccc(F)c(Nc2nc(Nc3cnn(C)c3)ncc2-c2cc(F)cc(C(F)(F)F)c2)c1] | K | 516.3 | δ 10.21 (s, 1H), 9.30 (bs, 1H), 8.62 (bs, 1H), 8.03 (s, 1H), 7.77 (bs, 1H), 7.65-7.60 (m, 4H), 7.30-7.19 (m, 3H), 6.45-6.38 (m, 1H), 6.27- 6.23 (m, 1H), 5.76 (d, J = 10.0 Hz, 1H), 3.56 (s, 3H). |
| 28 | 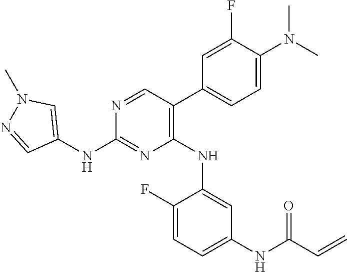 [SMILES: C=CC(=O)Nc1ccc(F)c(Nc2nc(Nc3cnn(C)c3)ncc2-c2ccc(N(C)C)c(F)c2)c1] | K₁ | 491.3 | δ 10.31 (s, 1H), 10.19 (s, 1H), 9.50 (s, 1H), 7.88 (s, 2H), 7.20 (s, 1H), 7.37 (s, 1H), 7.30-7.21 (m, 3H), 7.09 (t, J = 8.8 Hz, 2H), 6.46-6.39 (m, 1H), 6.26 (d, J = 16.8 Hz, 1H), 5.77 (d, J = 10.0 Hz, 1H), 3.61 (s, 3H), 2.86 (s, 6H). |
| 29 | 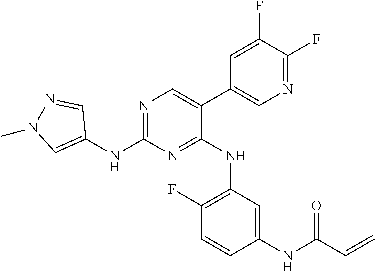 [SMILES: C=CC(=O)Nc1ccc(F)c(Nc2nc(Nc3cnn(C)c3)ncc2-c2cnc(F)c(F)c2)c1] | K₁ | 467.3 | δ 10.31 (s, 1H), 9.37 (s, 1H), 8.64 (s, 1H), 8.24-8.19 (m, 2H), 8.05 (s, 1H), 7.84 (d, J = 4.8 Hz, 1H), 7.63 (s, 1H), 7.37 (s, 1H), 7.20 (d, J = 20.0 Hz, 2H), 6.51-6.44 (m, 1H), 6.33-6.29 (m, 1H), 5.84- 5.80 (m, 1H), 3.60 (s, 3H) |
| 30 | 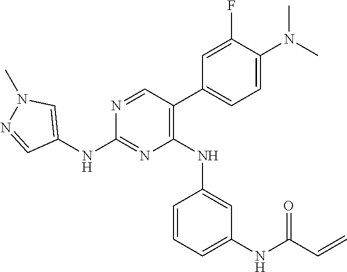 [SMILES: C=CC(=O)Nc1cccc(Nc2nc(Nc3cnn(C)c3)ncc2-c2ccc(N(C)C)c(F)c2)c1] | K₁ | 482.3 | δ 10.11 (s, 1H), 9.06 (s, 1H), 8.30 (s, 1H), 7.88 (s, 1H), 7.35 (s, 1H), 7.73-7.44 (m, 3H), 7.27-7.14 (m, 4H), 7.02 (t, J = 9.2 Hz, 1H), 6.46- 6.39 (m, 1H), 6.24-6.19 (m, 1H), 5.71 (t, J = 10.0 Hz, 1H), 3.58 (s, 3H), 2.80 (s, 6H). |
| 31 | 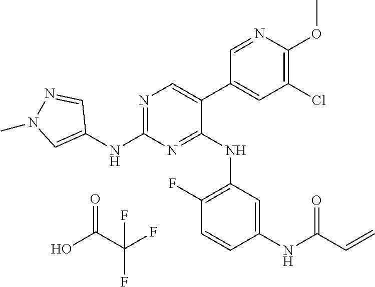 [SMILES: C=CC(=O)Nc1ccc(F)c(Nc2nc(Nc3cnn(C)c3)ncc2-c2cnc(OC)c(Cl)c2)c1 ; O=C(O)C(F)(F)F] | K₁ | 495.2 | δ 10.33-10.24 (m, 2H), 9.57 (s, 1H), 8.27-8.22 (s, 1H), 8.03- 7.99 (m, 2H), 7.87 (s, 1H), 7.56- 7.15 (m, 5H), 6.43-6.36 (m, 1H), 6.26-6.21 (m, 1H), 5.77-5.74 (m, 1H), 4.021-3.99 (s, 3H), 3.57 (s, 3H). |
| 32 | 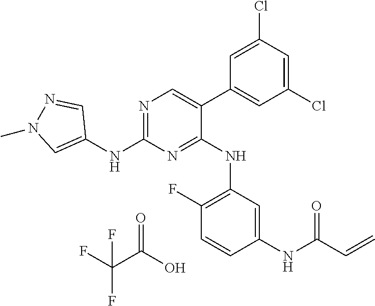 | K | 498.2 | δ 10.27 (s, 1H), 9.92 (bs, 1H), 9.28 (bs, 1H), 7.98 (s, 1H), 7.98 (d, J = 4.8 Hz, 1H), 7.64 (s, 1H), 7.54- 7.57 (m, 3H), 7.33 (bs, 2H), 7.21 (s, 1H), 7.12 (s, 1H), 6.36-6.43 (m, 1H), 6.21-6.26 (m, 1H), 5.73 (d, J = 5.0 Hz, 1H), 3.56 (s, 3H). |
| 33 | 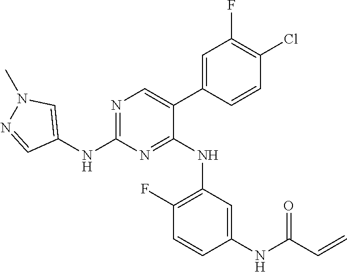 [SMILES: C=CC(=O)Nc1ccc(F)c(Nc2nc(Nc3cnn(C)c3)ncc2-c2ccc(Cl)c(F)c2)c1] | K₁ | 482.3 | δ 10.22 (s, 1H), 9.24 (s, 1H), 8.48 (s, 1H), 7.97 (s, 1H), 7.76 (d, J = 8.0 Hz, 1H), 7.68-7.51 (m, 3H), 7.36-7.30 (m, 2H), 7.17-7.10 (m, 2H), 6.45-6.38 (m, 1H), 6.25 (d, J = 16.0 Hz, 1H), 5.75 (d, J = 12.0 Hz, 1H), 3.55 (bs, 3H). |
| 34 | 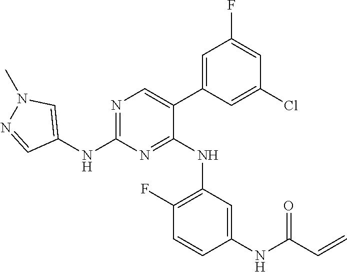 [SMILES: C=CC(=O)Nc1ccc(F)c(Nc2nc(Nc3cnn(C)c3)ncc2-c2cc(F)cc(Cl)c2)c1] | K₁ | 482.3 | δ 10.19 (s, 1H), 9.22 (s, 1H), 8.52 (s, 1H), 7.97 (s, 1H), 7.74 (d, J = 6.4 Hz, 1H), 7.57 (s, 1H), 7.37- 7.29 (m, 4H), 7.15 (s, 2H), 6.43- 6.36 (m, 1H), 6.23 (d, J = 16.8 Hz, 1H), 5.73 (d, J = 10.0 Hz, 1H), 3.54 (s, 3H). |
| 35 | 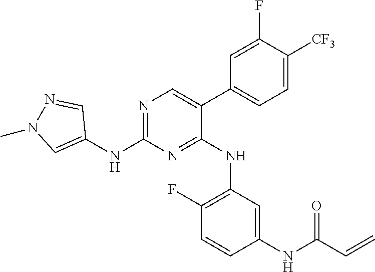 [SMILES: C=CC(=O)Nc1ccc(F)c(Nc2nc(Nc3cnn(C)c3)ncc2-c2ccc(C(F)(F)F)c(F)c2)c1] | K | 516.20 | δ 10.25 (bs, 1H), 9.23 (bs, 1H), 8.65 (s, 1H), 8.03 (s, 1H), 7.81 (t, J = 8.0 Hz, 1H), 7.76-7.77 (m, 1H), 7.61-7.64 (m, 2H), 7.52-7.54 (m, 1H), 7.31 (s, 1H), 7.08-7.15 (m, 2H), 6.38-6.44 (m, 1H), 6.20- 6.26 (m, 1H), 5.74 (d, J = 10.0 Hz, 1H), 3.53 (s, 3H). |
| 36 | 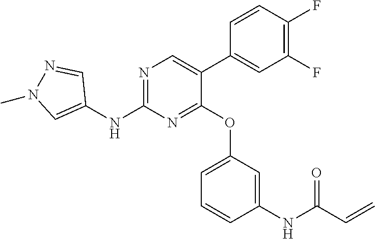 [SMILES: C=CC(=O)Nc1cccc(Oc2nc(Nc3cnn(C)c3)ncc2-c2ccc(F)c(F)c2)c1] | K | 448.88 | δ 10.36 (bs, 1H), 9.76 (bs, 1H), 8.46 (s, 1H), 7.78-7.85 (m, 1H), 7.70 (s, 1H), 7.62 (s, 1H), 7.48- 7.55 (m, 3H), 7.10 (s, 1H), 7.04 (d, J = 6.8 Hz, 1H), 6.85 (s, 1H), 6.39- 6.46 (m, 1H), 6.23-6.27 (m, 1H), 5.75 (d, J = 10.0 Hz, 1H), 3.50 (s, 3H) |
| 37 | 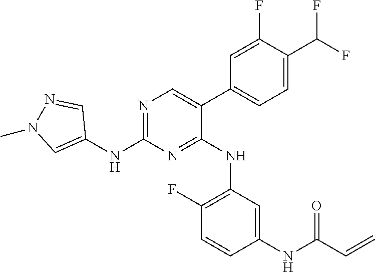 [SMILES: C=CC(=O)Nc1ccc(F)c(Nc2nc(Nc3cnn(C)c3)ncc2-c2ccc(C(F)F)c(F)c2)c1] | K₁ | 498.3 | δ 10.27 (s, 1H), 9.77 (s, 1H), 9.16 (s, 1H), 7.99 (s, 1H), 7.81 (s, 1H), 7.71-7.75 (m, 1H), 7.47-7.56 (m, 3H), 7.39-7.12 (m, 4H), 6.43-6.36 (m, 1H), 6.23 (d, J = 16.8 Hz, 1H), 5.75 (d, J = 10.4 Hz, 1H), 3.52 (3H) |
| 38 | 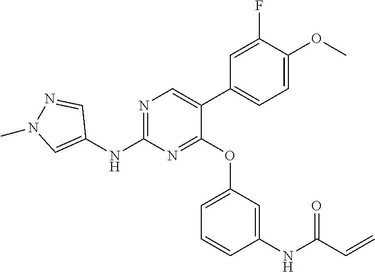 [SMILES: C=CC(=O)Nc1cccc(Oc2nc(Nc3cnn(C)c3)ncc2-c2ccc(OC)c(F)c2)c1] | K | 461.01 | δ 10.37 (bs, 1H), 9.68 (bs, 1H), 8.42 (s, 1H), 7.46-7.68 (m, 5H), 7.25 (t, J = 8.8 Hz, 1H), 7.10 (s, 1H), 7.03 (d, J = 6.8 Hz, 1H), 6.84 (s, 1H), 6.39-6.46 (m, 1H), 6.23- 6.27 (m, 1H), 5.75 (d, J = 10.0 Hz, 1H), 3.87 (s, 3H), 3.50 (s, 3H) |
| 39 | 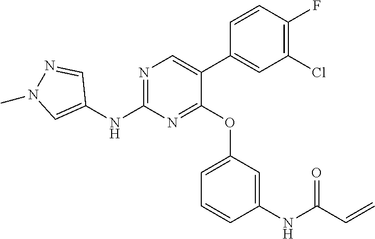 [SMILES: C=CC(=O)Nc1cccc(Oc2nc(Nc3cnn(C)c3)ncc2-c2ccc(F)c(Cl)c2)c1] | K | 464.98 | δ 10.37 (bs, 1H), 9.77 (bs, 1H), 8.46 (s, 1H), 7.91-7.92 (m, 1H), 7.49-7.70 (m, 5H), 7.10 (s, 1H), 7.03-7.05 (m, 1H), 6.82 (s, 1H), 6.39-6.46 (m, 1H), 6.23-6.27 (m, 1H), 5.75 (d, J = 10.0 Hz, 1H), 3.50 (s, 3H) |
| 40 | 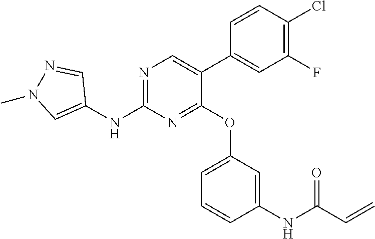 [SMILES: C=CC(=O)Nc1cccc(Oc2nc(Nc3cnn(C)c3)ncc2-c2ccc(Cl)c(F)c2)c1] | K | 465.05 | δ 10.37 (bs, 1H), 9.81 (bs, 1H), 8.51 (m, 1H), 7.51-7.79 (m, 6H), 7.04-7.15 (m, 2H), 6.85 (bs, 1H), 6.39-6.51 (m, 1H), 6.23-6.27 (m, 1H), 5.76 (d, J = 10.0 Hz, 1H), 3.50 (s, 3H) |
| 41 | 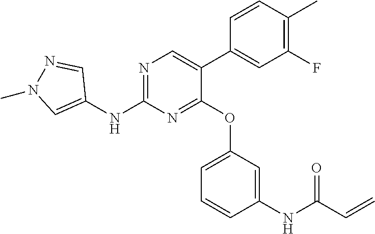 [SMILES: C=CC(=O)Nc1cccc(Oc2nc(Nc3cnn(C)c3)ncc2-c2ccc(C)c(F)c2)c1] | K | 445.05 | δ 10.35 (bs, 1H), 9.71 (bs, 1H), 8.45 (s, 1H), 7.38-7.91 (m, 6H), 7.10 (bs, 1H), 7.02-7.04 (m, 1H), 6.85 (bs, 1H), 6.39-6.46 (m, 1H), 6.23-6.27 (m, 1H), 5.76 (d, J = 10.0 Hz, 1H), 3.50 (s, 3H), 2.27 (s, 3H) |
| 42 | 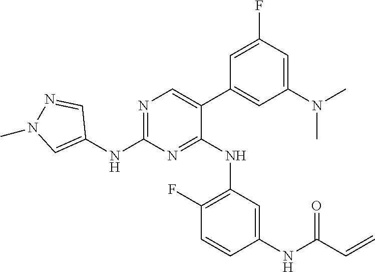 [SMILES: C=CC(=O)Nc1ccc(F)c(Nc2nc(Nc3cnn(C)c3)ncc2-c2cc(F)cc(N(C)C)c2)c1] | K₁ | 491.2 | δ 10.20 (s, 1H), 9.13 (s, 1H), 8.25 (s, 2H), 7.94 (s, 1H), 7.78 (s, 1H), 7.59 (s, 1H), 7.12-7.35 (m, 3H), 6.57-6.21 (m, 4H), 5.74 (d, J = 11.6 Hz, 1H), 3.53 (s, 3H), 2.94 (s, 6H). |
| 43 | 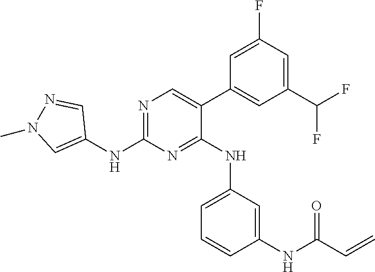 [SMILES: C=CC(=O)Nc1cccc(Nc2nc(Nc3cnn(C)c3)ncc2-c2cc(F)cc(C(F)F)c2)c1] | K₁ | 480.2 | δ 10.23 (s, 1H), 9.87 (bs, 1H), 9.35 (bs, 1H), 7.82-7.96 (m, 3H), 7.07- 7.58 (m, 7H), 6.95 (s, 1H), 6.39- 6.46 (m, 1H), 6.20-6.25 (m, 1H), 6.72-5.75 (m, 1H), 3.43 (s, 3H). |
| 44 | 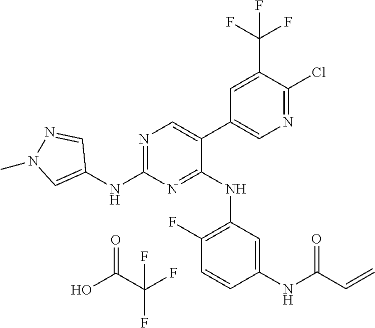 | K₁ | 531.0 | δ 10.31 (s, 1H), 9.8 (bs, 1H), 9.2 (bs, 1H), 8.79 (s, 1H), 8.42 (s, 1H), 8.09 (bs, 1H), 7.85 (bs, 1H), 7.58-7.36 (s, 3H), 7.22-7.18 (m, 2H), 6.45-6.38 (m, 1H), 6.25 (d, J = 17.2 Hz, 1H), 5.77 (d, J = 10.0 Hz, 1H), 3.57 (s, 3H) |
| 45 | 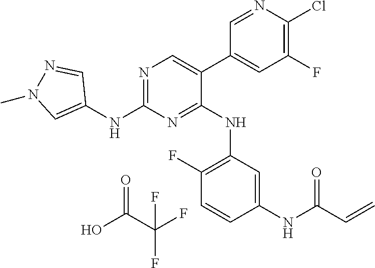 | K₁ | 483.1 | δ 10.32 (s, 1H), 9.96 (bs, 1H), 9.28 (bs, 1H), 8.43 (s, 1H), 8.25-8.06 (m, 2H), 7.87-7.16 (m, 6H), 6.45- 6.38 (m, 1H), 6.28-6.23 (m, 1H), 5.79-5.76 (m, 1H), 3.68 (s, 3H). |
| 46 | 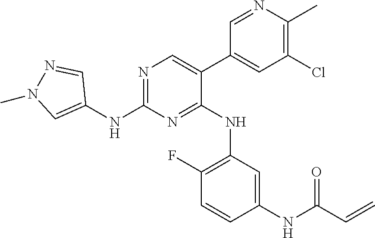 [SMILES: C=CC(=O)Nc1ccc(F)c(Nc2nc(Nc3cnn(C)c3)ncc2-c2cnc(C)c(Cl)c2)c1] | K₁ | 479.3 | δ 10.22 (bs, 1H), 9.26 (bs, 1H), 8.61 (bs, 1H), 8.47 (s, 1H), 7.94- 7.91 (m, 2H), 7.74-7.26 (m, 1H), 7.56 (bs, 1H), 7.29 (bs, 1H), 7.14- 7.08 (m, 2H), 6.43-6.36 (m, 1H), 6.25-6.21 (m, 1H), 5.75-5.72 (d, J = 11.6 Hz, 1H), 3.52 (s, 3H), 2.65 (s, 3H). |
| 47 | 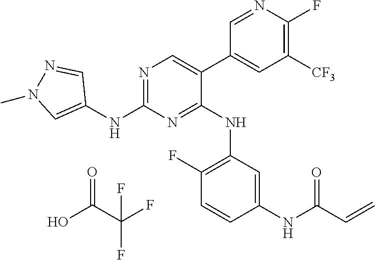 [SMILES: C=CC(=O)Nc1ccc(F)c(Nc2nc(Nc3cnn(C)c3)ncc2-c2cnc(F)c(C(F)(F)F)c2)c1 ; O=C(O)C(F)(F)F] | K₁ | 517.3 | δ 10.30 (s, 1H), 9.88 (s, 1H), 9.21 (s, 1H), 8.62 (s, 1H), 8.48 (d, J = 7.6 Hz, 1H), 8.04-7.85 (s, 3H), 7.55 (s, 1H), 7.19 (d, J = 19.6 Hz, 3H), 6.43-6.21 (m, 2H), 5.75 (d, J = 11.6 Hz, 1H), 3.47 (s, 3H) |
| 48 | 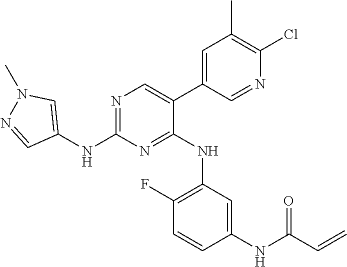 [SMILES: C=CC(=O)Nc1ccc(F)c(Nc2nc(Nc3cnn(C)c3)ncc2-c2cnc(Cl)c(C)c2)c1] | K₁ | 479.3 | δ 10.33 (s, 1H), 10.21 (s, 1H), 9.45 (s, 1H), 8.35 (s, 1H), 8.02 (s, 1H), 7.93 (s, 1H), 7.85 (s, 1H), 7.57 (s, 1H), 7.36 (s, 1H), 7.20-6.97 (m, 2H), 6.44-6.37 (m, 1H), 6.25 (d, J = 17.2 Hz, 1H), 5.75 (d, J = 1.6 Hz, 1H), 3.57 (s, 3 H), 2.48 (d, J = 2.8 Hz, 3H). |
| 49 | 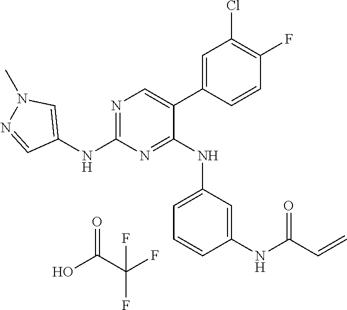 | K₁ | 464.2 | δ 10.24 (s, 1H), 10.09 (s, 1H), 9.40 (s, 1H), 7.93-7.74 (m, 3H), 7.57- 7.52 (m, 4H), 7.39-7.34 (m, 3H), 7.23-7.17 (m, 1H), 6.48- 6.42 (m, 1H), 6.25 (dd, J = 16.8 Hz, 1.6 Hz, 1H), 5.76 (dd, J = 10.4 Hz, 1.6 Hz, 1H), 3.62 (s, 3H). |
| 50 | 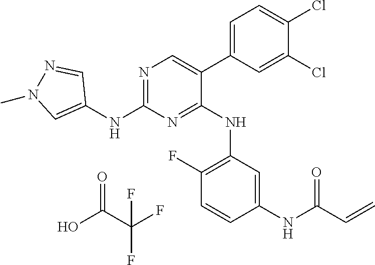 | K₁ | 498.1 | δ 10.29 (s, 1H), 9.90 (s, 1H), 9.24 (s, 1H), 7.96 (s, 1H), 7.82-7.73 (m, 3H), 7.55-7.11 (m, 6H), 6.42- 6.36 (m, 1H), 6.25-6.20 (m, 1H), 5.76-5.73 (m, 1H), 3.62 (m, 3H). |
| 51 | 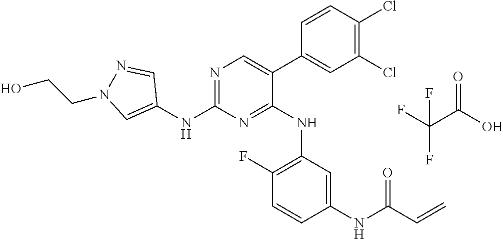 | K₂ | 528.4 | δ 10.26 (s, 1H), 9.91 (bs, 1H), 9.27 (bs, 1H), 7.96 (s, 1H), 7.82 (s, 1H), 7.74 (d, J = 8.8 Hz, 2H), 7.52 (s, 1H), 7.47 (d, J = 8.0 Hz, 1H), 7.32-7.31 (m, 2H), 7.22 (bs, 2H), 6.42-6.36 (m, 1H), 6.23 (dd, J = 18.8 Hz, 2.0 Hz, 1H), 5.74 (dd, J = 11.6 Hz, 1.6 Hz, 1H), 3.84 (bs, 2H), 3.55 (bs, 2H). OH peak is merged along with solvent peak. |
| 52 | 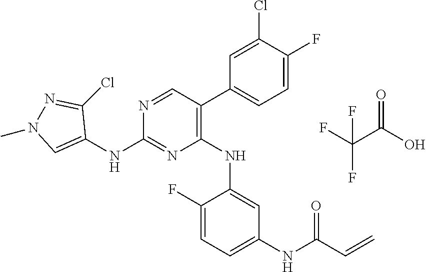 | K₁ | 516.0 | 10.26 (s, 1H), 9.21 (bs, 1H), 7.97 (bs, 1H), 7.83-7.85 (m, 1H), 7.73- 7.75 (m, 1H), 7.49-7.59 (m, 4H), 7.32-7.35 (m, 2H), 6.96- 6.26 (dd, J = 16.0 Hz, 1.9 Hz, 1H), 5.78 (dd, J = 8.0 Hz, 1.9 Hz, 1H), 3.63 (s, 3H). |
| 53 | 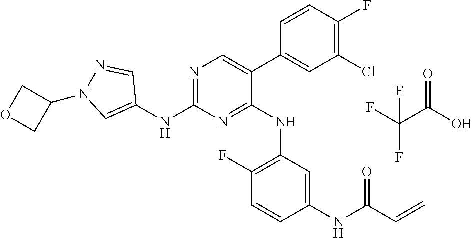 | K₂ | 524.9 | δ 10.33 (bs, 2H), 9.41 (bs, 1H), 8.00 (bs, 1H), 7.88 (bs, 1H), 7.75- 7.74 (m, 1H), 7.57-7.29 (m, 7H), 6.46-6.39 (m, 1H), 6.28- 6.24 (m, 1H), 5.79-5.77 (m, 1H), 5.18 (s, 1H), 4.83-4.73 (m, 4H) |
| 54 | 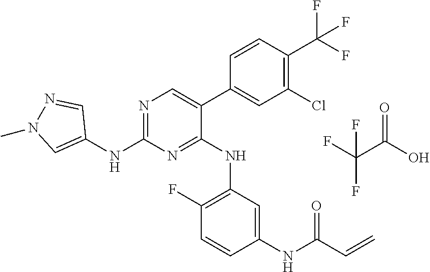 | K₂ | 532.9 | δ 10.33-10.29 (m, 2H), 9.46 (s, 1H), 8.09 (bs, 1H), 8.01-7.98 (m, 1H), 7.88 (bs, 2H), 7.70 (d, J = 8.0 Hz, 1H), 7.60 (s, 1H), 7.38 (bs, 2H), 7.26-7.24 (m, 1H), 7.16 (bs, 1H), 6.46-6.39 (m, 1H), 6.28- 6.23 (m, 1H), 5.78-5.75 (m, 1H), 3.59 (s, 3H merged with DMSO peak). |
| 55 | 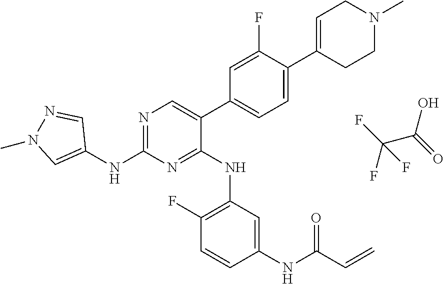 | K₂ | 541.2 [M − H]⁺ | δ 10.29 (s, 1H), 9.94 (bs, 1H), 9.70 (bs, 1H), 8.90 (bs, 1H), 7.99 (s, 1H), 7.85 (d, J = 4.8 Hz, 1H), 7.58 (s, 1H), 7.51 (t, J = 8.0 Hz, 1H), 7.42-7.37 (m, 3H), 7.42 (m, 2H), 6.46-6.39 (m, 1H), 6.27-6.32 (m, 1H), 6.09 (s, 1H), 5.78-5.75 (m, 1H), 4.05-4.01 (m, 2H), 3.82- 3.78 (m, 2H), 3.50 (s, 3H), 2.91- 2.87 (m, 3H), 2.72-2.67 (m, 2H). |
| 56 | 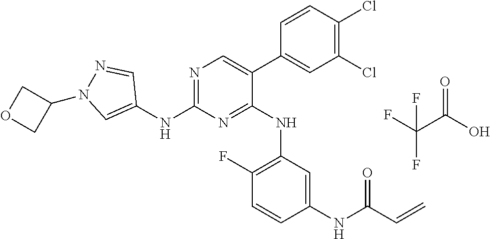 | K₂ | 540.1 | δ 10.32 (s, 1H), 10.20 (bs, 2H), 9.38 (bs, 1H), 8.01 (bs, 1H), 7.85 (bs, 2H), 7.57-7.24 (m, 6H), 6.43- 6.37 (m, 1H), 6.26-6.21 (m, 1H), 5.77-5.74 (m, 1H), 5.20- 5.10 (m, 1H), 4.80-4.69 (m, 4H). |
| 57 | 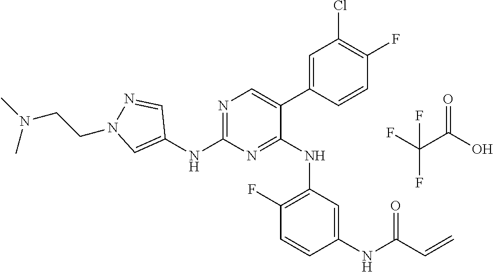 | K₁ | 539.1 | δ 9.95 (s, 1H), 9.0 (s, 1H), 8.19 (s, 1H), 7.96 (s, 1H), 7.74-7.71 (m, 1H), 7.62-7.44 (m, 6H), 7.24 (dd, J = 18.8 Hz, 9.6 Hz, 1H), 6.46- 6.39 (m, 1H), 6.26 (dd, J = 18.8 Hz, 1.6 Hz, 1H), 5.75 (dd, J = 12.0 Hz, 1.6 Hz, 1H), 4.32-4.29 (m, 2H), 3.49-3.32 (m, 3H), 2.797 (s, 6H). |
| 58 | 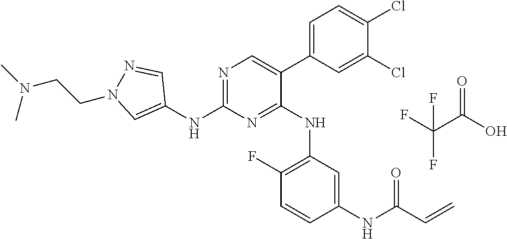 | K | 555.1 | δ 10.28 (s, 1H), 10.00 (bs, 1H), 9.39 (bs, 1H), 9.10 (bs, 1H), 7.99- 7.22 (m, 9H), 6.43-6.36 (m, 1H), 6.23 (dd, J = 18.4 Hz, 2.0 Hz, 1H), 5.75 (dd, J = 11.6 Hz, 1.6 Hz, 1H), 4.22 (s, 2H), 3.41 (s, 2H), 2.75-2.30 (m, 6H). |
| 59 | 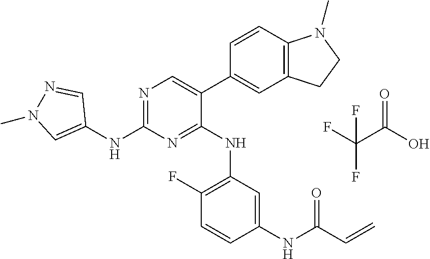 | K₂ | 485.2 | δ 10.33 (s, 1H), 9.95 (bs, 1H), 9.62 (bs, 1H), 7.93-7.81 (m, 2H), 7.67- 7.59 (m, 1H), 7.37-6.96 (m, 6H), 6.64 (d, J = 8.0 Hz, 1H), 6.43-6.39 (m, 1H), 6.26-6.21 (m, 1H), 5.76 (dd, J = 11.6 Hz, 1.2 Hz, 1H), 3.58-3.56 (m, 3H), 3.32 (t, J = 8.5 Hz, 2H), 2.94 (t, J = 8.5 Hz, 2H), 2.81 (s, 3H). |
| 60 | 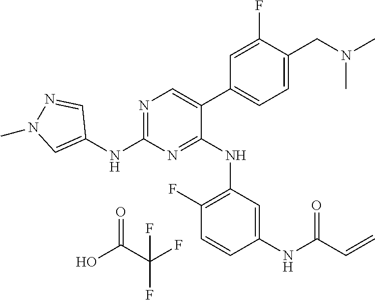 | K₁ | 503.2 [M − H]− | CD₃OD, δ 7.98-7.91 (m, 2H), 7.75-7.71 (m, 2H), 7.59-7.30 (m, 8H), 6.45-6.33 (m, 3H), 5.80- 5.77 (m, 1H), 4.49 (s, 2H), 3.65 (s, 3H), 2.94 (s, 6H). |
| 61 | 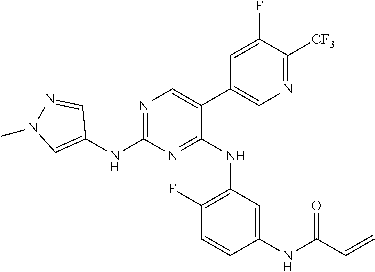 [SMILES: C=CC(=O)Nc1ccc(F)c(Nc2nc(Nc3cnn(C)c3)ncc2-c2cnc(C(F)(F)F)c(F)c2)c1] | K₁ | 516.9 | δ 10.26 (s, 1H), 9.43 (s, 1H), 9.04 (s, 1H), 8.80-8.55 (m, 2H), 8.19 (d, J = 11.6 Hz, 1H), 8.09 (s, 1H), 7.78 (s, 1H), 7.56 (s, 1H), 7.32 (s, 1H), 7.13 (d, J = 22.0 Hz, 1H), 6.43-6.36 (m, 1H), 6.23 (d, J = 16.4 Hz, 1H), 5.75 (d, J = 11.2 Hz, 1H), 3.52 (s, 3H). |
| 62 | 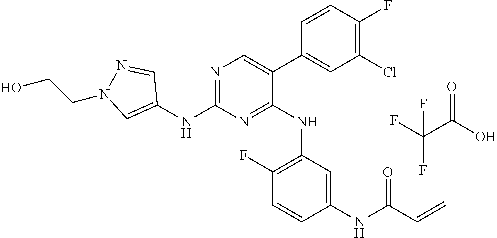 | K₂ | 511.9 | δ 10.26 (s, 1H), 9.92 (bs, 1H), 9.19 (bs, 1H), 7.96 (bs, 1H), 7.86-7.85 (m, 1H), 7.73-7.71 (m, 1H), 7.57- 7.51 (m, 3H), 7.32-7.27 (m, 4H), 6.45-6.38 (m, 1H), 6.25 (dd, J = 16.8 Hz, 1.6 Hz, 1H), 5.76 (dd, J = 10.0 Hz, 2.0 Hz, 1H), 3.88 (bs, 2H), 3.59 (bs, 2H). Note: OH peak is merged along with solvent peak. |

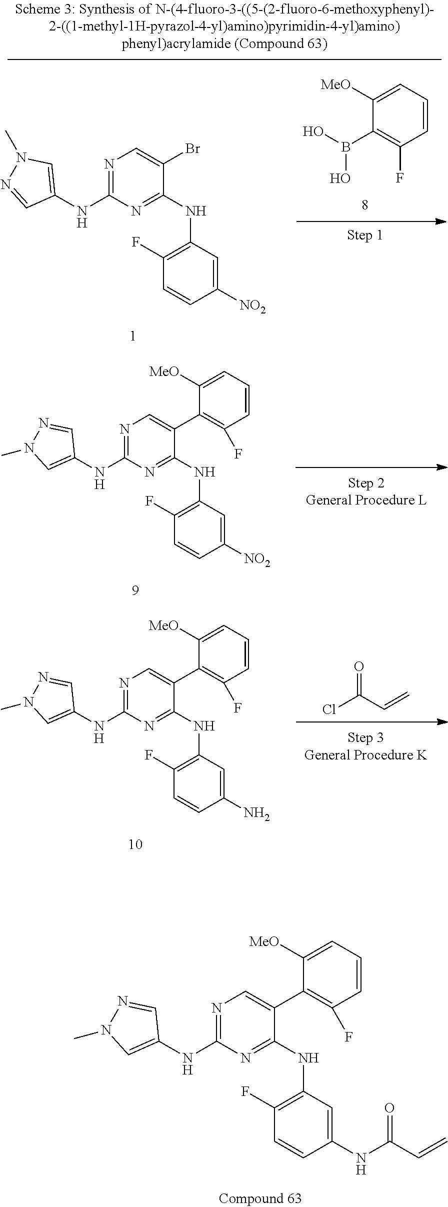

### Step 1: Synthesis of N4-(2-fluoro-5-nitrophenyl)-5-(2-fluoro-6-methoxyphenyl)-N2-(1-methyl-1H-pyrazol-4-yl)pyrimidine-2,4-diamine (9)

To a stirred solution of 5-bromo-N4-(2-fluoro-5-nitrophenyl)-N2-(1-methyl-1H-pyrazol-4-yl)pyrimidine-2,4-diamine (2) (350 mg, 0.85 mmol) in 1,4-dioxane (2.7 mL) and water (0.30 mL) was added tripotassium phosphate (546 mg, 2.57 mmol) and (2-fluoro-6-methoxyphenyl)boronic acid (8) (175 mg, 103 mmol). Then the reaction mixture was purged with nitrogen for 5 minutes, added XPhos Pd G2 (67.5 mg, 0.085 mmol) and the reaction mixture was heated to 100° C. for 16 hours. The progress of the reaction was monitored by TLC. Once the reaction was completed, the reaction mixture was quenched with water (50.0 mL) and extracted with dichloromethane (3×35 mL). The combined organic layer was dried over anhydrous sodium sulfate and evaporated under vacuum. The crude compound was purified by silica gel column chromatography using 18 to 22% ethyl acetate in hexane as eluent to afford the title compound (9) (0.33 g, Yield: 84.88%) as yellow solid. LCMS: [M+H]⁺ 454.2.

### Step 2: Synthesis of N4-(5-amino-2-fluorophenyl)-5-(2-fluoro-6-methoxyphenyl)-N2-(1-methyl-1H-pyrazol-4-yl)pyrimidine-2,4-diamine (10)

The title compound was prepared in a manner substantially similar to procedure mentioned in General Procedure L to afford yellow solid (0.25 g, crude). LCMS: [M+H]⁺ 424.2.

### Step 3: Synthesis of N-(4-fluoro-3-((5-(2-fluoro-6-methoxyphenyl)-2-((1-methyl-11H-pyrazol-4-yl)amino)pyrimidin-4-yl)amino)phenyl)acrylamide (Compound 63)

The title compound was prepared in a manner substantially similar to procedure mentioned in General Procedure K to afford off white solid (0.06 g, 21%). ¹H NMR (400 MHz, DMSO-d6): δ 10.19 (s, 1H), 9.10 (bs, 1H), 7.94 (s, 1H), 7.71-7.76 (m, 2H), 7.57 (s, 2H), 7.35-7.41 (m, 1H), 7.16-7.23 (m, 2H), 6.87-6.95 (m, 2H), 6.35-6.42 (m, 1H), 6.20-6.24 (m, 1H), 5.72-5.75 (m, 1H), 3.79 (s, 3H), 3.53 (s, 3H). LCMS: [M+H]⁺ 478.3.

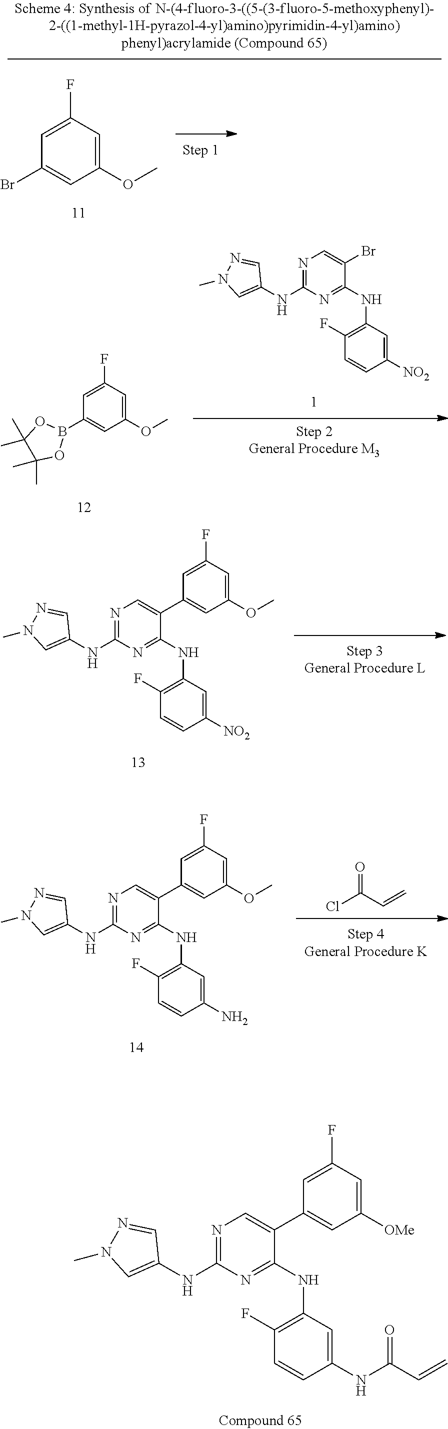

### Step 1: Preparation of 2-(3-fluoro-5-methoxyphenyl)-4,4,5,5-tetramethyl-1,3,2-dioxaborolane (12)

To a solution of 1-bromo-3-fluoro-5-methoxybenzene (1) (1.0 g, 4.88 mmol), 4,4,5,5-tetramethyl-2-(4,4,5,5-tetramethyl-1,3,2-dioxaborolan-2-yl)-1,3,2-dioxaborolane (0.40 g, 1.60 mmol) in N,N-dimethylformamide (5 mL) was added potassium acetate (0.57 g, 5.85 mmol) and the reaction mixture was degassed with nitrogen for 10 minutes. Then added [1,1′-bis(diphenylphosphino)ferrocene]dichloropalladium(II) (0.356 g, 0.488 mmol) and the reaction mixture was heated at 85° C. for 12 hours in a sealed tube. The reaction was monitored by LCMS and TLC. After completion of the reaction, the reaction mixture was diluted with water (20 mL) and extracted with ethyl acetate (2×20 mL). The combined organic layer was washed with brine (20 mL), dried over anhydrous sodium sulfate, filtered and evaporated under reduced pressure. The crude product was purified by combiflash purifier, the desire product was eluted with 20% ethyl acetate in hexane to afford the title compound (12) (1.0 g) as pale yellow liquid. LCMS [M+H]⁺ 253.1

### Step 2: Preparation of 5-(3-fluoro-5-methoxyphenyl)-N4-(2-fluoro-5-nitrophenyl)-N2-(1-methyl-1H-pyrazol-4-yl)pyrimidine-2,4-diamine (13)

Title compound was prepared in a manner substantially similar General Procedure M3 to afford the title compound (13) as white solid (0.15 g; Yield: 39%). LCMS: [M+H]⁺ 454.2

### Step 3: Preparation of N4-(5-amino-2-fluorophenyl)-5-(3-fluoro-5-methoxyphenyl)-N2-(1-methyl-1H-pyrazol-4-yl)pyrimidine-2,4-diamine (14)

Title compound was prepared in a manner substantially similar General Procedure L to afford the title compound (14) as white solid (0.12 g; Yield: 51%). LCMS: [M+H]⁺ 424.2

### Step 4: Preparation of N-(4-fluoro-3-{[5-(3-fluoro-5-methoxyphenyl)-2-[(1-methyl-1H-pyrazol-4-yl) amino]pyrimidin-4-yl]amino}phenyl)prop-2-enamide (Compound 65)

Title compound was prepared in a manner substantially similar General Procedure K to afford the title compound (Compound 65) as off white solid (0.01 g; Yield: 6%). ¹H NMR (400 MHz, DMSO-d6): δ 10.29 (s, 1H), 9.83 (bs, 1H), 9.19 (bs, 1H), 7.97 (s, 1H), 7.84 (s, 1H), 7.60 (s, 1H), 7.35 (bs, 1H), 7.23-7.13 (m, 2H), 6.93-6.89 (m, 3H), 6.45-6.38 (m, 1H), 6.25 (d, J=17.2 Hz, 1H), 5.77 (d, J=10.0 Hz, 1H), 3.84 (s, 3H), 3.55 (3H merged with DMSO water peak). LCMS: [M+H]⁺ 478.3

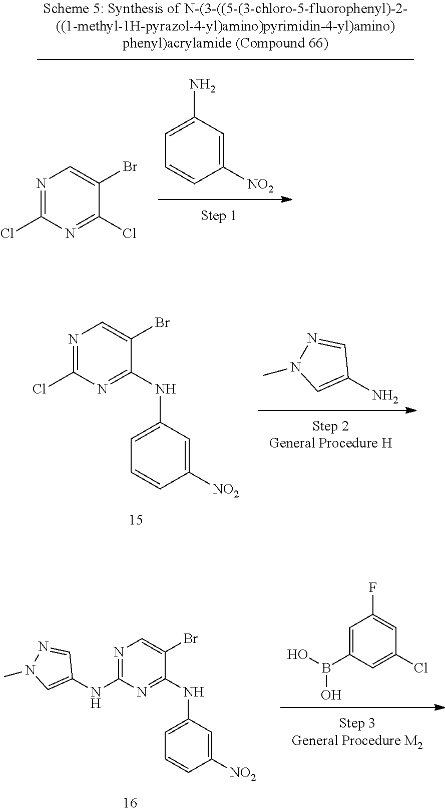

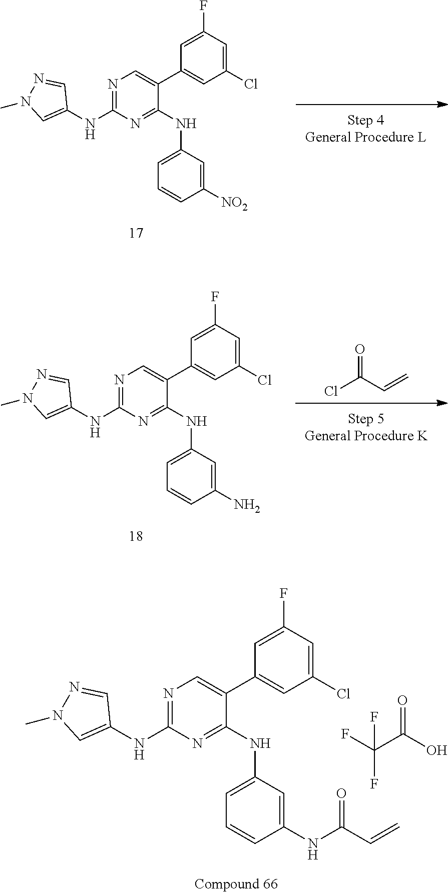

### Step 1: Synthesis of 5-bromo-2-chloro-N-(3-nitrophenyl)pyrimidin-4-amine (15)

To a stirred a solution of 3-nitroaniline (4.00 g, 29.0 mmol) and 5-bromo-2,4-dichloropyrimidine (7.92 g, 34.8 mmol) in N,N-dimethylformamide (40.0 mL) was added potassium carbonate (12.0 g, 86.9 mmol) at room temperature. The reaction mixture was heated at 100° C. for 36 hours. The reaction was monitored by TLC and LCMS. The reaction mixture was cooled to 0° C., diluted with ice-cold water (50 mL) and extracted with ethyl acetate (3×200 mL). The combined organic layer was washed with brine, dried over anhydrous sodium sulphate, filtered and concentrated under reduced pressure. The crude product was purified by flash chromatography by using combiflash purifier and was eluted with 10% ethyl acetate in hexane to afford 5-bromo-2-chloro-N-(3-nitrophenyl)pyrimidin-4-amine (15) (3.00 g) as yellow solid.

### Step 2: Synthesis of 5-bromo-N2-(1-methyl-1H-pyrazol-4-yl)-N4-(3-nitrophenyl)pyrimidine-2,4-diamine (16)

The title compound was prepared in a manner substantially similar to procedure mentioned in General Procedure H, to afford the desired compound (16) as yellow solid. LCMS [M+H]⁺ 390.2.

### Step 3: Synthesis of 5-(3-chloro-5-fluorophenyl)-N2-(1-methyl-1H-pyrazol-4-yl)-N4-(3-nitrophenyl)pyrimidine-2,4-diamine (17)

The title compound was prepared in a manner substantially similar to procedure mentioned in General Procedure M2, to afford the desired compound (17) as yellow solid. LCMS [M+H]⁺ 440.2

### Step 4: Synthesis of N4-(3-aminophenyl)-5-(3-chloro-5-fluorophenyl)-N2-(1-methyl-1H-pyrazol-4-yl)pyrimidine-2,4-diamine (18)

The title compound was prepared in a manner substantially similar to procedure mentioned in General Procedure L, to afford the desired compound (18) as yellow solid. LCMS [M+H]⁺ 410.1

### Step 5: Synthesis of N-(3-((5-(3-chloro-5-fluorophenyl)-2-((1-methyl-1H-pyrazol-4-yl)amino)pyrimidin-4-yl)amino)phenyl)acrylamide. TFA (Compound 66)

The title compound was prepared in a manner substantially similar to procedure mentioned in General Procedure K, to afford the desired compound (Compound 66) as off white solid. ¹H NMR (400 MHz, DMSO-d6): δ 10.22 (s, 1H), 9.89 (s, 1H), 9.32 (s, 1H), 7.97 (bs, 1H), 7.83 (bs, 1H), 7.33-7.16 (s, 9H), 6.41-6.48 (m, 1H), 6.25 (dd, J=17.2 Hz, 2.0 Hz, 1H), 5.76 (dd, J=10.0 Hz, 1.6 Hz, 1H), 3.60 (s, 3H); LCMS [M+H]⁺ 464.3

TABLE 3
The following compounds were prepared using the procedures described above:

| | | | LCMS | |
|---|---|---|---|---|
| Cmpd. No. | Structure | General Procedure | [M + H] | ¹H-NMR (400 MHz, DMSO-d₆) |
| 64 | 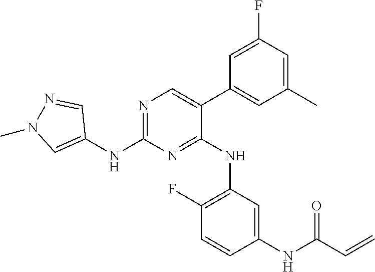 [SMILES: C=CC(=O)Nc1ccc(F)c(Nc2nc(Nc3cnn(C)c3)ncc2-c2cc(C)cc(F)c2)c1] | K | 462.3 | δ 10.29 (s, 1H), 9.89 (s, 1H), 9.19 (s, 1H), 7.95 (s, 1H), 7.83 (d, J = 5.2 Hz, 1H), 7.59 (s, 1H), 7.35 (s, 1H), 7.21-7.08 (m, 5H), 6.44- 6.37 (m, 1H), 6.24 (d, J = 16.4 Hz, 1H), 5.76 (d, J = 10.0 Hz, 1H), 3.56 (s, 3H), 2.38 (s, 3H). |
| 67 | 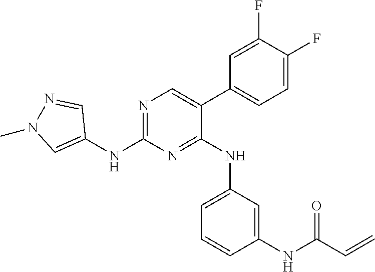 [SMILES: C=CC(=O)Nc1cccc(Nc2nc(Nc3cnn(C)c3)ncc2-c2ccc(F)c(F)c2)c1] | K₁ | 448.3 | δ 10.19 (m, 2H), 9.44 (s, 1H), 8.19 (s, 1H), 7.92-7.13 (m, 9H), 6.46- 6.39 (m, 1H), 6.23 (d, J = 16.0 Hz, 1H), 5.75 (d, J = 11.2 Hz, 1H), 3.60 (d, J = 23.6 Hz, 3H). |
| 68 | 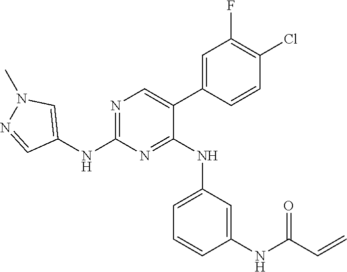 [SMILES: C=CC(=O)Nc1cccc(Nc2nc(Nc3cnn(C)c3)ncc2-c2ccc(Cl)c(F)c2)c1] | K₁ | 464.3 | δ 10.25 (s, 2H), 9.45 (s, 1H), 7.95- 7.12 (m, 10H), 6.46-6.39 (m, 1H), 6.25-6.21 (m, 1H), 5.75 (d, J = 11.2 Hz, 1H), 3.58 (s, 3H). |
| 69 | 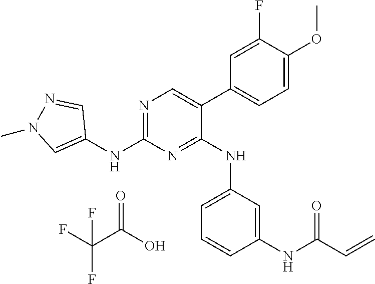 | K₁ | 460.0 | δ 10.28 (m, 2H), 9.58 (s, 1H), 7.90- 7.84 (m, 2H), 7.62-7.13 (m, 9H), 6.46-6.40 (m, 1H), 6.26-6.20 (m, 1H), 5.76-5.73 (m, 1H), 3.88 (s, 3H), 3.58 (s, 3H). |
| 70 | 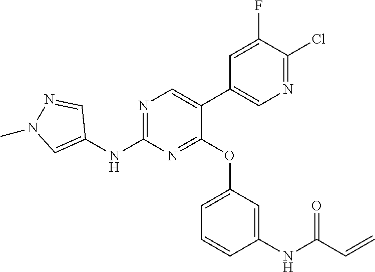 [SMILES: C=CC(=O)Nc1cccc(Oc2nc(Nc3cnn(C)c3)ncc2-c2cnc(Cl)c(F)c2)c1] | K₁ | 466.2 | At 90° C., δ 10.07 (s, 1H), 9.55 (s, 1H), 8.63 (d, J = 1.6 Hz, 1H), 8.57 (s, 1H), 8.23-8.20 (m, 1H), 7.68- 7.67 (m, 1H), 7.63 (d, J = 8.4 Hz, 1H), 7.49-7.45 (m, 1H), 7.25 (s, 2H), 7.05-7.02 (m, 1H), 6.47- 6.40 (m, 1H), 6.29-6.24 (m, 1H), 5.79-5.73 (m, 1H), 3.60 (s, 3H) |
| 71 | 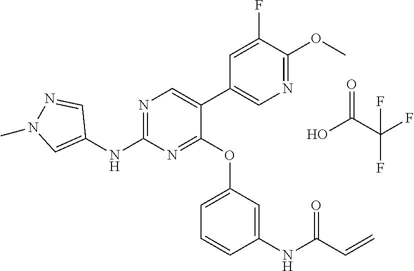 | K₁ | 462.5 | δ 10.06 (s, 1H), 9.37 (s, 1H), 8.47 (s, 1H), 8.29 (s, 1H), 7.97-7.94 (m, 1H), 7.65-7.60 (m, 2H), 7.48- 7.44 (m, 1H), 7.25-7.00 (m, 4H), 6.47-6.40 (m, 1H), 6.29- 6.24 (m, 1H), 5.76-5.75 (m, 1H), 4.03 (s, 3H), 3.61 (s, 3H). |
| 72 | 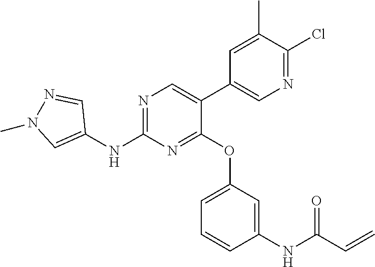 [SMILES: C=CC(=O)Nc1cccc(Oc2nc(Nc3cnn(C)c3)ncc2-c2cnc(Cl)c(C)c2)c1] | K₁ | 462.2 | δ 10.38 (s, 1H), 9.83 (s, 1H), 8.53 (d, J = 24.0 Hz, 2H), 8.12 (s, 1H), 7.61 (t, J = 36.0 Hz, 3H), 7.09- 7.02 (m, 2H), 6.85 (s, 1H), 6.45- 6.38 (m, 1H), 6.23 (d, J = 12.0 Hz, 1H), 5.75 (d, J = 12.0 Hz, 1H), 3.49 (s, 3H), 2.38 (s, 3H). |
| 73 | 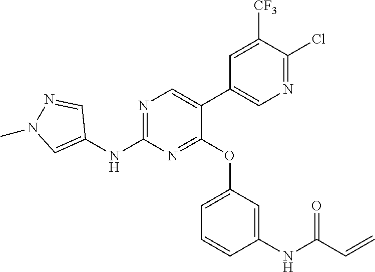 [SMILES: C=CC(=O)Nc1cccc(Oc2nc(Nc3cnn(C)c3)ncc2-c2cnc(Cl)c(C(F)(F)F)c2)c1] | K1 | 516.2 | At 90° C., δ 10.07 (s, 1H), 9.87 (s, 1H), 9.01-9.00 (m, 1H), 8.62 (s, 1H), 8.57-8.56 (m, 1H), 7.68- 7.66 (m, 2H), 7.48-7.46 (m, 1H), 7.31-7.21 (m, 2H), 7.12-7.02 (m, 1H), 6.47-6.40 (m, 1H), 6.28- 6.24 (m, 1H), 5.75 (d, J = 8.4 Hz, 1H), 3.61 (s, 3H). |
| 74 | 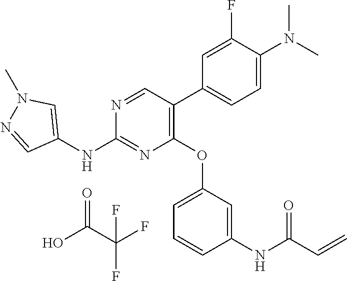 | K₁ | 474.2 | At 90° C., δ 10.06 (s, 1H), 9.28 (s, 1H), 8.41 (s, 1H), 7.63-7.61 (m, 2H), 7.48-7.38 (m, 3H), 7.24- 7.16 (m, 2H), 7.06-6.97 (m, 2H), 6.47-6.36 (m, 2H), 6.29-6.24 (m, 1H), 5.76-5.73 (m, 1H), 3.60 (s, 3H), 2.88 (s, 6H). |
| 75 | 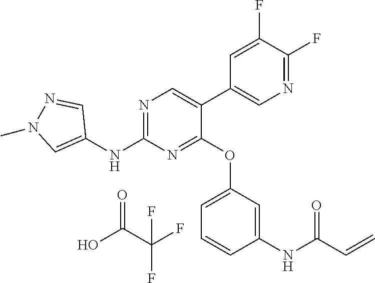 | K₁ | 450.3 | δ 10.07 (s, 1H), 9.49 (s, 1H), 8.53 (s, 1H), 8.36-8.28 (m, 2H), 7.67- 7.60 (m, 2H), 7.49-7.44 (m, 2H), 7.25-7.20 (m, 2H), 7.04- 7.02 (m, 1H,), 6.47-6.40 (m, 1H), 6.28-6.24 (m, 1H), 5.77-5.76 (d, J = 2.0 Hz, 1H), 3.60 (s, 3H). |

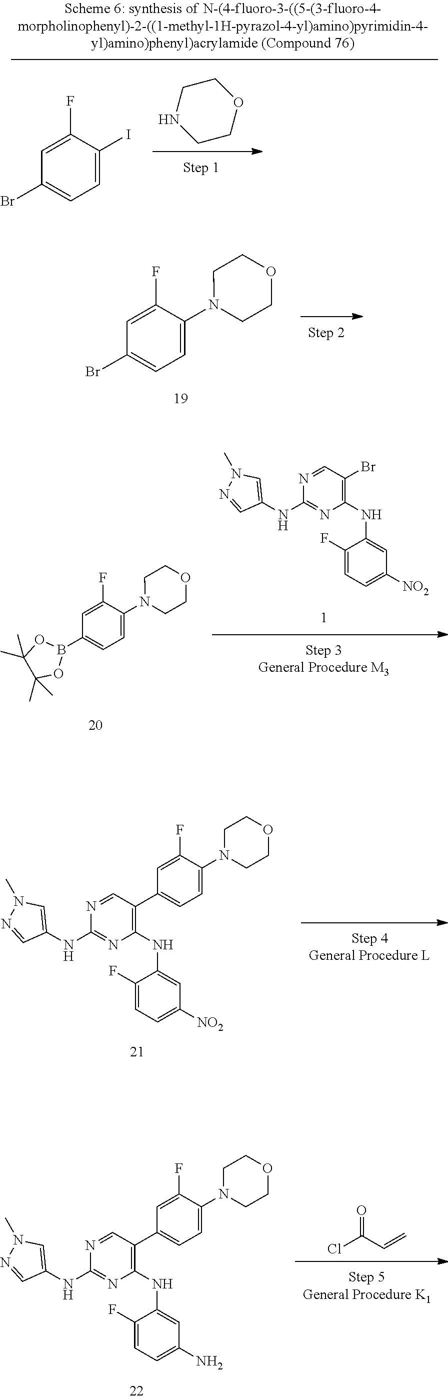

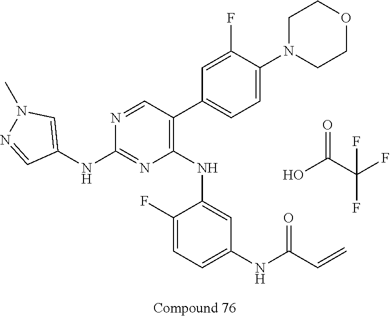

### Step 1: Synthesis of 4-(4-bromo-2-fluorophenyl) morpholine (19)

To a stirred solution of 4-bromo-2-fluoro-1-iodobenzene (5.00 g, 16.6 mmol) in toluene (50.0 mL) was added morpholine (1.45 g, 16.6 mmol), cesium carbonate (13.5 g, 41.5 mmol), xantphos (0.962 g, 1.66 mmol) and the reaction mixture was purged with argon for 10 minutes. Then added tris(dibenzylideneacetone)dipalladium(0) (0.457 g, 0.499 mmol) and the reaction mixture was heated at 100° C. for 12 hours. The progress of the reaction was monitored by TLC and LCMS. The reaction mixture was cooled, diluted with water (100 mL) and extracted with ethyl acetate (100 mL×3). The combined organic layer was dried over anhydrous sodium sulfate and evaporated under reduced pressure. The crude product was purified by column chromatography and was eluted with 10 to 20% ethyl acetate in hexane as eluent to afford 4-(4-bromo-2-fluorophenyl) morpholine (19) (1.80 g, 41%).

### Step 2: Synthesis of 4-[2-fluoro-4-(4,4,5,5-tetramethyl-1,3,2-dioxaborolan-2-yl) phenyl]morpholine (20)

To a stirred solution of 4-(4-bromo-2-fluorophenyl) morpholine (19) (1.20 g, 4.61 mmol) in 1,4-dioxane (10.0 mL) was added 4,4,5,5-tetramethyl-2-(4,4,5,5-tetramethyl-1,3,2-dioxaborolan-2-yl)-1,3,2-dioxaborolane (1.17 g, 4.61 mmol), potassium acetate (1.36 g, 13.8 mmol) and the reaction mixture was purged with argon for 10 minutes. Then added (1,1′-bis(diphenylphosphino)ferrocene) palladium(II) dichloride (0.376 g, 0.461 mmol) and the reaction mixture was heated at 100° C. for 12 hours. The progress of the reaction was monitored by LCMS. The reaction mixture was cooled, diluted with water (50 mL) and extracted with ethyl acetate (50 mL×3). The combined organic layer was dried over anhydrous sodium sulfate and concentrated under reduced pressure. The crude product was purified by column chromatography and was eluted with 10 to 20% ethyl acetate in hexane as eluent to afford 4-[2-fluoro-4-(4,4,5,5-tetramethyl-1,3,2-dioxaborolan-2-yl) phenyl] morpholine (20) (1.2 g, 52%). LCMS [M+H]⁺ 308.0

### Step 3: Synthesis of 5-[3-fluoro-4-(morpholin-4-yl) phenyl]-N4-(2-fluoro-5-nitrophenyl)-N2-(1-methyl-1H-pyrazol-4-yl) pyrimidine-2,4-diamine (21)

The title compound was prepared in a manner substantially similar to procedure mentioned in General Procedure M3 to get desired product (21) as white solid. LCMS [M+H]⁺ 509.2

### Step 4: synthesis of Synthesis of N4-(5-amino-2-fluorophenyl)-5-[3-fluoro-4-(morpholin-4-yl)phenyl]-N2-(1-methyl-1H-pyrazol-4-yl)pyrimidine-2,4-diamine (22)

The title compound was prepared in a manner substantially similar to procedure mentioned in General Procedure L to get desired product (22) as yellow solid. LCMS [M+H]⁺ 479.5

### Step 5: Synthesis of N-[4-fluoro-3-({5-[3-fluoro-4-(morpholin-4-yl)phenyl]-2-[(1-methyl-1H-pyrazol-4-yl)amino]pyrimidin-4-yl}amino)phenyl]prop-2-enamide (Compound 76)

The title compound was prepared in a manner substantially similar to procedure mentioned in General Procedure K₁ to get desired product (Compound 76) as off white solid. ¹H NMR (400 MHz, DMSO-d6): δ 10.32 (bs, 1H), 10.19 (bs, 1H), 9.39 (bs, 1H), 7.85 (s, 1H), 8.06-7.83 (m, 2H), 7.58 (bs, 1H), 7.48-6.97 (m, 6H), 6.44-6.38 (m, 1H), 6.26-6.21 (m, 1H), 5.77-5.74 (m, 1H), 3.76-3.74 (m, 4H), 3.56 (bs, 3H), 3.03 (m, 4H); LCMS [M+H]⁺ 533.3

TABLE 4
The following compounds were prepared using the procedures described above:

| | | General | | |
|---|---|---|---|---|
| Cmpd. No. | Structure | Procedure | LCMS [M + H] | ¹H-NMR (400 MHz, DMSO-d₆) |
| 77 | 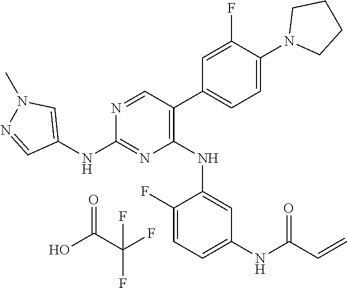 | K | 517.3 | δ 10.26 (s, 1H), 9.96 (s, 1H), 9.26 (s, 1H), 7.84 (d, J = 4.8 Hz, 2H), 7.57-7.52 (m, 2H), 7.40-7.16 (m, 5H), 6.83 (t, J = 9.2 Hz, 1H), 6.43-6.37 (m, 1H), 6.26-6.21 (m, 1H), 5.77-5.74 (m, 1H), 3.58 (s, 3H). 3.36 (d, J = 1.6 Hz, 4H), 1.91 (t, J = 6.0 Hz, 4H). |
| 78 | 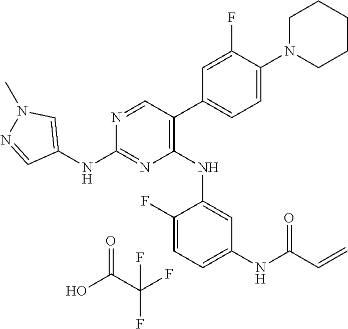 | K₁ | 531.0 | δ 10.33 (s, 2H), 9.59 (s, 1H), 7.92- 7.85 (m, 2H), 7.58 (s, 1H), 7.36- 7.12 (m, 7H), 6.43-6.36 (m, 1H), 6.22 (d, J = 16.4 Hz, 1H), 5.73 (d, J = 11.6 Hz, 1H), 3.66 (s, 3H), 3.02 (t, J = 4.8 Hz, 4H). 1.66 (s, 4H), 1.54 (d, J = 4.8 Hz, 2H). |
| 79 | 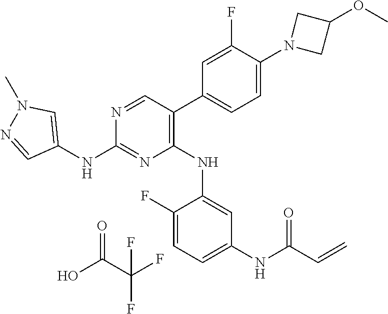 | K₁ | 533.2 | δ 10.31 (s, 1H), 10.14 (bs, 1H), 9.41 (bs, 1H), 7.85-7.84 (m, 2H), 7.57 (s, 1H), 7.35-7.08 (m, 5H), 7.08-6.95 (m, 1H), 6.68 (t, J = 9.0 Hz, 1H), 6.43-6.36 (m, 1H), 6.23 (dd, J = 16.8 Hz, 2.0 Hz, 1H), 5.75 (dd, J = 10.0 Hz, 2.0 Hz, 1H), 4.33-4.28 (m, 1H), 4.17 (t, J = 6.4 Hz, 2H), 3.74-3.56 (m, 2H), 3.56 (s, 3H), 3.23 (s, 3H). |
| 80 | 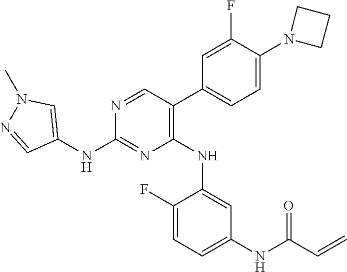 [SMILES: C=CC(=O)Nc1ccc(F)c(Nc2nc(Nc3cnn(C)c3)ncc2-c2ccc(N3CCC3)c(F)c2)c1] | K₁ | 503.3 | δ 10.16 (s, 1H), 9.00 (bs, 1H), 8.14 (s, 1H), 7.85 (s, 1H), 7.77 (d, J = 5.3 Hz, 1H), 7.57 (bs, 1H), 7.10- 7.27 (m, 5H), 6.60 (t, J = 8.8 Hz, 1H), 6.36-6.43 (m, 1H), 6.21- 6.25 (m, 1H), 5.72-5.75 (m, 1H), 3.90-3.93 (m, 4H), 3.55 (bs, 3H), 2.26-2.33 (m, 2H). |
| 81 | 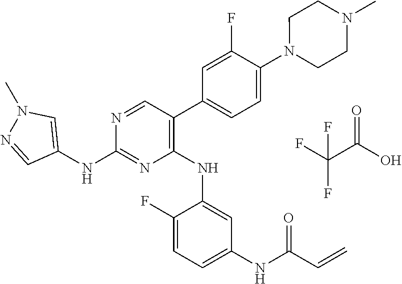 | K₁ | 546.3 | δ 10.29 (s, 1H), 9.80 (bs, 2H), 9.05 (bs, 1H), 7.92-7.86 (m, 2H), 7.57 (s, 1H), 7.38-6.97 (m, 6H), 6.46- 6.39 (m, 1H), 6.26 (dd, J = 16.8 Hz, 1.6 Hz, 1H), 5.77 (dd, J = 10.0 Hz, 1.6 Hz, 1H), 3.58-3.55 (m, 7H), 3.27 (bs, 2H), 3.17-3.04 (m, 2H), 2.90 (s, 3H) |
| 82 | 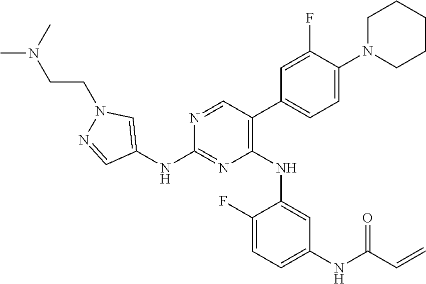 [SMILES: C=CC(=O)Nc1ccc(F)c(Nc2nc(Nc3cnn(CCN(C)C)c3)ncc2-c2ccc(N3CCCCC3)c(F)c2)c1] | K₂ | 588.3 | δ 10.27 (s, 1H), 9.17 (bs, 1H), 8.35 (bs, 1H), 7.99 (s, 1H), 7.85 (d, J = 5.2 Hz, 1H), 7.69 (s, 1H), 7.17- 7.37 (m, 5H), 6.46-6.53 (m, 1H), 6.33 (dd, J = 18.8 Hz, 2.0 Hz, 1H), 5.84 (dd, J = 12.0 Hz, 2.0 Hz, 2H), 3.95 (s, 2H), 3.09-3.14 (m, 6H), 2.19 (s, 6H), 1.76 (bs, 4H), 1.65 (d, J = 4.8 Hz, 2H). |
| 83 | 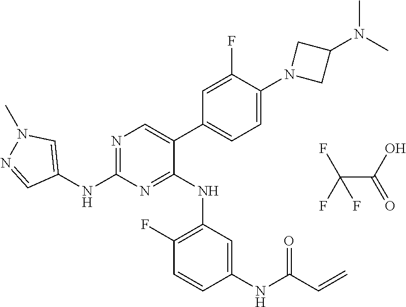 [SMILES: C=CC(=O)Nc1ccc(F)c(Nc2nc(Nc3cnn(C)c3)ncc2-c2ccc(N3CC(N(C)C)C3)c(F)c2)c1 ; O=C(O)C(F)(F)F] | K₁ | 546.2 | δ 10.70 (bs, 1H), 10.33 (s, 1H), 10.16 (bs, 1H), 9.21 (bs, 1H), 7.90 (d, J = 4.4 Hz, 2H), 7.59 (bs, 1H), 7.35-7.21 (m, 5H), 6.77-6.72 (m, 1H), 6.47-6.40 (m, 1H), 6.25 (dd, J = 17.2 Hz, 2.0 Hz, 1H), 5.77 (dd, J = 10.0 Hz, 1.6 Hz, 1H), 4.24 (bs, 4H), 4.15-4.13 (m, 1H), 3.60 (bs, 3H), 2.83 (s, 6H). |
| 84 | 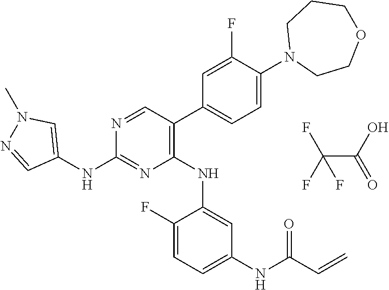 | K₂ | 547.2 | δ 10.28 (s, 1H), 9.96 (bs, 1H), 9.32 (bs, 1H), 7.85-7.84 (m, 2H), 7.57 (s, 2H), 7.35-7.34 (m, 1H), 7.25- 7.19 (m, 2H), 7.15 (d, J = 8.8 Hz, 1H), 7.07 (m, 2H), 6.43-6.36 (m, 1H), 6.23 (dd, J = 16.8 Hz, 1.6 Hz, 1H), 5.75 (dd, J = 10.0 Hz, 1.6 Hz, 1H), 3.75 (t, J = 4.6 Hz, 2H), 3.70 (t, J = 5.6 Hz, 2H), 3.57 (bs, 3H), 3.48-3.46 (m, 4H), 1.97-1.91 (m, 2H). |
| 85 | 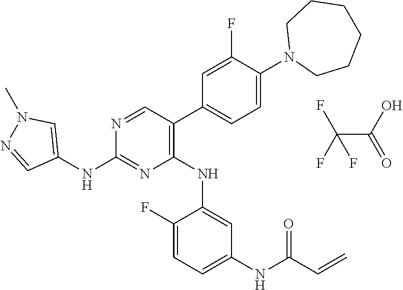 | K₂ | 545.2 | δ 10.22 (bs, 1H), 10.01 (bs, 1H), 9.31 (s, 1H), 7.80-7.78 (m, 2H), 7.52 (bs, 1H), 7.31-7.26 (m, 1H), 7.16-7.06 (m, 5H), 6.96-6.92 (s, 1H), 6.38-6.31 (m, 1H), 6.20- 6.10 (m, 1H), 5.71-5.68 (m, 1H), 3.52 (s, 3H merged with DMSO peak), 3.35-3.32 (m, 4H), 1.72 (bs, 4H), 1.50 (bs, 4H) |

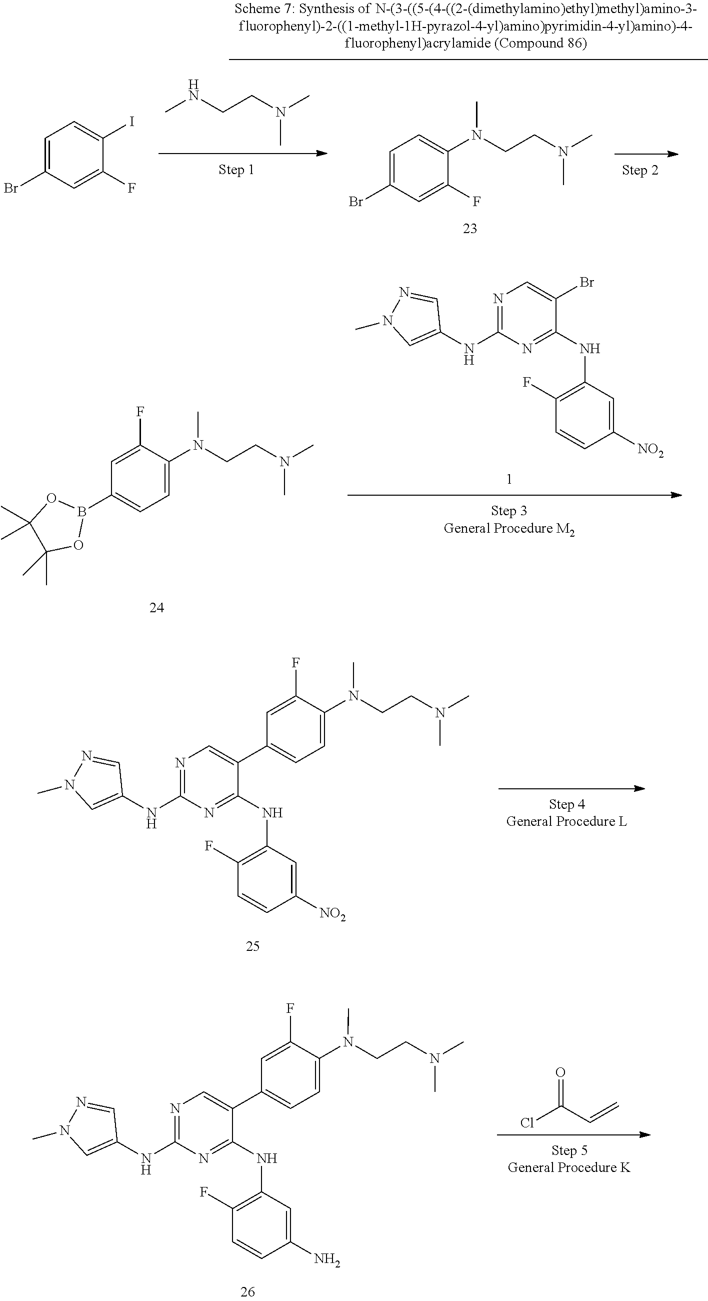

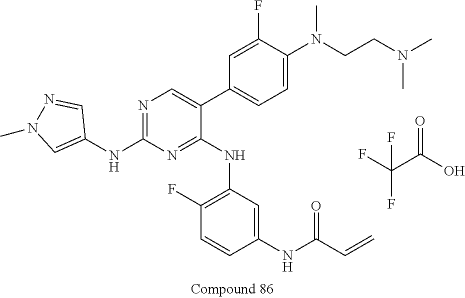

### Step 1: Synthesis of N1-(4-bromo-2-fluorophenyl)-N1,N2,N2-trimethylethane-1,2-diamine (23)

To a stirred solution of 4-bromo-2-fluoro-1-iodobenzene (5.00 g, 16.6 mmol), [2-(dimethylamino)ethyl](methyl)amine (1.70 g, 16.6 mmol), cesium carbonate (13.5 g, 41.5 mmol) in 1,4-dioxane (50.0 mL) was purged with nitrogen for 5 minutes. Then added tris(dibenzylideneacetone)dipalladium(O) (0.76 g, 0.831 mmol), 4,5-bis(diphenylphosphino)-9,9-dimethylxanthene (0.96 g, 1.66 mmol) and the reaction mixture was heated at 100° C. for 12 hours. The progress of the reaction was monitored by LCMS. The reaction mixture was cooled and concentrated under reduced pressure. The crude product was purified by combiflash purifier and was eluted with 5-10% methanol in dichloromethane to afford 4-bromo-N-[2-(dimethylamino)ethyl]-2-fluoro-N-methylaniline (23) (1.00 g, 3.63 mmol) as brown oil. LCMS [M+H]⁺ 275.0

### Step 2: Synthesis of N1-(2-fluoro-4-(4,4,5,5-tetramethyl-1,3,2-dioxaborolan-2-yl)phenyl)-N1,N2,N2-trimethylethane-1,2-diamine (24)

To a stirred solution of 4-bromo-N-[2-(dimethylamino)ethyl]-2-fluoro-N-methylaniline (23) (1.50 g, 5.45 mmol), 4,4,5,5-tetramethyl-2-(4,4,5,5-tetramethyl-1,3,2-dioxaborolan-2-yl)-1,3,2-dioxaborolane (1.52 g, 6.00 mmol), potassium acetate (1.60 g, 16.4 mmol) in 1,4-dioxane (20.0 mL) was purged with nitrogen for 5 minutes. Then added [1,1′-bis(diphenylphosphino)ferrocene]dichloropalladium(II), complex with dichloromethane (0.445 g, 0.545 mmol) and the reaction mixture was heated at 90° C. for 16 hours. The progress of the reaction was monitored by LCMS. After completion of the reaction, the reaction mixture was cooled and concentrated under reduced pressure. The crude product was purified by using combiflash purifier and was eluted with 10-18% methanol in dichloromethane to afford N-[2-(dimethylamino)ethyl]-2-fluoro-N-methyl-4-(4,4,5,5-tetramethyl-1,3,2-dioxaborolan-2-yl)aniline (24) (1.20 g, 2.20 mmol). LCMS [M+H]⁺ 323.3

### Step 3: Synthesis of 5-(4-((2-(dimethylamino)ethyl)(methyl)amino)-3-fluorophenyl)-N4-(2-fluoro-5-nitrophenyl)-N2-(1-methyl-1H-pyrazol-4-yl)pyrimidine-2,4-diamine (25)

The title compound was prepared in a manner substantially similar to procedure mentioned in General Procedure M2 to get desired product (25) as white solid. LCMS [M+H]⁺ 524.2

### Step 4: Synthesis of N4-(5-amino-2-fluorophenyl)-5-(4-((2-(dimethylamino)ethyl)(methyl)amino)-3-fluorophenyl)-N2-(1-methyl-1H-pyrazol-4-yl)pyrimidine-2,4-diamine (26)

The title compound was prepared in a manner substantially similar to procedure mentioned in General Procedure L, to afford the desired compound (26) as yellow solid.

LCMS [M+H]⁺ 494.3

### Step 5: Synthesis of N-(3-((5-(4-((2-(dimethylamino)ethyl)(methyl)amino)-3-fluorophenyl)-2-((1-methyl-1H-pyrazol-4-yl)amino)pyrimidin-4-yl)amino)-4-fluorophenyl)acrylamide (Compound 86)

The title compound was prepared in a manner substantially similar to procedure mentioned in General Procedure K, to afford the desired compound as off white solid. ¹H NMR (400 MHz, DMSO-d6): δ 10.47 (s, 1H), 9.80 (bs, 1H), 9.36 (bs, 1H), 7.88-8.16 (m, 2H), 7.58 (s, 1H), 7.02-7.34 (m, 7H), 6.25-6.44 (m, 1H), 6.23 (dd, J=17.2 Hz, 1.6 Hz 1H), 5.75 (dd, J=10.0 Hz, 1.6 Hz, 1H), 3.76 (s, 3H), 3.43 (t, J=13.6 Hz, 2H), 3.31 (s, 2H), 2.83 (s, 9H); LCMS [M+H]⁺ 548.5

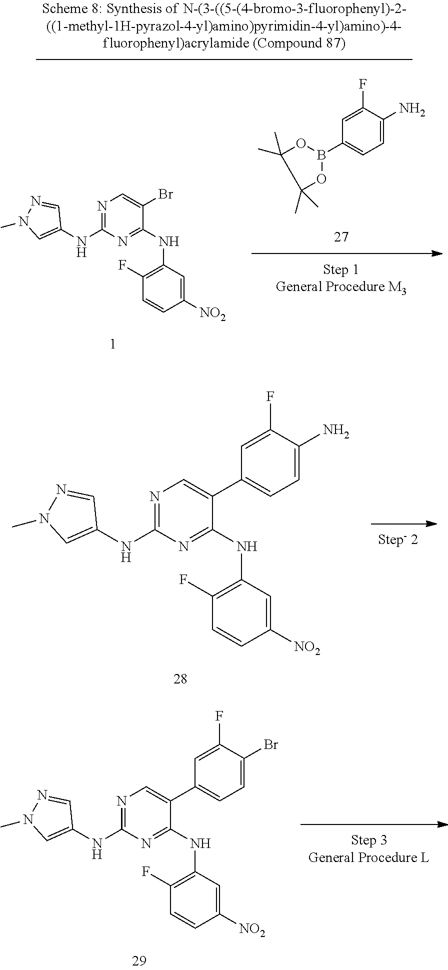 [SMILES: Cn1cc(Nc2ncc(Br)c(Nc3cc([N+](=O)[O-])ccc3F)n2)cn1 ; CC1(C)OB(c2ccc(N)c(F)c2)OC1(C)C ; Cn1cc(Nc2ncc(-c3ccc(N)c(F)c3)c(Nc3cc([N+](=O)[O-])ccc3F)n2)cn1 ; Cn1cc(Nc2ncc(-c3ccc(Br)c(F)c3)c(Nc3cc([N+](=O)[O-])ccc3F)n2)cn1]

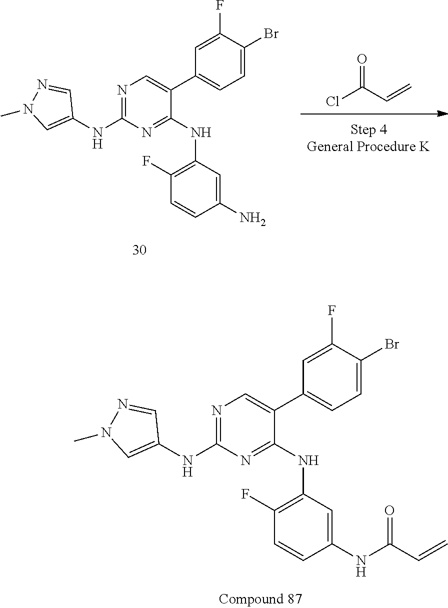

### Step 1: Preparation of 5-(4-amino-3-fluorophenyl)-N4-(2-fluoro-5-nitrophenyl)-N2-(1-methyl-1H-pyrazol-4-yl) pyrimidine-2,4-diamine (28)

The title compound was prepared in a manner substantially similar to procedure mentioned in General Procedure M3 to get desired product (28) as white solid. LCMS [M+H]⁺ 439.2

### Step 2: Synthesis of 5-(4-bromo-3-fluorophenyl)-N4-(2-fluoro-5-nitrophenyl)-N2-(1-methyl-1H-pyrazol-4-yl) pyrimidine-2,4-diamine (29)

To a solution of 5-(4-amino-3-fluorophenyl)-N4-(2-fluoro-5-nitrophenyl)-N2-(1-methyl-1H-pyrazol-4-yl)pyrimidine-2,4-diamine (28) (2.00 g, 4.56 mmol), bromotrichloromethane (4.50 mL, 45.6 mmol), sodium nitrite (1.57 g, 22.8 mmol) in dichloromethane (20.0 mL), water (20.0 mL) was added acetic acid (5.22 mL, 91.2 mmol) and the reaction mixture was stirred at room temperature for 16 hours. The reaction was monitored by TLC and LCMS. Upon completion of the reaction, the reaction mixture was extracted with ethyl acetate (50 mL×3). The combined organic layer was washed with brine (50 mL), dried over anhydrous sodium sulfate and evaporated under reduced pressure. The crude product was purified with silica gel column chromatography and was eluted in 60-65% ethyl acetate in hexane to give 5-(4-bromo-3-fluorophenyl)-N4-(2-fluoro-5-nitrophenyl)-N2-(1-methyl-1H-pyrazol-4-yl)pyrimidine-2,4-diamine (29) (1.20 g, 2.39 mmol) as yellow solid (1.2 g, 52%). LCMS [M+H]⁺ 502.0

### Step 3: Synthesis of N4-(5-amino-2-fluorophenyl)-5-(4-bromo-3-fluorophenyl)-N2-(1-methyl-1H-pyrazol-4-yl) pyrimidine-2,4-diamine (30)

The title compound was prepared in a manner substantially similar to procedure mentioned in General Procedure L to get desired product (30) as white solid. LCMS [M+H]⁺ 471.8

### Step 4: Synthesis of N-(3-{[5-(4-bromo-3-fluorophenyl)-2-[(1-methyl-1H-pyrazol-4-yl) amino] pyrimidin-4-yl] amino}-4-fluorophenyl) prop-2-enamide (Compound 87)

The title compound was prepared in a manner substantially similar to procedure mentioned in General Procedure K₁ to get desired product (Compound 87) as white solid. ¹H NMR (400 MHz, DMSO-d6): δ 10.23 (s, 1H), 9.24 (s, 1H), 8.50 (s, 1H), 7.95 (s, 1H), 7.75 (t, J=8.0 Hz, 2H), 7.57 (s, 1H), 7.47 (d, J=9.6 Hz, 1H), 7.27 (d, J=8.4 Hz, 2H), 7.13-7.06 (m, 2H), 6.43-6.36 (m, 1H), 6.23 (d, J=17.2 Hz, 1H), 5.74 (d, J=11.6 Hz, 1H), 3.52 (s, 3H); LCMS [M+H]⁺ 526.2.

TABLE 5
The following compounds were prepared using the procedures described above:

| | | General | | |
|---|---|---|---|---|
| Cmpd. | | Pro- | LCMS | |
| No. | Structure | cedure | [M + H] | ¹H-NMR (400 MHz, DMSO-d₆) |
| 88 | 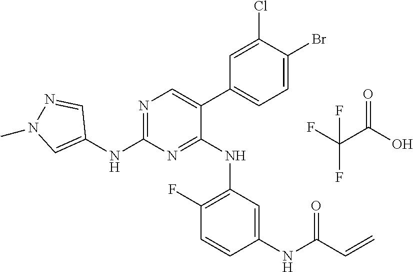 | K₁ | 542.0 | 10.30 (s, 1H), 9.92 (s, 1H), 9.30 (s, 1H), 7.97-7.11 (m, 10H), 6.43- 6.21 (m, 2H), 5.75 (d, J = 12.0 Hz, 1H), 3.17 (s, 3H). |
| 89 | 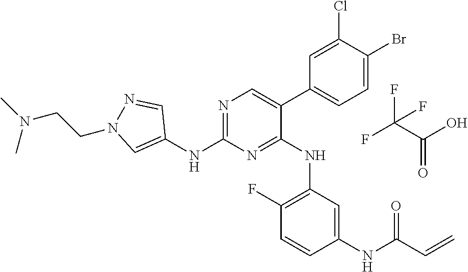 | K₂ | 601.1 | δ 10.20 (s, 1H), 9.24 (bs, 1H), 8.51 (bs, 1H), 7.94 (s, 1H), 7.80 (d, J = 7.6 Hz, 1H), 7.68-7.73 (m, 2H), 7.58 (s, 1H), 7.36 (dd, J = 10.4 Hz, 2.0 Hz, 1H), 7.10-7.27 (m, 4H), 6.35-6.42 (m, 1H), 6.22 (dd, J = 18.8 Hz, 1.6 Hz, 1H), 5.74 (dd, J = 11.6 Hz, 1.6 Hz, 1H), 3.81 (s, 4H), 2.05 (s, 6H). |
| 90 | 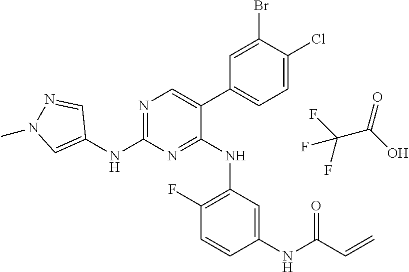 | K₁ | 542.0 | δ 10.24 (s, 1H), 9.43 (bs, 1H), 8.76 (bs, 1H), 7.95 (s, 1H), 7.91 (s, 1H), 7.79-7.78 (m, 1H), 7.72- 7.70 (m, 1H), 7.58-7.50 (m, 2H), 7.31-7.08 (m, 4H), 6.45-6.38 (m, 1H), 6.27-6.23 (m, 1H), 5.77- 5.75 (m, 1H), 3.57 (s, 3H). |
| 91 | 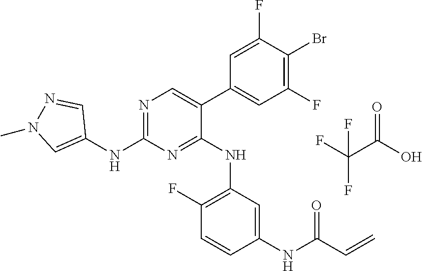 | K₂ | 544.0 | δ 10.28 (s, 1H), 9.85 (bs, 1H), 9.04 (bs, 1H), 8.00 (s, 1H), 7.81 (s, 1H), 7.57 (bs, 1H), 7.42 (d, J = 8.0 Hz, 2H), 7.33 (s, 1H), 7.18 (s, 1H), 7.10-6.90 (m, 2H), 6.43-6.39 (m, 1H), 6.25-6.21 (m, 1H), 5.76- 5.73 (m, 1H), 3.54 (s, 3H). |
| 92 | 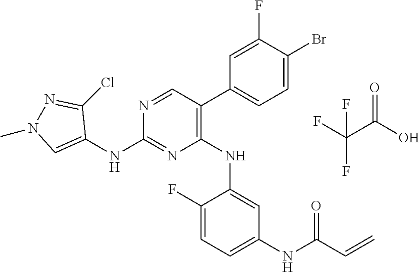 | K | 660.1 | δ 10.25 (s, 1H), 9.17 (bs, 2H), 7.97 (bs, 2H), 7.84-7.80 (m, 2H), 7.55- 7.52 (m, 2H), 7.32-7.28 (m, 2H), 6.93-7.19 (m, 1H), 6.42- 6.36 (m, 1H), 6.25-6.21 (m, 1H), 5.73-5.76 (m, 1H), 3.59 (s, 3H). |
| 93 | 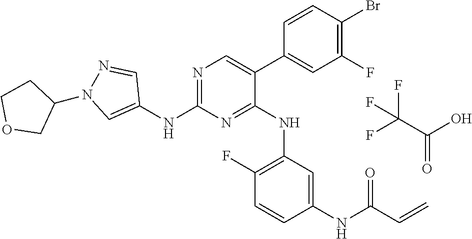 | K₂ | 584.0 | δ 10.29 (s, 1H), 9.98 (bs, 1H), 9.25 (bs, 1H), 8.00 (bs, 1H), 7.86-7.82 (m, 2H), 7.57-7.54 (m, 2H), 7.35- 6.90 (m, 5H), 6.45-6.38 (m, 1H), 6.28-6.23 (m, 1H), 5.79-5.60 (m, 1H), 4.63-4.60 (m, 1H), 3.89- 3.85 (m, 2H), 3.79-3.74 (m, 2H), 2.33-2.07 (m, 2H). |
| 94 | 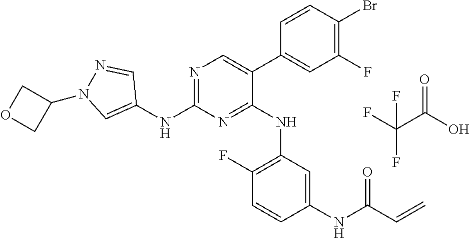 | K₂ | 568.0 | δ 10.30 (s, 1H), 9.87 (bs, 1H), 9.13 (bs, 1H), 7.99 (s, 1H), 7.85-7.81 (m, 2H), 7.51-7.53 (m, 2H), 7.42- 6.90 (m, 5H), 6.45-6.39 (m, 1H), 6.26 (dd, J = 18.8 Hz, 1.6 Hz, 1H), 5.77 (dd, J = 11.6 Hz, 1.6 Hz, 1H), 5.14 (bs, 1H), 4.90-4.72 (m, 4H). |
| 95 | 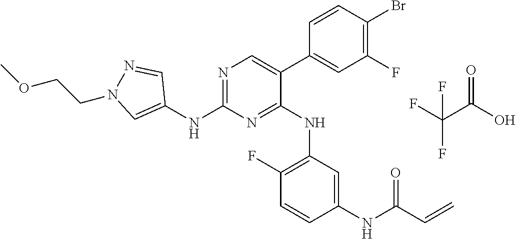 | K₂ | 570.0 | δ 10.23 (s, 1H), 9.44 (s, 1H), 8.71 (s, 1H), 7.95 (s, 1H), 7.79-7.75 (m, 2H), 756-7.48 (m, 3H), 7.28- 7.06 (m, 4H), 6.42-6.38 (m, 1H), 6.35-6.20 (m, 1H), 5.73 (d, J = 10.0 Hz, 1H), 3.90 (s, 2H), 3.35-3.45 (m, 2H), 3.11 (s, 3H). |
| 96 | 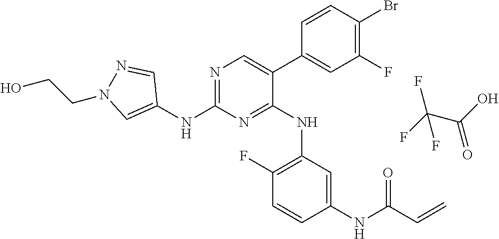 | K₂ | 556.0 | δ 10.23 (s, 1H), 9.63 (bs, 1H), 8.97 (bs, 1H), 7.97 (s, 1H), 7.83-7.79 (m, 3H), 7.52 (d, J = 10.8 Hz, 3H), 7.31-7.20 (m, 3H), 6.45-6.38 (m, 1H), 6.24 (dd, J = 12 Hz, J = 2 Hz, 1H), 5.75 (d, J = 12.0 Hz, 1H), 3.86 (bs, 1H), 3.58 (s, 4H) |
| 97 | 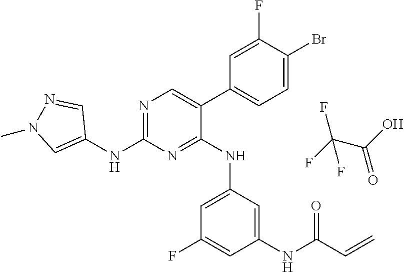 | K₂ | 526.1 | δ 10.32 (s, 1H), 9.62 (bs, 1H), 9.05 (bs, 1H), 7.97 (s, 1H), 7.80-7.76 (m, 1H), 7.52-7.50 (m, 2H), 7.38- 7.36 (m, 2H), 7.26 (d, J = 7.6 Hz, 1H), 7.18-6.93 (m, 3H), 6.43- 6.36 (m, 1H), 6.26-6.22 (m, 1H), 5.78-5.75 (m, 1H), 3.61 (bs, 3H). |

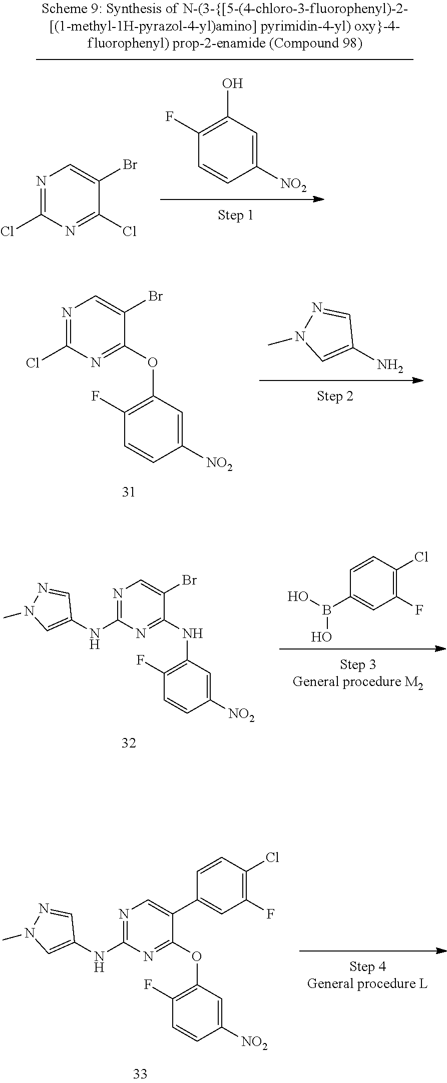

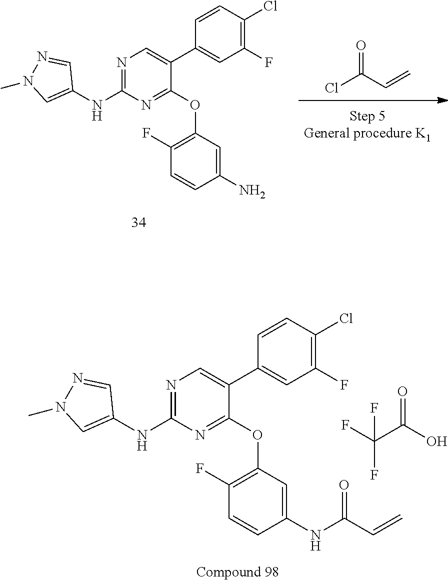

### Step 1: Synthesis of 5-bromo-2-chloro-4-(2-fluoro-5-nitrophenoxy) pyrimidine (31)

To a stirred solution of 5-bromo-2,4-dichloropyrimidine (3 g, 13.2 mmol) and 2-fluoro-5-nitrophenol (2.07 g, 13.2 mmol) in N,N-dimethylformamide (10 mL) was added potassium carbonate (2.73 g, 19.7 mmol) and the reaction mixture was stirred at 60° C. for 3 hours. Progress of the reaction was monitored by TLC and LCMS. The reaction mixture was diluted with cold water (15 mL), the precipitated solid was filtered, washed with cold water and dried to obtain 5-bromo-2-chloro-4-(2-fluoro-5-nitrophenoxy)pyrimidine (31) (4.20 g, 12.1 mmol) as off-white solid. LCMS [M+H]⁺ 347.9

### Step 2: Synthesis of 5-bromo-4-(2-fluoro-5-nitrophenoxy)-N-(1-methyl-1H-pyrazol-4-yl) pyrimidin-2-amine (32)

To a stirred solution of 5-bromo-2-chloro-4-(2-fluoro-5-nitrophenoxy)pyrimidine (296) (1 g, 2.87 mmol) in N,N-diisopropylethylamine (2.50 mL, 14.3 mmol) was added 1-methyl-1H-pyrazol-4-amine (0.33 g, 3.44 mmol) and trifluoroacetic acid (0.44 mL, 3.44 mmol). The reaction mixture was heated to 100° C. for 16 hours. Progress of the reaction was monitored by TLC and LCMS. After completion of the reaction, the reaction mixture was diluted with water (50 mL) and extracted with ethyl acetate (3×10 mL). The combined organic layer was washed with brine (25 mL), dried over sodium sulphate and evaporated under reduced pressure. The crude product was purified by silica gel flash column chromatography using combiflash purifier and was eluted in 40% ethyl acetate in hexane to obtain 5-bromo-4-(2-fluoro-5-nitrophenoxy)-N-(1-methyl-1H-pyrazol-4-yl)pyrimidin-2-amine (32) (0.7 g, 1.71 mmol) as an yellow solid. LCMS [M+H]⁺ 409.0

### Step 3: Synthesis of 5-(4-chloro-3-fluorophenyl)-4-(2-fluoro-5-nitrophenoxy)-N-(1-methyl-1H-pyrazol-4-yl) pyrimidin-2-amine (33)

The title compound was prepared in a manner substantially similar to procedure mentioned in General Procedure M2 to get desired product (33) as yellow solid. LCMS [M+H]⁺ 459.1

### Step 4: Synthesis of 4-(5-amino-2-fluorophenoxy)-5-(4-chloro-3-fluorophenyl)-N-(1-methyl-1H-pyrazol-4-yl) pyrimidin-2-amine (34)

The title compound was prepared in a manner substantially similar to procedure mentioned in General Procedure L to get desired product (34) as brown solid. LCMS [M+H]⁺ 429.8

### Step 5: Synthesis of N-(3-{[5-(4-chloro-3-fluorophenyl)-2-[(1-methyl-1H-pyrazol-4-yl) amino] pyrimidin-4-yl] oxy}-4-fluorophenyl) prop-2-enamide (Compound 98)

The title compound was prepared in a manner substantially similar to procedure mentioned in General Procedure K₁ to get desired product (Compound 98) as pale yellow solid. ¹H NMR (400 MHz, DMSO-d6): δ 10.11 (s, 1H), 9.50 (s, 1H), 8.53 (s, 1H), 7.94-7.47 (m, 7H), 7.23-7.15 (m, 2H), 6.45-6.38 (m, 1H), 6.27 (d, J=16.8 Hz, 1H), 5.77 (d, J=1.6 Hz, 1H), 3.61 (s, 3H); LCMS [M+H]⁺ 483.1.

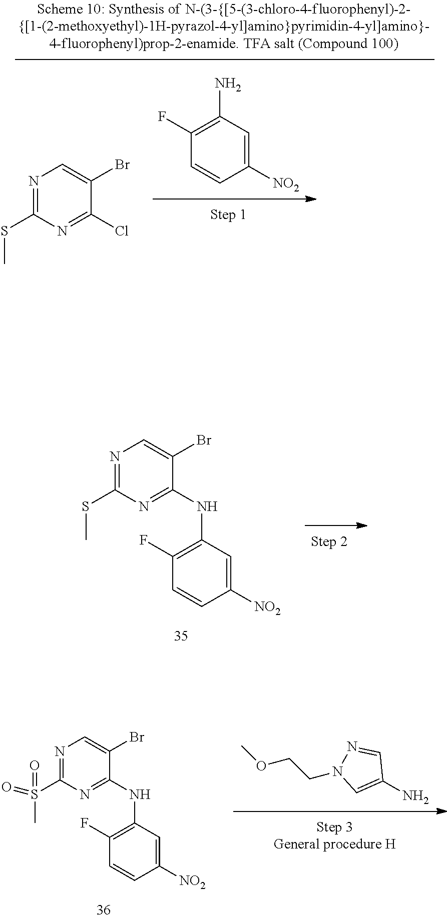

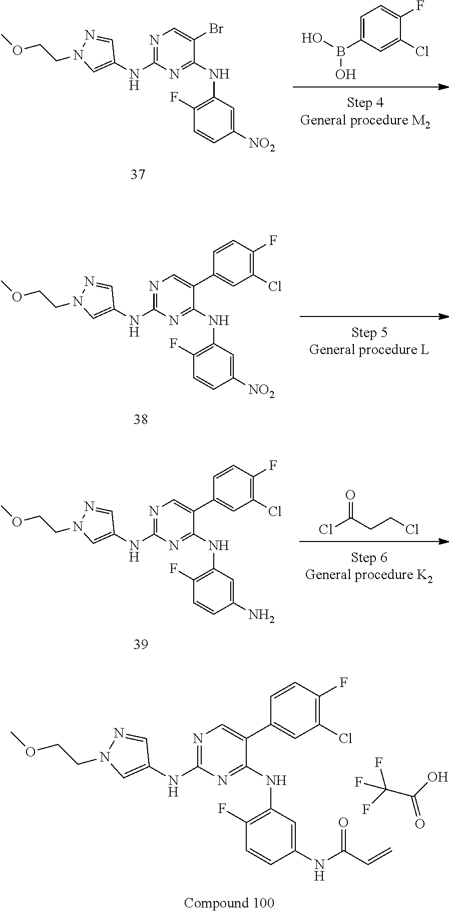

### Step 1: Synthesis of 5-bromo-N-(2-fluoro-5-nitrophenyl)-2-(methylthio)pyrimidin-4-amine (35)

To a stirred solution of 2-fluoro-5-nitroaniline (0.71 g, 4.59 mmol) in tetrahydrofuran (12 mL) at 0° C. was added sodium hydride (0.33 g, 8.35 mmol, 60% w/w) and the reaction mixture was stirred at room temperature for 30 min. Then the reaction mixture was cooled to 0° C. and was added a solution of 5-bromo-4-chloro-2-(methylsulfanyl)pyrimidine (1.00 g, 4.18 mmol) in tetrahydrofuran (3 mL) and the reaction mixture was stirred at room temperature for 2 hours. Then the reaction mixture was quenched with water (50 mL) and extracted with ethyl acetate (50 mL×3). The combined organic extract was washed with brine (50 mL), dried over anhydrous sulfate and evaporated. The crude product was purified by column chromatography using combiflash purifier and was eluted with 15% ethyl acetate in hexane to get the title compound (35) as brown solid (1.0 g, 66%). LCMS [M+H]⁺ 360.7

### Step 2: Synthesis of 5-bromo-N-(2-fluoro-5-nitrophenyl)-2-(methylsulfonyl)pyrimidin-4-amine (36)

To a stirred solution of 5-bromo-N-(2-fluoro-5-nitrophenyl)-2-(methylsulfanyl)pyrimidin-4-amine (35) (0.87 g, 2.42 mmol) in dichloromethane (10.0 mL) at 0° C. was added 3-chlorobenzene-1-carboperoxoic acid (1.67 g, 9.69 mmol) and the reaction mixture was stirred at room temperature for 4 hours. The reaction mixture was quenched with sodium bicarbonate solution (10 mL) and extracted with dichloromethane (10 mL×3). The combined organic layer was washed with brine (10 mL), dried over anhydrous sodium sulfate and evaporated. The crude product was purified by column chromatography using combiflash purifier and was eluted with 50% ethyl acetate in hexane to get the title compound (36) as yellow solid (0.69 g, 72%). LCMS [M+H]⁺ 391.0

### Step 3: Synthesis of 5-bromo-N4-(2-fluoro-5-nitrophenyl)-N2-(1-(2-methoxyethyl)-1H-pyrazol-4-yl)pyrimidine-2,4-diamine (37)

The title compound was prepared in a manner substantially similar to procedure mentioned in General Procedure H, to afford the desired compound (37) as yellow solid. LCMS [M+H]⁺ 452.0.

### Step 4: Synthesis of 5-(3-chloro-4-fluorophenyl)-N4-(2-fluoro-5-nitrophenyl)-N2-(1-(2-methoxyethyl)-1H-pyrazol-4-yl)pyrimidine-2,4-diamine (38)

The title compound was prepared in a manner substantially similar to procedure mentioned in General Procedure M2, to afford the desired compound (38) as off white solid. LCMS [M+H]⁺ 502.1

### Step 5: Synthesis of N4-(5-amino-2-fluorophenyl)-5-(3-chloro-4-fluorophenyl)-N2-(1-(2-methoxyethyl)-1H-pyrazol-4-yl)pyrimidine-2,4-diamine (39)

The title compound was prepared in a manner substantially similar to procedure mentioned in General Procedure L, to afford the desired compound (39) as brown solid. LCMS [M+H]⁺ 472.2.

### Step 6: Synthesis of N-(3-((5-(3-chloro-4-fluorophenyl)-2-((1-(2-methoxyethyl)-1H-pyrazol-4-yl)amino)pyrimidin-4-yl)amino)-4-fluorophenyl)acrylamide (Compound 100)

The title compound was prepared in a manner substantially similar to procedure mentioned in General Procedure K2, to afford the desired compound (Compound 100) as white solid. ¹H NMR (400 MHz, DMSO-d6): δ 10.28 (s, 1H), 9.93 (bs, 1H), 9.19 (bs, 1H), 7.96 (s, 1H), 7.85 (d, J=5.2 Hz, 1H), 7.73 (d, J=6.4 Hz, 1H), 7.51-7.57 (m, 3H), 7.22-7.35 (m, 4H), 6.38-6.45 (m, 1H), 6.24 (dd, J=18.4 Hz, 1.6 Hz, 1H), 5.76 (dd, J=11.6 Hz, 1.6 Hz, 1H), 3.97 (s, 4H), 3.51 (s, 3H). LCMS [M+H]⁺ 526.1

TABLE 6
The following compounds were prepared using the procedures described above:

| Cmpd. | | General | LCMS | |
|---|---|---|---|---|
| No. | Structure | Procedure | [M + H] | ¹H-NMR (400 MHz, DMSO-d₆) |
| 99 | 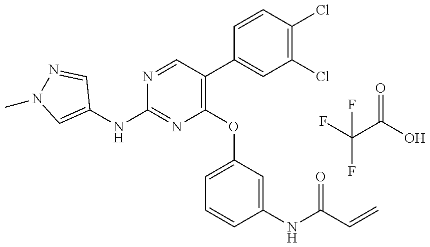 | K | 481.1 | At 90° C. δ 10.06 (s, 1H), 9.42 (bs, 1H), 8.48 (bs, 1H), 7.92 (s, 1H), 7.68-7.61 (m, 4H), 7.48-7.44 (m, 2H), 7.24-7.14 (m, 2H), 7.01 (d, J = 12.0 Hz, 1H), 6.46-6.44 (m, 1H), 6.29-6.24 (m, 1H), 5.75 (d, J = 12.0 Hz, 1H), 3.60 (s, 3H). |
| 101 | 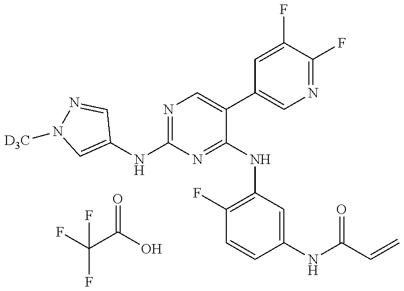 | K₂ | 470.2 | δ 10.29 (s, 1H), 9.93 (bs, 1H), 9.24 (bs, 1H), 8.25-8.18 (m, 3H), 8.03 (bs, 1H), 7.87 (bs, 1H), 7.57 (s, 1H), 7.36-7.08 (m, 3H), 6.45- 6.39 (m, 1H), 6.28-6.23 (m, 1H), 5.79-5.76 (m, 1H). |
| 102 | 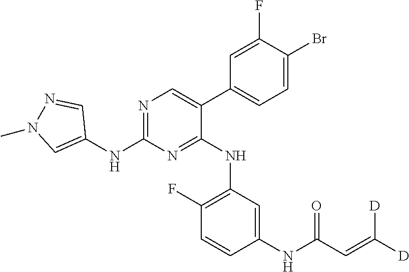 [SMILES: [2H]C([2H])=CC(=O)Nc1ccc(F)c(Nc2nc(Nc3cnn(C)c3)ncc2-c2ccc(Br)c(F)c2)c1] | | 528.0 | δ 10.21 (s, 1H), 9.21 (bs, 1H), 8.47 (bs, 1H), 7.97 (s, 1H), 7.70- 7.79 (m, 2H), 7.88-7.72 (m, 2H), 7.58 (s, 1H), 7.35-7.30 (m, 1H), 7.20-7.19 (m, 1H), 7.10-7.08 (m, 1H), 6.40 (s, 1H), 3.56 (s, 3H). |
| 103 | 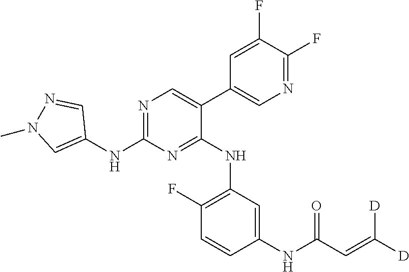 [SMILES: [2H]C([2H])=CC(=O)Nc1ccc(F)c(Nc2nc(Nc3cnn(C)c3)ncc2-c2cnc(F)c(F)c2)c1] | | 469.2 | δ 10.28 (s, 1H), 9.36 (s, 1H), 9.63 (s, 1H), 8.16-8.11 (m, 3H), 7.76 (s, 1H), 7.76 (bs, 1H), 7.55 (bs, 1H), 7.30 (bs, 1H), 7.13 (d, J = 19.2 Hz, 1H), 6.38 (s, 1H), 3.52 (s, 3H). |
| 104 | 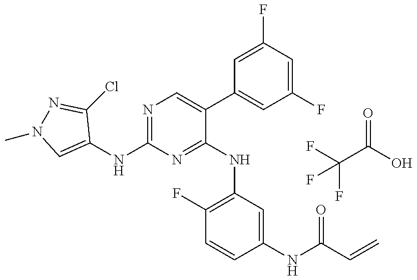 | K₂ | 500.1 | δ 10.26 (s, 1H), 9.30 (bs, 2H), 8.00 (bs, 1H), 7.83-7.81 (m, 1H), 7.55-7.08 (m, 7H), 6.43-6.36 (m, 1H), 6.26-6.21 (m, 1H), 5.76-5.73 (m, 1H), 3.60 (bs, 2H). |
| 105 | 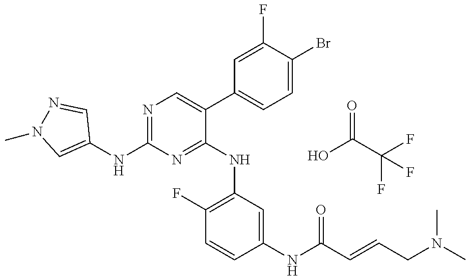 | J | 583.1 | δ 10.44 (s, 1H), 9.79 (bs, 1H), 8.91 (bs, 1H), 7.97 (s, 1H), 7.81- 7.77 (m, 3H), 7.58 (bs, 1H), 7.50 (d, J = 9.6 Hz, 1H), 7.33-7.27 (m, 1H), 7.16-7.08 (m, 3H), 6.74-6.67 (m, 1H), 6.43-6.39 (m, 1H), 3.92 (d, J = 6.4 Hz, 2H), 3.54 (bs, 3H), 2.77 (s, 6H). |
| 106 | 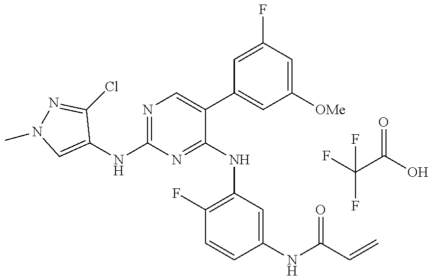 | K₂ | 512.2 | δ 10.25 (s, 1H), 9.17 (bs, 2H), 7.98 (bs, 1H), 7.85-7.84 (m, 1H), 7.58-7.56 (m, 1H), 7.34-7.30 (m, 1H), 7.21 (s, 1H), 6.95-6.90 (m, 4H), 6.45-6.38 (m, 1H), 6.28- 6.24 (m, 1H), 5.79-5.76 (m, 1H), 4.01 (s, 3H), 3.62 (s, 3H). |
| 107 | 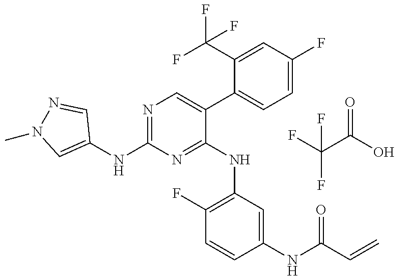 | K₂ | 516.2 | 10.26 (s, 1H), 9.69 (bs, 1H), 8.70 (bs, 2H), 7.79 (d, J = 9.2 Hz, 2H), 7.68-7.59 (m, 4H), 7.31 (s, 1H), 7.19-7.12 (m, 2H), 6.41-6.34 (m, 1H), 6.22 (d, J = 15.2 Hz, 1H), 5.74 (d, J = 9.2 Hz, 1H), 3.56 (s, 3H). |
| 108 | 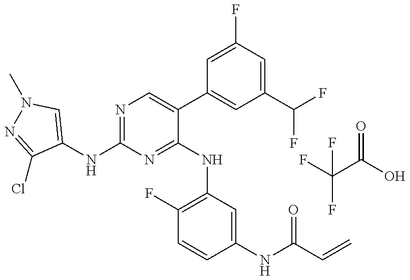 | K₂ | 532.1 | δ 10.27 (s, 1H), 9.29 (s, 1H), 8.03 (bs, 1H), 7.84 (dd, J = 2.4 Hz, J = 2.4 Hz, 1H), 7.59-7.50 (m, 4H), 7.35-7.30 (m, 1H), 7.24 (s, J = 12.0 Hz, 1H), 7.10-6.97 (m, 3H), 6.45-6.38 (m, 1H), 6.28-6.23 (m, 1H), 5.79-5.76 (m, 1H), 3.82 (s, 3H). |
| 109 | 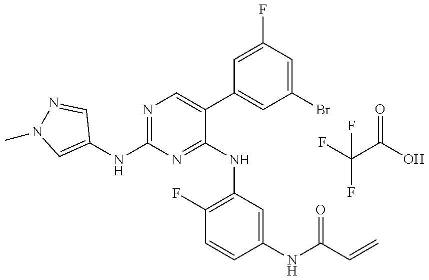 | K₂ | 526.1 | δ 10.29 (s, 1H), 9.80 (bs, 1H), 9.18 (s, 1H), 8.02 (bs, 1H), 7.84 (d, J = 4.0 Hz, 1H), 7.61-7.65 (m, 3H), 7.42-7.36 (m, 2H), 7.21 (s, 1H), 7.13 (bs, 1H), 7.08 (bs, 1H), 6.46-6.38 (m, 1H), 6.28-6.23 (m, 1H), 5.79-5.76 (m, 1H), 3.57 (s, 3H). |
| 110 | 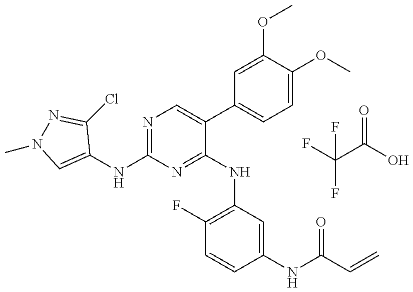 | K₂ | 525.2 | δ 10.25 (s, 1H), 9.18 (bs, 2H), 7.89 (bs, 2H), 7.58-7.56 (m, 1H), 7.34-7.30 (m, 2H), 7.12-7.10 (m, 3H), 7.04-7.02 (m, 1H), 6.46- 6.39 (m, 1H), 6.28-6.24 (m, 1H), 5.79-5.76 (m, 1H), 3.82 (s, 9H). |
| 111 | 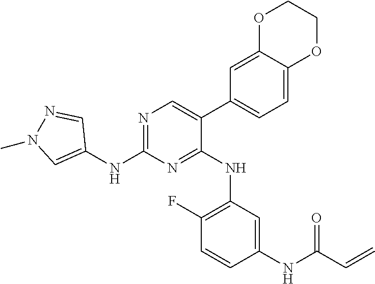 [SMILES: C=CC(=O)Nc1ccc(F)c(Nc2nc(Nc3cnn(C)c3)ncc2-c2ccc3c(c2)OCCO3)c1] | K₂ | 488.2 | δ 10.19 (s, 1H), 9.06 (bs, 2H), 8.17 (bs, 2H), 7.84 (s, 1H), 7.76 (bs, 1H), 7.57 (bs, 1H), 7.25- 6.89 (m, 4H), 6.43-6.36 (m, 1H), 6.25-6.20 (m, 1H), 5.75- 5.72 (m, 1H), 4.25 (s, 4H), 3.53 (bs, 3H). |
| 112 | 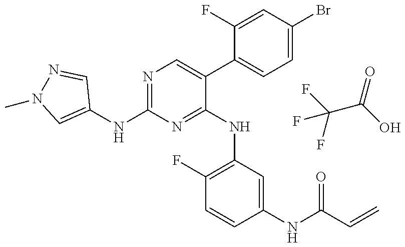 | K₂ | 526.1 | δ 10.30 (s, 1H), 10.06 (bs, 1H), 9.36 (bs, 1H), 7.99 (bs, 1H), 7.82-7.77 (m, 2H), 7.59 (bs, 1H), 7.50-7.47 (m, 1H), 7.38-6.95 (m, 5H), 6.42-6.35 (m, 1H), 6.26- 6.21 (m, 1H), 5.76-5.73 (m, 1H), 3.56 (bs, 3H). |
| 113 | 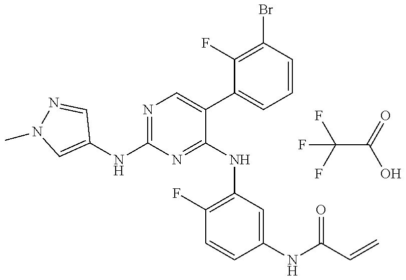 | K₂ | 526.1 | δ 10.33 (s, 1H), 10.24 (bs, 1H), 9.44 (bs, 1H), 8.01 (s, 1H), 7.83 (bs, 1H), 7.73-7.70 (m, 1H), 7.59- 7.57 (m, 2H), 7.48 (t, J = 8.0 Hz, 1H), 7.37 (bs, 1H), 7.37-7.12 (m, 3H), 6.45-6.38 (m, 1H), 6.28- 6.23 (m, 1H), 5.79-5.76 (m, 1H), 3.59 (s, 3H). |
| 114 | 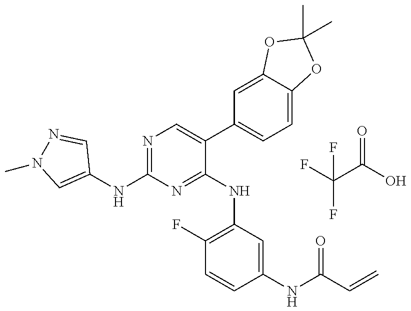 | K₂ | 502.2 | δ 10.31 (s, 1H), 9.93 (bs, 1H), 9.28 (bs, 1H), 7.87-7.85 (m, 2H), 7.58 (bs, 1H), 7.36-6.95 (m, 4H), 6.98-6.91 (m, 3H), 6.45-6.38 (m, 1H), 6.28-6.23 (m, 1H), 5.79- 5.76 (m, 1H), 3.58 (bs, 3H), 1.73 (s, 6H). |
| 115 | 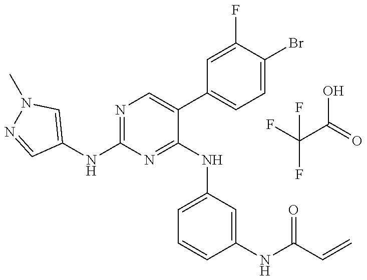 | K₂ | 509.1 | δ 10.74-10.69 (m, 2H), 9.97 (bs, 1H), 8.39 (s, 1H), 8.33-8.24 (m, 2H), 8.02-8.00 (m, 2H), 7.92- 7.82 (m, 4H), 7.75-7.40 (m, 1H), 6.90-6.83 (m, 2H), 6.69-6.64 (m, 1H), 6.20-6.17 (m, 1H), 4.04 (bs, 3H). |
| 116 | 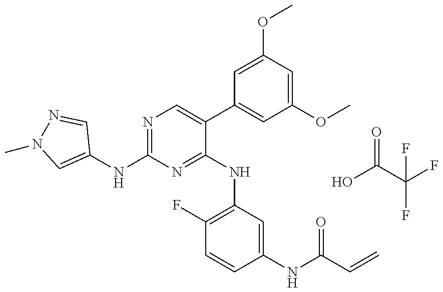 | K₂ | 490.2 | δ 10.31 (bs, 1H), 9.91 (bs, 1H), 9.23 (bs, 1H), 7.96 (bs, 1H), 7.87- 7.85 (m, 1H), 7.62 (bs, 1H), 7.40- 7.00 (m, 4H), 6.67 (bs, 2H), 6.58 (s, 1H), 6.46-6.39 (m, 1H), 6.28- 6.24 (m, 1H), 5.78 (dd, J = 9.6 Hz, J = 1.6 Hz, 1H), 3.86 (s, 6H), 3.46 (s, 3H, merged with DMSO- H₂O peak) |
| 117 | 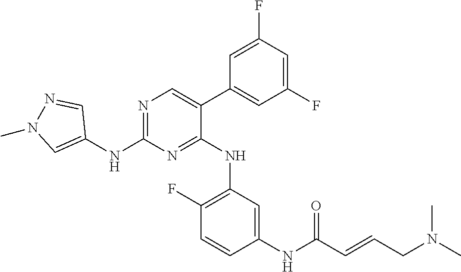 | J | 523.2 | δ 10.15 (bs, 1H), 9.27 (bs, 1H), 8.53 (bs, 1H), 7.99 (s, 1H), 7.76- 7.65 (m, 1H), 7.65-7.59 (m, 1H), 7.30-7.09 (m, 5H), 6.76-6.59 (m, 2H), 6.25 (d, J = 15.2 Hz, 1H), 3.54 (bs, 3H), 3.06-3.04 (m, 2H), 2.12 (s, 6H). |
| 118 | 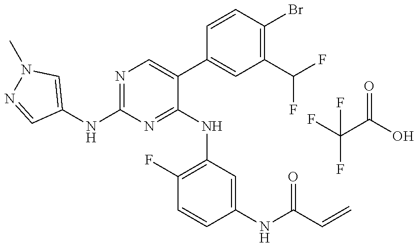 | K₂ | 558.1 | δ 10.28 (s, 1H), 9.89 (bs, 1H), 9.26 (bs, 1H), 7.97 (s, 1H), 7.87 (d, J = 8.0 Hz, 1H), 7.82- 7.74 (m, 2H), 7.61-7.58 (m, 2H), 7.34-7.01 (m, 5H), 6.42- 6.36 (m, 1H), 6.25-6.20 (m, 1H), 5.76-5.73 (m, 1H), 3.55 (bs, 3H). |
| 119 | 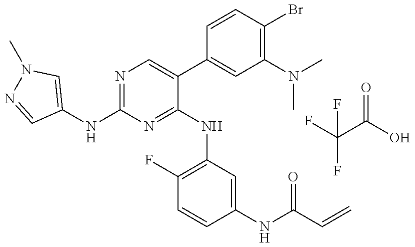 | K₂ | 551.2 | δ 10.29 (s, 1H), 10.01 (bs, 1H), 9.40 (bs, 1H), 7.94 (s, 1H), 7.82 (d, J = 5.2 Hz, 1H), 7.68 (d, J = 8.4 Hz, 1H), 7.51 (bs, 2H), 7.34- 6.93 (m, 5H), 6.43-6.36 (m, 1H), 6.25-6.21 (m, 1H), 5.77-5.74 (m, 1H), 3.56 (bs, 3H), 2.77-2.60 (m, 6H). |
| 120 | 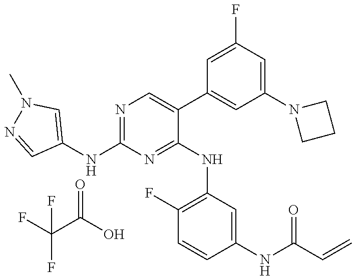 | K₂ | 503.2 | δ 10.29 (bs, 1H), 9.93 (bs, 1H), 9.28 (bs, 1H), 7.93-7.85 (m, 2H), 7.61 (bs, 1H), 7.35-6.95 (m, 5H), 6.59-6.23 (m, 4H), 5.79-5.76 (m, 1H), 3.88-3.84 (m, 6H), 3.57 (s, 3H). |
| 121 | 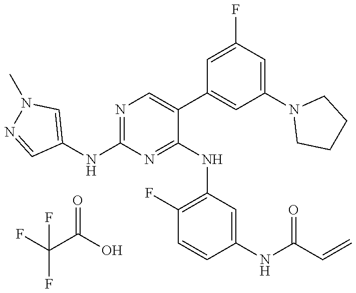 | K₂ | 517.3 | δ 10.27 (bs, 1H), 9.77 (bs, 1H), 9.11 (s, 1H), 7.94-7.84 (m, 2H), 7.61 (bs, 1H), 7.34-6.97 (m, 4H), 6.51-6.36 (m, 4H), 6.28-6.23 (m, 1H), 5.79-5.76 (m, 1H), 3.59 (bs, 3H), 3.29-3.17 (m, 4H), 1.99- 1.96 (m, 4H). |

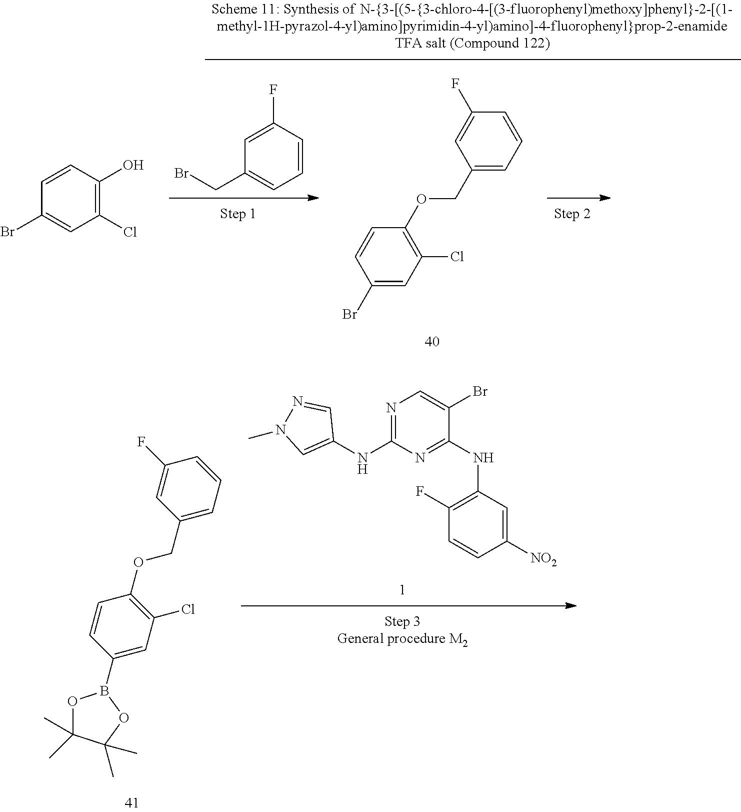 [SMILES: Oc1ccc(Br)cc1Cl ; Fc1cccc(CBr)c1 ; Fc1cccc(COc2ccc(Br)cc2Cl)c1 ; CC1(C)OB(c2ccc(OCc3cccc(F)c3)c(Cl)c2)OC1(C)C ; Cn1cc(Nc2ncc(Br)c(Nc3cc([N+](=O)[O-])ccc3F)n2)cn1]

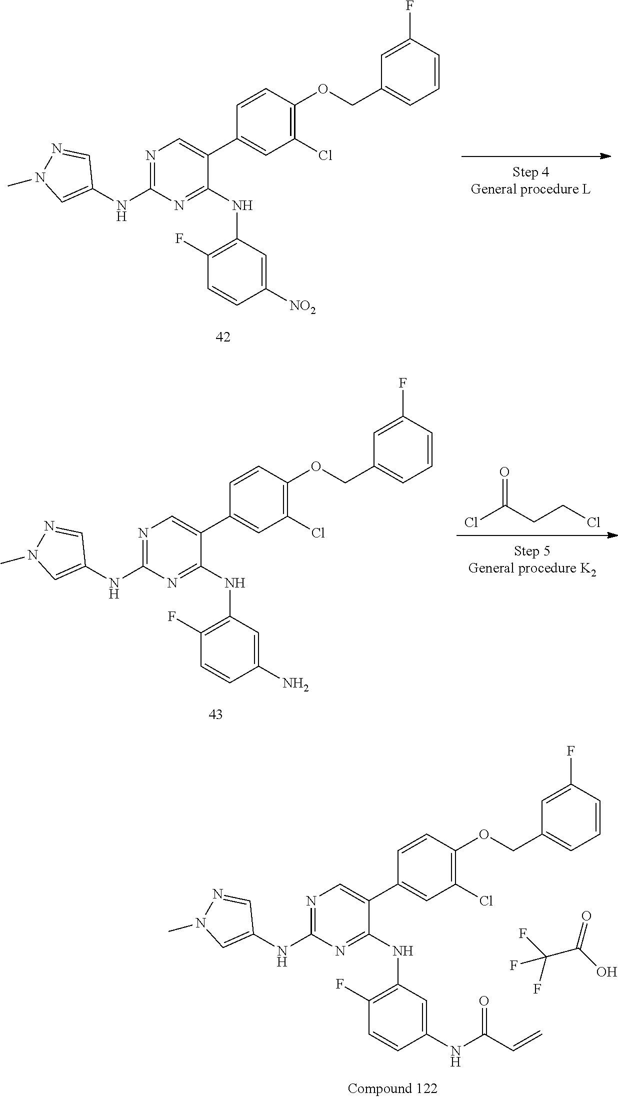

### Step 1: Synthesis of 4-bromo-2-chloro-1-[(3-fluorophenyl)methoxy]benzene (40)

To a stirred solution of 4-bromo-2-chlorophenol (1.20 g, 5.78 mmol) in N,N-dimethylformamide (10 mL) was added potassium carbonate (2.40 g, 17.3 mmol) and allowed to stir at room temperature for 10 minutes. To this reaction mixture was added 1-(bromomethyl)-3-fluorobenzene (1.31 g, 6.94 mmol) and stirred the reaction at room temperature for 12 hours.

The progress of the reaction was monitored by TLC. After the reaction completion, reaction mixture was quenched with ice water and extracted with ethyl acetate (50 mL×2). The combined organic layer was washed with brine (50 mL) and dried over sodium sulfate and concentrated under vacuum to the desired product (40) as off white solid (1.4 g, 76%). LCMS [M−H]⁺ 313.0.

### Step 2: Synthesis of 2-{3-chloro-4-[(3-fluorophenyl)methoxy]phenyl}-4,4,5,5-tetramethyl-1,3,2-dioxaborolane (41)

To a stirred solution of 4-bromo-2-chloro-1-[(3-fluorophenyl)methoxy]benzene (40) (1.00 g, 3.17 mmol), 4,4,5,5-tetramethyl-2-(4,4,5,5-tetramethyl-1,3,2-dioxaborolan-2-yl)-1,3,2-dioxaborolane (1.21 g, 4.75 mmol) in 1,4-dioxane (10.0 mL) was added potassium acetate (0.933 g, 9.51 mmol) and the mixture was purged with nitrogen for 5 minutes, followed by addition of 1,1′-bis(diphenylphosphino)ferrocenedichloropalladium(II), complex with dichloromethane (0.129 g, 0.158 mmol) and the reaction mixture was heated at 90° C. for 2 hours. After completion (TLC monitoring), reaction mixture was cooled and filtered through celite. The filtrate was concentrated to get black colored gum, which was diluted with water (10 mL) and extracted with ethyl acetate (30 mL×2). The combined organic layer was washed with brine (20 mL), dried over anhydrous sodium sulfate and concentrated to get titled compound (41) as black solid. (0.7 g, 60%). LCMS [M−H]⁺ 361.1.

### Step 3: Synthesis of 5-{3-chloro-4-[(3-fluorophenyl)methoxy]phenyl}-N4-(2-fluoro-5-nitrophenyl)-N2-(1-methyl-1H-pyrazol-4-yl)pyrimidine-2,4-diamine (42)

The title compound was prepared in a manner substantially similar to procedure mentioned in General Procedure M2, to afford the desired compound (42) as off white solid. LCMS [M+H]⁺ 564.2.

### Step 4: Synthesis of N4-(5-amino-2-fluorophenyl)-5-{3-chloro-4-[(3-fluorophenyl)methoxy]phenyl}-N2-(1-methyl-1H-pyrazol-4-yl)pyrimidine-2,4-diamine (43)

The title compound was prepared in a manner substantially similar to procedure mentioned in General Procedure L, to afford the desired compound (43) as brown solid. LCMS [M+H]⁺ 534.2.

### Step 5: Synthesis of N-{3-[(5-{3-chloro-4-[(3-fluorophenyl)methoxy]phenyl}-2-[(1-methyl-1H-pyrazol-4-yl)amino]pyrimidin-4-yl)amino]-4-fluorophenyl}prop-2-enamide TFA salt (Compound 122)

The title compound was prepared in a manner substantially similar to procedure mentioned in General Procedure K2, to afford the desired compound (Compound 122) as off white solid.

¹H NMR (400 MHz, DMSO-d6): δ 10.27 (s, 1H), 9.74 (bs, 1H), 9.15 (bs, 1H), 7.89 (bs, 1H), 7.82 (d, J=4.8 Hz, 1H), 7.59 (bs, 2H), 7.51-7.43 (m, 1H), 7.43-7.41 (m, 1H), 7.36-7.24 (m, 5H), 7.22-7.17 (m, 3H), 6.45-6.38 (m, 1H), 6.28-6.23 (m, 1H), 5.78-5.75 (m, 1H), 5.31 (s, 2H), 3.59 (bs, 3H). LCMS [M+H]⁺ 588.2.

Table 7: The following compounds were prepared using the procedures described above:

TABLE 7
The following compounds were prepared using the procedures described above:

| | | Gen- | | |
|---|---|---|---|---|
| | | eral | | |
| | | Pro- | LCMS | |
| Cmpd. | | ce- | [M + | ¹H-NMR (400 |
| No. | Structure | dure | H] | MHz, DMSO-d₆) |
| 123 | 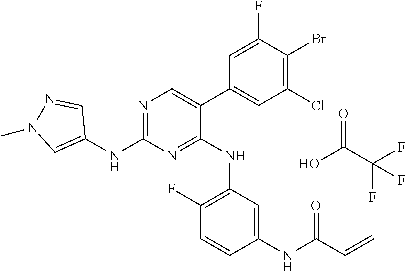 | K₂ | 560.1 | δ 10.29 (s, 1H), 9.83 (bs, 1H), 9.16 (bs, 1H), 8.02 (bs, 1H), 7.85 (d, J = 4.0 Hz, 1H), 7.69- 7.47 (m, 3H), 7.35 (bs, 1H), 7.22- 6.94 (m, 3H), 6.45-6.38 (m, 1H), 6.28-6.23 (m, 1H), 5.78- 5.75 (m, 1H), 3.57 (s, 3H). |
| 124 | 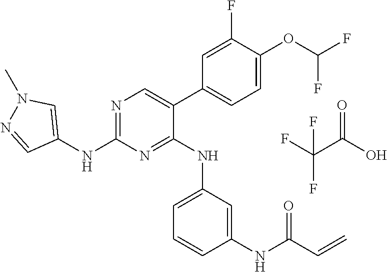 | K₂ | 496.2 | δ 10.24 (s, 1H), 9.93 (bs, 1H), 9.40 (s, 1H), 7.93- 7.85 (m, 2H), 7.61 (d, J = 11.2 Hz, 1H), 7.52- 7.49 (m, 3H), 7.39- 7.31 (m, 5H), 7.16-7.13 (m, 1H), 6.48-6.41 (m, 1H), 6.27-6.22 (m, 1H), 5.78- 5.75 (m, 1H), 3.52 (bs, 3H merged with DMSO peak). |
| 126 | 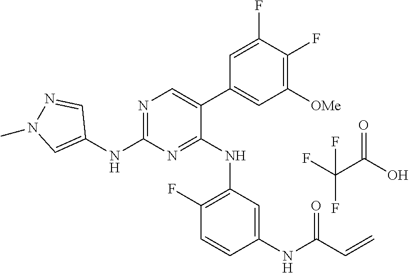 | K₂ | 496.2 | δ 10.27 (s, 1H), 9.80 (bs, 1H), 9.14 (bs, 1H), 7.93 (bs, 1H), 7.79 (d, J = 4.4 Hz, 1H), 7.59 (bs, 1H), 7.34 (bs, 2H), 7.26-7.14 (m, 4H), 6.44-6.37 (m, 1H), 6.25 (dd, J = 16.8 Hz, J = 1.6 Hz, 1H), 5.77 (dd, J = 10.0 Hz, J = 2.0 Hz, 1H), |
| | | | | 3.99 (s, 3H), |
| | | | | 3.58 (bs, 3H). |
| 127 | 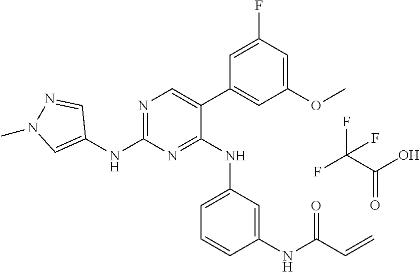 | K₂ | 460.2 | δ 10.26 (s, 1H), 10.10 (bs, 1H), 9.25 (bs, 1H), 7.93-7.82 (s, 2H), 7.50-7.37 (m, 4H), 7.22-6.88 (m, 5H), 6.46- 6.39 (m, 1H), 6.25- 6.21 (m, 1H), 6.25-6.21 (m, 1H) 3.81 (s, 3H), 3.57 (bs, 3H) |
| 129 | 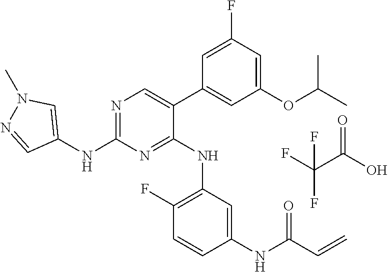 | K₂ | 506.3 | δ 10.28 (s, 1H), 9.91 (bs, 1H), 9.24 (bs, 1H), 7.96-7.85 (m, 2H), 7.60 (s, 1H), 7.35-7.09 (m, 4H), 6.96-6.85 (m, 3H), 6.45-6.39 (m, 1H), 6.28- 6.23 (m, 1H), 5.79- 5.76 ( m, 1H), 4.74-4.68 (m, 1H), 3.45 (s, 3H), 1.37-1.25 (m, 6H). |
| 130 | 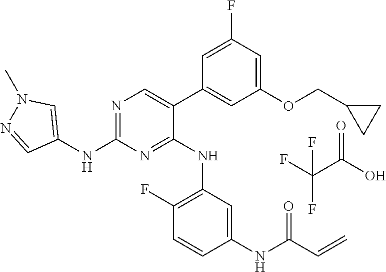 | K₂ | 518.2 | δ 10.21 (s, 1H), 9.16 (bs, 1H), 8.35 (bs, 1H), 7.96 (s, 1H), 7.78- 7.77 (m, 1H), 7.70-7.50 (m, 1H), 7.70-7.18 (m, 3H), 6.87-6.76 (m, 4H), 6.45- 6.38 (m, 1H), 6.27- 6.23 (m, 1H), 5.77-5.74 (m, 1H), 3.93-3.84 (m, 2H), 3.56 (bs, |
| | | | | 3H), 1.27-1.19 |
| | | | | (m, 1H), 0.61- |
| | | | | 0.56 (m, 2H), |
| | | | | 0.35-0.31 |
| | | | | (m, 2H). |
| 132 | 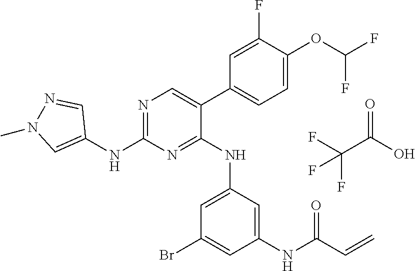 | K₂ | 574.1 | δ 10.33 (s, 1H), 9.87 (bs, 1H), 9.29 (bs, 1H), 7.96 (bs, 1H), 7.77 (d, J = 14.4 Hz, 2H), 7.59 (d, J = 11.6 Hz, 1H), 7.51-7.47 (m, 3H), 7.38-7.36 (m, 2H), 7.30 (s, 1H), 7.26-6.95 (m, 1H), 6.45- 6.38 (m, 1H), 6.29- 6.25 (m, 1H), 5.81-5.78 (m, 1H), 3.68 (bs, 3H). |
| 133 | 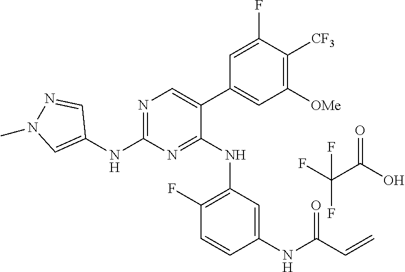 | K₂ | 546.2 | δ 10.28 (s, 1H), 9.77 (bs, 1H), 9.09 (bs, 1H), 8.09 (s, 1H), 7.85- 7.84 (m, 1H), 7.60 (bs, 1H), 7.35 (bs, 1H), 7.45 (bs, 1H), 7.23-7.08 (m, 4H), 6.95 (s, 1H), 6.28-6.23 (m, 1H), 5.76 (d, J = 12.0 Hz, 1H), 4.03 (s, 3H), 3.57 (bs, 3H). |
| 134 | 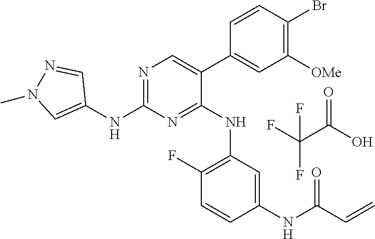 | K₂ | 538.1 | δ 10.30 (s, 1H), 9.98 (bs, 1H), 9.30 (s, 1H), 8.57 (bs, 1H), 7.98 (s, 1H), 7.86 (d, J = 4.8 Hz, 1H), 7.71 (d, J = 8.0 Hz, 1H), 7.59 (s, 1H), 7.35 (bs, 1H), 7.25 (s, 2H), 7.16 (s, 1H), 7.04 (d, J = 8.0 Hz, 1H), 6.42 (dd, J = 16.8 Hz, J = 8.8 Hz, |
| | | | | 1H), 6.25 (dd, J = |
| | | | | 16.8 Hz, J = 2.0 |
| | | | | Hz, 1H), 5.77 (dd, |
| | | | | J = 12.0 Hz, J = |
| | | | | 4.0 Hz, 1H), 3.92 |
| | | | | (s, 3H), 3.50 |
| | | | | (s, 3H, merged |
| | | | | with DMSO |
| | | | | peak). |
| 141 | 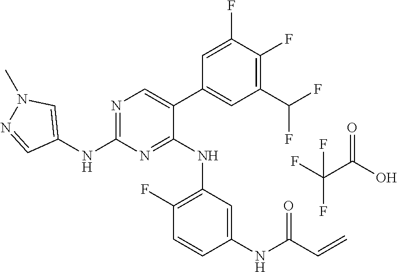 | K₂ | 516.2 | δ 10.32 (s, 1H), 10.04 (s, 1H), 9.30 (s, 1H), 8.02 (s, 1H), 7.86 (s, 2H), 7.58 (s, 2H), 7.45-7.32 (m, 2H), 7.23-7.18 (m, 3H), 6.45- 6.39 (m, 1H), 6.26 (d, J = 16.0 Hz, 1H), 5.79-5.76 (m, 1H), 3.59 (s, 3H). |
| 142 | 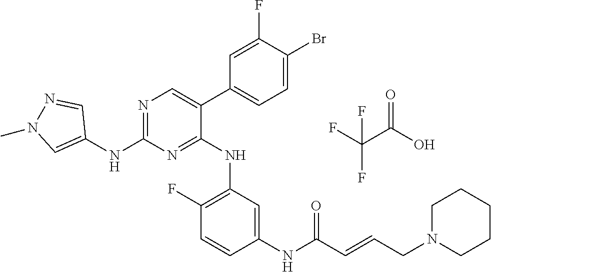 | J | 623.1 | δ 10.43 (s, 1H), 9.65 (bs, 1H), 9.40 (bs, 1H), 8.91 (bs, 1H), 8.07 (s, 1H), 7.83-7.79 (m, 2H), 7.59 (bs, 1H), 7.52 (d, J = 8.4 Hz, 1H), 7.35-7.29 (m, 2H), 7.2 (d, J = 7.6 Hz, 1H), 7.09 (bs, 1H), 6.80-6.72 (m, 1H), 6.43 (d, J = 16.0 Hz, 1H), 3.94 (bs, 2H), 3.71 (bs, 3H), 2.94-2.87 (m, |
| | | | | 2H), 1.84 (d, J = |
| | | | | 12.0 Hz, |
| | | | | 2H), 1.76- |
| | | | | 1.58 (m, 6H). |
| 144 | 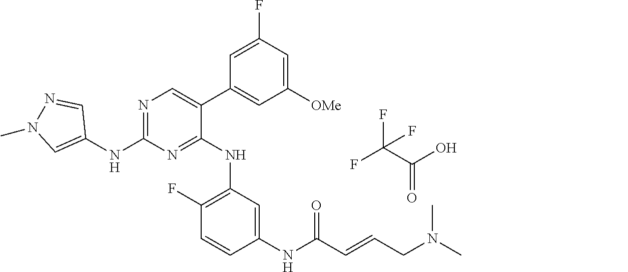 | K₂ | 535.3 | δ 10.47 (s, 1H), 9.84 (bs, 1H), 9.07 (bs, 1H), 8.05 (bs, 1H), 7.84- 7.81 (s, 1H), 7.62 (bs, 1H), 7.38- 7.36 (m, 1H), 7.24-7.12 (m, 2H), 6.98-6.87 (m, 4H), 6.78- 6.70 (m, 1H), 6.48- 6.43 (m, 1H), 4.04-3.99 (m, 2H), 3.84 (s, 3H), 3.69 (bs, 3H), 2.85 (s, 6H). |

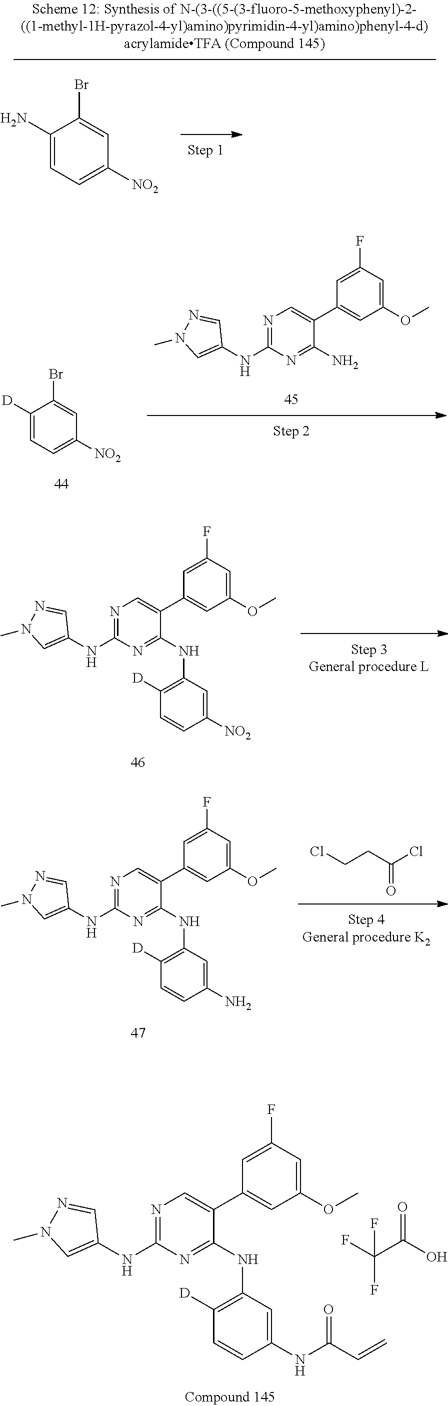

### Step 1: Synthesis of 1-bromo-3-nitrobenzene-6-d (44)

To a stirred solution of 2-bromo-4-nitroaniline (0.1 g, 0.461 mmol) in N,N-dimethylformamide D₇ (0.3 mL) was added tert-butyl nitrite (0.111 mL, 0.922 mmol) drop wise and the reaction mixture was stirred at room temperature for 15 min. The reaction mixture was quenched with water and was extracted with ethyl acetate (20 mL×3). The combined organic layer was washed with water (20 mL×3), brine (20 mL), dried over anhydrous sulfate and evaporated. The crude product was purified by column chromatography using combiflash purifier and was eluted with 5% ethyl acetate in hexane to get the title compound (44) as colourless liquid (0.05 g, 53%). ¹H NMR (400 MHz, CDCl₃): δ 8.41 (d, J=2.0 Hz, 1H), 8.20 (dd, J=8.4 Hz, J=2.0 Hz, 1H), 7.48-7.45 (m, 1H).

### Step 2: Synthesis of 5-(3-fluoro-5-methoxyphenyl)-N2-(1-methyl-1H-pyrazol-4-yl)-N4-(3-nitrophenyl-6-d)pyrimidine-2,4-diamine (46)

To a stirred solution of 5-(3-fluoro-5-methoxyphenyl)-N2-(1-methyl-1H-pyrazol-4-yl)pyrimidine-2,4-diamine (45) (0.150 g, 0.477 mmol) in 1,4-dioxane (5 mL) were added 1-bromo-3-nitro(6-²H)benzene (44) (0.145 g, 0.716 mmol), caesium carbonate (0.466 g, 1.43 mmol). The reaction mixture was degasified and purged with argon for 5 minutes then was added Tris(dibenzylideneacetone)dipalladium(0) (0.021 g, 0.023 mmol) and [5-(diphenylphosphanyl)-9,9-dimethyl-9H-xanthen-4-yl]diphenylphosphane (0.013 g, 0.023 mmol) and the reaction mixture was heated at 100° C. for 15 hours in a sealed tube. The reaction mixture was cooled, diluted with water (10 mL) and extracted with ethyl acetate (10 mL×3). The combined organic layer was washed with brine (10 mL), dried over anhydrous sodium sulfate and evaporated. The crude product was purified by column chromatography using combiflash purifier and was eluted with 50% ethyl acetate in hexane to get the title compound (46) as yellow solid (0.15 g, 72%). LCMS [M+H]⁺ 437.0.

### Step 3: Synthesis of N4-(3-aminophenyl-6-d)-5-(3-fluoro-5-methoxyphenyl)-N2-(1-methyl-1H-pyrazol-4-yl)pyrimidine-2,4-diamine (47)

The title compound was prepared in a manner substantially similar to procedure mentioned in General Procedure L, to afford the desired compound (47) as brown solid. LCMS [M+H]⁺ 407.2.

### Step 4: Synthesis of N-(3-((5-(3-fluoro-5-methoxyphenyl)-2-((1-methyl-1H-pyrazol-4-yl)amino)pyrimidin-4-yl)amino)phenyl-4-d)acrylamide. TFA (Compound 145)

The title compound was prepared in a manner substantially similar to procedure mentioned in General Procedure K2, to afford the desired compound (Compound 145) as off white solid. ¹H NMR (400 MHz, DMSO-d6): δ 10.23 (s, 1H), 10.00 (bs, 1H), 9.40 (bs, 1H), 7.93 (bs, 1H), 7.82 (s, 1H), 7.49-7.07 (m, 5H), 6.94-6.87 (m, 3H), 6.46-6.39 (m, 1H), 6.25-6.24 (m, 1H), 5.75-5.72 (m, 1H), 3.92 (s, 3H), 3.58 (bs, 3H merged with DMSO peak). LCMS [M+H]⁺ 461.2.

Table 8: The following compounds were prepared using the procedures described above:

TABLE 8
The following compounds were prepared using the procedures described above:

| | | Gen | | |
|---|---|---|---|---|
| | | eral | | |
| | | Pro- | LCMS | ¹H-NMR |
| Cmpd. | | ce- | [M + | (400 MHz, |
| No. | Structure | dure | H] | DMSO-d₆) |
| 148 | 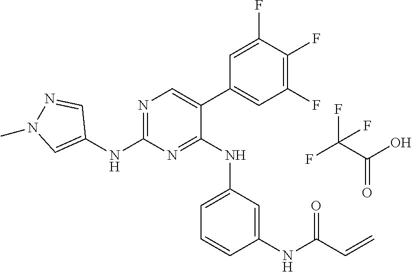 | K₂ | 466.2 | δ 10.27 (s, 2H), 9.42 (bs, 1H), 8.99 (bs, 1H), 7.97-7.84 (m, 2H), 7.52-7.13 (m, 7H), 6.46-6.40 (m, 1H), 6.25-6.21 (m, 1H), 5.76- 5.73 (m, 1H), 3.75 (s, 3H) |
| 149 | 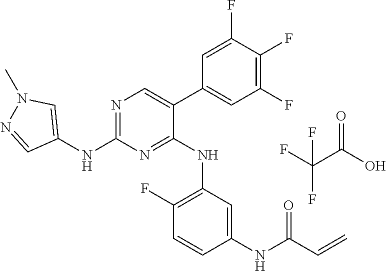 | K₂ | 484.2 | δ 10.31 (s, 1H), 10.06 (s, 1H), 9.24 (s, 1H), 7.99 (s, 1H), 7.86 (s, 1H), 7.57-7.49 (m, 3H), 7.36 (s, 1H), 7.20-7.12 (m, 3H), 6.44- 6.37 (m, 1H), 6.24 (d, J = 8.0 Hz, 1H), 5.75 (m, J = 12.0 Hz, 1H), 3.56 (s, 3H). |
| 150 | 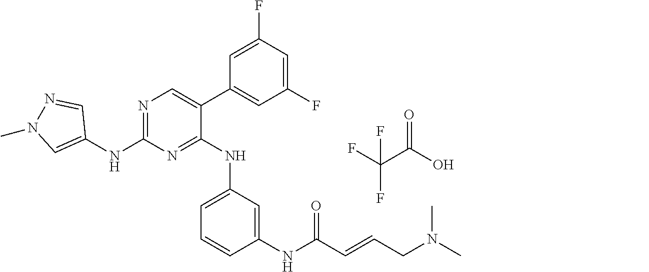 | J | 503.3 | δ 10.41 (s, 1H), 10.07 (bs, 1H), 9.85 (s, 1H), 9.34 (bs, 1H), 8.00 (s, 1H), 7.81 (s, 1H), 7.53 (bs, 1H), 7.40 (bs, 2H), 7.31-7.23 (m, 5H), 6.77-6.69 (m, 1H), 6.49- 6.45 (m, 1H), 3.95 (d, J = 6.8 Hz, 2H), 3.61 (bs, 3H), 2.80 (bs, 6H). |
| 152 | 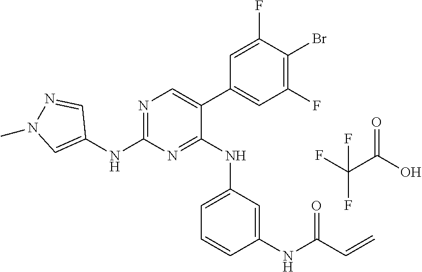 | K₂ | 526.1 | δ 10.24 (bs, 1H), 10.07 (bs, 1H), 9.43 (bs, 1H), 7.98-7.77 (m, 2H), 7.47-6.99 (m, 7H), 6.46-6.42 (m, 2H), 6.25- 6.20 (m, 1H), 5.76- 5.73 (m, 1H), 3.50 (s, 3H) |
| 153 | 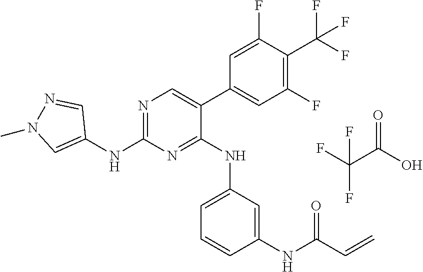 | K₂ | 516.2 | δ 10.21 (s, 1H), 9.80 (bs, 1H), 9.24 (bs, 1H), 8.05 (s, 1H), 7.83 (s, 1H), 7.57 (d, J = 11.2 Hz, 3H), 7.49-7.32 (m, 4H), 7.21-7.08 (m, 1H), 6.48- 6.41 (m, 1H), 6.25 (dd, J = 16.8 Hz, J = 2.0 Hz, 1H), |
| | | | | 5.76 (dd, |
| | | | | J = 10.0 |
| | | | | Hz, J = 1.6 Hz, |
| | | | | 1H), 3.49 |
| | | | | (bs, 3H). |
| 154 | 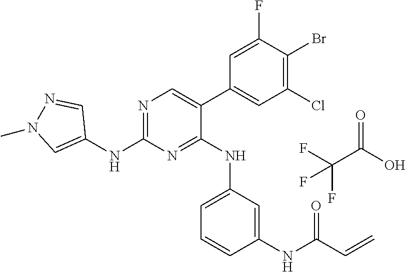 | K₂ | 544.0 | δ 10.25 (s, 1H), 10.01 (bs, 1H), 9.40 (bs, 1H), 7.98 (s, 1H), 7.83 (s, 1H), 7.64 (s, 1H), 7.56 (d, J = 8.8 Hz, 1H), 7.47-6.46 (m, 6H), 6.43-6.39 (m, 1H), 6.25- 6.20 (m, 1H), 5.76- 5.73 (m, 1H), 3.48 (bs, 3H). |
| 155 | 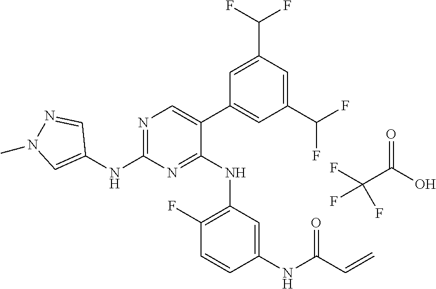 | K₂ | 529.5 | δ 10.44 (bs, 1H), 10.34 (s, 1H), 9.69 (bs, 1H), 8.07 (bs, 1H), 7.89- 7.81 (m, 4H), 7.59 (bs, 1H), 7.39- 7.24 (m, 2H), 7.19-7.12 (m, 1H), 7.05 (s, 2H), 6.99 (s, 1H), 6.45-6.39 (m, 1H), 6.28-6.23 (m, 1H), 5.79- 5.76 (m, |
| | | | | 1H), 3.61 |
| | | | | (s, 3H). |
| 156 | 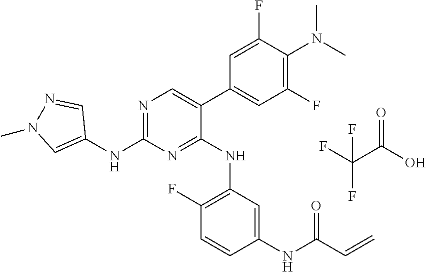 | K₂ | 509.2 | δ 10.31 (s, 1H), 10.12 (bs, 1H), 9.46 (bs, 1H), 7.94-7.85 (m, 3H), 7.58-7.52 (m, 1H), 7.36 (bs, 1H), 7.19-7.07 (m, 3H), 6.94 (bs, 1H), 6.43-6.36 (m, 1H), 6.26-6.21 (m, 1H), 5.76 (d, J = 12.0 Hz, 1H), |
| | | | | 3.58 (bs, 3H |
| | | | | merged |
| | | | | with DMSO |
| | | | | peak), 2.85 |
| | | | | (s, 6H). |

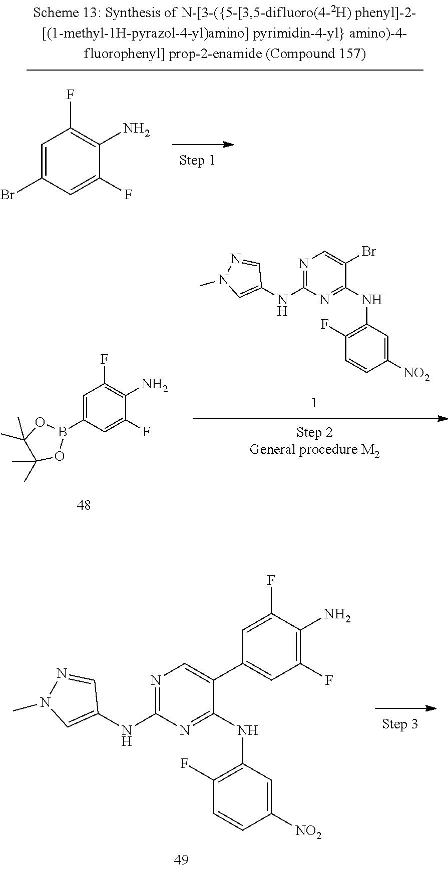 [SMILES: Nc1c(F)cc(Br)cc1F ; CC1(C)OB(c2cc(F)c(N)c(F)c2)OC1(C)C ; Cn1cc(Nc2ncc(Br)c(Nc3cc([N+](=O)[O-])ccc3F)n2)cn1 ; Cn1cc(Nc2ncc(-c3cc(F)c(N)c(F)c3)c(Nc3cc([N+](=O)[O-])ccc3F)n2)cn1]

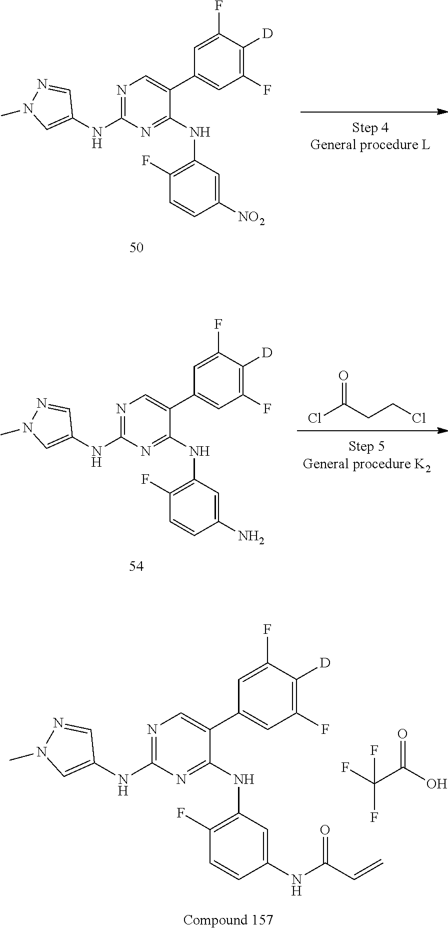 [SMILES: [2H]c1c(F)cc(-c2cnc(Nc3cnn(C)c3)nc2Nc2cc([N+](=O)[O-])ccc2F)cc1F ; [2H]c1c(F)cc(-c2cnc(Nc3cnn(C)c3)nc2Nc2cc(N)ccc2F)cc1F ; O=C(Cl)CCCl ; [2H]c1c(F)cc(-c2cnc(Nc3cnn(C)c3)nc2Nc2cc(NC(=O)C=C)ccc2F)cc1F ; O=C(O)C(F)(F)F]

### Step 1: Synthesis of 2,6-difluoro-4-(4,4,5,5-tetramethyl-1,3,2-dioxaborolan-2-yl) aniline (48)

To a stirred solution of 4-bromo-2,6-difluoroaniline (2 g, 9.62 mmol) in 1,4-dioxane (30 mL) was added 4,4,5,5-tetramethyl-2-(4,4,5,5-tetramethyl-1,3,2-dioxaborolan-2-yl)-1,3,2-dioxaborolane (2.69 g, 10.6 mmol), potassium acetate (2.83 g, 28.8 mmol), the reaction mixture was purged in nitrogen for 5 min and added [1,1′-Bis(diphenylphosphino)ferrocene]palladium(II) chloride in dichloromethane (0.704 g, 0.962 mmol) and the reaction mixture was heated at 100° C. for 12 hours. The progress of the reaction was monitored by TLC/LCMS. After the reaction completion, the reaction mixture was filtered through the celite and the filtrate was evaporated under reduced pressure to afford 2,6-difluoro-4-(4,4,5,5-tetramethyl-1,3,2-dioxaborolan-2-yl) aniline (48) (2 g, 82%) as dark brown liquid. ¹H NMR (400 MHz, DMSO-d6): δ 7.39 (s, 1H), 7.28 (s, 1H), 3.95 (s, 2H), 1.30 (s, 12H).

### Step 2: Synthesis of 5-(4-amino-3,5-difluorophenyl)-N4-(2-fluoro-5-nitrophenyl)-N2-(1-methyl-1H-pyrazol-4-yl) pyrimidine-2,4-diamine (49)

The title compound was prepared in a manner substantially similar to procedure mentioned in General Procedure M2, to afford the desired compound (49) as pale yellow solid. LCMS [M+H]⁺ 457.1.

### Step 3: Synthesis of 5-[3,5-difluoro(4-²H) phenyl]-N4-(2-fluoro-5-nitrophenyl)-N2-(1-methyl-1H-pyrazol-4-yl) pyrimidine-2,4-diamine (50)

To a stirred solution of 5-(4-amino-3,5-difluorophenyl)-N4-(2-fluoro-5-nitrophenyl)-N2-(1-methyl-1H-pyrazol-4-yl) pyrimidine-2,4-diamine (49) (0.2 g, 0.438 mmol) in dimethyl formamide-d7 (0.8 mL) was added tert-butyl nitrite (0.226 g, 2.19 mmol) at room temperature. The reaction mixture was stirred at room temperature for 1 hour. The progress of the reaction was monitored by LCMS. After reaction completion, reaction mass was diluted with water (10 mL) and extracted with ethyl acetate (10 mL×2). The combined organic layer was washed with water (10 mL×3), brine (10 mL), dried over anhydrous sodium sulfate and concentrated under reduced pressure. The crude product was purified by column chromatography by using combiflash purifier and was eluted with 30-50% ethyl acetate in hexane to afford 5-[3,5-difluoro(4-²H) phenyl]-N4-(2-fluoro-5-nitrophenyl)-N2-(1-methyl-1H-pyrazol-4-yl) pyrimidine-2,4-diamine (50) (0.05 g, 26%) as a pale brown solid. LCMS [M+H]⁺ 443.1.

### Step 4: Synthesis of N4-(5-amino-2-fluorophenyl)-5-[3,5-difluoro(4-²H) phenyl]-N2-(1-methyl-1H-pyrazol-4-yl) pyrimidine-2,4-diamine (51)

The title compound was prepared in a manner substantially similar to procedure mentioned in General Procedure L, to afford the desired compound (51) as brown solid. LCMS [M+H]⁺ 413.2.

### Step 5: Synthesis of N-[3-({5-[3,5-difluoro(4-²H) phenyl]-2-[(1-methyl-1H-pyrazol-4-yl) amino] pyrimidin-4-yl} amino)-4-fluorophenyl] prop-2-enamide (Compound 157)

The title compound was prepared in a manner substantially similar to procedure mentioned in General Procedure K2, to afford the desired compound (Compound 157) as off white solid. ¹H NMR (400 MHz, DMSO-d6): δ 10.26 (s, 1H), 9.63 (s, 1H), 8.98 (s, 1H), 8.01 (s, 1H), 7.83 (d, J=5.6 Hz, 1H), 7.59 (s, 1H), 7.34 (s, 1H), 7.26-7.20 (m, 5H), 6.45-6.38 (m, 1H), 6.28-6.23 (m, 1H), 5.78-5.75 (m, 1H), 3.47 (s, 3H). LCMS [M+H]⁺ 467.2.

TABLE 9
The following compounds were prepared using the procedures described above:

| Cmpd. | | General | LCMS | |
|---|---|---|---|---|
| No. | Structure | Procedure | [M + H] | ¹H-NMR (400 MHz, DMSO-d₆) |
| 158 | 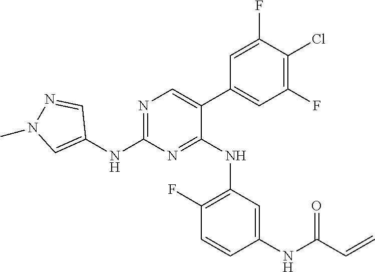 [SMILES: C=CC(=O)Nc1ccc(F)c(Nc2nc(Nc3cnn(C)c3)ncc2-c2cc(F)c(Cl)c(F)c2)c1] | K₂ | 500.2 | δ 10.28 (s, 1H), 9.77 (bs, 1H), 9.07 (bs, 1H), 7.99 (s, 1H), 7.82 (bs, 1H), 7.56 − 7.47 (m, 2H), 7.33 (bs, 1H), 7.19 − 7.06 (m, 3H), 6.43 − 6.36 (m, 1H), 6.25 − 6.21 (m, 1H), 5.76 − 5.73 (m, 2H), 3.54 (s, 3H). |
| 159 | 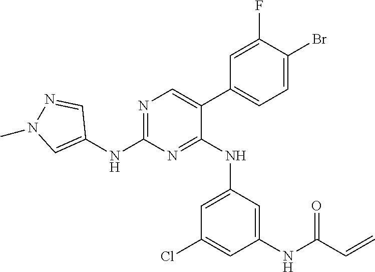 [SMILES: C=CC(=O)Nc1cc(Cl)cc(Nc2nc(Nc3cnn(C)c3)ncc2-c2ccc(Br)c(F)c2)c1] | K₂ | 542.1 | δ 10.27 (s, 1H), 9.83 (bs, 1H), 9.21 (bs, 1H), 7.91 (bs, 1H), 7.76 − 7.72 (m, 1H), 7.63 (s, 1H), 7.57 (s, 1H), 7.49 (d, J = 9.2 Hz, 2H), 7.30 − 7.21 (m, 4H), 6.38 − 6.31 (m, 1H), 6.19 (dd, J = 16.8 Hz, J = 1.6 Hz, 1H), 5.72 (dd, J = 10.0 Hz, J = 1.6 Hz, 1H), 3.60 (s, 3H). |
| 160 | 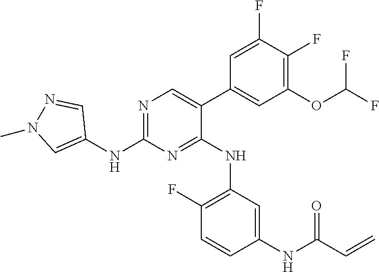 [SMILES: C=CC(=O)Nc1ccc(F)c(Nc2nc(Nc3cnn(C)c3)ncc2-c2cc(F)c(F)c(OC(F)F)c2)c1] | K₂ | 532.2 | δ 10.22 (s, 1H), 9.79 (bs, 1H), 9.03 (bs, 1H), 7.92 (s, 1H), 7.79 (d, J = 4.8 Hz, 1H), 7.50 − 7.46 (m, 2H), 7.32 − 7.29 (m, 3H), 7.15 − 6.90 (m, 3H), 6.38 − 6.31 (m, 1H), 6.20 − 6.16 (m, 1H), 5.71 − 5.68 (m, 1H), 3.51 (bs, 3H). |
| 162 | 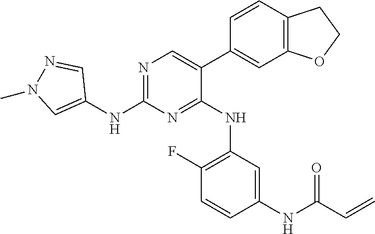 [SMILES: C=CC(=O)Nc1ccc(F)c(Nc2nc(Nc3cnn(C)c3)ncc2-c2ccc3c(c2)OCC3)c1] | K₂ | 472.3 | δ 10.29 (s, 1H), 10.04 (bs, 1H), 9.37 (bs, 1H), 7.87 − 7.85 (m, 2H), 7.60 (s, 1H), 7.39 − 7.09 (m, 5H), 6.98 − 6.93 (m, 2H), 6.45 − 6.38 (m, 1H), 6.28 − 6.23 (m, 1H), 5.79 − 5.76 (m, 1H), 4.60 ( t, J = 8.0 Hz, 2H), 3.60 (bs, 3H), 3.24 (t, J = 8.8 Hz, 2H). |
| 163 | 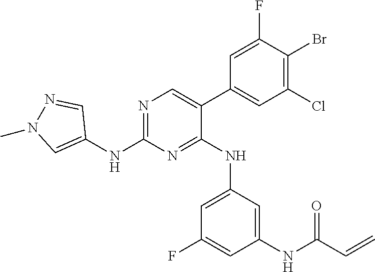 [SMILES: C=CC(=O)Nc1cc(F)cc(Nc2nc(Nc3cnn(C)c3)ncc2-c2cc(F)c(Br)c(Cl)c2)c1] | K₂ | 560.1 | δ 10.33 (s, 1H), 9.64 (bs, 1H), 9.10 (bs, 1H), 8.01 (s, 1H), 7.62 − 7.0 (m, 8H), 6.46 − 6.24 (m, 2H), 5.80 − 5.77 (m, 1H), 3.67 (bs, 3H). |
| 164 | 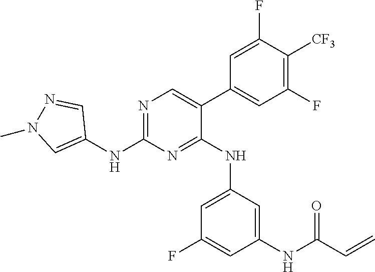 [SMILES: C=CC(=O)Nc1cc(F)cc(Nc2nc(Nc3cnn(C)c3)ncc2-c2cc(F)c(C(F)(F)F)c(F)c2)c1] | K₂ | 534.2 | δ 10.35 (s, 1H), 9.82 (bs, 1H), 9.21 (bs, 1H), 8.07 (s, 1H), 7.56 − 7.34 (m, 7H), 7.12 − 7.07 (m, 1H), 6.44 − 6.37 (m, 1H), 6.27 − 6.23 (m, 1H), 5.78 − 5.75(m, 1H), 3.62 (s, 3H). |
| 165 | 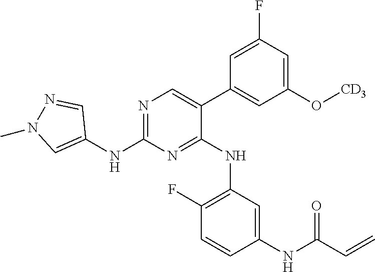 [SMILES: [2H]C([2H])([2H])Oc1cc(F)cc(-c2cnc(Nc3cnn(C)c3)nc2Nc2cc(NC(=O)C=C)ccc2F)c1] | K₂ | 481.3 | δ 10.29 (s, 1H), 9.22 (bs, 1H), 7.98 (bs, 1H), 7.86 − 7.84 (m, 1H), 7.61 (bs, 1H), 7.35 (bs, 1H), 7.23 (bs, 1H), 7.10 (bs, 1H), 6.97 − 6.88 (m, 4H), 6.45 − 6.39 (m, 1H), 6.28 − 6.23 (m, 1H), 5.79 − 5.76 (m, 2H), 3.59 (bs, 3H). |
| 166 | 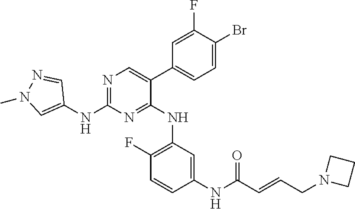 [SMILES: Cn1cc(Nc2ncc(-c3ccc(Br)c(F)c3)c(Nc3cc(NC(=O)/C=C/CN4CCC4)ccc3F)n2)cn1] | J | 596.2 | δ 10.39 (s, 1H), 9.93 (bs, 1H), 9.45 (bs, 1H), 8.74 (bs, 1H), 7.96 (s, 1H), 7.80 − 7.76 (m, 2H), 7.56 − 7.27 (m, 4H), 7.15 − 7.08 (m, 2H), 6.60 − 6.57 (m, 1H), 6.37 − 6.33 (m, 1H), 4.13 − 3.99 (m, 4H), 3.53 (bs, 3H), 2.48 (s, 2H), 2.38 (s, 2H). |
| 167 | 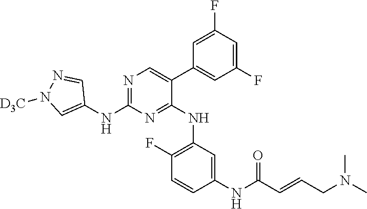 [SMILES: [2H]C([2H])([2H])n1cc(Nc2ncc(-c3cc(F)cc(F)c3)c(Nc3cc(NC(=O)/C=C/CN(C)C)ccc3F)n2)cn1] | J | 524.3 | δ 10.56 (s, 2H), 10.13 (bs, 1H), 9.50 (s, 1H), 8.09 (s, 1H), 7.88 − 7.86 (m, 1H), 7.65 (s, 1H), 7.40 − 7.14 (m, 6H), 6.82 − 6.71 (m, 1H), 6.48 − 6.45 (m, 1H), 3.96 − 3.94 (s, 2H), 2.85 − 2.75 (m, 6H). |
| 168 | 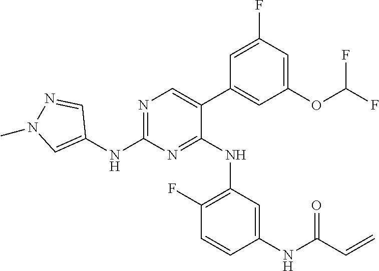 [SMILES: C=CC(=O)Nc1ccc(F)c(Nc2nc(Nc3cnn(C)c3)ncc2-c2cc(F)cc(OC(F)F)c2)c1] | K₂ | 514.3 | δ 10.37 (s, 1H), 9.95 (bs, 1H), 9.27 (bs, 1H), 8.09 (bs, 1H), 7.87 − 7.86 (m, 1H), 7.60 (bs, 1H), 7.37 (bs, 2H), 7.30 − 7.14 (m, 6H), 6.45 − 6.39 (m, 1H), 6.28 − 6.23 (m, 1H), 5.79 − 5.76 (d, J = 12.0 Hz, 1H), 3.59 (bs, 3H). |
| 169 | 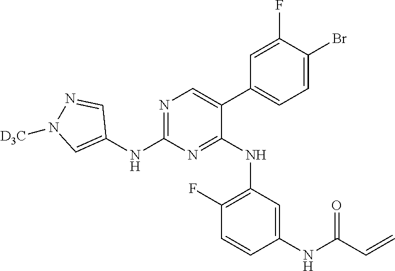 [SMILES: [2H]C([2H])([2H])n1cc(Nc2ncc(-c3ccc(Br)c(F)c3)c(Nc3cc(NC(=O)C=C)ccc3F)n2)cn1] | K₂ | 529.2 | δ 10.29 (s, 1H), 9.92 (bs, 1H), 9.27 (bs, 1H), 7.97 (s, 1H), 7.84 − 7.80 (m, 2H), 7.54 (d, J = 9.6 Hz, 2H), 7.34 − 7.28 (m, 2H), 7.19 − 7.06 (m, 3H), 6.43 − 6.36 (m, 1H), 6.25 − 6.21 (dd, J = 16.8 Hz, J = 3.2 Hz, 1H), 5.76 − 5.73 (m, 1H). |
| 170 | 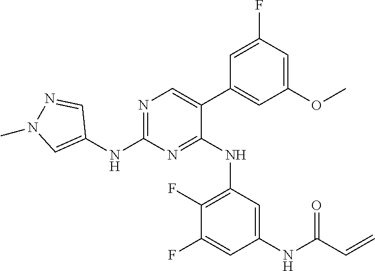 [SMILES: C=CC(=O)Nc1cc(F)c(F)c(Nc2nc(Nc3cnn(C)c3)ncc2-c2cc(F)cc(OC)c2)c1] | K₂ | 496.3 | δ 9.82 (s, 1H), 9.76 (bs, 1H), 9.21 (s, 1H), 8.01 (s, 1H), 7.80 (s, 1H), 7.52 − 7.45 (m, 3H), 6.94 − 6.89 (m, 4H), 6.51 − 6.44 (m, 1H), 6.29 − 6.25 (m, 1H), 5.82 − 5.79 (m, 1H), 3.83 (s, 3H), 3.78 (s, 3H). |
| 171 | 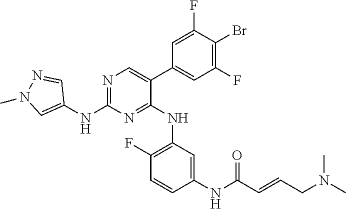 | J | 601.2 | δ 10.55 (s, 1H), 10.23 (bs, 1H), 10.10 (bs, 1H), 9.31 (bs, 1H), 8.07 (s, 1H), 7.85 (d, J = 5.2 Hz, 1H), 7.63 (bs, 1H), 7.47 − 7.38 (m, 2H), 7.26 (s, 1H), 7.14 (s, 1H), 7.01 (s, 1H), 6.79 − 6.72 (m, 1H), 6.46 (d, J = 16.0 Hz, 1H), 3.94 (d, J = 8.0 Hz, 2H), 3.58 (s, 3H), 2.79 (s, 6H). |
| 172 | 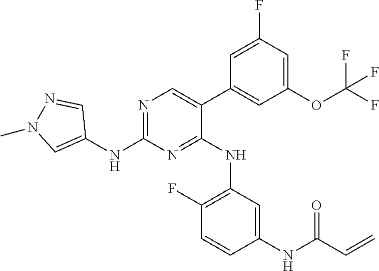 [SMILES: C=CC(=O)Nc1ccc(F)c(Nc2nc(Nc3cnn(C)c3)ncc2-c2cc(F)cc(OC(F)(F)F)c2)c1] | K₂ | 532.3 | δ 10.22 (s, 1H), 9.28 (bs, 1H), 8.59 (s, 1H), 8.01 (s, 1H), 7.78 − 7.77 (m, 1H), 7.59 (bs, 1H), 7.43 − 7.40 (m, 1H), 7.33 (bs, 3H), 7.25 − 7.0 (m, 2H), 6.45 − 6.38 (m, 1H), 6.28 − 6.23 (m, 1H), 5.77 − 5.74 (m, 1H), 3.56 (bs, 3H). |
| 173 | 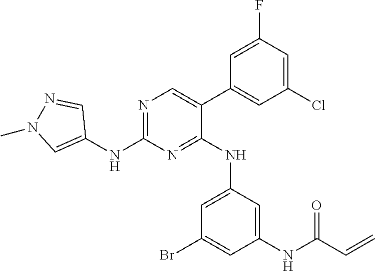 [SMILES: C=CC(=O)Nc1cc(Br)cc(Nc2nc(Nc3cnn(C)c3)ncc2-c2cc(F)cc(Cl)c2)c1] | K₂ | 542.2 | δ 10.30 (s, 1H), 9.55 (bs, 1H), 8.95 (bs, 1H), 8.02 (s, 1H), 7.77 (s, 1H), 7.66 (s, 1H), 7.46 − 7.34 (m, 6H), 7.22 − 6.96 (m, 1H), 6.45 − 6.39 (m, 1H), 6.29 − 6.24 (m, 1H), 5.80 − 5.77 (m, 1H), 3.67 (s, 3H). |
| 174 | 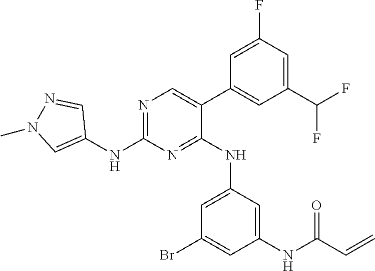 [SMILES: C=CC(=O)Nc1cc(Br)cc(Nc2nc(Nc3cnn(C)c3)ncc2-c2cc(F)cc(C(F)F)c2)c1] | K₂ | 558.1 | δ 10.35 (bs, 1H), 10.04 (bs, 1H), 9.40 (s, 1H), 8.01 (s, 1H), 7.92 − 7.78 (m, 2H), 7.73 − 7.46 (m, 6H), 7.08 (s, 2H), 6.43 − 6.36 (m, 1H), 6.25 (d, J = 16, 1H), 5.77 (d, J = 12.0 Hz, 1H), 3.65 (s, 3H). |
| 175 | 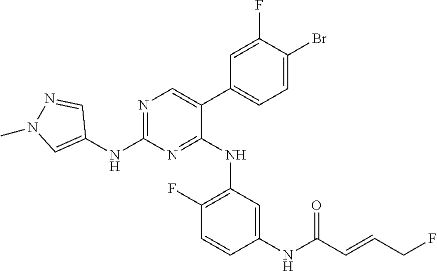 [SMILES: Cn1cc(Nc2ncc(-c3ccc(Br)c(F)c3)c(Nc3cc(NC(=O)/C=C/CF)ccc3F)n2)cn1] | J | 558.0 | 10.31 (s, 1H), 10.14 (s, 1H), 9.80 (s, 1H), 9.00 (s, 1H), 7.98 (s, 1H), 7.84 − 7.80 (m, 2H), 7.53 (d, J = 10.0 Hz, 2H), 7.30 (d, J = 8.0 Hz, 2H), 7.22 (bs, 2H), 6.92 − 6.82 (m, 1H), 6.35 (d, J = 15.2 Hz, 1H), 5.24 (s, 1H), 5.12 (s, 1H), 3.50 (s, 3H, merged in solvent peak). |
| 176 | 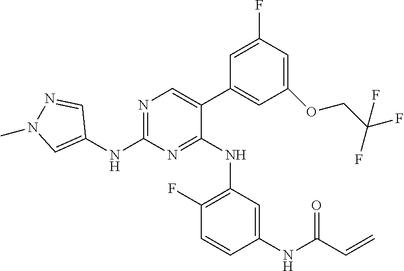 [SMILES: C=CC(=O)Nc1ccc(F)c(Nc2nc(Nc3cnn(C)c3)ncc2-c2cc(F)cc(OCC(F)(F)F)c2)c1] | K₂ | 546.3 | δ 10.28 (s, 1H), 9.79 (bs, 1H), 9.06 (bs, 1H), 8.00 (s, 1H), 7.84 (d, J = 4.8 Hz, 1H), 7.61 (bs, 1H), 7.34 (bs, 1H), 7.22 (bs, 1H), 7.11 − 6.96 (m, 5H), 6.46 − 6.39 (m, 1H), 6.26 (dd, J = 16.8 Hz, J = 1.6 Hz, 1H), 5.77 (dd, J = 10.4 Hz, J = 2.0 Hz, 1H), 4.90 − 4.84 (m, 2H), 3.65 (bs, 3H). |
| 177 | 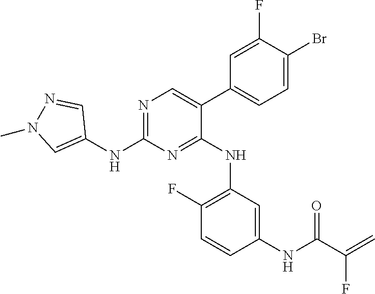 [SMILES: C=C(F)C(=O)Nc1ccc(F)c(Nc2nc(Nc3cnn(C)c3)ncc2-c2ccc(Br)c(F)c2)c1] | J | 544.1 | δ 10.45 (s, 1H), 9.92 (bs, 1H), 9.26 (bs, 1H), 7.97 (bs, 1H), 7.86 − 7.80 (m, 2H), 7.66 (bs, 1H), 7.54 − 7.52 (m, 1H), 7.37 (bs, 1H), 7.30 (bs, 1H), 7.28 − 7.20 (m, 2H), 7.07 (bs, 1H), 5.75 − 5.62 (m, 1H), 5.45 − 5.40 (m, 1H), 3.50 (bs, 3H). |
| 178 | 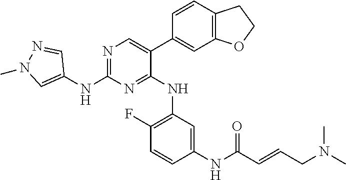 | J | 529.0 | 10.53 (s, 1H), 10.00 (bs, 1H), 7.93 (bs, 1H), 7.83 − 7.81 (s, 1H), 7.62 (bs, 1H), 7.37 − 7.35 (s, 2H), 7.20 − 7.10 (m, 2H), 6.96 − 6.90 (m, 2H), 6.75 − 6.68 (m, 1H), 6.45 − 6.41 (m, 1H), 4.59 − 4.55 (m, 2H), 3.93 − 3.91 (m, 2H), 3.57 (s, 3H), 3.24 − 3.20 (m, 2H), 2.77 (s, 6H). |

### Example 2: Cellular Proliferation (Alamar Blue) Assays

Cell line details:
1. EGFR(D770_N771insSVD) expressing Ba/F3 stable cell line
2. EGFR (A767_dupASV) expressing Ba/F3 stable cell line
3. A431 cells
4. EGFR (H773insNPH) expressing Ba/F3 stable cell line
5. HER2 (A775_G776insYVMA) expressing Ba/F3 stable cell line

### Assay Procedure:

1. Seed cells at 5000 for A431 and 15,000 cells for Ba/F3 in 100 μL/well in complete media (for A431: DMEM with 10% FBS and for Ba/F3 cells: RPMI with 10% FBS) in 96-well tissue culture plate. Leave outer wells without cells for background measurements. Incubate at 37 degree Celsius in 5% CO₂ humidified incubator for 16-18 hours.
2. Add 0.025 ml of 5X concentration compound dilution or DMSO control. Final compound concentration range is 10-0.0005 μM prepared in 3-fold serial dilutions. Incubate for 72 hr at 37 degree Celsius in 5% CO₂ humidified incubator.
3. Add 0.0125 ml Alamar Blue™ reagent to each well with multi-channel pipette and tap gently on each side of the plate to mix. Incubate for 3 hours at 37 degree Celsius in 5% CO₂ humidified incubator.
4. Read plates on fluorescence reader (Tecan Spark Control, Device: Spark, Serial #: 1801006040) at 540 nm excitation, 590 nm emission wavelength.
5. Data analysis was performed using XLfit 5.5.0.5.

Table 10 shows the activity of compounds of the present disclosure in the EGFR and HER2 cellular proliferation assays.

TABLE 10
Cellular proliferation data.

| | | A431 | A767 | D770 | NPH | YVMA |
|---|---|---|---|---|---|---|
| Cmpd | | IC₅₀ | IC₅₀ | IC₅₀ | IC₅₀ | IC₅₀ |
| No. | Structure | (nM) | (nM) | (nM) | (nM) | (nM) |
| 1 | 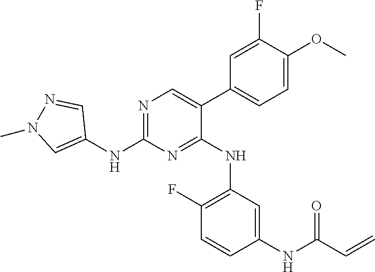 [SMILES: C=CC(=O)Nc1ccc(F)c(Nc2nc(Nc3cnn(C)c3)ncc2-c2ccc(OC)c(F)c2)c1] | 269 | 36 | 36 | 36 | 91 |
| 2 | 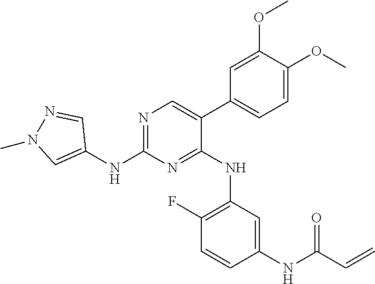 [SMILES: C=CC(=O)Nc1ccc(F)c(Nc2nc(Nc3cnn(C)c3)ncc2-c2ccc(OC)c(OC)c2)c1] | >10000 | 896 | 1050 | ND | ND |
| 3 | 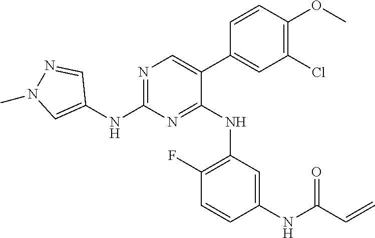 [SMILES: C=CC(=O)Nc1ccc(F)c(Nc2nc(Nc3cnn(C)c3)ncc2-c2ccc(OC)c(Cl)c2)c1] | 2317 | 91 | 90 | 103 | 432 |
| 4 | 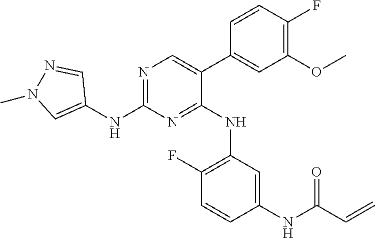 [SMILES: C=CC(=O)Nc1ccc(F)c(Nc2nc(Nc3cnn(C)c3)ncc2-c2ccc(F)c(OC)c2)c1] | >10000 | 293 | 295 | ND | 262 |
| 5 | 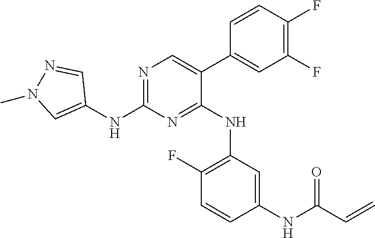 [SMILES: C=CC(=O)Nc1ccc(F)c(Nc2nc(Nc3cnn(C)c3)ncc2-c2ccc(F)c(F)c2)c1] | 200 | 39 | 30 | 79 | 208 |
| 6 | 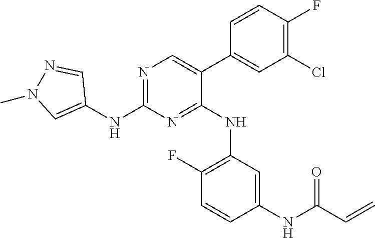 [SMILES: C=CC(=O)Nc1ccc(F)c(Nc2nc(Nc3cnn(C)c3)ncc2-c2ccc(F)c(Cl)c2)c1] | 810 | 80 | 51 | 83 | 187 |
| 7 | 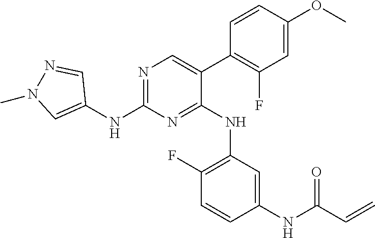 [SMILES: C=CC(=O)Nc1ccc(F)c(Nc2nc(Nc3cnn(C)c3)ncc2-c2ccc(OC)cc2F)c1] | 229 | 97 | 94 | 97 | 377 |
| 8 | 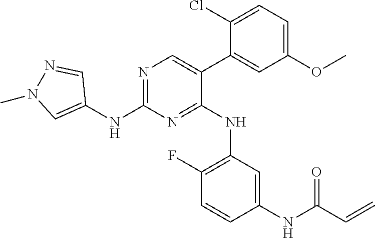 [SMILES: C=CC(=O)Nc1ccc(F)c(Nc2nc(Nc3cnn(C)c3)ncc2-c2cc(OC)ccc2Cl)c1] | >10000 | 749 | 1591 | ND | 644 |
| 9 | 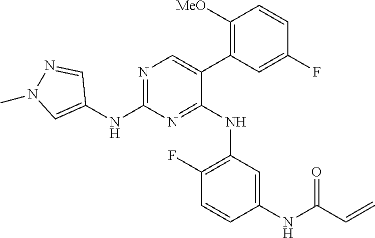 [SMILES: C=CC(=O)Nc1ccc(F)c(Nc2nc(Nc3cnn(C)c3)ncc2-c2cc(F)ccc2OC)c1] | >10000 | 721 | 930 | ND | 838 |
| 10 | 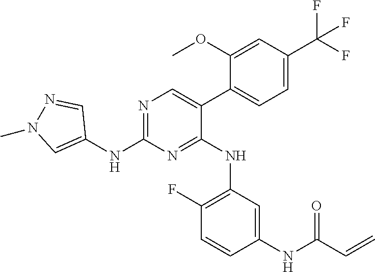 [SMILES: C=CC(=O)Nc1ccc(F)c(Nc2nc(Nc3cnn(C)c3)ncc2-c2ccc(C(F)(F)F)cc2OC)c1] | >10000 | 3232 | 3936 | ND | ND |
| 11 | 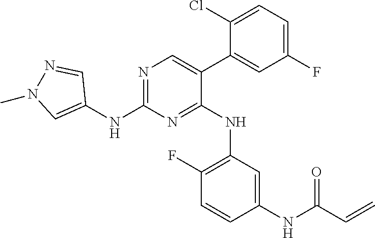 [SMILES: C=CC(=O)Nc1ccc(F)c(Nc2nc(Nc3cnn(C)c3)ncc2-c2cc(F)ccc2Cl)c1] | 1951 | 262 | 194 | 124 | 1067 |
| 12 | 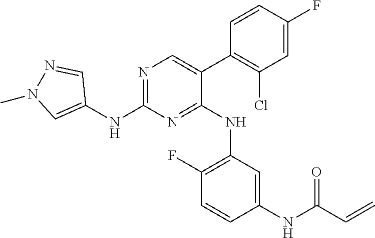 [SMILES: C=CC(=O)Nc1ccc(F)c(Nc2nc(Nc3cnn(C)c3)ncc2-c2ccc(F)cc2Cl)c1] | ND | 919 | 363 | ND | ND |
| 13 | 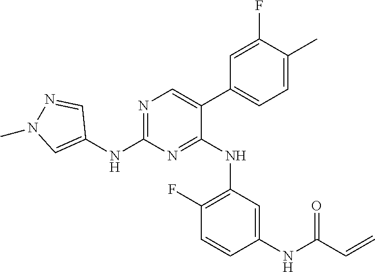 [SMILES: C=CC(=O)Nc1ccc(F)c(Nc2nc(Nc3cnn(C)c3)ncc2-c2ccc(C)c(F)c2)c1] | 147 | 42 | 28 | 79 | 114 |
| 14 | 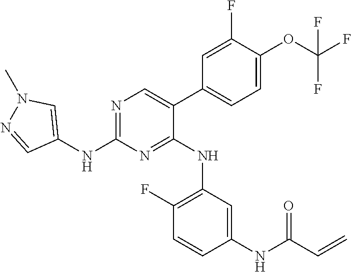 [SMILES: C=CC(=O)Nc1ccc(F)c(Nc2nc(Nc3cnn(C)c3)ncc2-c2ccc(OC(F)(F)F)c(F)c2)c1] | 140 | 26 | 33 | 59 | 107 |
| 15 | 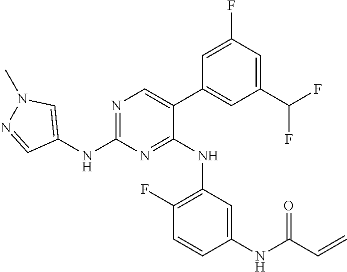 [SMILES: C=CC(=O)Nc1ccc(F)c(Nc2nc(Nc3cnn(C)c3)ncc2-c2cc(F)cc(C(F)F)c2)c1] | 84 | 45 | 36 | 102 | 124 |
| 16 | 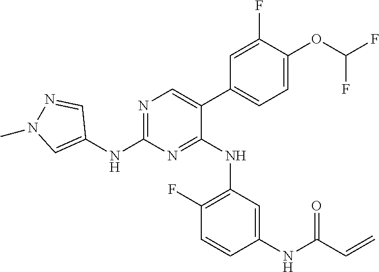 [SMILES: C=CC(=O)Nc1ccc(F)c(Nc2nc(Nc3cnn(C)c3)ncc2-c2ccc(OC(F)F)c(F)c2)c1] | 460 | 9 | 10 | 24 | 43 |
| 17 | 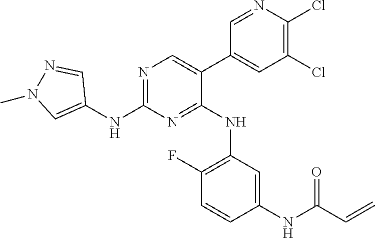 [SMILES: C=CC(=O)Nc1ccc(F)c(Nc2nc(Nc3cnn(C)c3)ncc2-c2cnc(Cl)c(Cl)c2)c1] | 75 | 32 | 35 | 96 | 42 |
| 18 | 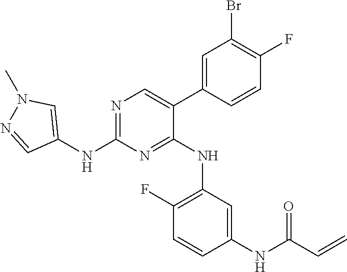 [SMILES: C=CC(=O)Nc1ccc(F)c(Nc2nc(Nc3cnn(C)c3)ncc2-c2ccc(F)c(Br)c2)c1] | 251 | 166 | 113 | 56 | 220 |
| 19 | 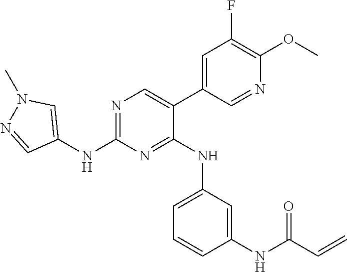 [SMILES: C=CC(=O)Nc1cccc(Nc2nc(Nc3cnn(C)c3)ncc2-c2cnc(OC)c(F)c2)c1] | 108 | 12 | 12 | 33 | 35 |
| 20 | 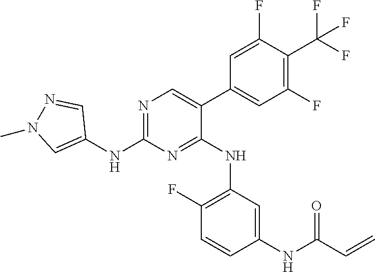 [SMILES: C=CC(=O)Nc1ccc(F)c(Nc2nc(Nc3cnn(C)c3)ncc2-c2cc(F)c(C(F)(F)F)c(F)c2)c1] | 13 | 61 | 33 | ND | 164 |
| 21 | 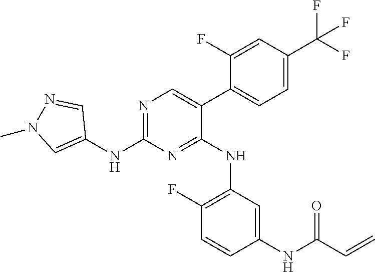 [SMILES: C=CC(=O)Nc1ccc(F)c(Nc2nc(Nc3cnn(C)c3)ncc2-c2ccc(C(F)(F)F)cc2F)c1] | >10000 | 181 | 107 | 261 | 1029 |
| 22 | 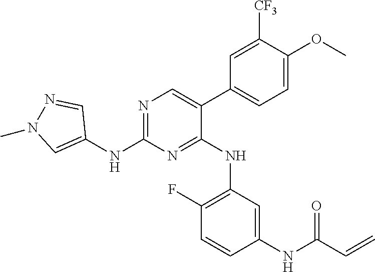 [SMILES: C=CC(=O)Nc1ccc(F)c(Nc2nc(Nc3cnn(C)c3)ncc2-c2ccc(OC)c(C(F)(F)F)c2)c1] | >10000 | 428 | 329 | ND | 890 |
| 23 | 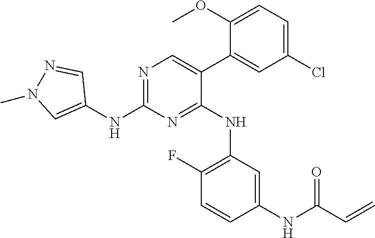 [SMILES: C=CC(=O)Nc1ccc(F)c(Nc2nc(Nc3cnn(C)c3)ncc2-c2cc(Cl)ccc2OC)c1] | 2892 | 2764 | 2731 | ND | ND |
| 24 | 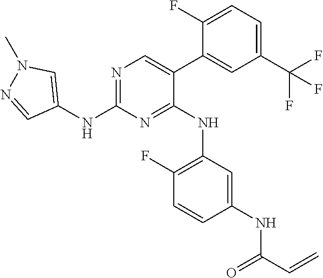 [SMILES: C=CC(=O)Nc1ccc(F)c(Nc2nc(Nc3cnn(C)c3)ncc2-c2cc(C(F)(F)F)ccc2F)c1] | 9421 | 321 | 300 | ND | 588 |
| 25 | 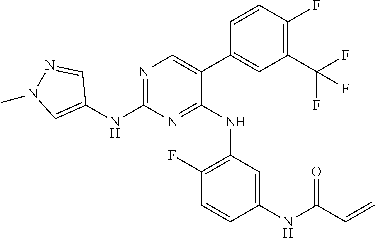 [SMILES: C=CC(=O)Nc1ccc(F)c(Nc2nc(Nc3cnn(C)c3)ncc2-c2ccc(F)c(C(F)(F)F)c2)c1] | 49 | 87 | 102 | 91 | 341 |
| 26 | 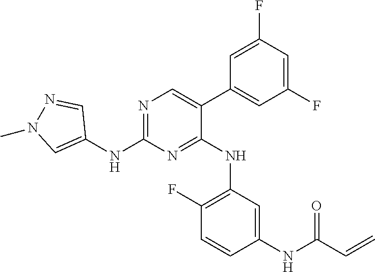 [SMILES: C=CC(=O)Nc1ccc(F)c(Nc2nc(Nc3cnn(C)c3)ncc2-c2cc(F)cc(F)c2)c1] | 443 | 35 | 27 | 31 | 112 |
| 27 | 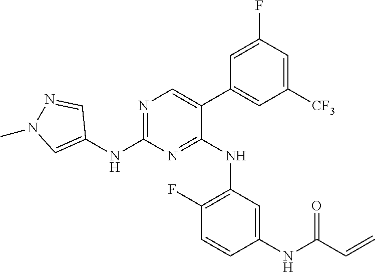 [SMILES: C=CC(=O)Nc1ccc(F)c(Nc2nc(Nc3cnn(C)c3)ncc2-c2cc(F)cc(C(F)(F)F)c2)c1] | 73 | 31 | 35 | 55 | 150 |
| 28 | 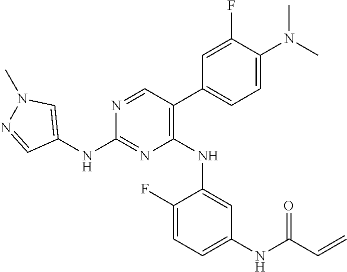 [SMILES: C=CC(=O)Nc1ccc(F)c(Nc2nc(Nc3cnn(C)c3)ncc2-c2ccc(N(C)C)c(F)c2)c1] | 193 | 27 | 29 | 47 | 271 |
| 29 | 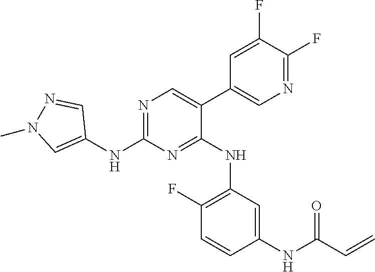 [SMILES: C=CC(=O)Nc1ccc(F)c(Nc2nc(Nc3cnn(C)c3)ncc2-c2cnc(F)c(F)c2)c1] | 20 | 7 | 8 | 33 | 21 |
| 30 | 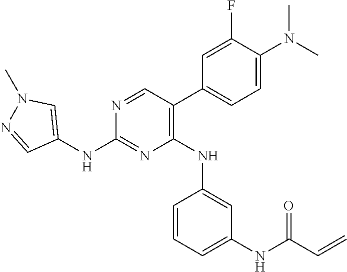 [SMILES: C=CC(=O)Nc1cccc(Nc2nc(Nc3cnn(C)c3)ncc2-c2ccc(N(C)C)c(F)c2)c1] | 299 | 33 | 35 | 40 | 91 |
| 31 | 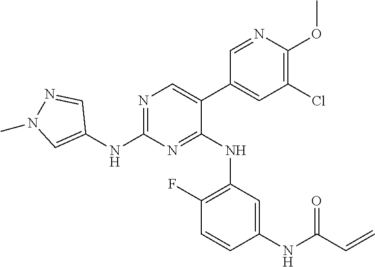 [SMILES: C=CC(=O)Nc1ccc(F)c(Nc2nc(Nc3cnn(C)c3)ncc2-c2cnc(OC)c(Cl)c2)c1] | 582 | 34 | 50 | 92 | 94 |
| 32 | 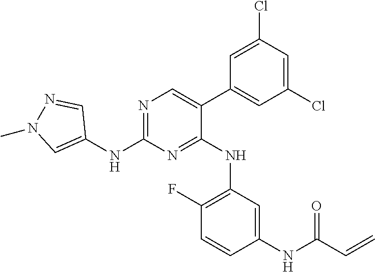 [SMILES: C=CC(=O)Nc1ccc(F)c(Nc2nc(Nc3cnn(C)c3)ncc2-c2cc(Cl)cc(Cl)c2)c1] | >10000 | 159 | 294 | 160 | 1156 |
| 33 | 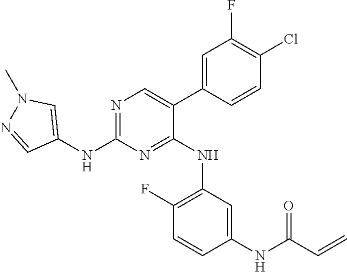 [SMILES: C=CC(=O)Nc1ccc(F)c(Nc2nc(Nc3cnn(C)c3)ncc2-c2ccc(Cl)c(F)c2)c1] | 35 | 21 | 20 | 52 | 73 |
| 34 | 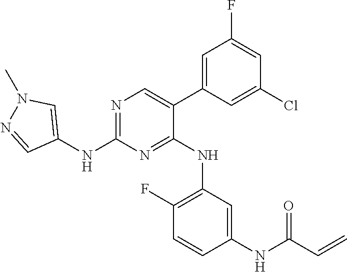 [SMILES: C=CC(=O)Nc1ccc(F)c(Nc2nc(Nc3cnn(C)c3)ncc2-c2cc(F)cc(Cl)c2)c1] | 303 | 39 | 33 | 50 | 84 |
| 35 | 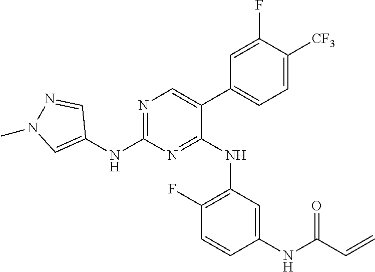 [SMILES: C=CC(=O)Nc1ccc(F)c(Nc2nc(Nc3cnn(C)c3)ncc2-c2ccc(C(F)(F)F)c(F)c2)c1] | 45 | 99 | 35 | 84 | 99 |
| 36 | 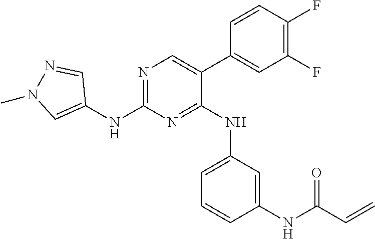 [SMILES: C=CC(=O)Nc1cccc(Nc2nc(Nc3cnn(C)c3)ncc2-c2ccc(F)c(F)c2)c1] | 767 | 91 | 71 | 140 | 212 |
| 37 | 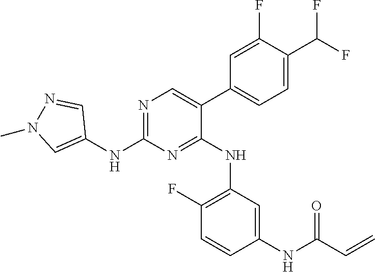 [SMILES: C=CC(=O)Nc1ccc(F)c(Nc2nc(Nc3cnn(C)c3)ncc2-c2ccc(C(F)F)c(F)c2)c1] | 51 | 34 | 30 | 51 | 93 |
| 38 | 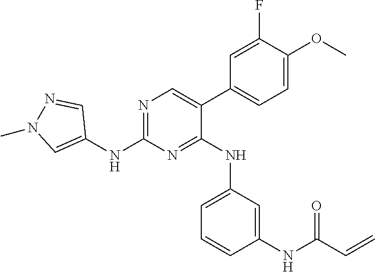 [SMILES: C=CC(=O)Nc1cccc(Nc2nc(Nc3cnn(C)c3)ncc2-c2ccc(OC)c(F)c2)c1] | 195 | 17 | 24 | 52 | 62 |
| 39 | 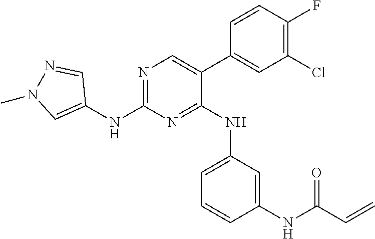 [SMILES: C=CC(=O)Nc1cccc(Nc2nc(Nc3cnn(C)c3)ncc2-c2ccc(F)c(Cl)c2)c1] | 1319 | 136 | 271 | 310 | 483 |
| 40 | 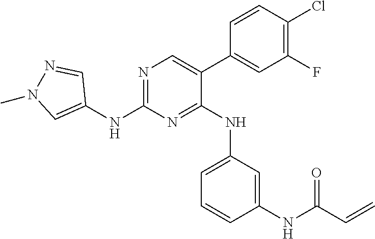 [SMILES: C=CC(=O)Nc1cccc(Nc2nc(Nc3cnn(C)c3)ncc2-c2ccc(Cl)c(F)c2)c1] | 482 | 42 | 35 | 82 | 146 |
| 41 | 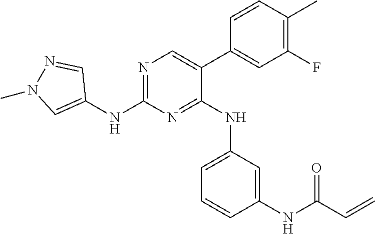 [SMILES: C=CC(=O)Nc1cccc(Nc2nc(Nc3cnn(C)c3)ncc2-c2ccc(C)c(F)c2)c1] | 196 | 83 | 37 | 105 | 129 |
| 42 | 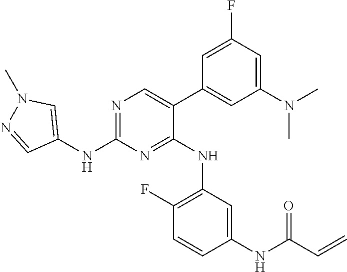 [SMILES: C=CC(=O)Nc1ccc(F)c(Nc2nc(Nc3cnn(C)c3)ncc2-c2cc(F)cc(N(C)C)c2)c1] | 4510 | 737 | 942 | ND | ND |
| 43 | 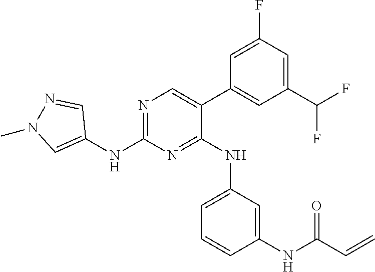 [SMILES: C=CC(=O)Nc1cccc(Nc2nc(Nc3cnn(C)c3)ncc2-c2cc(F)cc(C(F)F)c2)c1] | 707 | 41 | 35 | 24 | 84 |
| 44 | 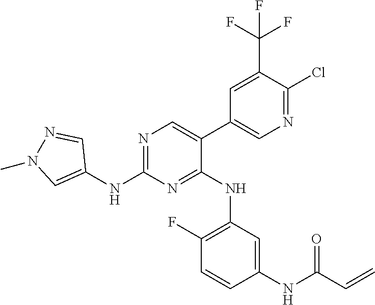 [SMILES: C=CC(=O)Nc1ccc(F)c(Nc2nc(Nc3cnn(C)c3)ncc2-c2cnc(Cl)c(C(F)(F)F)c2)c1] | 52 | 15 | 24 | 33 | 36 |
| 45 | 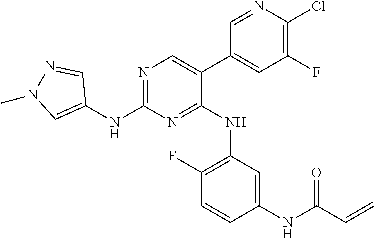 [SMILES: C=CC(=O)Nc1ccc(F)c(Nc2nc(Nc3cnn(C)c3)ncc2-c2cnc(Cl)c(F)c2)c1] | 35 | 10 | 7 | 25 | 16 |
| 46 | 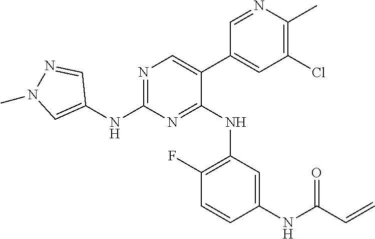 [SMILES: C=CC(=O)Nc1ccc(F)c(Nc2nc(Nc3cnn(C)c3)ncc2-c2cnc(C)c(Cl)c2)c1] | 102 | 29 | 17 | 30 | 34 |
| 47 | 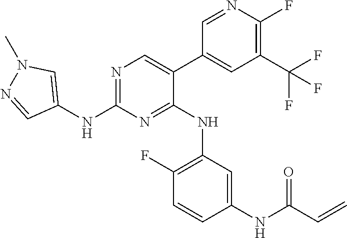 [SMILES: C=CC(=O)Nc1ccc(F)c(Nc2nc(Nc3cnn(C)c3)ncc2-c2cnc(F)c(C(F)(F)F)c2)c1] | 46 | 34 | 21 | 20 | 25 |
| 48 | 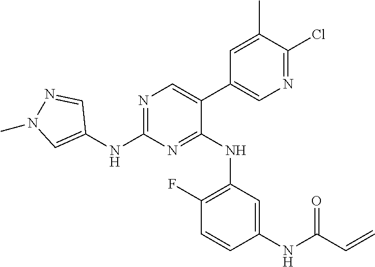 [SMILES: C=CC(=O)Nc1ccc(F)c(Nc2nc(Nc3cnn(C)c3)ncc2-c2cnc(Cl)c(C)c2)c1] | 68 | 12 | 12 | 31 | 36 |
| 49 | 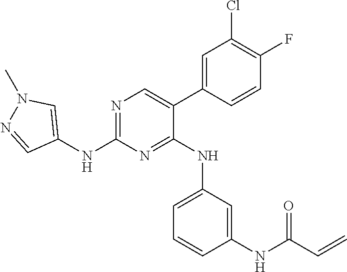 [SMILES: C=CC(=O)Nc1cccc(Nc2nc(Nc3cnn(C)c3)ncc2-c2ccc(F)c(Cl)c2)c1] | 246 | 44 | 38 | 27 | 65 |
| 50 | 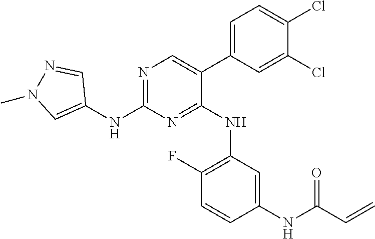 [SMILES: C=CC(=O)Nc1ccc(F)c(Nc2nc(Nc3cnn(C)c3)ncc2-c2ccc(Cl)c(Cl)c2)c1] | 70 | 26 | 32 | 61 | 86 |
| 51 | 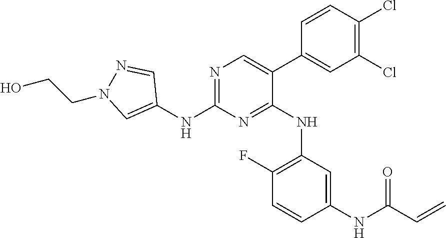 [SMILES: C=CC(=O)Nc1ccc(F)c(Nc2nc(Nc3cnn(CCO)c3)ncc2-c2ccc(Cl)c(Cl)c2)c1] | 110 | 56 | 38 | 79 | ND |
| 52 | 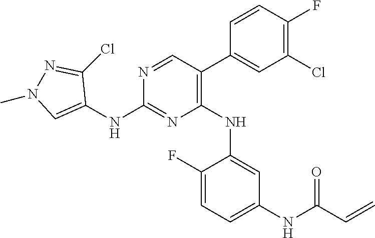 [SMILES: C=CC(=O)Nc1ccc(F)c(Nc2nc(Nc3cn(C)nc3Cl)ncc2-c2ccc(F)c(Cl)c2)c1] | 194 | 307 | 104 | ND | ND |
| 53 | 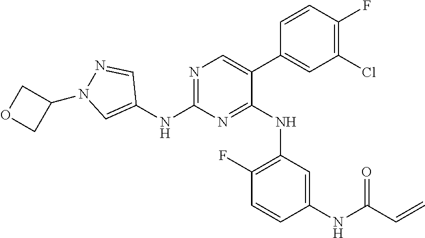 [SMILES: C=CC(=O)Nc1ccc(F)c(Nc2nc(Nc3cnn(C4COC4)c3)ncc2-c2ccc(F)c(Cl)c2)c1] | 1219 | 119 | 104 | 153 | 211 |
| 54 | 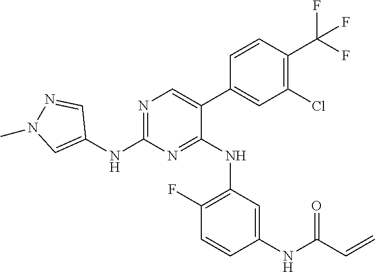 [SMILES: C=CC(=O)Nc1ccc(F)c(Nc2nc(Nc3cnn(C)c3)ncc2-c2ccc(C(F)(F)F)c(Cl)c2)c1] | ND | 184 | 85 | 186 | 260 |
| 55 | 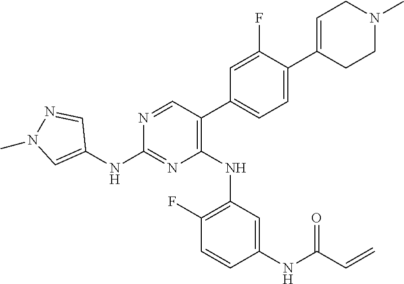 | 2229 | 800 | 2237 | 3643 | ND |
| 56 | 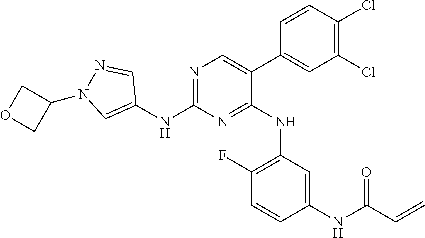 [SMILES: C=CC(=O)Nc1ccc(F)c(Nc2nc(Nc3cnn(C4COC4)c3)ncc2-c2ccc(Cl)c(Cl)c2)c1] | 587 | 95 | 104 | 84 | 118 |
| 57 | 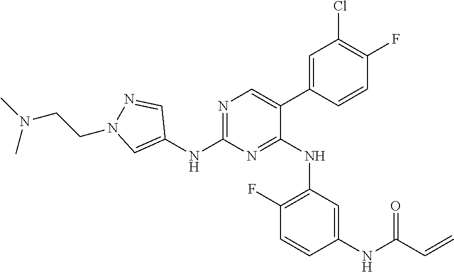 [SMILES: C=CC(=O)Nc1ccc(F)c(Nc2nc(Nc3cnn(CCN(C)C)c3)ncc2-c2ccc(F)c(Cl)c2)c1] | 699 | 207 | 111 | 62 | 112 |
| 58 | 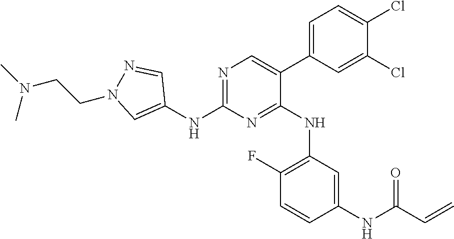 [SMILES: C=CC(=O)Nc1ccc(F)c(Nc2nc(Nc3cnn(CCN(C)C)c3)ncc2-c2ccc(Cl)c(Cl)c2)c1] | 85 | 257 | 130 | 214 | 100 |
| 59 | 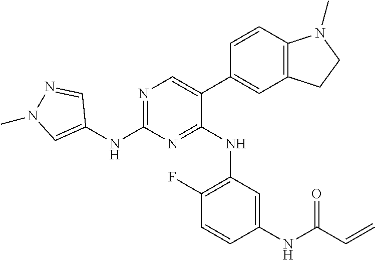 [SMILES: C=CC(=O)Nc1ccc(F)c(Nc2nc(Nc3cnn(C)c3)ncc2-c2ccc3c(c2)CCN3C)c1] | 575 | 957 | 300 | ND | ND |
| 60 | 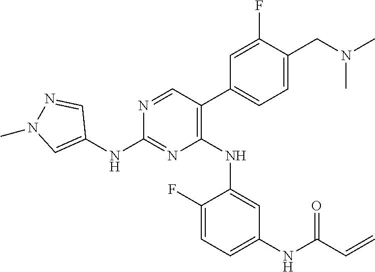 | 1185 | 150 | 170 | 462 | 952 |
| 61 | 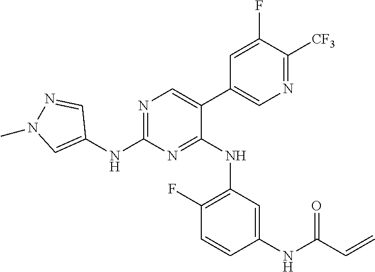 [SMILES: C=CC(=O)Nc1ccc(F)c(Nc2nc(Nc3cnn(C)c3)ncc2-c2cnc(C(F)(F)F)c(F)c2)c1] | ND | 11 | 12 | 33 | 37 |
| 62 | 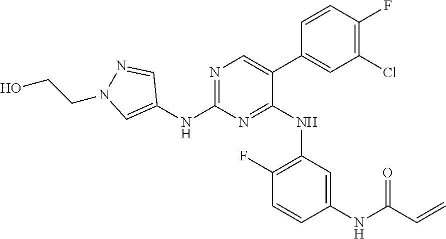 [SMILES: C=CC(=O)Nc1ccc(F)c(Nc2nc(Nc3cnn(CCO)c3)ncc2-c2ccc(F)c(Cl)c2)c1] | 1002 | 80 | 75 | 91 | 103 |
| 63 | 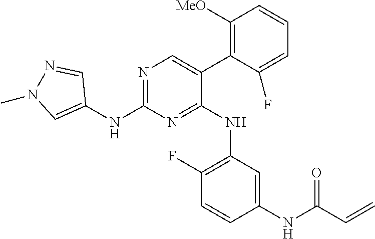 [SMILES: C=CC(=O)Nc1ccc(F)c(Nc2nc(Nc3cnn(C)c3)ncc2-c2c(F)cccc2OC)c1] | >10000 | 3089 | 3270 | ND | 884 |
| 64 | 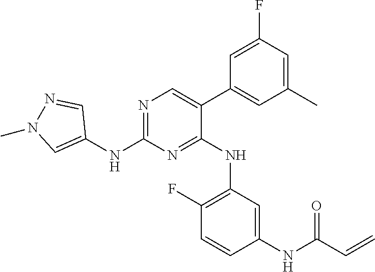 [SMILES: C=CC(=O)Nc1ccc(F)c(Nc2nc(Nc3cnn(C)c3)ncc2-c2cc(C)cc(F)c2)c1] | 554 | 104 | 61 | 95 | 159 |
| 65 | 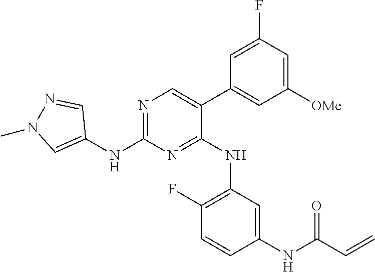 [SMILES: C=CC(=O)Nc1ccc(F)c(Nc2nc(Nc3cnn(C)c3)ncc2-c2cc(F)cc(OC)c2)c1] | 1073 | 226 | 114 | 304 | 112 |
| 66 | 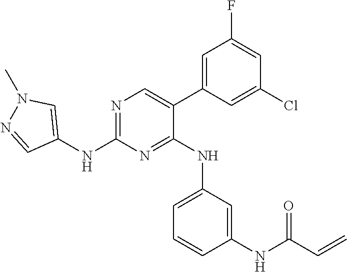 [SMILES: C=CC(=O)Nc1cccc(Nc2nc(Nc3cnn(C)c3)ncc2-c2cc(F)cc(Cl)c2)c1] | 55 | 26 | 15 | 14 | 47 |
| 67 | 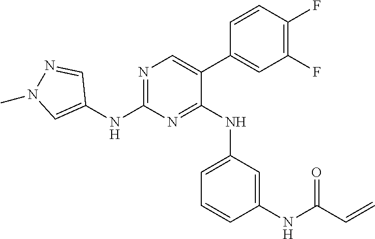 [SMILES: C=CC(=O)Nc1cccc(Nc2nc(Nc3cnn(C)c3)ncc2-c2ccc(F)c(F)c2)c1] | 213 | 41 | 36 | 100 | 60 |
| 68 | 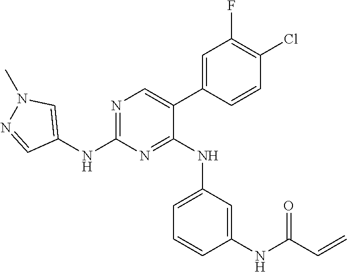 [SMILES: C=CC(=O)Nc1cccc(Nc2nc(Nc3cnn(C)c3)ncc2-c2ccc(Cl)c(F)c2)c1] | 60 | 23 | 14 | 21 | 35 |
| 69 | 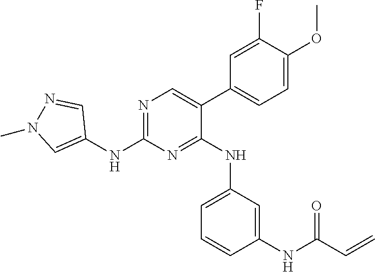 [SMILES: C=CC(=O)Nc1cccc(Nc2nc(Nc3cnn(C)c3)ncc2-c2ccc(OC)c(F)c2)c1] | 73 | 14 | 12 | 21 | 27 |
| 70 | 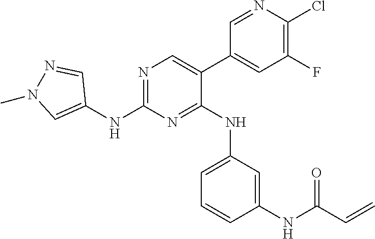 [SMILES: C=CC(=O)Nc1cccc(Nc2nc(Nc3cnn(C)c3)ncc2-c2cnc(Cl)c(F)c2)c1] | 608 | 34 | 37 | 101 | 57 |
| 71 | 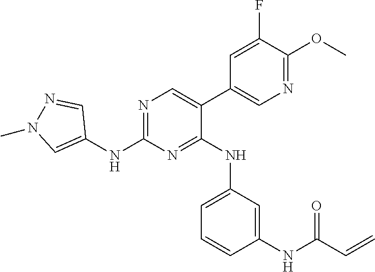 [SMILES: C=CC(=O)Nc1cccc(Nc2nc(Nc3cnn(C)c3)ncc2-c2cnc(OC)c(F)c2)c1] | 150 | 30 | 25 | 38 | 62 |
| 72 | 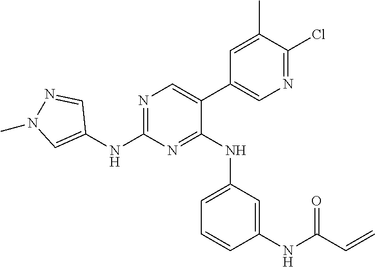 [SMILES: C=CC(=O)Nc1cccc(Nc2nc(Nc3cnn(C)c3)ncc2-c2cnc(Cl)c(C)c2)c1] | 1675 | 96 | 84 | 61 | 227 |
| 73 | 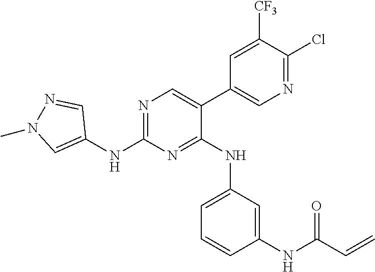 [SMILES: C=CC(=O)Nc1cccc(Nc2nc(Nc3cnn(C)c3)ncc2-c2cnc(Cl)c(C(F)(F)F)c2)c1] | 985 | 95 | 106 | 153 | 104 |
| 74 | 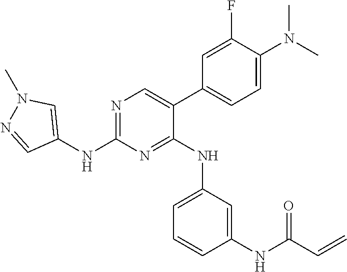 [SMILES: C=CC(=O)Nc1cccc(Nc2nc(Nc3cnn(C)c3)ncc2-c2ccc(N(C)C)c(F)c2)c1] | 108 | 91 | 50 | 44 | 197 |
| 75 | 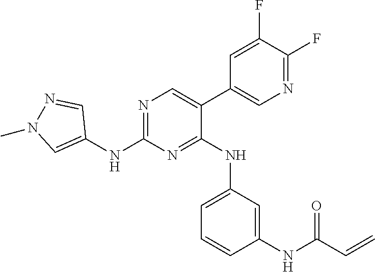 [SMILES: C=CC(=O)Nc1cccc(Nc2nc(Nc3cnn(C)c3)ncc2-c2cnc(F)c(F)c2)c1] | 586 | 25 | 34 | 23 | 65 |
| 76 | 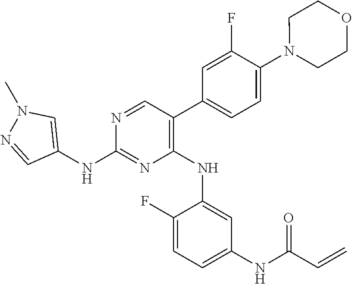 [SMILES: C=CC(=O)Nc1ccc(F)c(Nc2nc(Nc3cnn(C)c3)ncc2-c2ccc(N3CCOCC3)c(F)c2)c1] | 1265 | 72 | 220 | 236 | 240 |
| 77 | 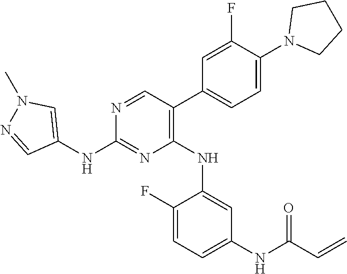 [SMILES: C=CC(=O)Nc1ccc(F)c(Nc2nc(Nc3cnn(C)c3)ncc2-c2ccc(N3CCCC3)c(F)c2)c1] | 297 | 113 | 340 | 353 | 584 |
| 78 | 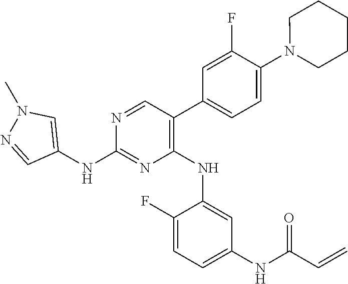 [SMILES: C=CC(=O)Nc1ccc(F)c(Nc2nc(Nc3cnn(C)c3)ncc2-c2ccc(N3CCCCC3)c(F)c2)c1] | 102 | 59 | 111 | 144 | 286 |
| 79 | 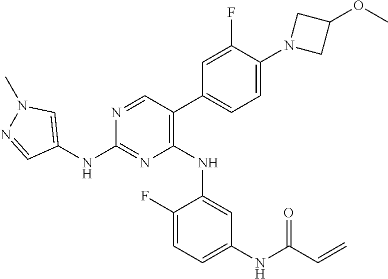 [SMILES: C=CC(=O)Nc1ccc(F)c(Nc2nc(Nc3cnn(C)c3)ncc2-c2ccc(N3CC(OC)C3)c(F)c2)c1] | 105 | 248 | 312 | ND | ND |
| 80 | 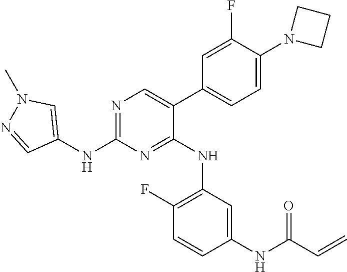 [SMILES: C=CC(=O)Nc1ccc(F)c(Nc2nc(Nc3cnn(C)c3)ncc2-c2ccc(N3CCC3)c(F)c2)c1] | 54 | 43 | 95 | ND | 171 |
| 81 | 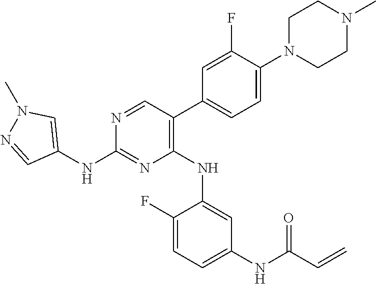 [SMILES: C=CC(=O)Nc1ccc(F)c(Nc2nc(Nc3cnn(C)c3)ncc2-c2ccc(N3CCN(C)CC3)c(F)c2)c1] | 3014 | 343 | 906 | ND | ND |
| 82 | 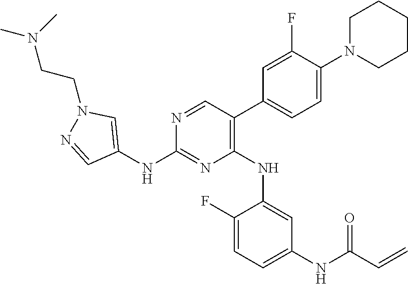 [SMILES: C=CC(=O)Nc1ccc(F)c(Nc2nc(Nc3cnn(CCN(C)C)c3)ncc2-c2ccc(N3CCCCC3)c(F)c2)c1] | 397 | 239 | 313 | ND | 149 |
| 83 | 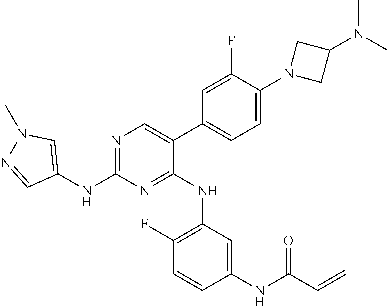 [SMILES: C=CC(=O)Nc1ccc(F)c(Nc2nc(Nc3cnn(C)c3)ncc2-c2ccc(N3CC(N(C)C)C3)c(F)c2)c1] | 1064 | 376 | 1007 | ND | ND |
| 84 | 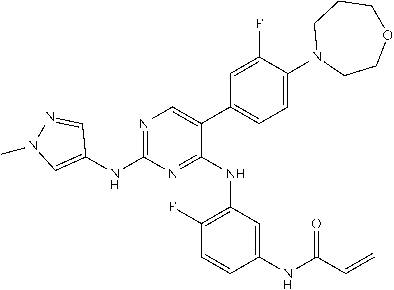 [SMILES: C=CC(=O)Nc1ccc(F)c(Nc2nc(Nc3cnn(C)c3)ncc2-c2ccc(N3CCCOCC3)c(F)c2)c1] | 522 | 101 | 122 | 92 | ND |
| 85 | 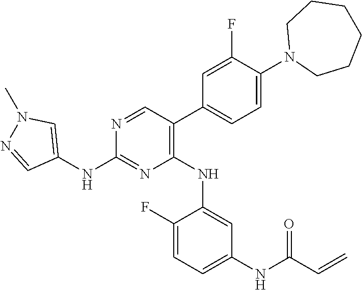 [SMILES: C=CC(=O)Nc1ccc(F)c(Nc2nc(Nc3cnn(C)c3)ncc2-c2ccc(N3CCCCCC3)c(F)c2)c1] | 2822 | 268 | 314 | ND | 884 |
| 86 | 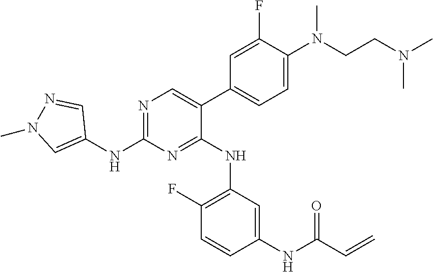 | 3575 | 2244 | 2938 | ND | ND |
| 87 | 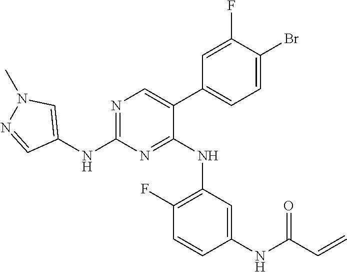 [SMILES: C=CC(=O)Nc1ccc(F)c(Nc2nc(Nc3cnn(C)c3)ncc2-c2ccc(Br)c(F)c2)c1] | 18 | 33 | 12 | 116 | 83 |
| 88 | 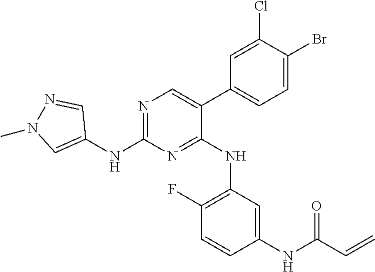 [SMILES: C=CC(=O)Nc1ccc(F)c(Nc2nc(Nc3cnn(C)c3)ncc2-c2ccc(Br)c(Cl)c2)c1] | 280 | 87 | 66 | 57 | 118 |
| 89 | 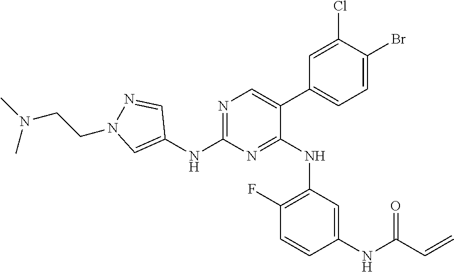 [SMILES: C=CC(=O)Nc1ccc(F)c(Nc2nc(Nc3cnn(CCN(C)C)c3)ncc2-c2ccc(Br)c(Cl)c2)c1] | 343 | 108 | 102 | 56 | 123 |
| 90 | 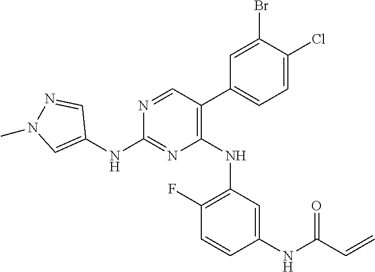 [SMILES: C=CC(=O)Nc1ccc(F)c(Nc2nc(Nc3cnn(C)c3)ncc2-c2ccc(Cl)c(Br)c2)c1] | 185 | 122 | 100 | 156 | 164 |
| 91 | 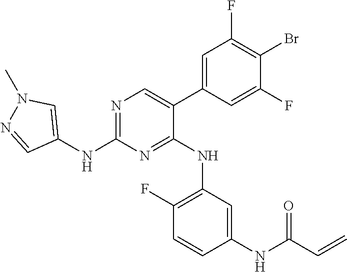 [SMILES: C=CC(=O)Nc1ccc(F)c(Nc2nc(Nc3cnn(C)c3)ncc2-c2cc(F)c(Br)c(F)c2)c1] | 11 | 31 | 15 | ND | 59 |
| 92 | 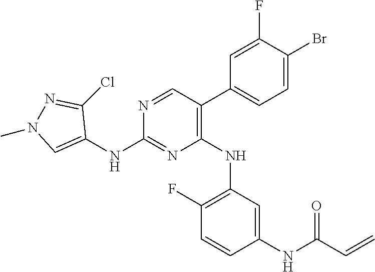 [SMILES: C=CC(=O)Nc1ccc(F)c(Nc2nc(Nc3cn(C)nc3Cl)ncc2-c2ccc(Br)c(F)c2)c1] | 45 | 102 | 74 | 54 | 113 |
| 93 | 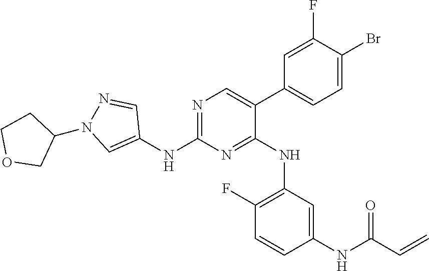 [SMILES: C=CC(=O)Nc1ccc(F)c(Nc2nc(Nc3cnn(C4CCOC4)c3)ncc2-c2ccc(Br)c(F)c2)c1] | 33 | 46 | 35 | ND | 173 |
| 94 | 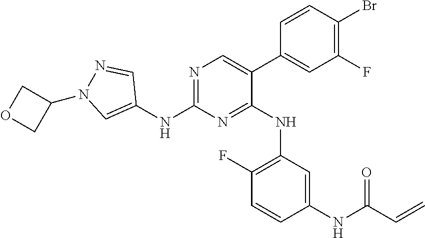 [SMILES: C=CC(=O)Nc1ccc(F)c(Nc2nc(Nc3cnn(C4COC4)c3)ncc2-c2ccc(Br)c(F)c2)c1] | 31 | 40 | 33 | 35 | 144 |
| 95 | 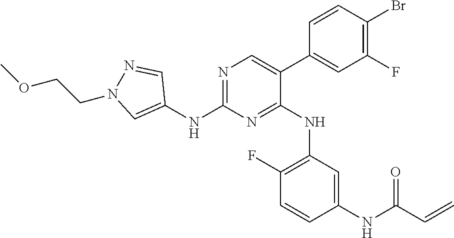 [SMILES: C=CC(=O)Nc1ccc(F)c(Nc2nc(Nc3cnn(CCOC)c3)ncc2-c2ccc(Br)c(F)c2)c1] | 4770 | 43 | 29 | ND | 197 |
| 96 | 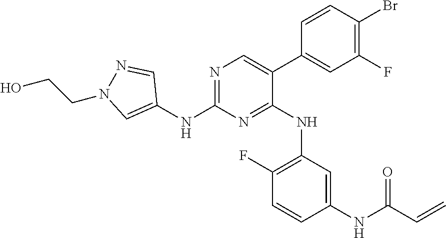 [SMILES: C=CC(=O)Nc1ccc(F)c(Nc2nc(Nc3cnn(CCO)c3)ncc2-c2ccc(Br)c(F)c2)c1] | 37 | 28 | 25 | 96 | 56 |
| 97 | 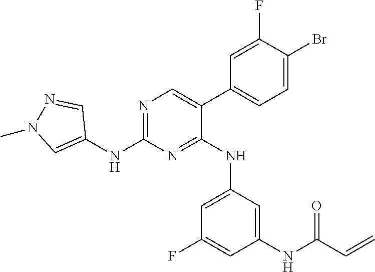 [SMILES: C=CC(=O)Nc1cc(F)cc(Nc2nc(Nc3cnn(C)c3)ncc2-c2ccc(Br)c(F)c2)c1] | 12 | 24 | 13 | ND | 62 |
| 98 | 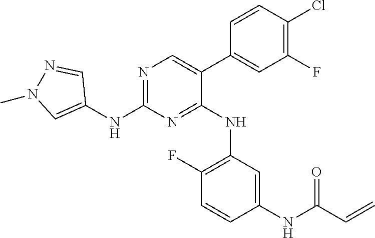 [SMILES: C=CC(=O)Nc1ccc(F)c(Nc2nc(Nc3cnn(C)c3)ncc2-c2ccc(Cl)c(F)c2)c1] | 990 | 104 | 36 | 153 | 252 |
| 99 | 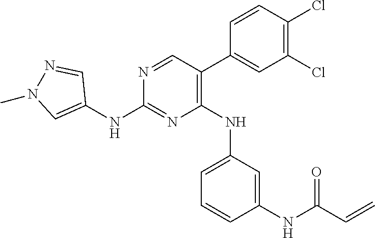 [SMILES: C=CC(=O)Nc1cccc(Nc2nc(Nc3cnn(C)c3)ncc2-c2ccc(Cl)c(Cl)c2)c1] | 821 | 294 | 168 | ND | ND |
| 100 | 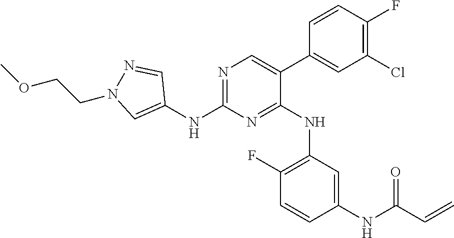 [SMILES: C=CC(=O)Nc1ccc(F)c(Nc2nc(Nc3cnn(CCOC)c3)ncc2-c2ccc(F)c(Cl)c2)c1] | 1198 | 301 | 274 | ND | 860 |
| 101 | 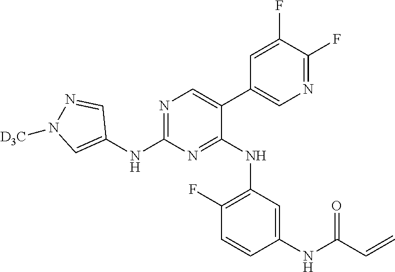 [SMILES: [2H]C([2H])([2H])n1cc(Nc2ncc(-c3cnc(F)c(F)c3)c(Nc3cc(NC(=O)C=C)ccc3F)n2)cn1] | ND | 12 | 15 | ND | ND |
| 102 | 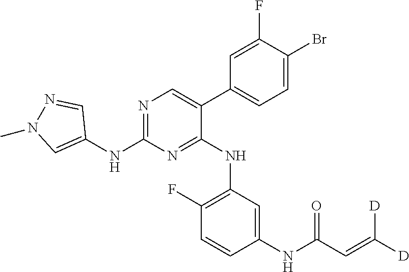 [SMILES: [2H]C([2H])=CC(=O)Nc1ccc(F)c(Nc2nc(Nc3cnn(C)c3)ncc2-c2ccc(Br)c(F)c2)c1] | 69 | 30 | 35 | ND | 117 |
| 103 | 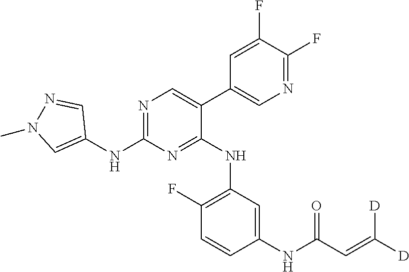 [SMILES: [2H]C([2H])=CC(=O)Nc1ccc(F)c(Nc2nc(Nc3cnn(C)c3)ncc2-c2cnc(F)c(F)c2)c1] | 20 | 14 | 25 | ND | ND |
| 104 | 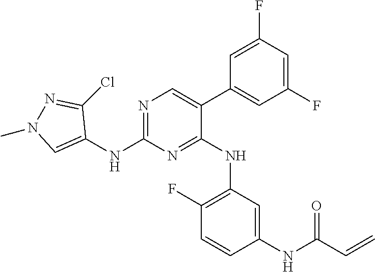 [SMILES: C=CC(=O)Nc1ccc(F)c(Nc2nc(Nc3cn(C)nc3Cl)ncc2-c2cc(F)cc(F)c2)c1] | 17 | 44 | 34 | ND | 64 |
| 105 | 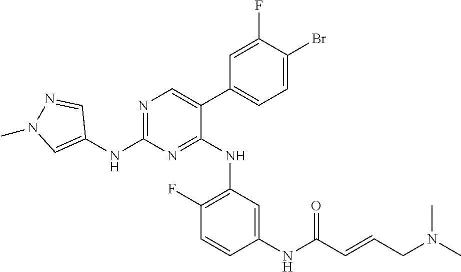 | 85 | 663 | 129 | ND | 125 |
| 106 | 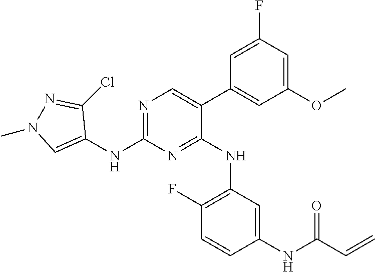 [SMILES: C=CC(=O)Nc1ccc(F)c(Nc2nc(Nc3cn(C)nc3Cl)ncc2-c2cc(F)cc(OC)c2)c1] | 125 | 35 | 285 | ND | 568 |
| 107 | 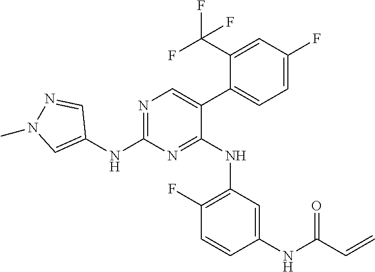 [SMILES: C=CC(=O)Nc1ccc(F)c(Nc2nc(Nc3cnn(C)c3)ncc2-c2ccc(F)cc2C(F)(F)F)c1] | 3129 | ND | ND | ND | 7345 |
| 108 | 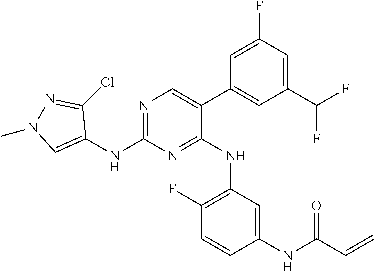 [SMILES: C=CC(=O)Nc1ccc(F)c(Nc2nc(Nc3cn(C)nc3Cl)ncc2-c2cc(F)cc(C(F)F)c2)c1] | 23 | 96 | 39 | ND | 243 |
| 109 | 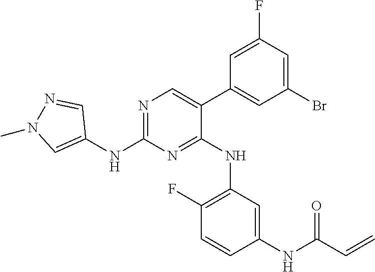 [SMILES: C=CC(=O)Nc1ccc(F)c(Nc2nc(Nc3cnn(C)c3)ncc2-c2cc(F)cc(Br)c2)c1] | 534 | 97 | 53 | ND | 192 |
| 110 | 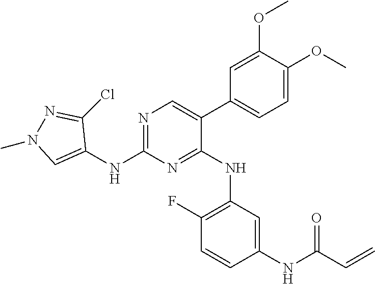 [SMILES: C=CC(=O)Nc1ccc(F)c(Nc2nc(Nc3cn(C)nc3Cl)ncc2-c2ccc(OC)c(OC)c2)c1] | 819 | ND | ND | ND | 5779 |
| 111 | 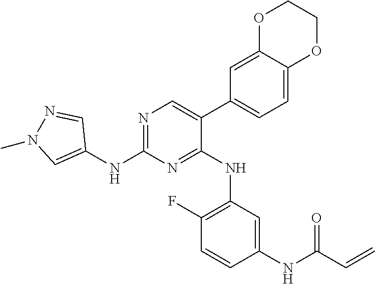 [SMILES: C=CC(=O)Nc1ccc(F)c(Nc2nc(Nc3cnn(C)c3)ncc2-c2ccc3c(c2)OCCO3)c1] | 164 | ND | ND | ND | 1077 |
| 112 | 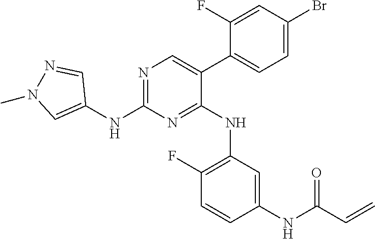 [SMILES: C=CC(=O)Nc1ccc(F)c(Nc2nc(Nc3cnn(C)c3)ncc2-c2ccc(Br)cc2F)c1] | 497 | ND | ND | ND | 923 |
| 113 | 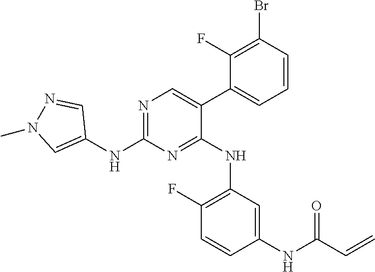 [SMILES: C=CC(=O)Nc1ccc(F)c(Nc2nc(Nc3cnn(C)c3)ncc2-c2cccc(Br)c2F)c1] | 73 | 92 | 70 | ND | 463 |
| 114 | 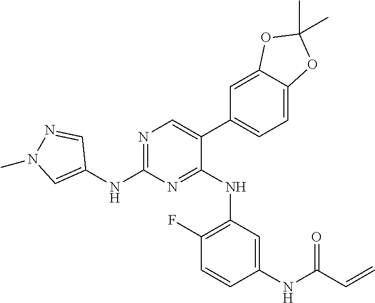 [SMILES: C=CC(=O)Nc1ccc(F)c(Nc2nc(Nc3cnn(C)c3)ncc2-c2ccc3c(c2)OC(C)(C)O3)c1] | 77 | ND | ND | ND | 4568 |
| 115 | 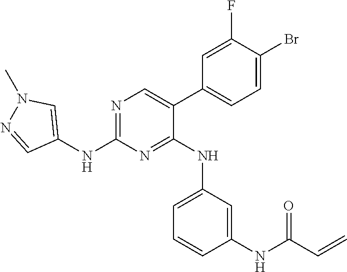 [SMILES: C=CC(=O)Nc1cccc(Nc2nc(Nc3cnn(C)c3)ncc2-c2ccc(Br)c(F)c2)c1] | 11 | 36 | 32 | ND | 86 |
| 116 | 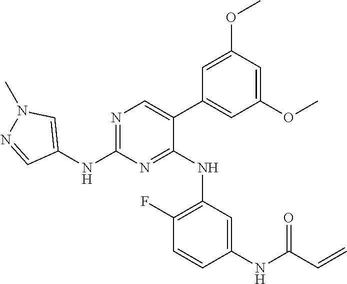 [SMILES: C=CC(=O)Nc1ccc(F)c(Nc2nc(Nc3cnn(C)c3)ncc2-c2cc(OC)cc(OC)c2)c1] | 3522 | ND | ND | ND | >10000 |
| 117 | 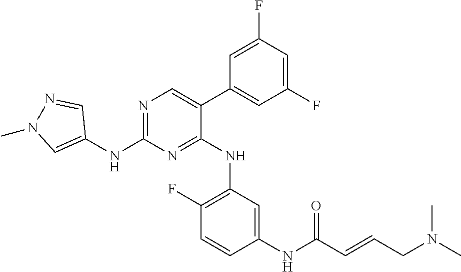 | 668 | 328 | 135 | ND | 141 |
| 118 | 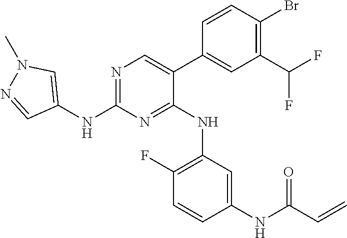 [SMILES: C=CC(=O)Nc1ccc(F)c(Nc2nc(Nc3cnn(C)c3)ncc2-c2ccc(Br)c(C(F)F)c2)c1] | 215 | ND | ND | ND | 352 |
| 119 | 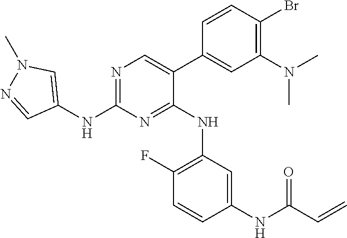 [SMILES: C=CC(=O)Nc1ccc(F)c(Nc2nc(Nc3cnn(C)c3)ncc2-c2ccc(Br)c(N(C)C)c2)c1] | 1159 | ND | ND | ND | 5198 |
| 120 | 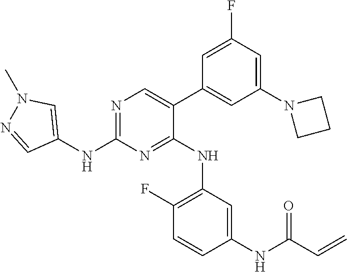 [SMILES: C=CC(=O)Nc1ccc(F)c(Nc2nc(Nc3cnn(C)c3)ncc2-c2cc(F)cc(N3CCC3)c2)c1] | 9276 | ND | ND | ND | >10000 |
| 121 | 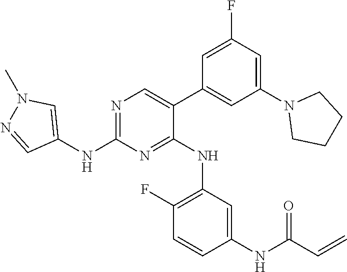 [SMILES: C=CC(=O)Nc1ccc(F)c(Nc2nc(Nc3cnn(C)c3)ncc2-c2cc(F)cc(N3CCCC3)c2)c1] | >10000 | ND | ND | ND | >10000 |
| 122 | 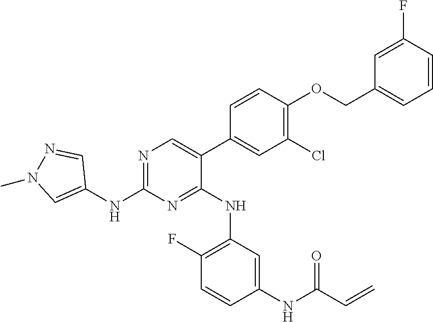 [SMILES: C=CC(=O)Nc1ccc(F)c(Nc2nc(Nc3cnn(C)c3)ncc2-c2ccc(OCc3cccc(F)c3)c(Cl)c2)c1] | 290 | ND | ND | ND | 669 |
| 123 | 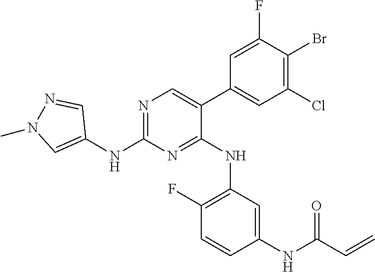 [SMILES: C=CC(=O)Nc1ccc(F)c(Nc2nc(Nc3cnn(C)c3)ncc2-c2cc(F)c(Br)c(Cl)c2)c1] | 71 | 50 | 40 | ND | 66 |
| 124 | 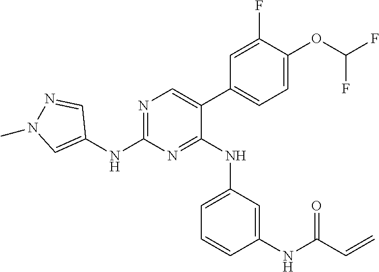 [SMILES: C=CC(=O)Nc1cccc(Nc2nc(Nc3cnn(C)c3)ncc2-c2ccc(OC(F)F)c(F)c2)c1] | 6 | 21 | 11 | ND | 49 |
| 126 | 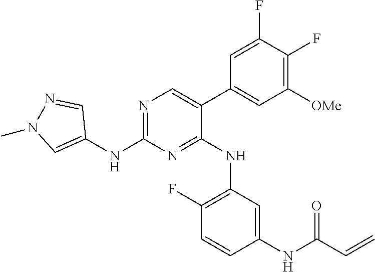 [SMILES: C=CC(=O)Nc1ccc(F)c(Nc2nc(Nc3cnn(C)c3)ncc2-c2cc(F)c(F)c(OC)c2)c1] | 43 | 100 | 40 | ND | 153 |
| 127 | 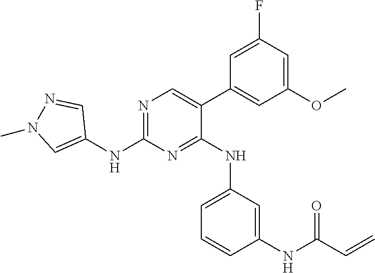 [SMILES: C=CC(=O)Nc1cccc(Nc2nc(Nc3cnn(C)c3)ncc2-c2cc(F)cc(OC)c2)c1] | 123 | 312 | 89 | ND | 167 |
| 128 | 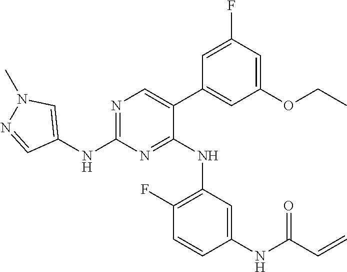 [SMILES: C=CC(=O)Nc1ccc(F)c(Nc2nc(Nc3cnn(C)c3)ncc2-c2cc(F)cc(OCC)c2)c1] | ND | ND | ND | ND | ND |
| 129 | 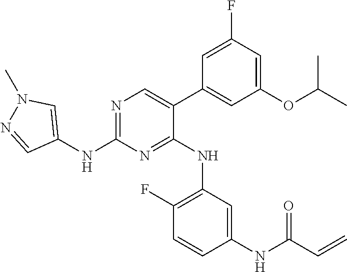 [SMILES: C=CC(=O)Nc1ccc(F)c(Nc2nc(Nc3cnn(C)c3)ncc2-c2cc(F)cc(OC(C)C)c2)c1] | >10000 | ND | ND | ND | >10000 |
| 130 | 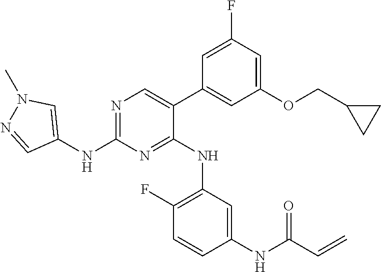 [SMILES: C=CC(=O)Nc1ccc(F)c(Nc2nc(Nc3cnn(C)c3)ncc2-c2cc(F)cc(OCC3CC3)c2)c1] | >10000 | ND | ND | ND | >10000 |
| 131 | 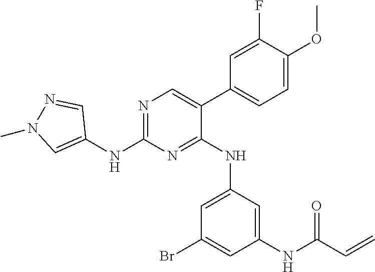 [SMILES: C=CC(=O)Nc1cc(Br)cc(Nc2nc(Nc3cnn(C)c3)ncc2-c2ccc(OC)c(F)c2)c1] | ND | ND | ND | ND | ND |
| 132 | 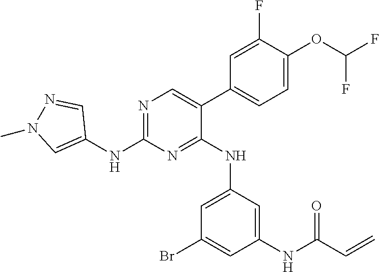 [SMILES: C=CC(=O)Nc1cc(Br)cc(Nc2nc(Nc3cnn(C)c3)ncc2-c2ccc(OC(F)F)c(F)c2)c1] | 51 | 40 | 34 | ND | 345 |
| 133 | 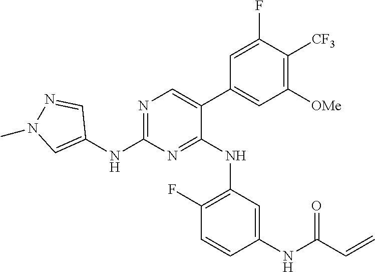 [SMILES: C=CC(=O)Nc1ccc(F)c(Nc2nc(Nc3cnn(C)c3)ncc2-c2cc(F)c(C(F)(F)F)c(OC)c2)c1] | 394 | 322 | 251 | ND | 1272 |
| 134 | 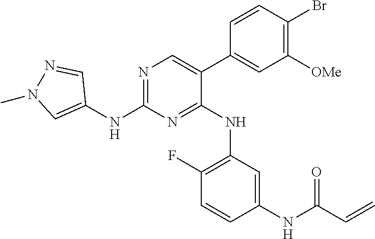 [SMILES: C=CC(=O)Nc1ccc(F)c(Nc2nc(Nc3cnn(C)c3)ncc2-c2ccc(Br)c(OC)c2)c1] | 238 | ND | ND | ND | 453 |
| 135 | 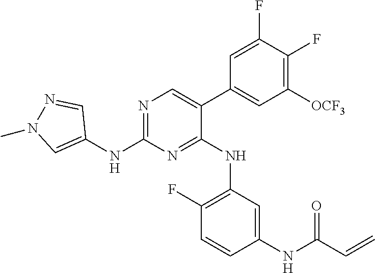 [SMILES: C=CC(=O)Nc1ccc(F)c(Nc2nc(Nc3cnn(C)c3)ncc2-c2cc(F)c(F)c(OC(F)(F)F)c2)c1] | ND | ND | ND | ND | ND |
| 136 | 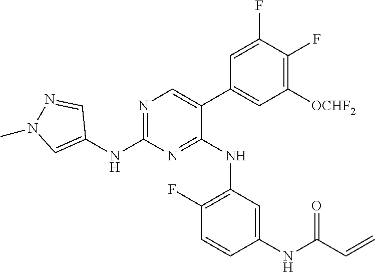 [SMILES: C=CC(=O)Nc1ccc(F)c(Nc2nc(Nc3cnn(C)c3)ncc2-c2cc(F)c(F)c(OC(F)F)c2)c1] | ND | ND | ND | ND | ND |
| 137 | 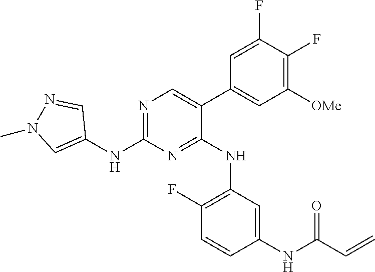 [SMILES: C=CC(=O)Nc1ccc(F)c(Nc2nc(Nc3cnn(C)c3)ncc2-c2cc(F)c(F)c(OC)c2)c1] | ND | ND | ND | ND | ND |
| 138 | 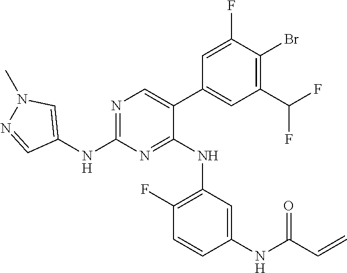 [SMILES: C=CC(=O)Nc1ccc(F)c(Nc2nc(Nc3cnn(C)c3)ncc2-c2cc(F)c(Br)c(C(F)F)c2)c1] | ND | ND | ND | ND | ND |
| 139 | 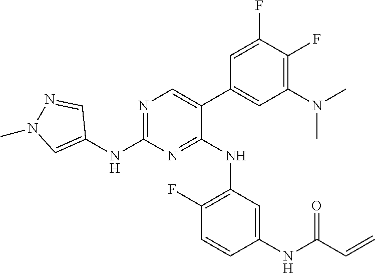 [SMILES: C=CC(=O)Nc1ccc(F)c(Nc2nc(Nc3cnn(C)c3)ncc2-c2cc(F)c(F)c(N(C)C)c2)c1] | ND | ND | ND | ND | ND |
| 140 | 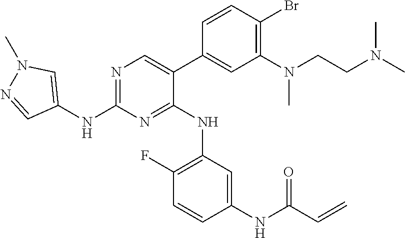 [SMILES: C=CC(=O)Nc1ccc(F)c(Nc2nc(Nc3cnn(C)c3)ncc2-c2ccc(Br)c(N(C)CCN(C)C)c2)c1] | ND | ND | ND | ND | ND |
| 141 | 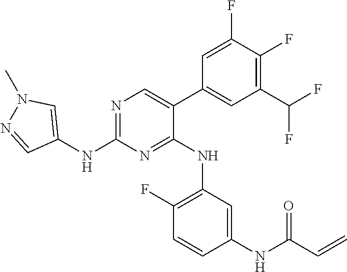 [SMILES: C=CC(=O)Nc1ccc(F)c(Nc2nc(Nc3cnn(C)c3)ncc2-c2cc(F)c(F)c(C(F)F)c2)c1] | 13 | 25 | 16 | ND | 167 |
| 142 | 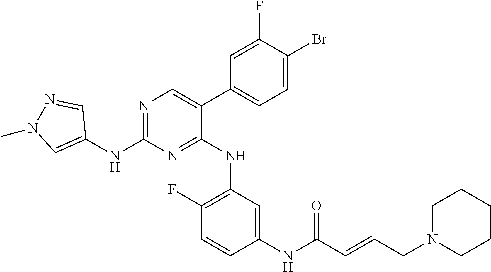 [SMILES: Cn1cc(Nc2ncc(-c3ccc(Br)c(F)c3)c(Nc3cc(NC(=O)/C=C/CN4CCCCC4)ccc3F)n2)cn1] | 301 | ND | ND | ND | 738 |
| 143 | 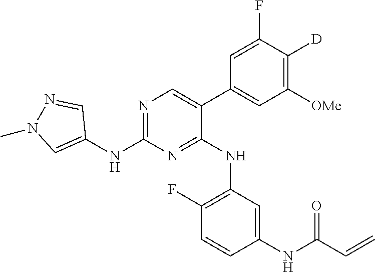 [SMILES: [2H]c1c(F)cc(-c2cnc(Nc3cnn(C)c3)nc2Nc2cc(NC(=O)C=C)ccc2F)cc1OC] | ND | ND | ND | ND | ND |
| 144 | 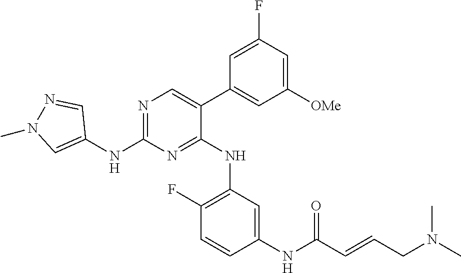 | 4881 | ND | ND | ND | 970 |
| 145 | 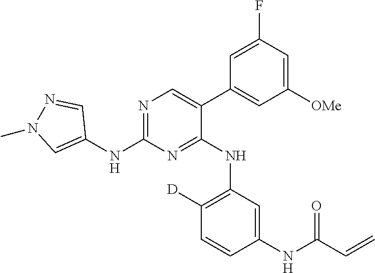 [SMILES: [2H]c1ccc(NC(=O)C=C)cc1Nc1nc(Nc2cnn(C)c2)ncc1-c1cc(F)cc(OC)c1] | 388 | ND | ND | ND | 264 |
| 146 | 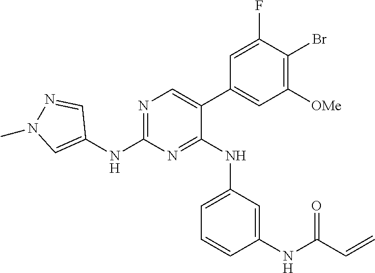 [SMILES: C=CC(=O)Nc1cccc(Nc2nc(Nc3cnn(C)c3)ncc2-c2cc(F)c(Br)c(OC)c2)c1] | ND | ND | ND | ND | ND |
| 147 | 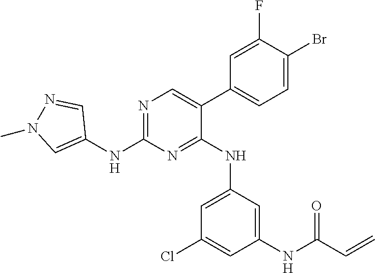 [SMILES: C=CC(=O)Nc1cc(Cl)cc(Nc2nc(Nc3cnn(C)c3)ncc2-c2ccc(Br)c(F)c2)c1] | ND | ND | ND | ND | ND |
| 148 | 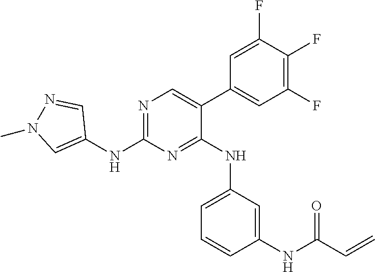 [SMILES: C=CC(=O)Nc1cccc(Nc2nc(Nc3cnn(C)c3)ncc2-c2cc(F)c(F)c(F)c2)c1] | 29 | 33 | 10 | ND | 45 |
| 149 | 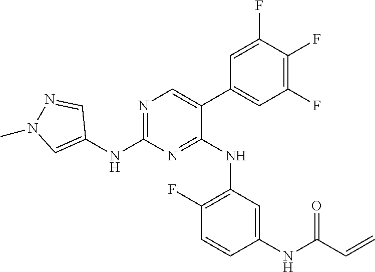 [SMILES: C=CC(=O)Nc1ccc(F)c(Nc2nc(Nc3cnn(C)c3)ncc2-c2cc(F)c(F)c(F)c2)c1] | 11 | 34 | 11 | ND | 92 |
| 150 | 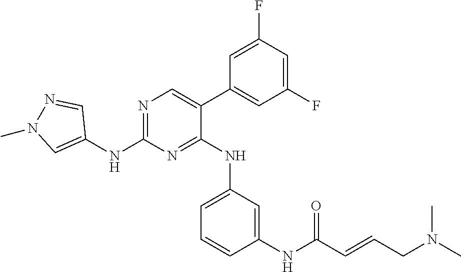 | 214 | 110 | 126 | ND | 237 |
| 151 | 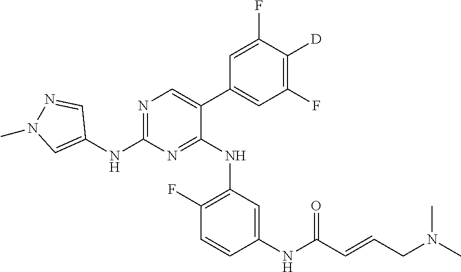 | ND | ND | ND | ND | ND |
| 152 | 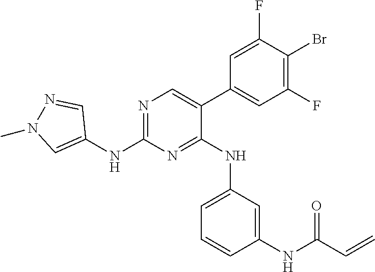 [SMILES: C=CC(=O)Nc1cccc(Nc2nc(Nc3cnn(C)c3)ncc2-c2cc(F)c(Br)c(F)c2)c1] | 8 | 34 | 11 | ND | 40 |
| 153 | 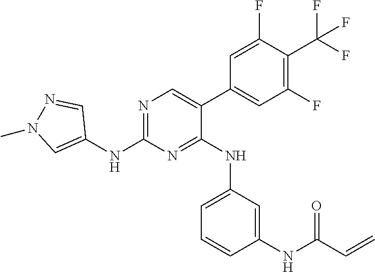 [SMILES: C=CC(=O)Nc1cccc(Nc2nc(Nc3cnn(C)c3)ncc2-c2cc(F)c(C(F)(F)F)c(F)c2)c1] | 1084 | 34 | 13 | ND | 98 |
| 154 | 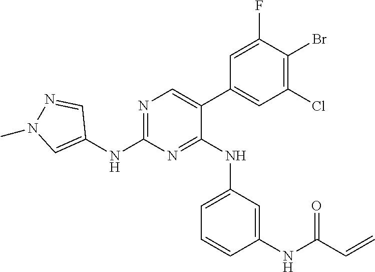 [SMILES: C=CC(=O)Nc1cccc(Nc2nc(Nc3cnn(C)c3)ncc2-c2cc(F)c(Br)c(Cl)c2)c1] | 1761 | 81 | 34 | ND | 154 |
| 155 | 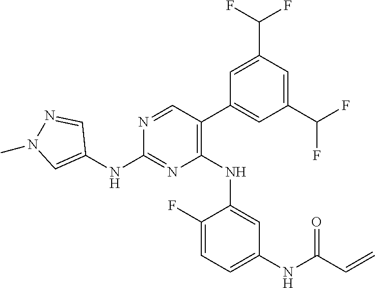 [SMILES: C=CC(=O)Nc1ccc(F)c(Nc2nc(Nc3cnn(C)c3)ncc2-c2cc(C(F)F)cc(C(F)F)c2)c1] | 338 | 133 | 107 | ND | 1670 |
| 156 | 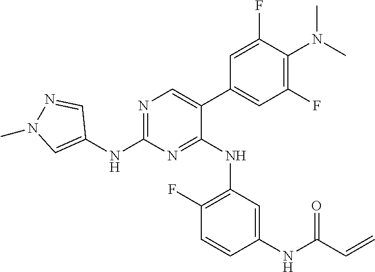 [SMILES: C=CC(=O)Nc1ccc(F)c(Nc2nc(Nc3cnn(C)c3)ncc2-c2cc(F)c(N(C)C)c(F)c2)c1] | 19 | ND | ND | ND | 309 |
| 157 | 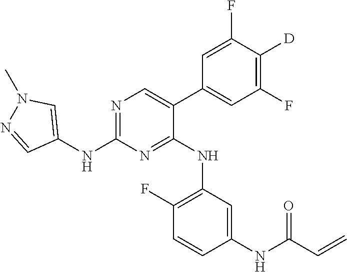 [SMILES: [2H]c1c(F)cc(-c2cnc(Nc3cnn(C)c3)nc2Nc2cc(NC(=O)C=C)ccc2F)cc1F] | 23 | 35 | 16 | ND | 151 |
| 158 | 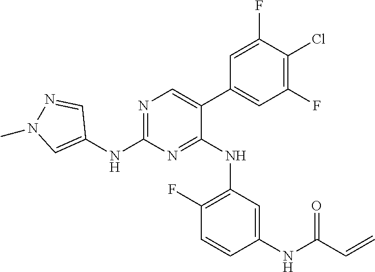 [SMILES: C=CC(=O)Nc1ccc(F)c(Nc2nc(Nc3cnn(C)c3)ncc2-c2cc(F)c(Cl)c(F)c2)c1] | 10 | 15 | 11 | ND | 40 |
| 159 | 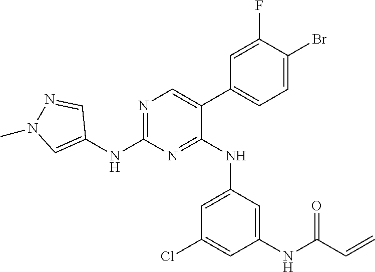 [SMILES: C=CC(=O)Nc1cc(Cl)cc(Nc2nc(Nc3cnn(C)c3)ncc2-c2ccc(Br)c(F)c2)c1] | 23 | 29 | 24 | ND | 125 |
| 160 | 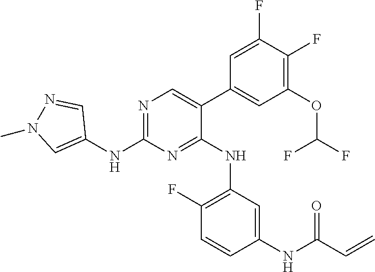 [SMILES: C=CC(=O)Nc1ccc(F)c(Nc2nc(Nc3cnn(C)c3)ncc2-c2cc(F)c(F)c(OC(F)F)c2)c1] | 261 | 63 | 68 | ND | 163 |
| 161 | 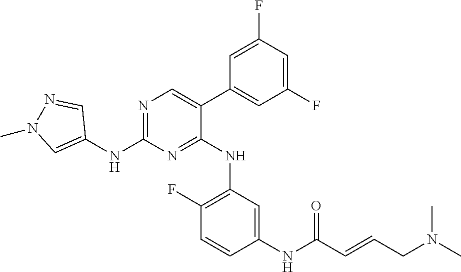 | 759 | 140 | 217 | ND | 62 |
| 162 | 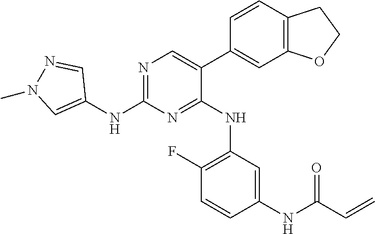 [SMILES: C=CC(=O)Nc1ccc(F)c(Nc2nc(Nc3cnn(C)c3)ncc2-c2ccc3c(c2)OCC3)c1] | 84 | 116 | 38 | ND | 428 |
| 163 | 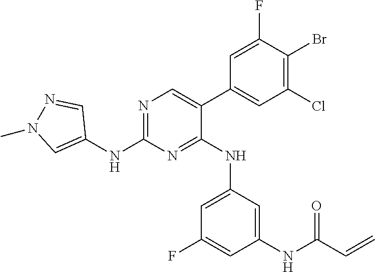 [SMILES: C=CC(=O)Nc1cc(F)cc(Nc2nc(Nc3cnn(C)c3)ncc2-c2cc(F)c(Br)c(Cl)c2)c1] | 128 | 22 | 26 | ND | 104 |
| 164 | 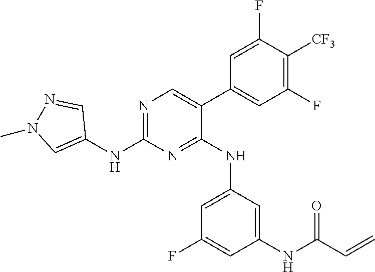 [SMILES: C=CC(=O)Nc1cc(F)cc(Nc2nc(Nc3cnn(C)c3)ncc2-c2cc(F)c(C(F)(F)F)c(F)c2)c1] | 103 | 18 | 22 | ND | 157 |
| 165 | 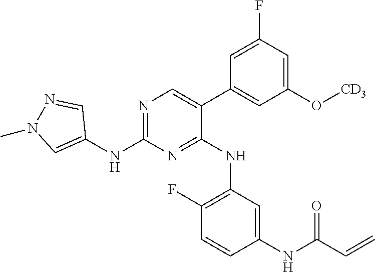 [SMILES: [2H]C([2H])([2H])Oc1cc(F)cc(-c2cnc(Nc3cnn(C)c3)nc2Nc2cc(NC(=O)C=C)ccc2F)c1] | 387 | 110 | 70 | ND | 208 |
| 166 | 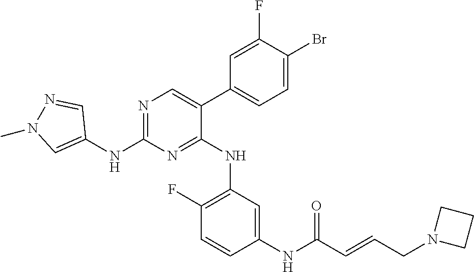 [SMILES: Cn1cc(Nc2ncc(-c3ccc(Br)c(F)c3)c(Nc3cc(NC(=O)/C=C/CN4CCC4)ccc3F)n2)cn1] | 613 | 299 | 75 | ND | 555 |
| 167 | 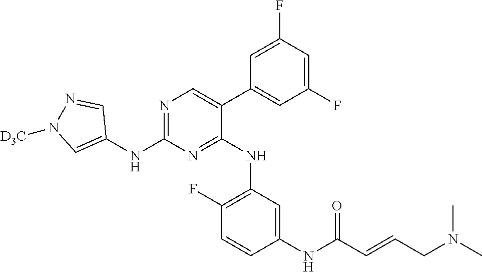 [SMILES: [2H]C([2H])([2H])n1cc(Nc2ncc(-c3cc(F)cc(F)c3)c(Nc3cc(NC(=O)/C=C/CN(C)C)ccc3F)n2)cn1] | 512 | 234 | 84 | ND | 142 |
| 168 | 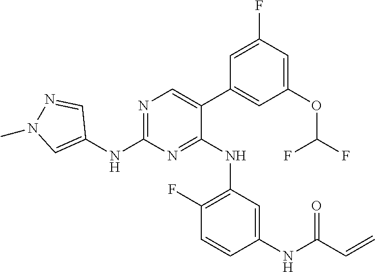 [SMILES: C=CC(=O)Nc1ccc(F)c(Nc2nc(Nc3cnn(C)c3)ncc2-c2cc(F)cc(OC(F)F)c2)c1] | 67 | 132 | 262 | ND | 574 |
| 169 | 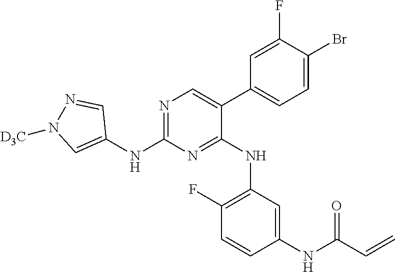 [SMILES: [2H]C([2H])([2H])n1cc(Nc2ncc(-c3ccc(Br)c(F)c3)c(Nc3cc(NC(=O)C=C)ccc3F)n2)cn1] | 11 | 33 | 36 | ND | 147 |
| 170 | 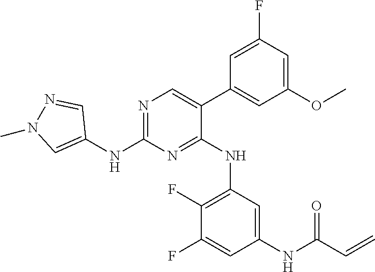 [SMILES: C=CC(=O)Nc1cc(F)c(F)c(Nc2nc(Nc3cnn(C)c3)ncc2-c2cc(F)cc(OC)c2)c1] | 5248 | >10000 | >10000 | ND | >9036 |
| 171 | 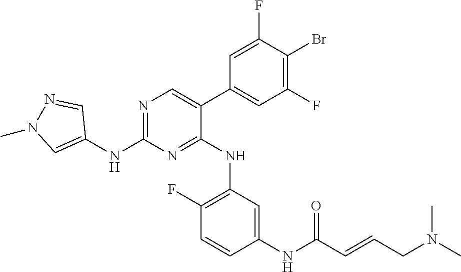 | 57 | 99 | 79 | ND | 113 |
| 172 | 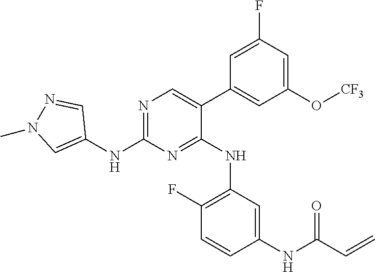 [SMILES: C=CC(=O)Nc1ccc(F)c(Nc2nc(Nc3cnn(C)c3)ncc2-c2cc(F)cc(OC(F)(F)F)c2)c1] | 585 | 967 | 843 | ND | 5982 |
| 173 | 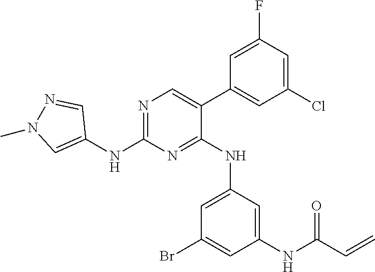 [SMILES: C=CC(=O)Nc1cc(Br)cc(Nc2nc(Nc3cnn(C)c3)ncc2-c2cc(F)cc(Cl)c2)c1] | 776 | 311 | 101 | ND | 1521 |
| 174 | 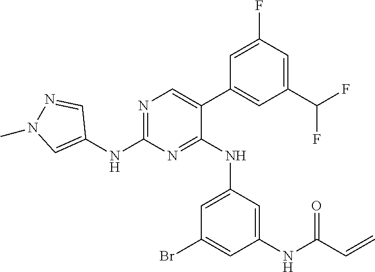 [SMILES: C=CC(=O)Nc1cc(Br)cc(Nc2nc(Nc3cnn(C)c3)ncc2-c2cc(F)cc(C(F)F)c2)c1] | 358 | 120 | 42 | ND | 759 |
| 175 | 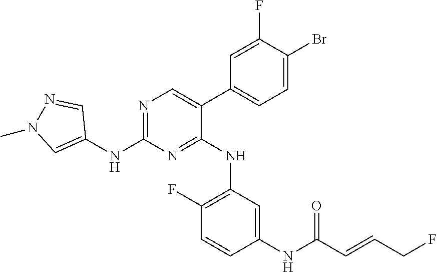 [SMILES: Cn1cc(Nc2ncc(-c3ccc(Br)c(F)c3)c(Nc3cc(NC(=O)/C=C/CF)ccc3F)n2)cn1] | 347 | 224 | 136 | ND | 1616 |
| 176 | 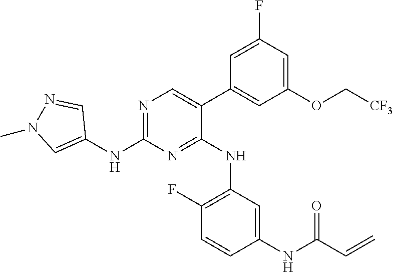 [SMILES: C=CC(=O)Nc1ccc(F)c(Nc2nc(Nc3cnn(C)c3)ncc2-c2cc(F)cc(OCC(F)(F)F)c2)c1] | ND | 859 | 1132 | ND | >10000 |
| 177 | 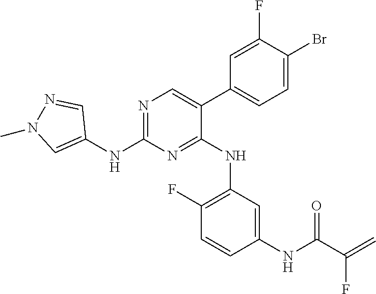 [SMILES: C=C(F)C(=O)Nc1ccc(F)c(Nc2nc(Nc3cnn(C)c3)ncc2-c2ccc(Br)c(F)c2)c1] | 326 | 1725 | 939 | ND | ND |
| 178 | 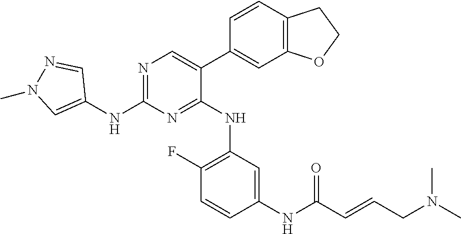 | 350 | 722 | 927 | ND | ND |

ND: Not determined

What is claimed is:

1. A compound of Formula I: 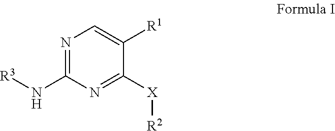
or a pharmaceutically acceptable salt or stereoisomer thereof, wherein:
X is —NH— or —O—;
R¹ is —(C(R⁴)₂)ₙR⁵, wherein R⁵ is substituted with 2 or 3 R^{5′};
n is 0, 1, 2, or 3;
each R⁴ is independently hydrogen, alkyl, halo, haloalkyl, hydroxy, alkoxy, or heteroalkyl;
R⁵ is C₄₋₁₀cycloalkyl, aryl, or heteroaryl;
each R^{5′} is independently deuterium, aryl, heteroaryl, alkyl, C₃-C₆ cycloalkyl, 3-8 membered heterocycloalkyl, oxo, halo, heteroalkyl, haloalkyl, cyano, hydroxy, amino, —NH₂, —NHR⁶, —N(CH₃)R₆, —N(R⁶)₂, —C(═O)NH₂, —C(═O)NHR⁶, —C(═O)N(R⁶)₂, —NR⁶C(═O)R⁶, —NHC(═O)R⁶, —S(═O)₂alkyl, —S(═O)₂aryl, —S(═O)₂NH₂, —S(═O)₂NHR⁶, —S(═O)₂N(R⁶)₂, —S(═O)₂heteroaryl, alkoxy, or haloalkoxy; or
two adjacent R^{5′} groups come together to form a 5- to 10-membered heterocycle, wherein the 5- to 10-membered heterocycle is unsubstituted or substituted with alkyl;
each R⁶ is independently alkyl, aminoalkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
each R⁷ is independently 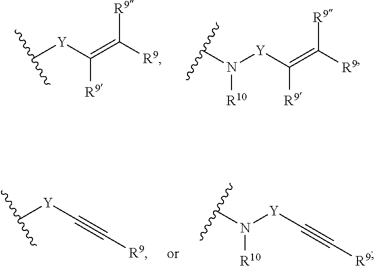
Y is —C(═O)—, —S(═O)—, or —S(═O)₂—;
R⁹, R^{9′}, and R^{9″} are independently hydrogen, deuterium, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
each R¹¹ is independently alkyl, cycloalkyl, aryl, or heteroaryl;
R³ is heteroaryl substituted with 0, 1, 2, or 3 R¹²;
each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently unsubstituted or substituted with 0, 1, or 2 R¹⁴;
each R¹³ is independently hydrogen, alkyl, cycloalkyl, aryl, or heteroaryl;
each R¹⁴ is independently deuterium, aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy; and
each R¹⁵ is independently alkyl, cycloalkyl, aryl, or heteroaryl.

2. The compound of claim 1, wherein X is —NH—.

3. The compound of either of claim 1 or 2, wherein n is 0.

4. The compound of any one of claims 1 to 3, wherein R⁵ is phenyl, naphthyl, anthracenyl, phenanthrenyl, C-linked pyridyl, C-linked pyrimidinyl, C-linked pyrazolyl, C-linked imidazolyl, or C-linked indolyl; wherein R⁵ is substituted with 2 or 3 R^{5′}.

5. The compound of any one of claims 1 to 4, wherein R⁵ is substituted with 2 R^{5′}.

6. The compound of any one of claims 1 to 4, wherein R⁵ is substituted with 3 R^{5′}.

7. The compound of any one of claims 1 to 6, wherein each R^{5′} is independently alkyl, haloalkyl, heterocycloalkyl, halo, cyano, hydroxy, —N(R⁶)₂, —C(═O)NHR⁶, —NHC(═O)R⁶, —S(═O)₂NH₂, alkoxy, or haloalkoxy.

8. The compound of any one of claims 1 to 7, wherein each R^{5′} is independently methyl, ethyl, tert-butyl, pyrrolidinyl, piperidinyl, piperazinyl, morpholinyl, fluoro, chloro, cyano, hydroxy, —N(R⁶)₂, —N(CH₃)R₆, —C(═O)NHR⁶, —NHC(═O)R⁶, —S(═O)₂NH₂, methoxy, ethoxy, fluoromethyl, difluoromethyl, trifluoromethyl, fluoromethoxy, difluoromethoxy, or trifluoromethoxy.

9. The compound of any one of claims 1-8, wherein each R¹¹ is independently methyl, morpholinyl, fluoro, chloro, cyano, —C(═O)NHMe, —NHC(═O)Me, —S(═O)₂NH₂, methoxy, fluoromethyl, difluoromethyl, trifluoromethyl, difluoromethoxy, or trifluoromethoxy.

10. The compound of any one of claims 1 to 8, wherein each R⁶ is independently alkyl or aryl.

11. The compound of claim 10, wherein each R⁶ is independently methyl, ethyl, iso-propyl, tert-butyl, phenyl, or naphthyl.

12. The compound of claim 11, wherein each R⁶ is independently methyl or phenyl.

13. The compound of any one of claims 1 to 12, wherein R² is monocyclic.

14. The compound of any one of claims 1 to 13, wherein R² is phenyl, cyclopropyl, cyclobutyl, cyclopentyl, cyclohexyl, pyrrolyl, imidazolyl, pyrazolyl, triazolyl, tetrazolyl, thiazolyl, isothiazolyl, oxazolyl, isoxazolyl, pyridinyl, pyrimidinyl, pyridazinyl, pyrazinyl, or triazinyl; wherein R² is substituted with at least one R⁷ and 0, 1, or 2 R⁸.

15. The compound of any one of claims 1 to 14, wherein R² is phenyl, cyclohexyl, or pyrrolyl; wherein R² is substituted with at least one R⁷ and 0, 1, or 2 R⁸.

16. The compound of any one of claims 1 to 15, wherein R⁷ is

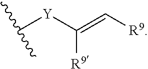

17. The compound of any one of claims 1 to 15, wherein R⁷ is

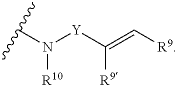

18. The compound of any one of claims 1 to 15, wherein R⁷ is

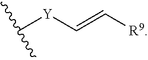

19. The compound of any one of claims 1 to 15, wherein R⁷ is

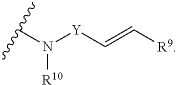

20. The compound of any one of claims 1 to 19, wherein Y is —C(═O)—.

21. The compound of any one of claims 1 to 19, wherein Y is —S(═O)₂—.

22. The compound of any one of claims 1 to 21, wherein R⁹ and R^{9′} are independently hydrogen, halo, alkyl, heteroalkyl, haloalkyl, or (alkyl)heterocycloalkyl.

23. The compound of any one of claims 1 to 22, wherein R⁹ and R^{9′} are independently hydrogen, fluoro, chloro, methyl, hydroxyethyl, methoxyethyl, methoxymethyl, dimethylaminomethyl, 1-piperidinylmethyl, 1-morpholinylmethyl, or fluoromethyl.

24. The compound of any one of claims 1 to 15, 17, or 19 to 23, wherein R¹⁰ is hydrogen, methyl, ethyl n-propyl, iso-propyl, n-butyl, sec-butyl, tert-butyl, trifluoromethyl, or cyclopropyl.

25. The compound of claim 24, wherein R¹⁰ is hydrogen or methyl.

26. The compound of any one of claims 1 to 25, wherein R² is substituted with 1 or 2 R⁸.

27. The compound of any one of claims 1 to 26, wherein each R⁸ is independently methyl, ethyl, n-propyl, iso-propyl, n-butyl, iso-butyl, sec-butyl, tert-butyl, fluoro, chloro, heteroalkyl, cyano, hydroxy, amino, —N(R¹¹)₂, methoxy, ethoxy, or trifluoromethoxy.

28. The compound of any one of claims 1 to 27, wherein each R⁸ is independently methyl, ethyl, iso-propyl, tert-butyl, fluoro, chloro, —N(R¹¹)₂, hydroxyethyl, methoxyethyl, or cyano.

29. The compound of any one of claims 1 to 28, wherein each R¹¹ is independently alkyl or aryl.

30. The compound of any one of claims 1 to 29, wherein each R¹¹ is independently methyl, ethyl, n-propyl, iso-propyl, n-butyl, iso-butyl, sec-butyl, tert-butyl, phenyl, naphthyl, anthracenyl, or phenanthrenyl.

31. The compound of any one of claims 1 to 30, wherein each R¹¹ is independently methyl, ethyl, iso-propyl, tert-butyl, phenyl, or naphthyl.

32. The compound of any one of claims 1 to 31, wherein each R¹¹ is independently methyl or phenyl.

33. The compound of any one of claims 1 to 25, wherein R² is not substituted with R⁸.

34. The compound of any one of claims 1 to 33, wherein R³ is phenyl, pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl; wherein R³ is substituted with 0, 1, 2, or 3 R¹².

35. The compound of any one of claims 1 to 34, wherein R³ is phenyl, imidazolyl, pyrazolyl, triazolyl, indolyl, indazolyl, thiazolyl, isothiazolyl, or pyridinyl; wherein R³ is substituted with 0, 1, 2, or 3 R¹².

36. The compound of any one of claims 1 to 35, wherein R³ is: 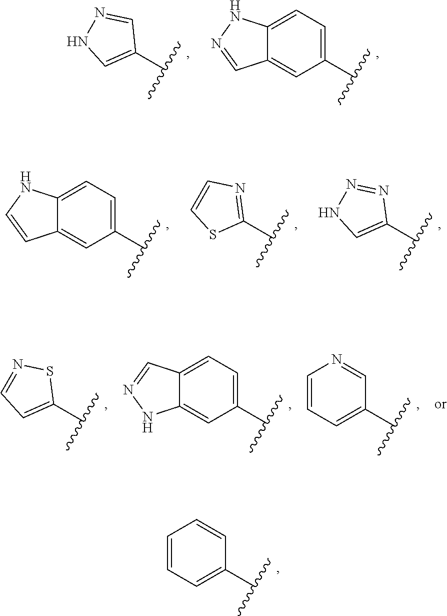
wherein R³ is substituted with 0 to 3 R¹².

37. The compound of any one of claims 1 to 33, wherein R³ is:

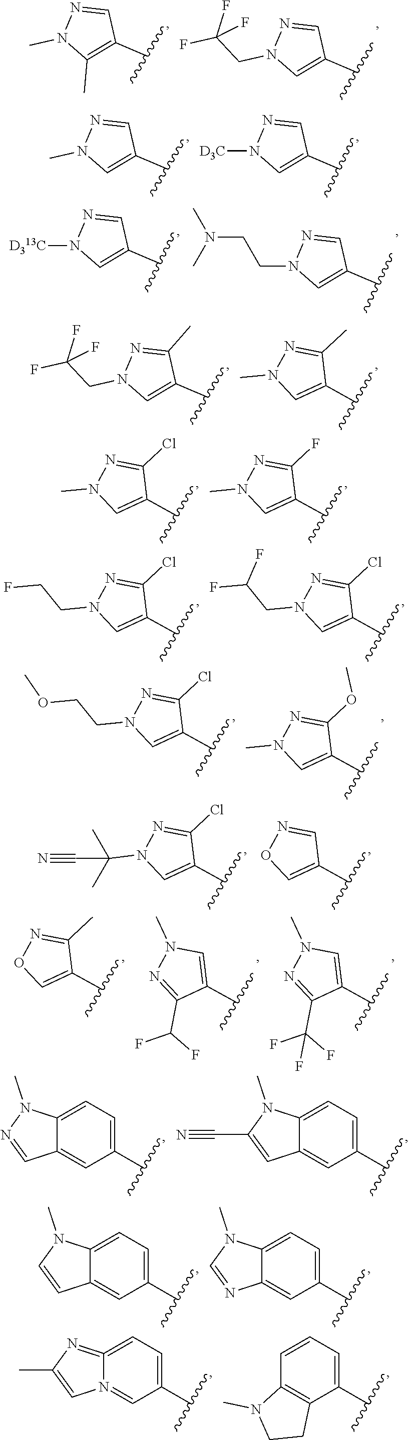

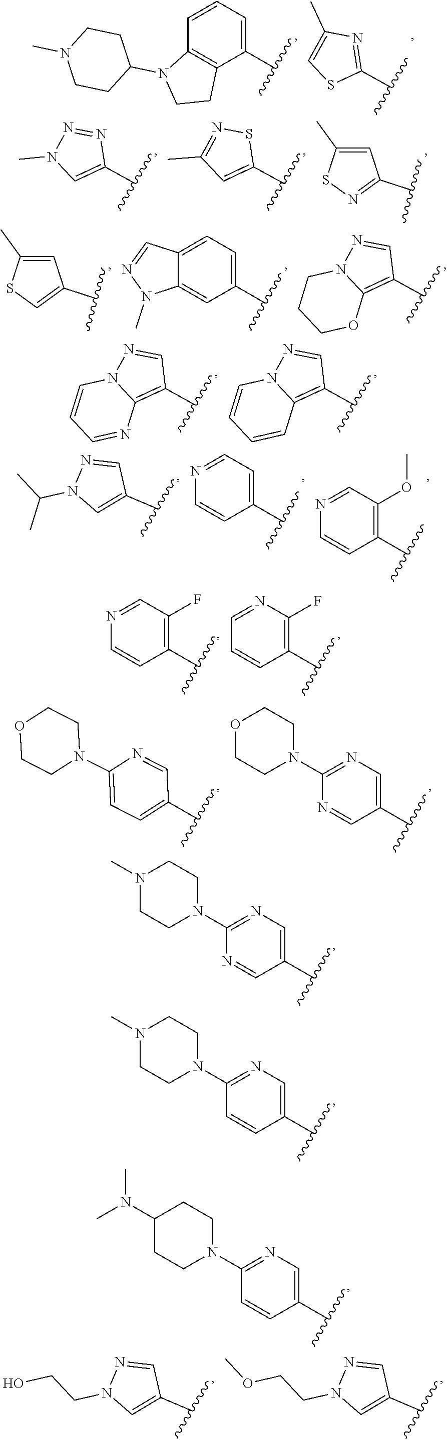

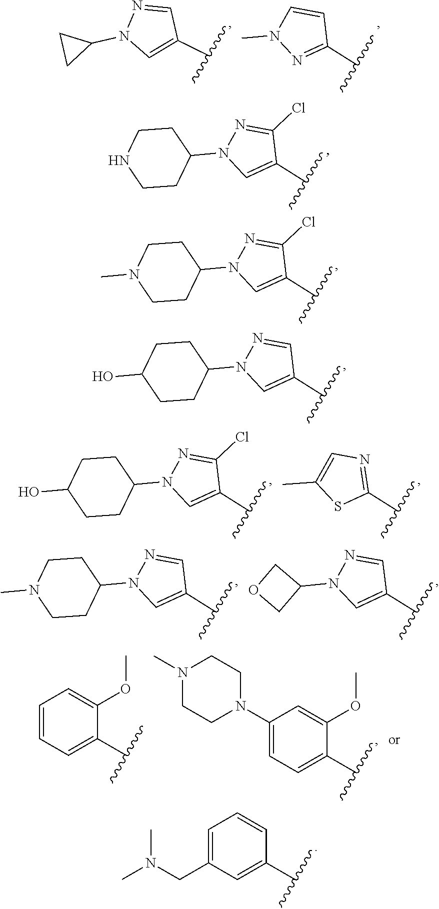

38. The compound of any one of claim 37, wherein R³ is:

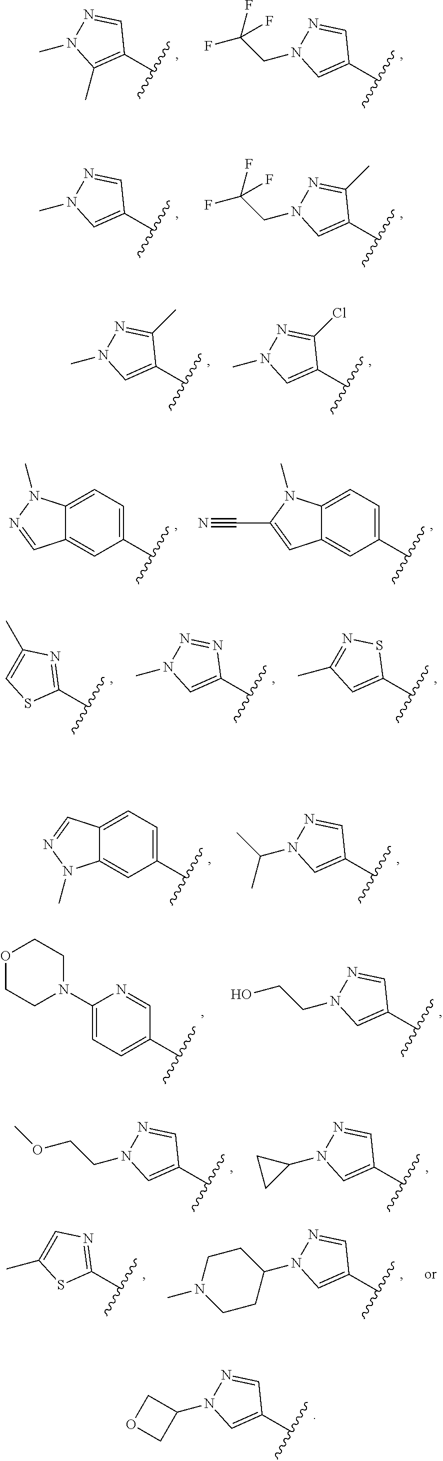

39. The compound of any one of claims 1 to 36, wherein R³ is unsubstituted.

40. The compound of any one of claims 1 to 36, wherein R³ is substituted with at least 1 R¹².

41. The compound of claim 40, wherein R³ is substituted with at least 2 R¹².

42. The compound of any one of claims 1 to 36, claim 40, or claim 41, wherein each R¹² is independently aryl, heteroaryl, alkyl, heteroalkyl, haloalkyl, halo, cyano, heterocycloalkyl, —N(R¹³)₂, —S(═O)₂NH₂, or cycloalkyl.

43. The compound of claim 42, wherein each R¹² is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, azetidinyl, oxetanyl, pyrrolidinyl, imidazolidinyl, tetrahydrofuranyl, piperidinyl, piperazinyl, tetrahydropyranyl, morpholinyl, —N(R¹³)₂, cyclopropyl, cyclobutyl, cyclopentyl, or cyclohexyl.

44. The compound of claim 43, wherein each R¹² is independently methyl, iso-propyl, tert-butyl, hydroxyethyl, methoxyethyl, trifluoromethyl, trifluoroethyl, chloro, cyano, morpholinyl, or cyclopropyl.

45. The compound of claim 44, wherein each R¹² is independently methyl, hydroxyethyl, methoxyethyl, trifluoroethyl, or chloro.

46. The compound of claim 45, wherein each R¹² is independently methyl or chloro.

47. The compound of any one of claims 1 to 36 or 40 to 43, wherein each R¹³ is independently alkyl or cycloalkyl.

48. The compound of claim 47, wherein each R¹³ is independently methyl, ethyl, n-propyl, iso-propyl, n-butyl, iso-butyl, sec-butyl, tert-butyl, cyclopropyl, cyclobutyl, cyclopentyl, or cyclohexyl.

49. The compound of claim 48, wherein each R¹³ is independently methyl, ethyl, iso-propyl, tert-butyl, cyclopropyl, cyclopentyl, or cyclohexyl.

50. The compound of claim 49, wherein each R¹³ is independently methyl, cyclopropyl, or cyclohexyl.

51. The compound of any one of claims 1 to 42, wherein the aryl, heteroaryl, heterocycloalkyl, or cycloalkyl of R¹² is unsubstituted.

52. The compound of any one of claims 1 to 42, wherein the aryl, heteroaryl, heterocycloalkyl, or cycloalkyl of R¹² is substituted with 1 or 2 R¹⁴.

53. The compound of any one of claims 1 to 42 or claim 52, wherein each R¹⁴ is independently alkyl, cycloalkyl, heterocycloalkyl, halo, cyano, —N(R¹⁵)₂, or alkoxy.

54. The compound of claim 53, wherein each R¹⁴ is independently methyl, ethyl, n-propyl, iso-propyl, n-butyl, iso-butyl, sec-butyl, tert-butyl, cyclopropyl, cyclobutyl, cyclopentyl, cyclohexyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, —N(R¹⁵)₂ methoxy, ethoxy, or trifluoromethoxy.

55. The compound of claim 54, wherein each R¹⁴ is independently methyl, ethyl, iso-propyl, tert-butyl, pyrrolidinyl, piperidinyl, morpholinyl, fluoro, chloro, —N(R¹⁵)₂, or methoxy.

56. The compound of any one of claims 1 to 42 or claims 52 to 55, wherein each R¹⁵ is independently alkyl or cycloalkyl.

57. The compound of claim 56, wherein each R¹⁵ is methyl, ethyl, n-propyl, iso-propyl, n-butyl, iso-butyl, sec-butyl, tert-butyl, cyclopropyl, cyclobutyl, cyclopentyl, or cyclohexyl.

58. The compound of any one of claims 1 to 42 or claims 52 to 57, wherein each R¹³ is independently methyl, ethyl, iso-propyl, tert-butyl, cyclopropyl, cyclopentyl, or cyclohexyl.

59. The compound of claim 58, wherein each R¹³ is independently methyl, cyclopropyl, or cyclohexyl.

60. The compound of any one of claims 1 to 59, wherein:
X is —NH— or —O—;
n is 0;
R⁵ is phenyl substituted with 2 or 3 R^{5′};
R² is phenyl substituted with at least one R⁷ and 0, 1, or 2 R⁸; and
R³ is pyrazolyl substituted with 0, 1, 2, or 3 R¹².

61. The compound of claim 60, wherein X is —NH—.

62. The compound of either of claim 60 or 61, wherein R^{5′} is fluoromethyl, difluoromethyl, or trifluoromethyl.

63. The compound of any one of claims 60 to 62, wherein:
R⁷ is 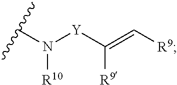
and
R⁸ is halo.

64. The compound of any one of claims 60 to 63, wherein:
R⁸ is fluoro;
Y is —C(═O)—;
R⁹ and R^{9′} are hydrogen; and
R¹⁰ is hydrogen.

65. The compound of any one of claims 60 to 64, wherein R¹² is alkyl.

66. The compound of any one of claims 60 to 65, wherein R¹² is methyl.

67. The compound of any one of claims 60 to 66, wherein the compound is of Formula I-A, Formula I-B, Formula I-C, Formula I-D, Formula I-E, Formula I-F, or Formula I-G: 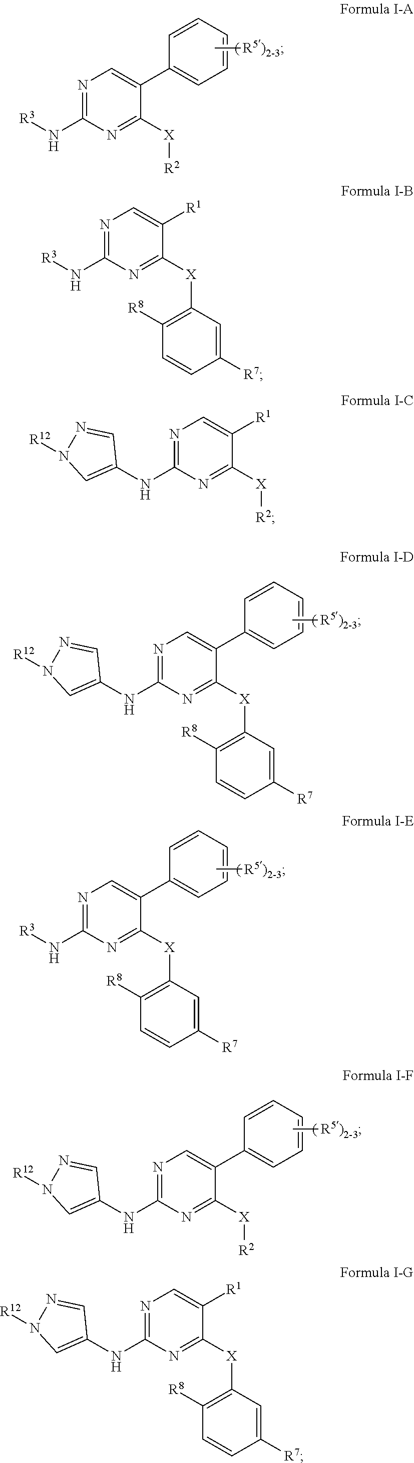
or a pharmaceutically acceptable salt or stereoisomer thereof.

68. The compound of claim 1, wherein the compound is: 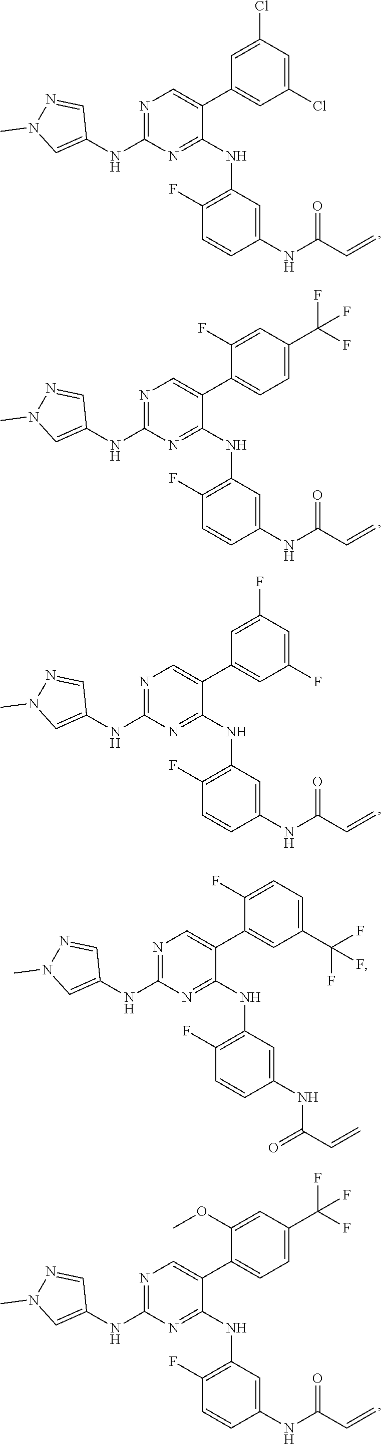 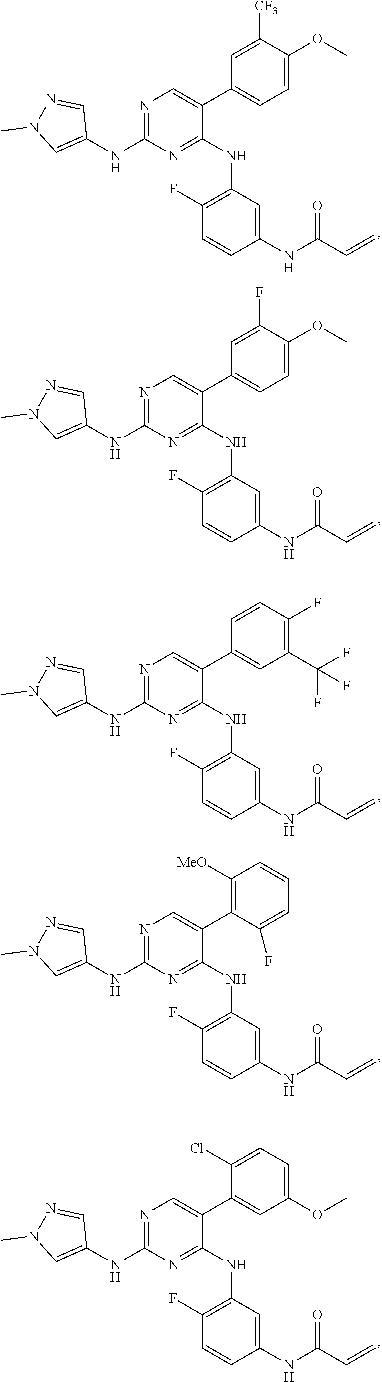 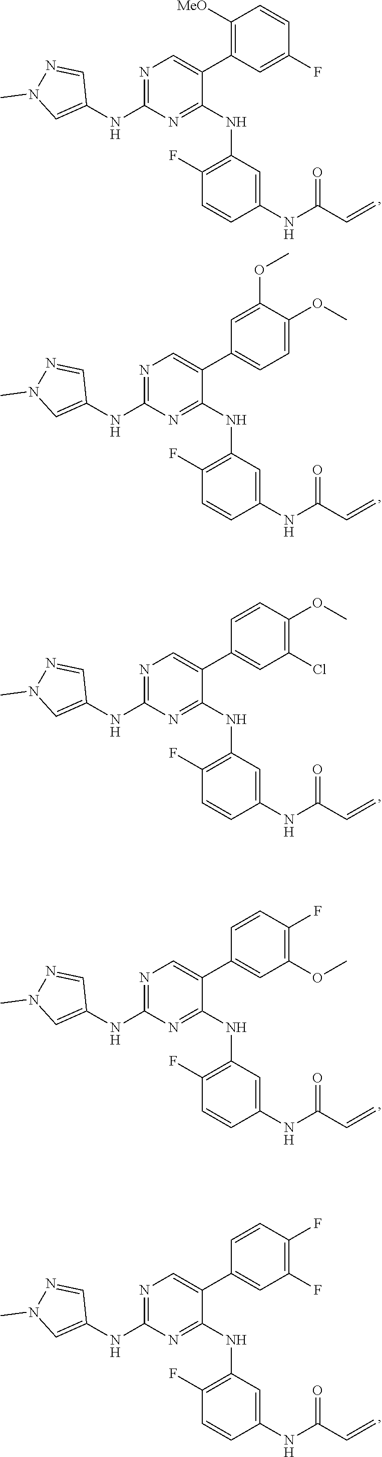 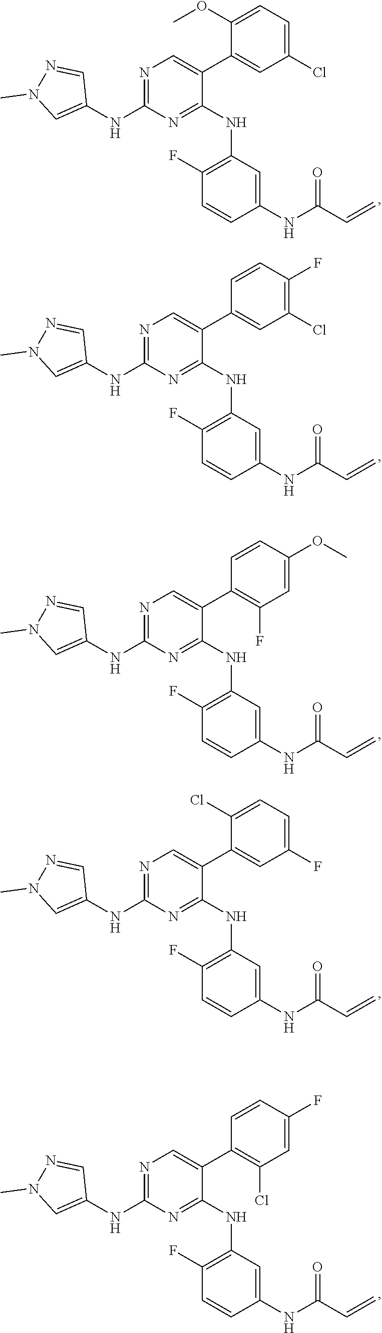 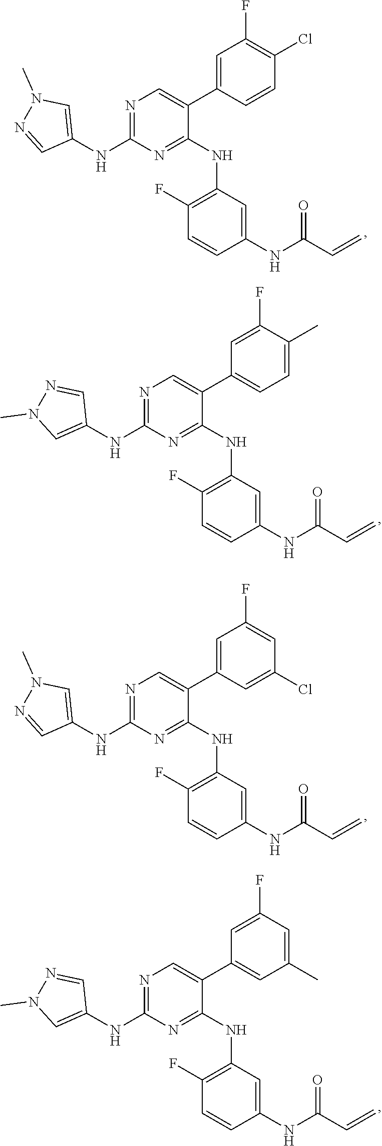 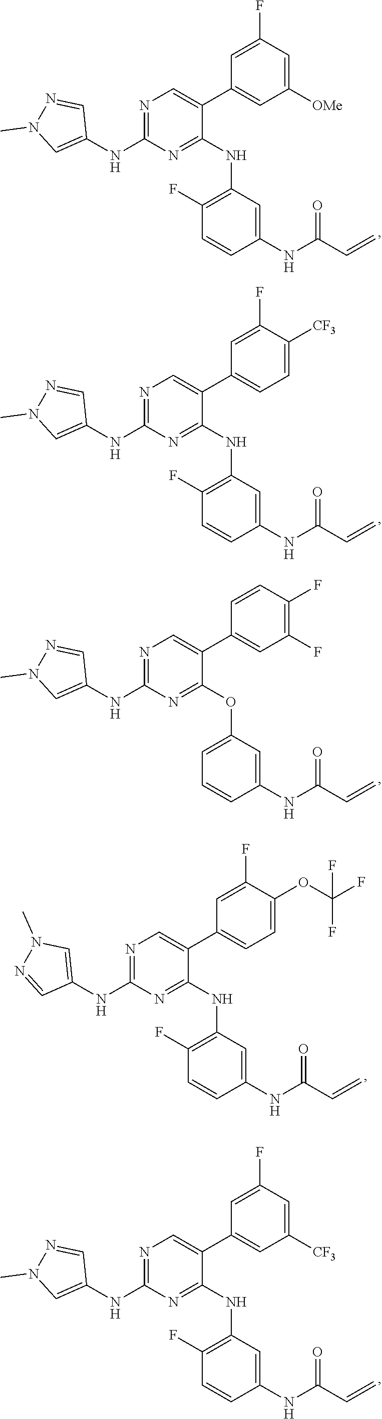 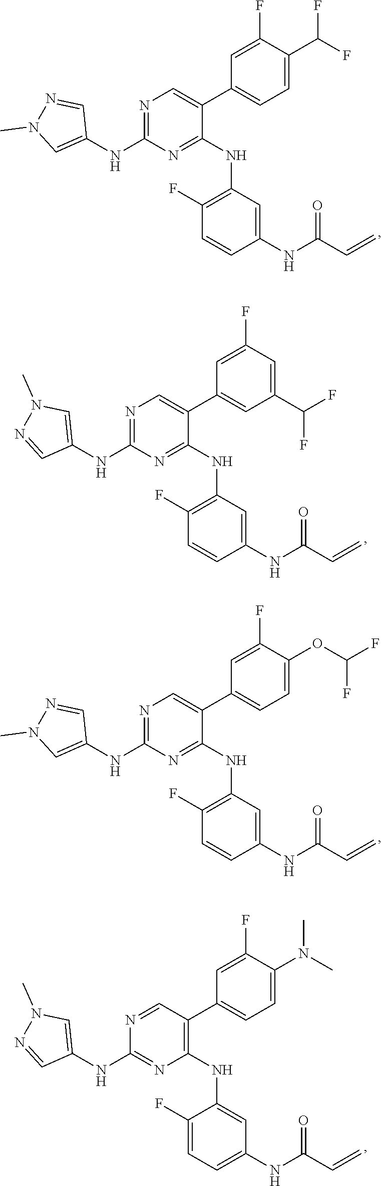 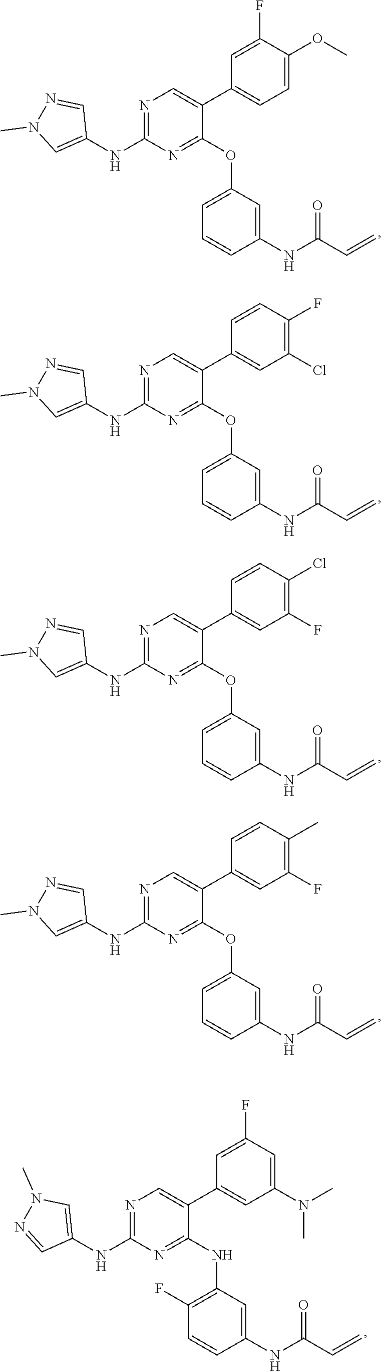 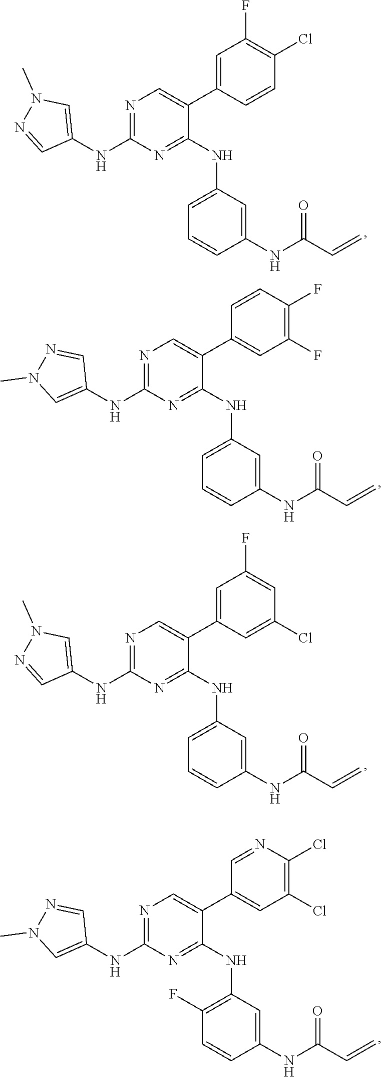 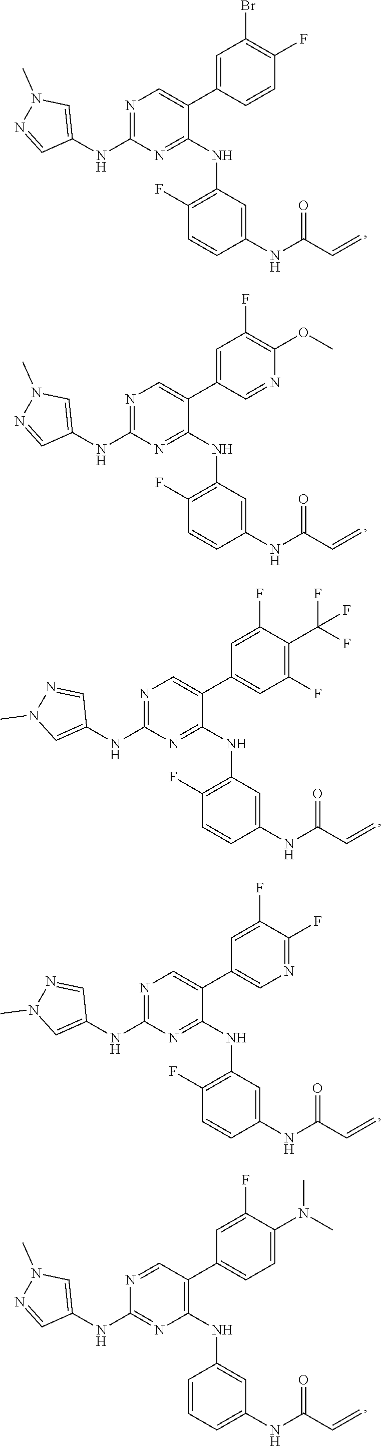 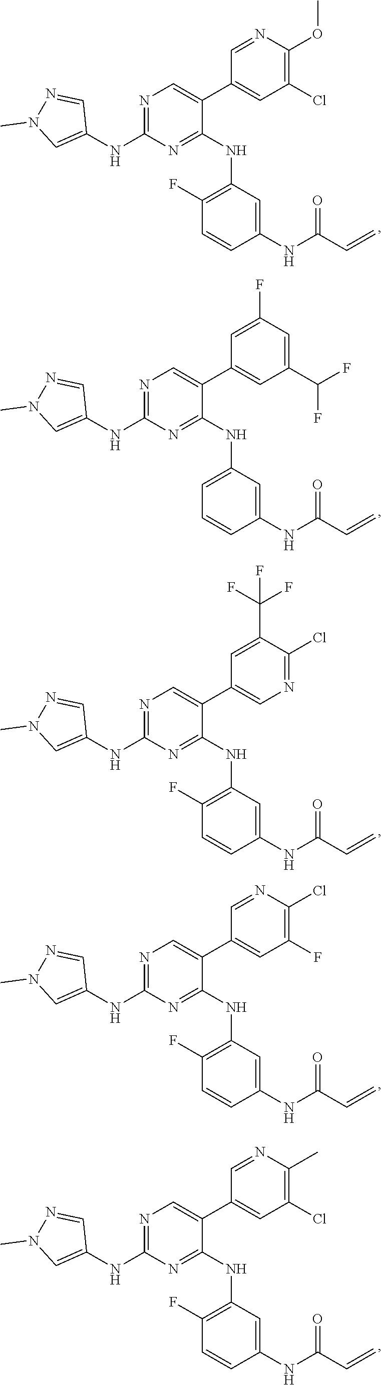 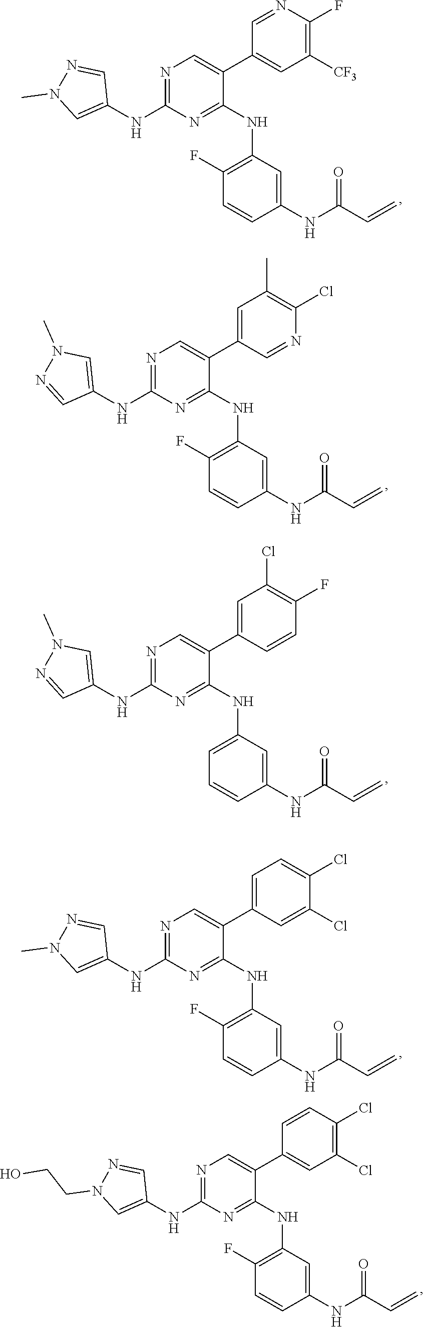 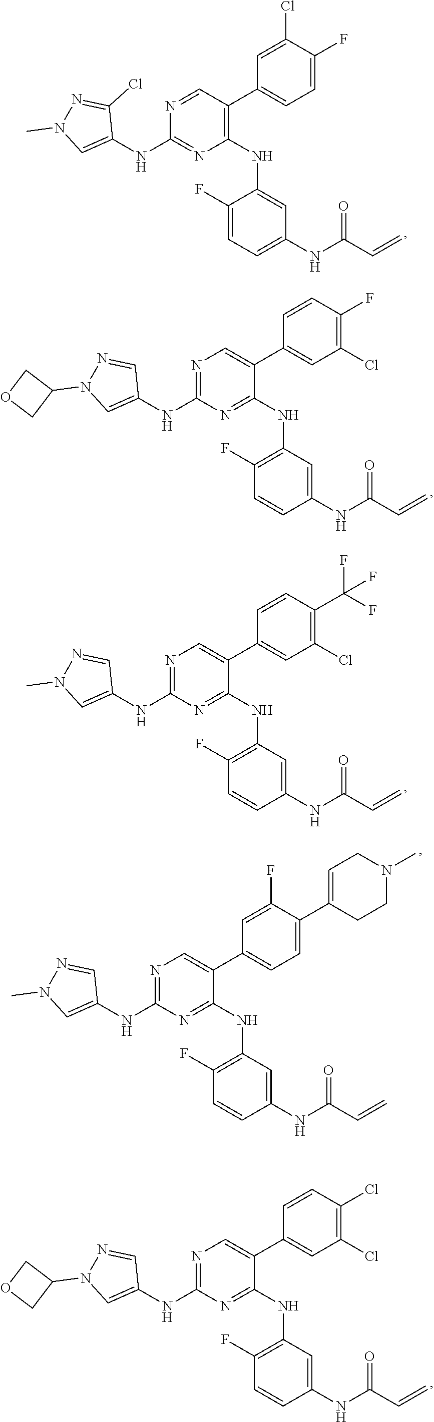 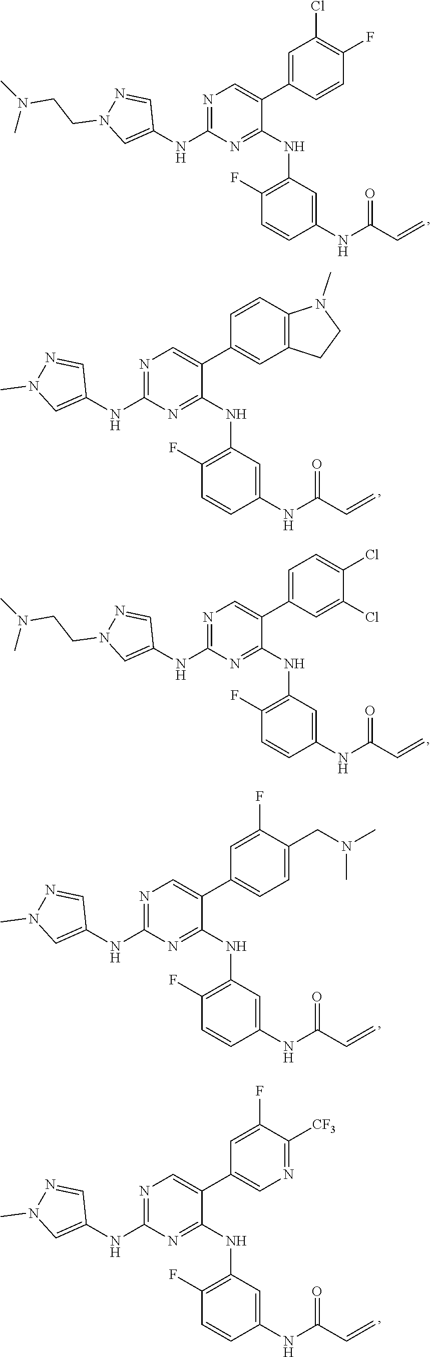 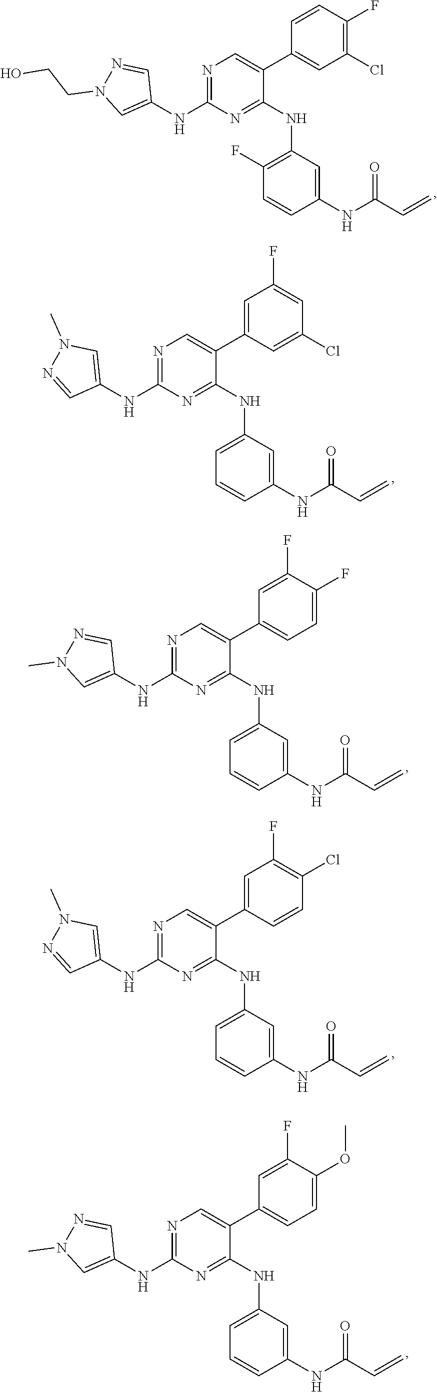 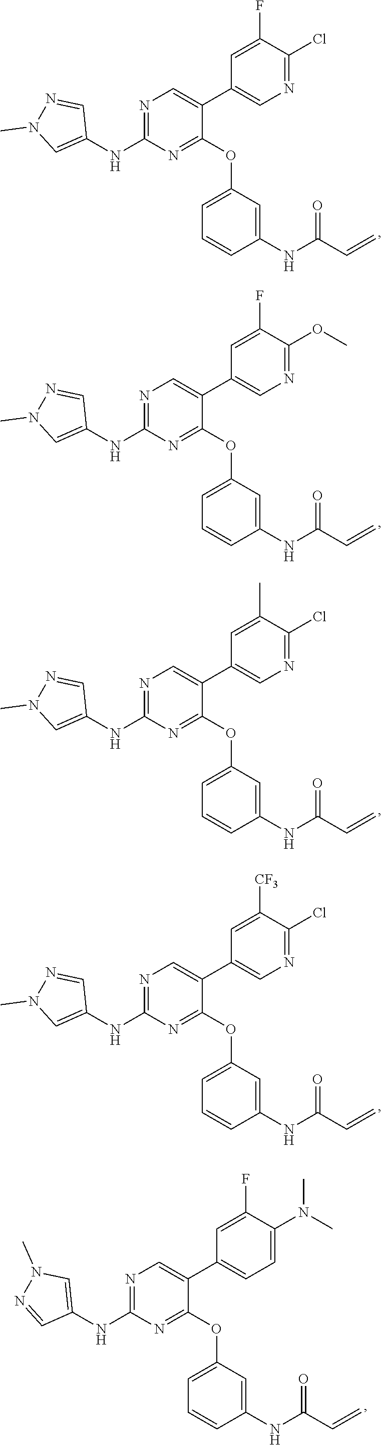 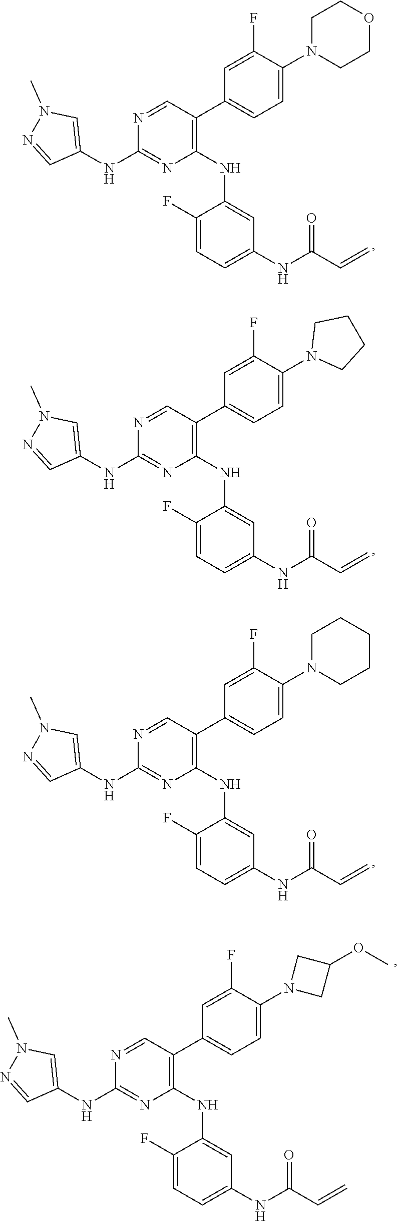 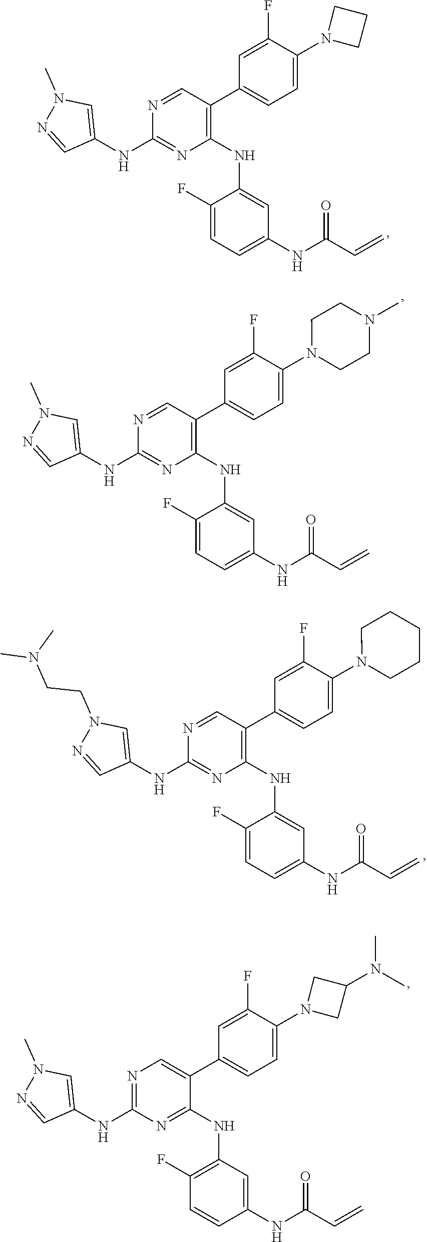 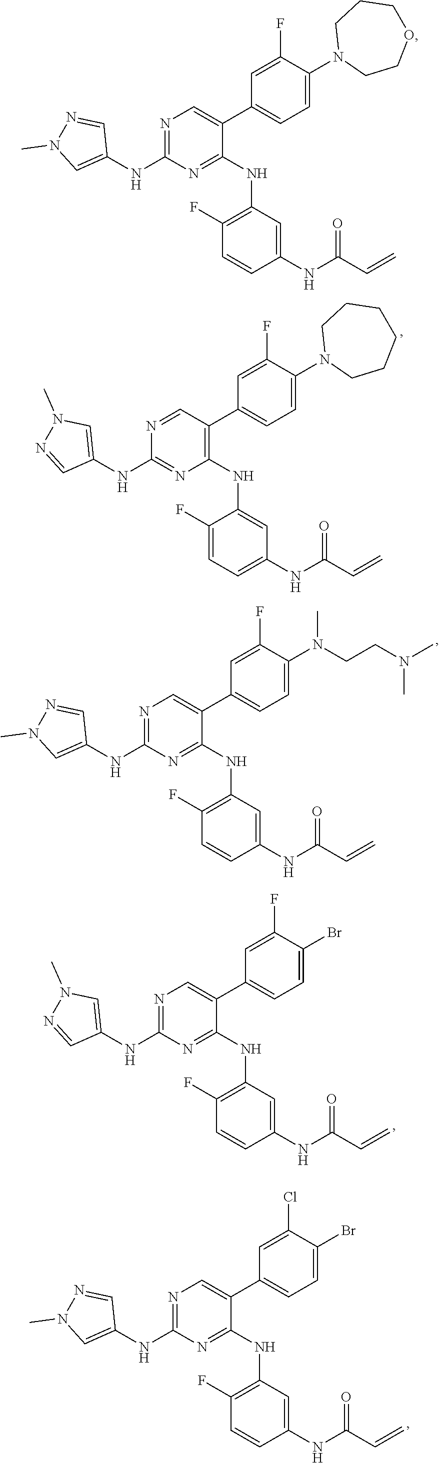 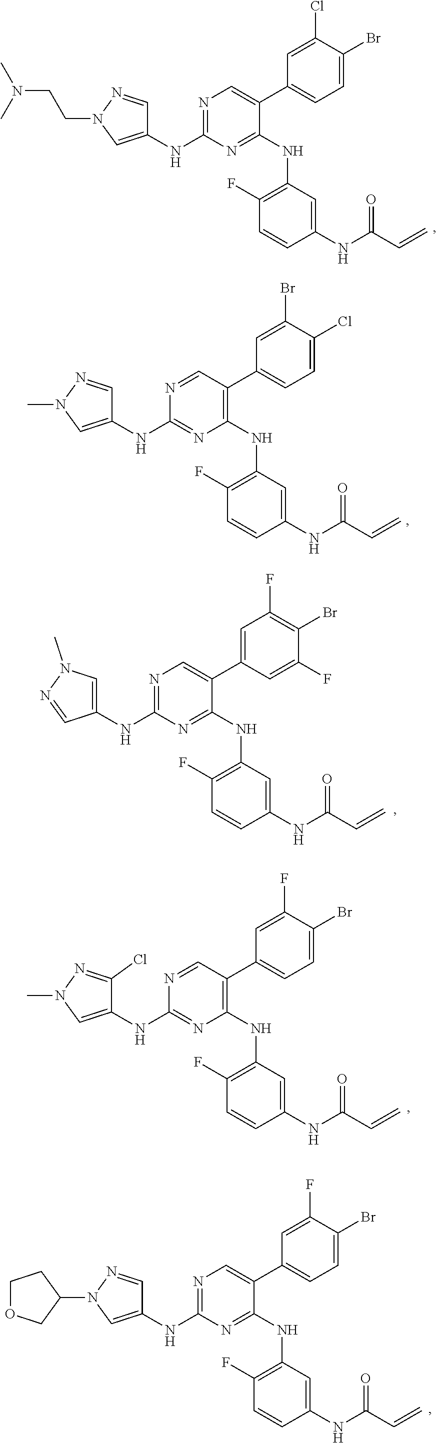 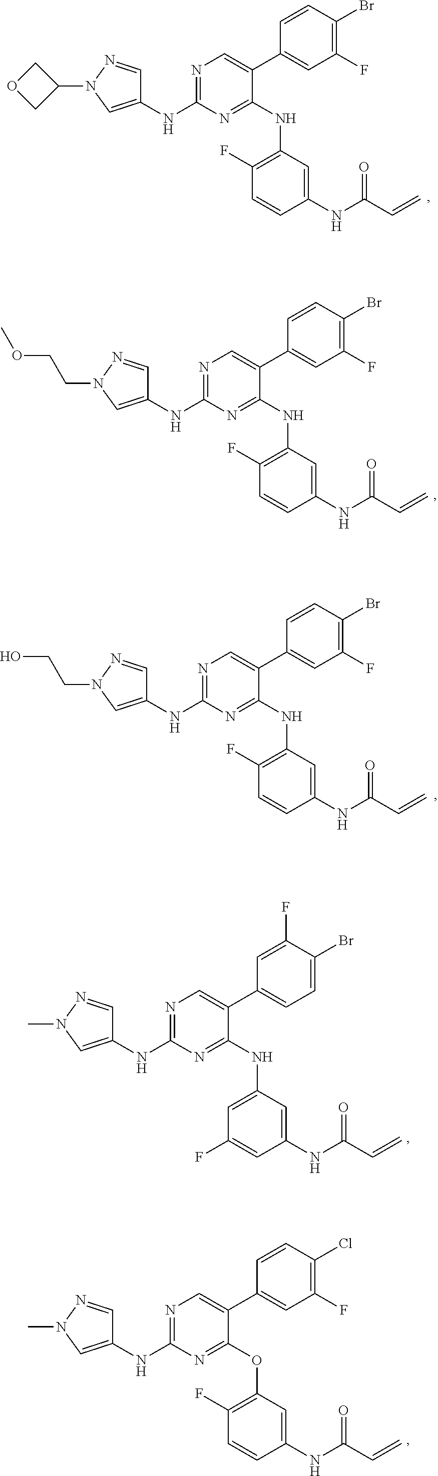 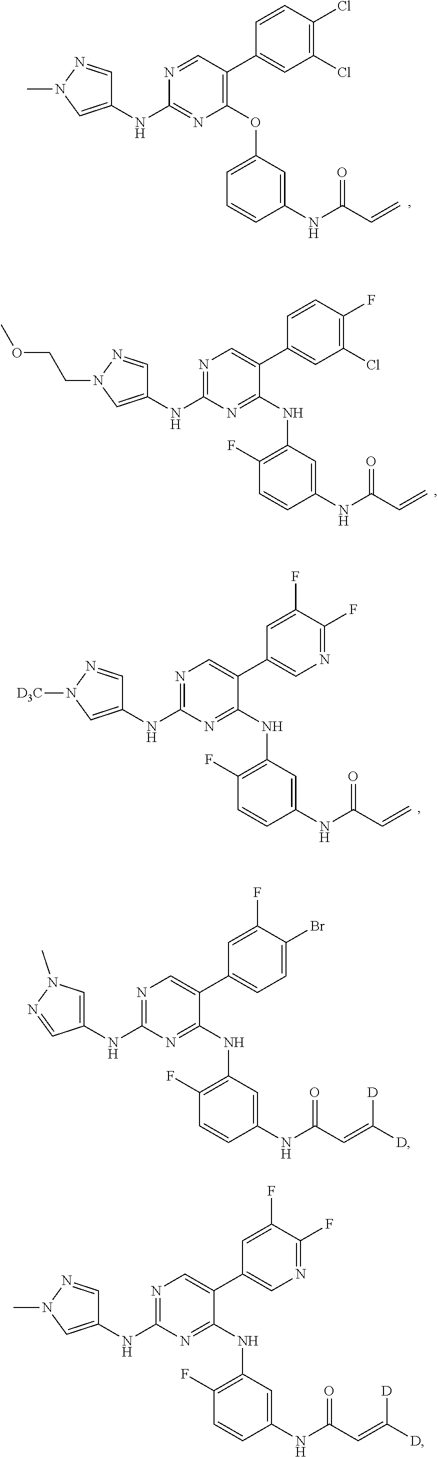 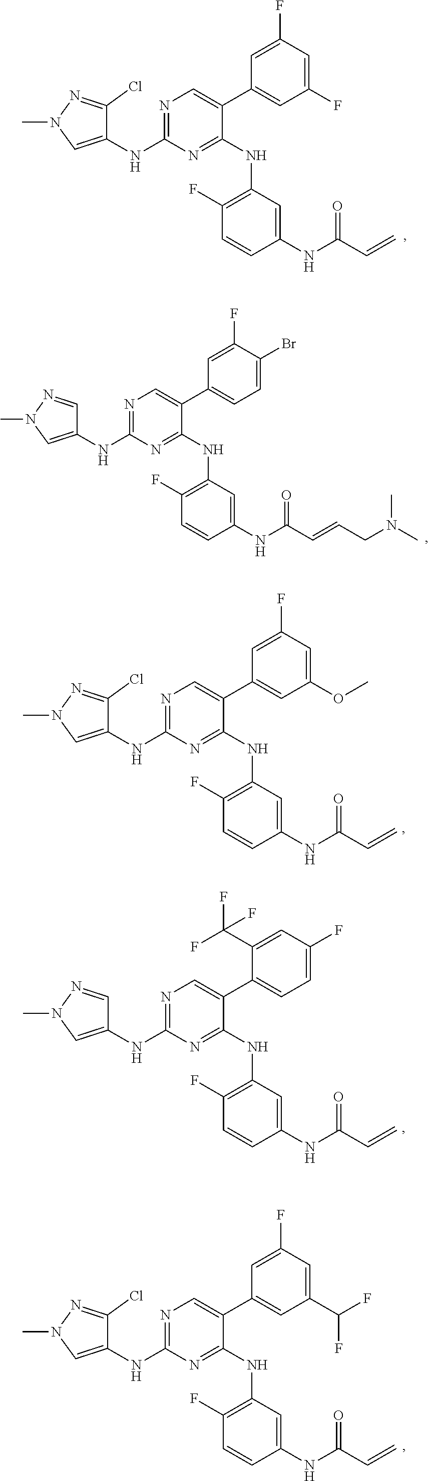 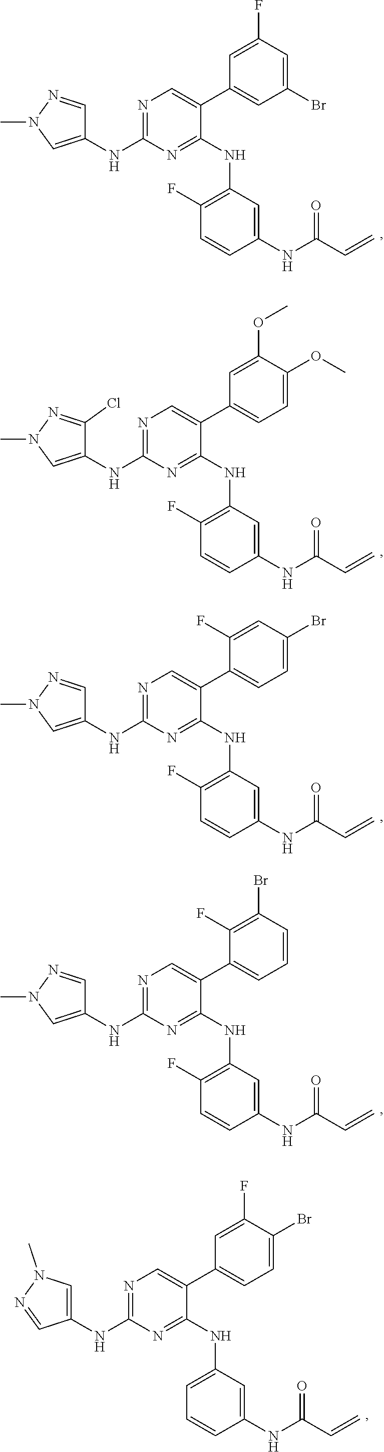 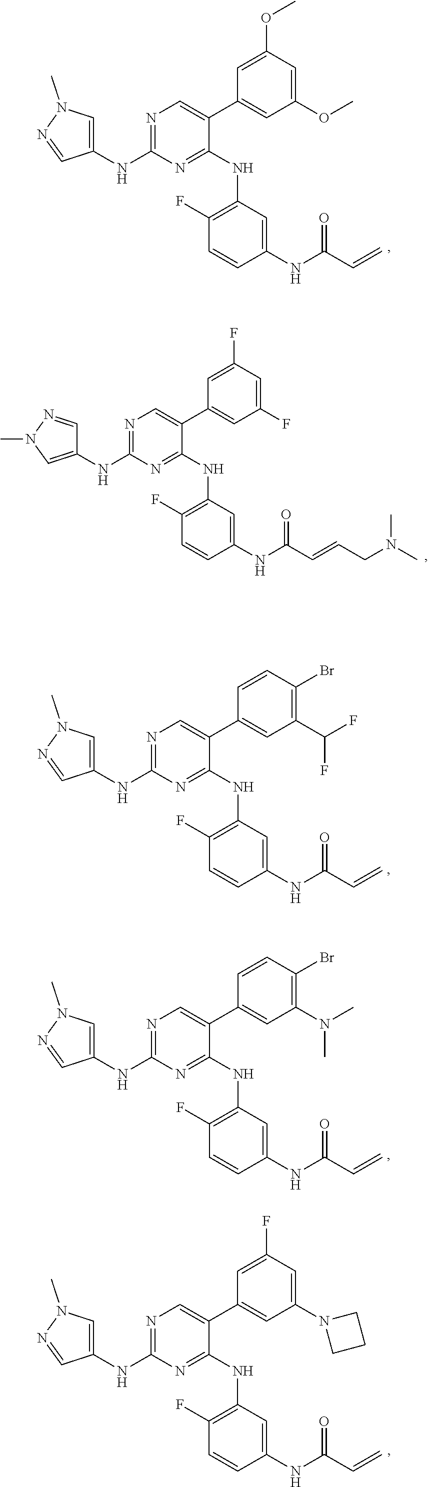 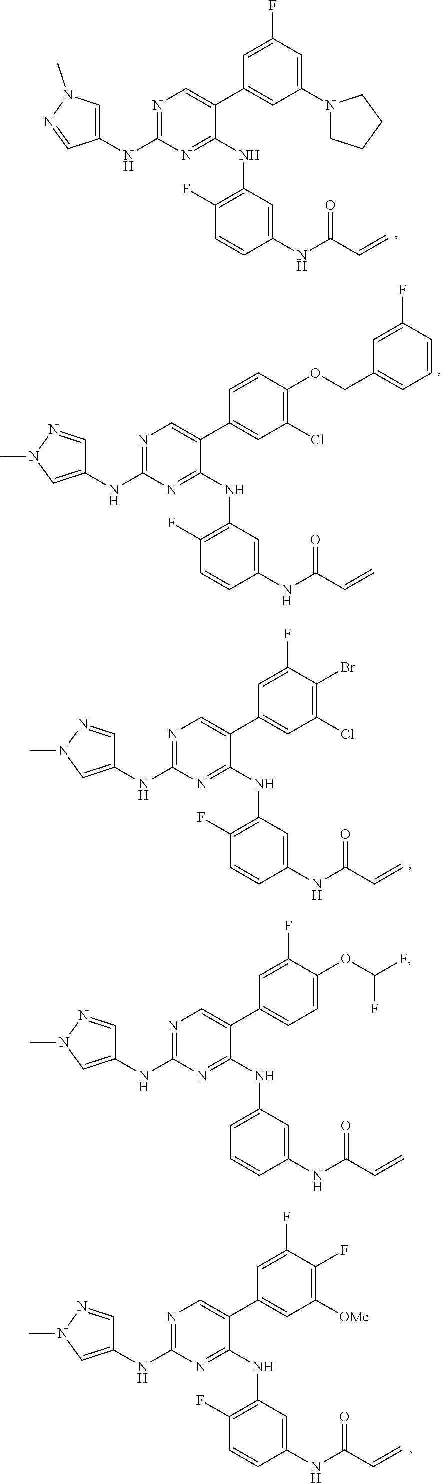 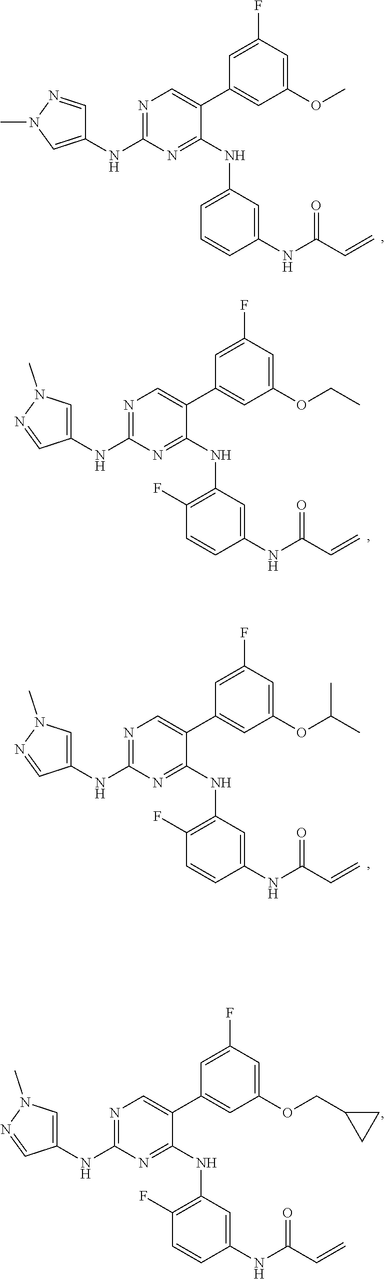 [SMILES: C=CC(=O)Nc1cccc(Nc2nc(Nc3cnn(C)c3)ncc2-c2cc(F)cc(OC)c2)c1 ; C=CC(=O)Nc1ccc(F)c(Nc2nc(Nc3cnn(C)c3)ncc2-c2cc(F)cc(OCC)c2)c1 ; C=CC(=O)Nc1ccc(F)c(Nc2nc(Nc3cnn(C)c3)ncc2-c2cc(F)cc(OC(C)C)c2)c1 ; C=CC(=O)Nc1ccc(F)c(Nc2nc(Nc3cnn(C)c3)ncc2-c2cc(F)cc(OCC3CC3)c2)c1] 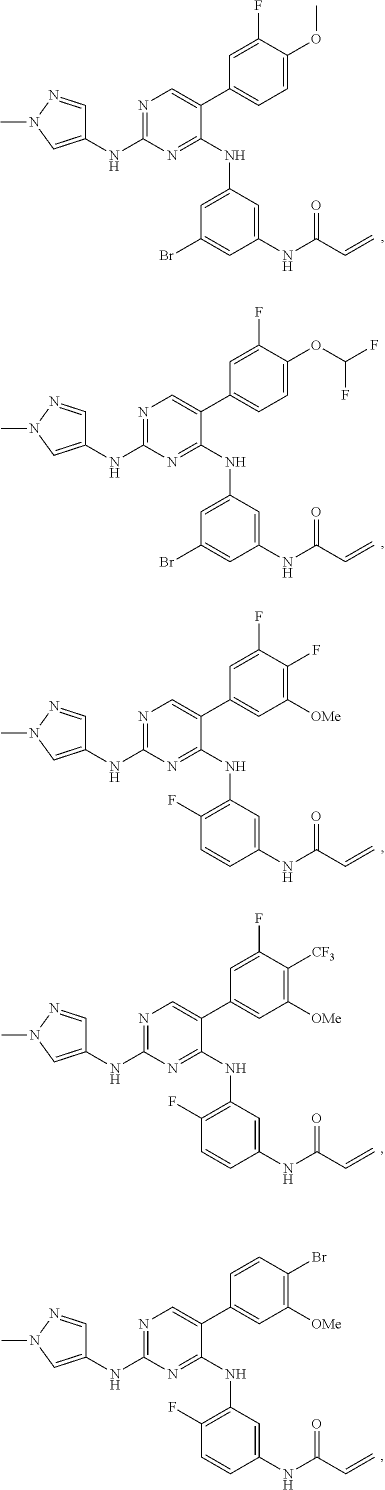 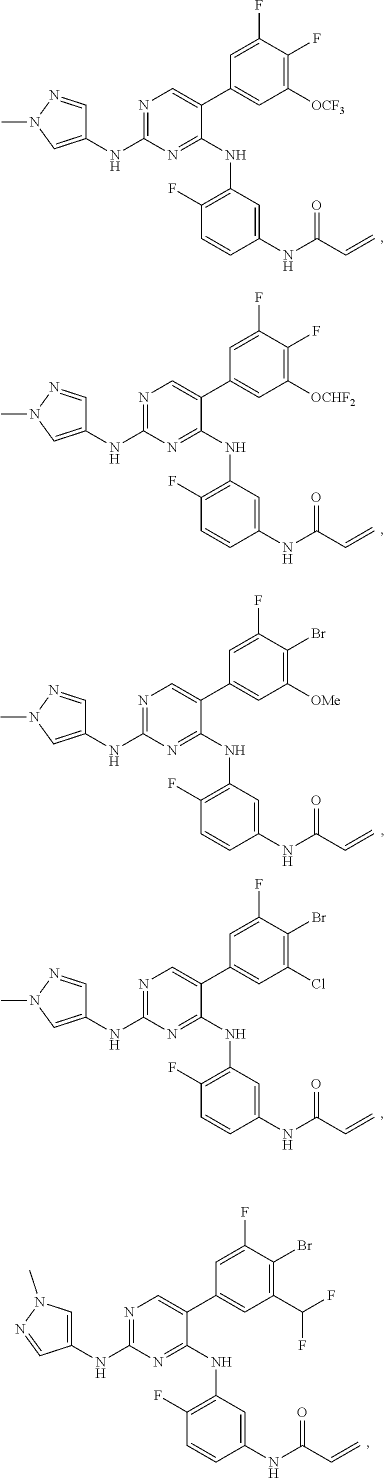 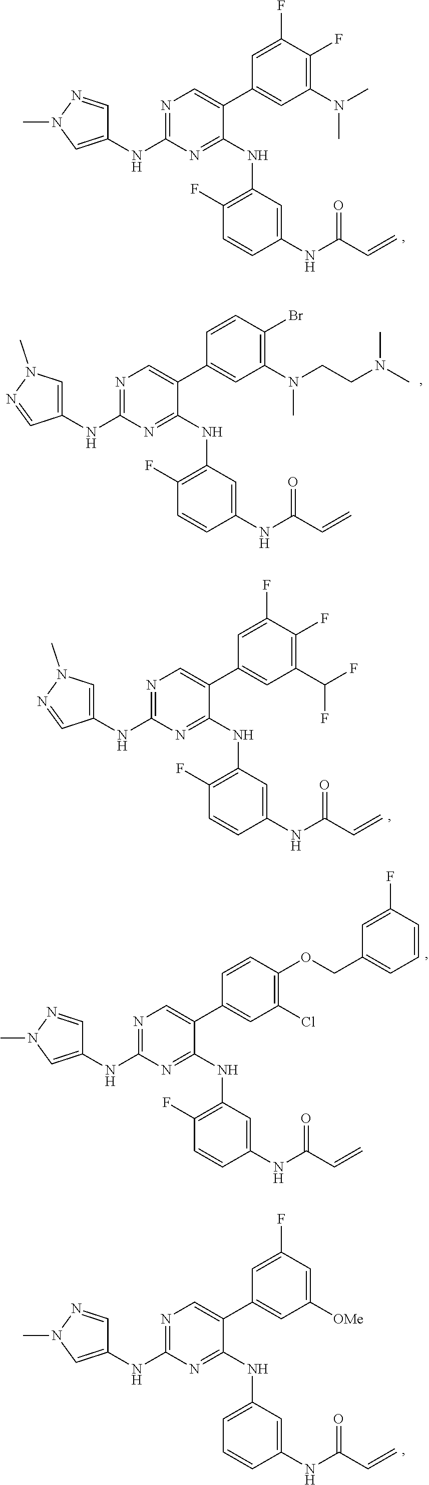 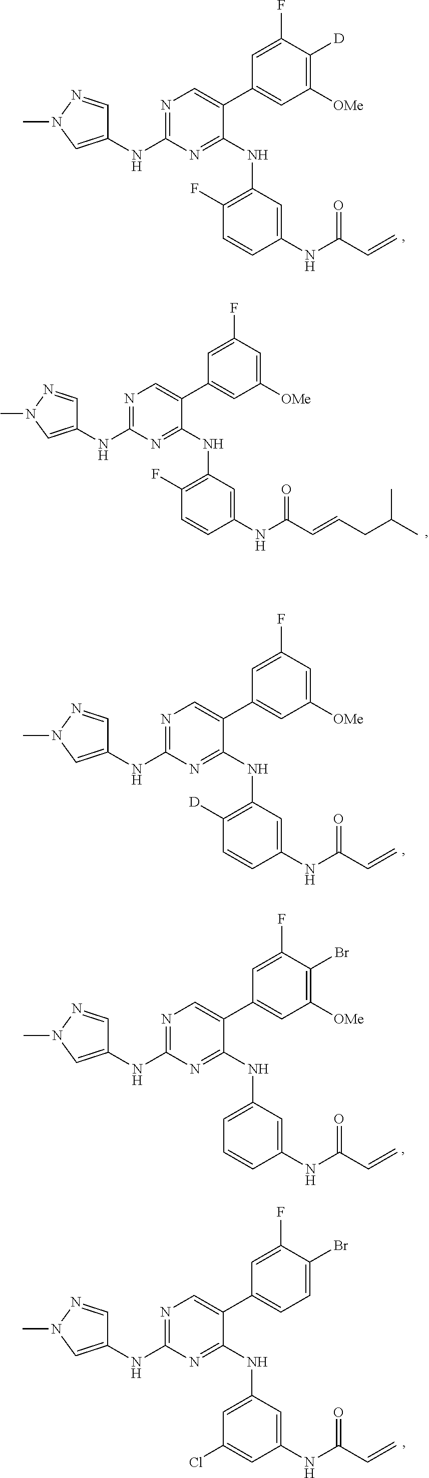 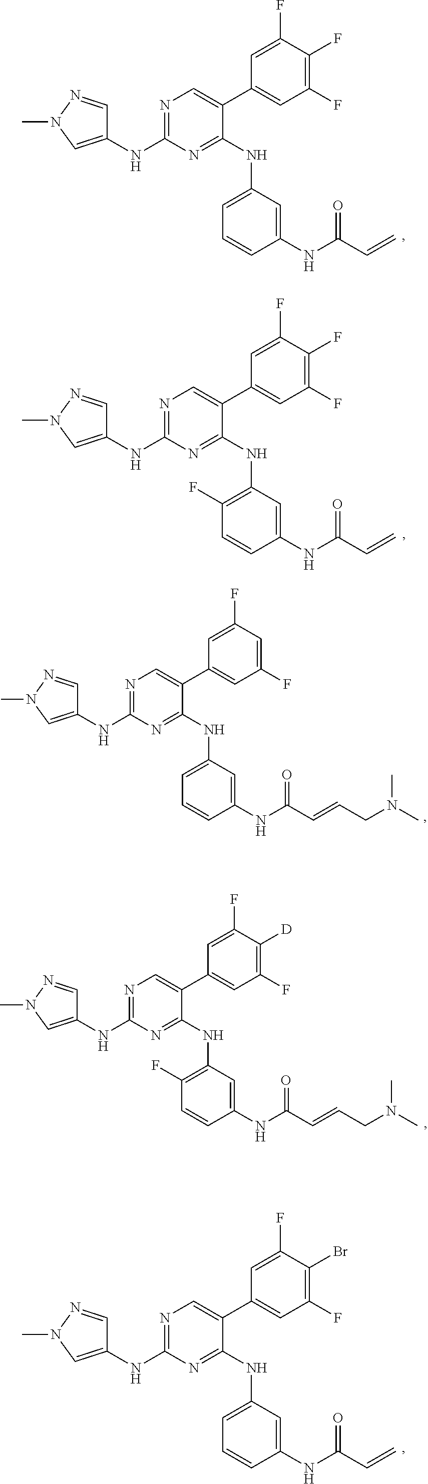 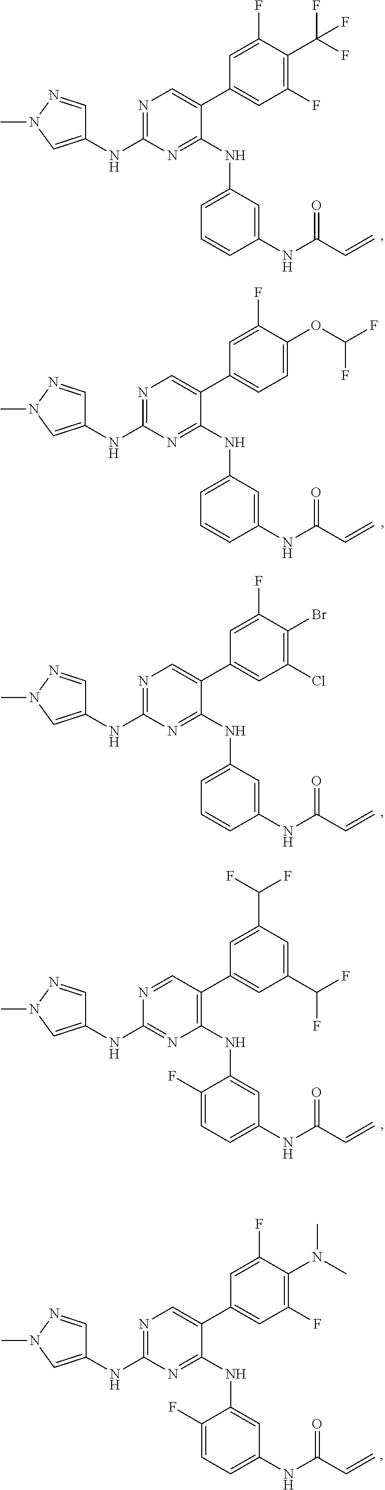 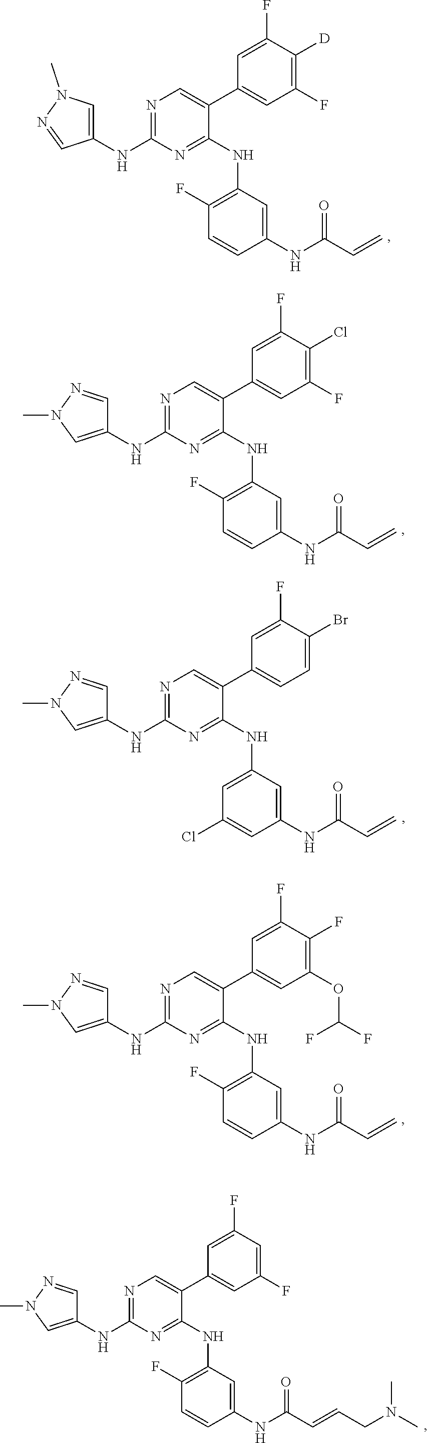 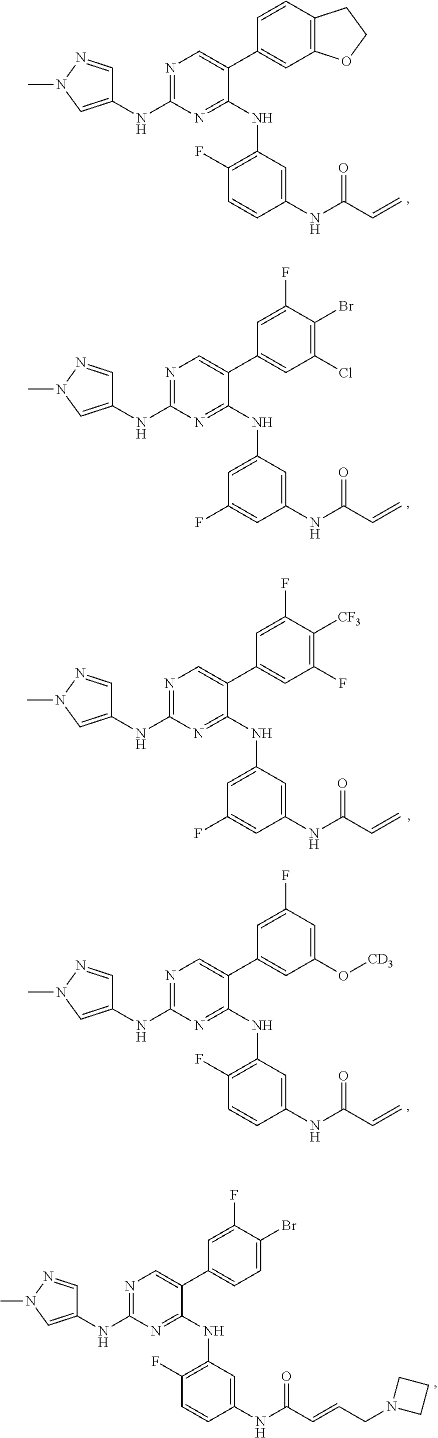 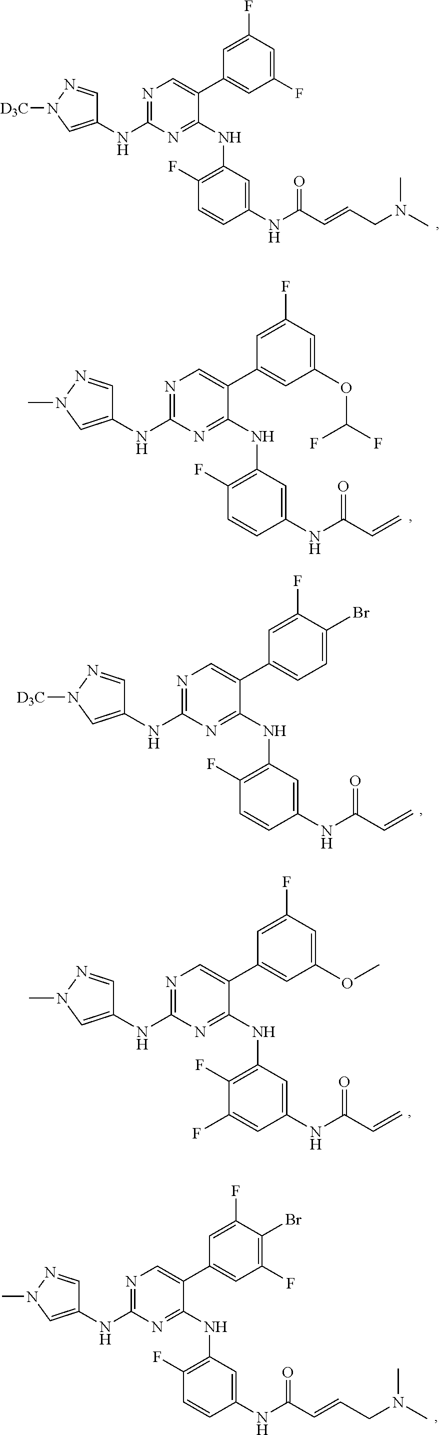 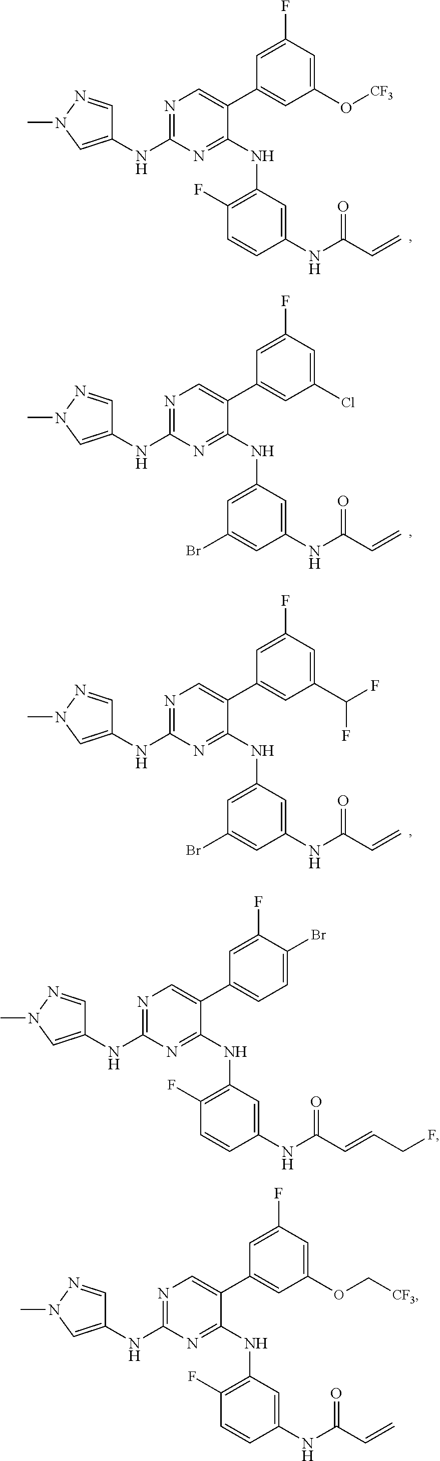 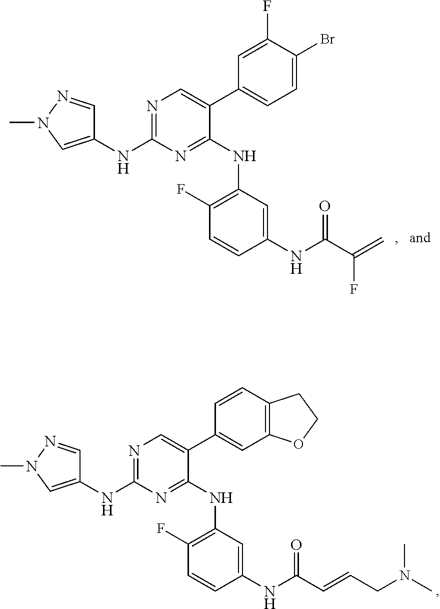
or a pharmaceutically acceptable salt or stereoisomer thereof.

69. A pharmaceutical composition comprising a compound of any one of claims 1 to 68, or a pharmaceutically acceptable salt or stereoisomer thereof, and a pharmaceutically acceptable carrier.

70. A method of inhibiting a human epidermal growth factor receptor 2 (HER2) mutant and and an epidermal growth factor receptor (EGFR) mutant in a subject in need thereof, comprising administering to the subject a therapeutically effective amount of a compound of any one of claims 1 to 68, or a pharmaceutically acceptable salt or stereoisomer thereof.

71. The method of claim 70, wherein the HER2 mutant comprises an insertion in exon 20, an in-frame deletion and insertion in exon 20, a substitution in the extracellular domain, an extracellular truncation, or a substitution in exon 30.

72. The method of claim 71, wherein the HER2 mutant is selected from A775_G776insYVMA, A775_G776insSVMA, A775_G776insVVMA, G776del insVC, G776del insLC, G776del insAV, G776del insAVGC, S310F, S310Y, p95, V842I, P780_Y781insGSP, and any combination thereof.

73. The method of any one of claims 70-72, wherein the EGFR mutant comprises a substitution in exon 18, a deletion in exon 19, a substitution in exon 20, an insertion in exon 20, a mutation in the extracellular domain, or a substitution in exon 21.

74. The method of any one of claims 70-73, wherein the EGFR mutant is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, A767_dupASV EGFR, 773insAH EGFR, M766_A767insAI EGFR, and any combination thereof.

75. A method of treating one or more cancer cells in a subject in need thereof, comprising administering to the subject a therapeutically effective amount of a compound of any one of claims 1 to 68, or a pharmaceutically acceptable salt or stereoisomer thereof.

76. A method of treating cancer in a subject in need thereof, comprising administering to the subject a therapeutically effective amount of a compound of any one of claims 1 to 68, or a pharmaceutically acceptable salt or stereoisomer thereof.

77. The method of either of claim 75 or 76, wherein the cancer is bladder cancer, prostate cancer, breast cancer, cervical cancer, colorectal cancer, endometrial cancer, gastric cancer, glioblastoma, head and neck cancer, lung cancer, or non-small cell lung cancer.

78. The method of claim 77, wherein the cancer is non-small cell lung cancer, prostate cancer, head and neck cancer, breast cancer, colorectal cancer, or glioblastoma.

79. The method of any one of claims 75-78, wherein the cancer in the subject comprises a HER2 mutation.

80. The method of claim 79, wherein the HER2 mutation comprises an insertion in exon 20, an in-frame deletion and insertion in exon 20, a substitution in the extracellular domain, an extracellular truncation, or a substitution in exon 30.

81. The method of claim 80, wherein the HER2 mutation is selected from A775_G776insYVMA, A775_G776insSVMA, A775_G776insVVMA, G776del insVC, G776del insLC, G776del insAV, G776del insAVGC, S310F, S310Y, p95, V842I, P780_Y781insGSP, and any combination thereof.

82. The method of any one of claims 75 to 81, wherein the cancer in the subject comprises an EGFR mutation.

83. The method of claim 82, wherein the EGFR mutation comprises a substitution in exon 18, a deletion in exon 19, a substitution in exon 20, an insertion in exon 20, a mutation in the extracellular domain, or a substitution in exon 21.

84. The method of claim 83, wherein the EGFR mutation is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, A767_dupASV EGFR, 773insAH EGFR, M766_A767insAI EGFR, and any combination thereof.

85. A method of treating an inflammatory disease in a subject in need thereof, comprising administering to the subject a therapeutically effective amount of a compound of any one of claims 1 to 68, or a pharmaceutically acceptable salt or stereoisomer thereof.

86. The method of claim 85, wherein the inflammatory disease is psoriasis, eczema, or atherosclerosis.

87. The method of claim 85, wherein the inflammatory disease in the subject comprises a HER2 mutation.

88. The method of claim 87, wherein the HER2 mutation comprises an insertion in exon 20, an in-frame deletion and insertion in exon 20, a substitution in the extracellular domain, an extracellular truncation, or a substitution in exon 30.

89. The method of claim 88, wherein the HER2 mutation is selected from A775_G776insYVMA, A775_G776insSVMA, A775_G776insVVMA, G776del insVC, G776del insLC, G776del insAV, G776del insAVGC, S310F, S310Y, p95, V842I, P780_Y781insGSP, and any combination thereof.

90. The method of any one of claims 85-89, wherein the inflammatory disease in the subject comprises an EGFR mutation.

91. The method of claim 90, wherein the EGFR mutation comprises a substitution in exon 18, a deletion in exon 19, a substitution in exon 20, an insertion in exon 20, a mutation in the extracellular domain, or a substitution in exon 21.

92. The method of claim 91, wherein the EGFR mutation is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, A767_dupASV EGFR, 773insAH EGFR, M766_A767insAI EGFR, and any combination thereof.
